(19) United States
(12) Patent Application Publication
(10) Pub. No.: US 2026/0166117 A1
(43) Pub. Date: Jun. 18, 2026

(54) COMPOSITIONS COMPRISING POLYINOSINIC-POLYCYTIDYLIC ACID, EPSTEIN-BARR (EBV) VIRUS-LIKE PARTICLES AND EBV-SPECIFIC CAR-T CELLS FOR IMPROVING IMMUNE CELL THERAPY

(71) Applicant: Zeno Therapeutics Pte. Ltd., Singapore (SG)

(72) Inventor: Lietao LI, Singapore (SG)

(73) Assignee: Zeno Therapeutics Pte. Ltd., Singapore (SG)

(21) Appl. No.: 19/128,896

(22) PCT Filed: Nov. 9, 2023
(86) PCT No.: PCT/SG2023/050746

§ 371 Date: May 9, 2025

(30) Foreign Application Priority Data
Nov. 9, 2022 (SG) 10202260024S
May 9, 2023 (SG) 10202301293Q

(51) Int. Cl. A61K 38/00

(52) U.S. Cl. CPC A61K 38/00

(57) ABSTRACT

The disclosure relates to an immunogenic composition comprising polyinosinic-polycytidylic acid (PIC), a stabilizer comprising an aminoglycoside or non-aminoglycoside antibiotic, at least one cation and optionally, an immunogen. It also relates an immunogenic composition comprising Epstein-Barr (EBV) virus-like particles. It further describes EBV-specific CAR-T cells and the use of the immunogenic composition comprising PIC alone or in combination with VLP or CAR-T cells for the treatment of cancer.

### TECHNICAL FIELD

The invention relates to compositions for improving immune cell therapy; as well as CARs and immune cell comprising said CAR for cell therapy. The invention also relates to uses of said compositions or said CAR-comprising immune cell as medicaments, alone or in combination therapy, in particular for treating cancer.

### BACKGROUND

Recent years have provided advancements in the treatment of cancers. Among these advancements are the use of immune cell therapy, where a patient's immune response is harnessed to treat cancer. Such immune cell therapy treatment methods include the use of cell-based immunotherapy, where cells of the immune system are utilized for therapeutic treatment. Immune system cells such as T cells and other immune cells can be modified to target tumor antigens.

Recent developments using chimeric antigen receptor (CAR) modified T cell (CART) therapy, which relies on redirecting T cells to a suitable cell-surface molecule on cancer cells, show promising results in harnessing the power of the immune system to treat cancers (see, e.g., Sadelain et al., Cancer Discovery 3:388-398 (2013)). Most notably, CD19-specific CAR (CD19CAR) T-cell therapies have had remarkable results including long-term remissions in B-cell malignancies (e.g. Maude, Frey et al. 2014). Despite the success of CAR therapy in hematologic cancers such as B-cell leukemia and lymphoma, only limited success was obtained in solid tumors.

While cell-based therapies using immune cells such as T cells have provided new modalities for cancer treatment, limitations have been found for the effectiveness of such treatments especially for solid tumors. For example, cancer cells can adapt to generate an immunosuppressive microenvironment that protects the cells from immune recognition and elimination. This microenvironment poses a challenge to methods of treatment involving stimulation of an immune response, including immunotherapy methods such as targeted T cell therapies.

Given the ongoing need for improved strategies for cancer treatment, new compositions and methods for improving cell-based immunotherapy in particular CART therapies are highly desirable.

### SUMMARY

The present disclosure provides compositions for improving immune cell therapy. Said compositions are useful for the eliciting, inducing, enhancing and/or potentiating an immune response, which may be an innate and/or adaptive immune response mediated by an immune cell therapy. In some embodiments, said immune cell is immune effector cells (e.g., T cells or NK cells) that express a chimeric antigen receptor (CAR) molecule, e.g., a CAR molecule that binds to a tumor antigen, e.g., an antigen expressed on the surface of a solid tumor or a hematological tumor.

In some aspects, the present disclosure relates to an immunogenic composition comprising: (a) a polyinosinic-polycytidylic acid (PIC), (b) a stabilizer which is an aminoglycoside antibiotic (preferably kanamycin) or non-aminoglycoside amine, (c) at least one cation such as calcium ion. In some embodiments, said composition further comprises at least one immunogen or antigen, wherein the immunogen or antigen is a recombinant protein, virus-like particle (VLP), peptide, mRNA or vaccine. Said immunogenic composition may be for use in inducing activation and/or increasing of an immune response in an individual, such as immune response of an immune cell therapy (preferably CAR-T cell therapy) for cancer treatment. Said immunogenic composition may be used in combination with an engineered immune cell (e.g. CAR-T cell or engineered TCR-T cell), in the treatment of cancer. In some examples, said immunogenic composition comprises polyinosinic-polycytidylic acid, kanamycin, calcium, and a VLP vaccine of Epstein Barr virus (EBV). In some examples, said immunogenic composition is to be administered in combination (simultaneously or sequentially) with an engineered immune cell, preferably CAR-T cell, more preferably a CAR-T cell that targets EBV-associated cancers.

In another aspects, the present disclosure relates to an immunogenic composition comprising a VLP of Epstein Barr virus (EBV). In some examples, said VLP comprises glycoprotein 350/220 (gp350) protein or fragment thereof, optionally wherein the VLP comprises one, two, three or more polypeptide sequences that is/are at least 80%, at least 90%, at least 95%, at least 96%, at least 97%, at least 98%, at least 99% identical or 100% identical to polypeptide sequences selected from the group consisting of SEQ ID NOs: 102 to 117. In one preferred embodiment, said VLP comprises one, two, three, four, or more EBV proteins selected from the group consisting of gp350, BKRF4, BVRF1, BDLF3, BZLF2, BXLF2, BNRF1, BALF4 and BZLF1; or their functional variant thereof that is at least 80%, at least 90%, at least 95%, at least 96%, at least 97%, at least 98%, at least 99% identical or 100% identical to the polypeptide sequence described herein. In another preferred embodiment, said VLP comprises the EBV proteins of gp350, BKRF4, BVRF1, BDLF3, BZLF2, BXLF2, BNRF1, BALF4 and BZLF1; or their functional variants thereof that is at least 80%, at least 90%, at least 95%, at least 96%, at least 97%, at least 98%, at least 99% identical or 100% identical to the polypeptide sequences described herein. In some preferred embodiments, said VLP does not comprise LMP1, EBNA2, EBNA3a, EBNA3b and EBNA3c. Said immunogenic composition may be for use in inducing activation and/or increasing of an immune response in an individual, such as immune response of an immune cell therapy (preferably CAR-T cell therapy) for cancer treatment. Said immunogenic composition may be used in combination with an engineered immune cell (e.g. CAR-T cell or engineered TCR-T cell), in the treatment of cancer. In some specific examples, said immunogenic composition is to be administered in combination (simultaneously or sequentially) with an engineered immune cell, preferably CAR-T cell, more preferably a CAR-T cell that targets EBV-associated cancers, even more preferably a CAR-T cell that binds glycoprotein 350/220 (gp350) protein.

In another aspects, the present disclosure provides a CAR, or an immune cell (preferably CAR-T cell) which comprises the CAR, wherein the CAR comprises an EBV glycoprotein 350/220 antigen binding domain, wherein the antigen binding domain comprises a heavy chain variable region having a polypeptide sequence at least 80%, at least 90%, at least 95%, at least 96%, at least 97%, at least 98%, or at least 99% identical to any one of SEQ ID NOs: 1, 2, 3, 4, 5, 138, 139 or 140, and/or a light chain variable region having a polypeptide sequence at least 80%, at least 90%, at least 95%, at least 96%, at least 97%, at least 98%, or at least 99% identical to any one of SEQ ID NOs: 6, 7, 8, 9, 10, 141, 142 or 143. In some examples, the antigen binding domain that binds EBV gp350/220 comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively; or
(5) SEQ ID NOs: 23, 24, 25, 38, 39 and 40, respectively; or
   an EBV glycoprotein 350/220 antigen-binding domain which competes therewith or binds to the same epitope as that in the EBV glycoprotein 350/220 to which any one of the antigen binding domain (1) to (5) of the CAR as described herein bind.

In some examples, the EBV glycoprotein 350/220 antigen binding domain comprises any one of:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO: 4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO: 5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence;
(6) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence; or
(15) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence.

In some aspects, the present disclosure provides:

[1] An immunogenic composition comprising:
(a) a polyinosinic-polycytidylic acid (PIC);
(b) a stabilizer which is an aminoglycoside antibiotic or non-aminoglycoside amine;
(c) at least one cation; and 5
(d) optionally an immunogen, wherein the immunogen is a recombinant protein, virus-like particle (VLP), peptide, mRNA or vaccine.

[1A] An immunogenic composition comprising a VLP of Epstein Barr virus (EBV), preferably said VLP comprises glycoprotein 350/220 (gp350) protein or fragment thereof, optionally wherein the VLP comprises one, two, three or more polypeptide sequences that is/are at least 80%, at least 90%, at least 95%, at least 96%, at least 97%, at least 98%, at least 99% identical or 100% identical to the polypeptide sequences selected from the group consisting of SEQ ID NOs: 102 to 117.

[1B] The composition of [1A], wherein the VLP comprises one, two, three, four, or more EBV proteins selected from the group consisting of gp350, BKRF4, BVRF1, BDLF3, BZLF2, BXLF2, BNRF1, BALF4 and BZLF1; or their functional variant thereof that is at least 80%, at least 90%, at least 95%, at least 96%, at least 97%, at least 98%, at least 99% identical or 100% identical to the polypeptide sequence described herein.

[1C] The composition of [1A], wherein the VLP comprises the EBV proteins of gp350, BKRF4, BVRF1, BDLF3, BZLF2, BXLF2, BNRF1, BALF4 and BZLF1; or their functional variants thereof that is at least 80%, at least 90%, at least 95%, at least 96%, at least 97%, at least 98%, at least 99% identical or 100% identical to the polypeptide sequences described herein.

[1D] The composition of any one of [1A] to [1C], wherein the VLP does not comprise LMP1, EBNA2, EBNA3a, EBNA3b and EBNA3c.

[1E] The composition of [1A], wherein the VLP comprises (i) at least one EBV structural polypeptide, (ii) at least one EBV lytic polypeptide, (iii) membrane lipids, wherein preferably said VLP has one of more of the following properties:
(a) being devoid of EBV DNA;
(b) the B-cell transformation capacity of one of more EBV required for B-cell transformation is disabled or removed while their immunogenicity is maintained; and/or
(c) said EBV does not comprise one or more EBV polypeptides required for B-cell transformation which is selected from the group consisting of LMP1, EBNA2, EBNA3a, EBNA3b and EBNA3c.

[2] The composition according to any one of [1] to [1E], for use in cancer treatment. [2A] The composition according to any one of [1] to [1E], for use in inducing immune response, wherein preferably said immune response is immune response of a CAR-T cell against tumor cell.

[3] Use of the composition according to any one of [1] to [2A] in manufacture of a medicament for cancer treatment.

[4] The composition or the use according to any one of [2] to [3], wherein said cancer treatment is an immune cell therapy (preferably CAR-T cell therapy), and optionally said composition increases anti-cancer effect of the cell therapy.

[5] The composition or the use according to any one of [2] to [4], for use in combination with an engineered immune cell (preferably CAR-T cell or engineered TCR-T cell), for the treatment of cancer wherein optionally said immunogenic composition is to be administered to the subject before, after or simultaneously with the immune cell.

[6] The composition or the use according to any one of [1] to [5], wherein the immunogen is a tumor-associated antigen, an allergen, an antigen associate with a virus, or the fragment thereof.

[6A] The composition or the use according to any one of [1] to [5], wherein the immunogen a recombinant protein, virus-like particle (VLP), peptide, mRNA, DNA, vaccine or dendritic cell vaccine.

[6B] The composition or the use according to any one of [1] to [5], wherein the immunogen is a virus-like particle (VLP).

[6C] The composition or the use according to [6B], wherein the immunogen is a VLP of Epstein Barr virus (EBV), preferably said VLP comprises glycoprotein 350/220 (gp350) protein or fragment thereof.

[6D] The composition or the use according to [6B], wherein the VLP further comprises one, two, three or more protein sequences selected from the group consisting of SEQ ID NOs: 102 to 117.

[7] The composition or the use according to any one of [1] to [6D], wherein said stabilizer is an aminoglycoside antibiotic which is selected from kanamycin, Streptomycin, Dihydrostreptomycin, Mannoside Streptomycin, Amikacin, Amikacin, Dibekacin, Vietomycin, Gentamycin, and any combination thereof; and preferably said stabilizer is kanamycin.

[7A] The composition or the use according to any one of [1] to [7], wherein said composition comprises 0.5 mg/ml to 10 mg/ml of PIC.

[7B] The composition or the use according to any one of [1] to [7A], wherein said PIC has a molecular weight range from about 66,000 to 2,000,000 Daltons.

[7C] The composition or the use according to any one of [1] to [7A], wherein said PIC has a molecular weight range from about 300,000 to 1,200,000 Daltons or size from about 6.4 to 24.0 Svedbergs.

[7D] The composition or the use according to any one of [1] to [7A], wherein said PIC has a molecular weight range from about 66,000 to 660,000 Daltons or molecular size range from about 6.4 to 18.3 Svedbergs.

[7E] The composition or the use according to any one of [1] to [7A], wherein said PIC has a molecular weight range from about 300,000 to 660,000 Daltons or molecular size range from about 12.8 to 18.3 Svedbergs.

[7F] The composition or the use according to any one of [1] to [7A], wherein said PIC has an average molecular weight equal to or greater than 150,000 Daltons or average molecular size equal to or greater than 9.3 Svedbergs.

[7G] The composition or the use according to any one of [1] to [7A], wherein said PIC has an average molecular weight equal to or greater than 250,000 Daltons or average molecular size equal to or greater than 11.8 Svedbergs.

[7H] The composition or the use according to any one of [1] to [7A], wherein said PIC has an average molecular weight equal to or greater than 350,000 Daltons or average molecular size equal to or greater than 15.3 Svedbergs.

[8] The composition or the use according to any one of [1] to [6B], wherein said stabilizer is a non-aminoglycoside amine which is selected from the group consisting of a polyethylene glycol monomethyl ether, polyethylene glycol, polyethyleneimine, folic acid, galactose, polylysine, protamine, shell oligosaccharide, chitosan, spermine, glucosamine, and any combination thereof; preferably said stabilizer is polylysine, chitin, chitosan or glucosamine; more preferably said stabilizer is ε-polylysine, hexylglucosamine or acetylglucosamine.

[9] The composition or the use according to any one of [1] to [8], wherein said cation is selected from the group consisting of calcium, cadmium, lithium, magnesium, cerium, cesium, chromium, cobalt, deuterium, gallium, iodine, iron, zinc, and any combination thereof; and preferably said cation is calcium.

[10] The composition or the use according to any one of [1] to [9], wherein said immune cell is a T cell that expresses a CAR comprising an extracellular domain, a transmembrane domain, and an intracellular domain, wherein the extracellular domain comprises an antigen-binding domain that specifically binds an antigen expressed on the surface of the cancer, wherein preferably said antigen-binding domain is a scFv domain.

[11] The composition or the use according to [1], wherein the intracellular domain comprises at least one costimulatory domain.

[12] The composition or the use according to [11], wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, HVEM (LIGHTR), KIRDS2, SLAMF7, NKp80 (KLRF1), NKp44, NKp30, NKp46, CD19, CD4, CD8alpha., CD8beta, IL-2R beta, IL-2R gamma, IL-7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

[13] The composition or the use according to [12], wherein the costimulatory domain comprises a signaling region of 4-1BB/CD137.

[14] The composition or the use according to [12], wherein the costimulatory domain comprises a signaling region of CD28.

[15] The composition or the u-se according to any one of [11] to [14], wherein the CAR comprises two or more costimulatory domains.

[16] The composition or the use according to [15], wherein the CAR comprises two costimulatory domains, one of the two costimulatory domains is CD28 and the other costimulatory domain is selected from 4-1BB/CD137 or OX40.

[17] The composition or the use according to any one of [10] to [16], wherein the intracellular domain of the CAR comprises at least one activating domain.

[18] The composition or the use according to [17], wherein the activating domain comprises CD3, preferably said CD3 is CD3 zeta.

[19] The composition or the use according to any one of [4] to [9], wherein the immune cell is a T cell, Natural Killer (NK) cell, TCR-expressing cell, dendritic cell, gamma delta T cell, or NK-T cell.

[20] The composition, or the use according to any one of [1] to [19], wherein the cell is an autologous T cell.

[21] The composition, or the use according to any one of [4] to [19], wherein the cell is an allogeneic T cell.

[22] The composition, or the use according to any one of [4] to [19], wherein the cell is engineered to express at least one cytokine.

[23] The composition, or the use according to [22], wherein said at least one cytokine is at least one selected from the group consisting of IL-2, IL-4, IL-7, IL-10, IL-12, IL-15, IL-15/IL-15sushi, IL-15/IL-15sushi anchor, IL-18, IL-21, GM-CSF, and TGF-β.

[24] The composition, or the use according to any one of [4] to [19], wherein the cell is engineered to further express at least one chemokine.

[25] The composition, or the use according to [24], wherein said at least one chemokine is selected from the group consisting of CCL2, CCL3, CCL4, CCL5, CCL7, CCL8, CCL19, CXCL1, CXCL2, CXCL9, CXCL10, CCL21, and CXCL12.

[25A] The composition, the use, or the method according to any one of [5] to [25], wherein the cell comprises a CAR that comprises an antigen-binding domain that binds EBV gp350/220 comprising a heavy chain variable region (VH) and/or light chain variable region (VL) as provided in Table 1 and Table 2, or a specific VH and VL combination as provided in Table 1 and Table 2.

[25B] The composition, the use, or the method according to any one of [5] to [25], wherein the cell comprises a CAR that comprises an antigen-binding domain that binds EBV gp350/220 comprising one, two, three or more HCDRs and/or one, two, three or more LCDR as provided in Table 3 and Table 4, or a specific HCDR1-3 and LCDR1-3 combination as provided in Table 3 and Table 4.

[25C] The composition, the use, or the method according to any one of [5] to [25], wherein the cell comprises a CAR comprising any one of the CAR amino acid sequences as provided in Table 5.

[25D] The composition, the use, or the method according to any one of [5] to [25], wherein the cell comprises a CAR comprising an antigen binding domain that binds EBV gp350/220 which comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively;
(5) SEQ ID NOs: 23, 24, 25, 38, 39 and 40, respectively;
(6) SEQ ID NOs: 118, 119, 120, 121, 122, 123, respectively; or
(7) SEQ ID NOs: 124, 125, 126, 127, 128, 129, respectively.

[25E] The composition, the use, or the method according to any one of [5] to [25], wherein the cell comprises a CAR comprising an antigen binding domain that binds EBV gp350/220, wherein the antigen binding domain is an antigen binding domain which competes therewith or binds to the same epitope as that in the Epstein-Barr virus (EBV) glycoprotein 350/220 to which any one of the antigen binding domain (1) to (5) of the CAR described in [25D] bind.

[25F] The composition, the use, or the method according to any one of [5] to [25], wherein the cell comprises a CAR comprising an antigen binding domain that binds EBV gp350/220, wherein the antigen binding domain comprises a heavy chain variable region having a polypeptide sequence at least 80%, at least 90%, at least 95%, at least 96%, at least 97%, at least 98%, or at least 99% identical to any one of SEQ ID NOs: 1, 2, 3, 4, 5 138, 139 or 140, and/or a light chain variable region having a polypeptide sequence at least 80%, at least 90%, at least 95%, at least 96%, at least 97%, at least 98%, or at least 99% identical to any one of SEQ ID NOs: 6, 7, 8, 9, 10, 141, 142 or 143.

[25G] The composition, the use, or the method according to any one of [5] to [25], wherein the cell comprises a CAR comprising an antigen binding domain that binds EBV gp350/220, wherein the antigen binding domain comprises any one of:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO: 4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO: 5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence;
(6) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(15) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(16) a heavy chain variable region (VH) having the polypeptide sequence of SEQ ID NO: 130 or a variant in which 1, 2 or 3 amino acids are substituted related to the sequence, and a light chain variable region (VL) having the polypeptide sequence of SEQ ID NO: 131 or a variant in which 1, 2 or 3 amino acids are substituted related to the sequence; or
(17) a heavy chain variable region (VH) having the polypeptide sequence of SEQ ID NO: 132 or a variant in which 1, 2 or 3 amino acids are substituted related to the sequence, and a light chain variable region (VL) having the polypeptide sequence of SEQ ID NO: 133 or a variant in which 1, 2 or 3 amino acids are substituted related to the sequence.

In some aspects, the present disclosure relates to:

[26] A method of increasing anti-cancer response of an immune cell therapy in an individual, the method comprising administering to the individual an effective amount of the immunogenic composition of any one of [1] to [25G] described above.

In some aspects, the present disclosure relates to:

[27] A method of treating an individual having cancer, the method comprising:
(a) administering to the individual an effective amount of the immunogenic composition described in any one of [1] to [9] above; and
(b) administering to the individual an effective amount of the cell described in any one of [10] to [25G] above; wherein optionally said immunogenic composition is administered to the subject before, after or simultaneously with the immune cell.

In some further aspects, the present disclosure relates to:

[28] A VLP of Epstein Barr virus (EBV), for use in the treatment of cancer, wherein the VLP is to be administered in combination with an engineered immune cell (preferably CAR-T cell or engineered TCR-T cell), in the treatment of cancer, wherein optionally said VLP is to be administered to the subject before, after or simultaneously with the immune cell.

[29] The VLP for use of [28], wherein said VLP comprises glycoprotein 350/220 (gp350) protein or fragment thereof, wherein preferably the VLP further comprises one, two, three or more protein sequences selected from the group consisting of SEQ ID NOS: 102 to 117.

[30] The VLP for use of [28] or [29], wherein said immune cell is a T cell that expresses a CAR comprising an extracellular domain, a transmembrane domain, and an intracellular domain, wherein the extracellular domain comprises an antigen-binding domain that specifically binds an antigen expressed on the surface of the cancer, wherein preferably said antigen-binding domain is a scFv domain.

[31] The VLP for use of [30], wherein the intracellular domain of the CAR comprises at least one costimulatory domain.

[32] The VLP for use of [30] or [31], wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, HVEM (LIGHTR), KIRDS2, SLAMF7, NKp80 (KLRF1), NKp44, NKp30, NKp46, CD19, CD4, CD8alpha., CD8beta, IL-2R beta, IL-2R gamma, IL-7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

[33] The VLP for use of [32], wherein the costimulatory domain comprises a signaling region of 4-1BB/CD137.

[34] The VLP for use according to [32], wherein the costimulatory domain comprises a signaling region of CD28.

[35] The VLP for use according to any one of [30] to [34], wherein the CAR comprises two or more costimulatory domains.

[36] The VLP for use according to [35], wherein the CAR comprises two costimulatory domains, one of the two costimulatory domains is CD28 and the other costimulatory domain is selected from 4-1BB/CD137 or OX40.

[37]. The VLP for use according to any one of [30]-[36], wherein the intracellular domain of the CAR comprises at least one activating domain.

[38]. The VLP for use according to [37], wherein the activating domain comprises CD3, preferably said CD3 is CD3 zeta.

[39]. The VLP for use according to any one of [34] to [38], wherein the immune cell is a T cell, Natural Killer (NK) cell, TCR-expressing cell, dendritic cell, gamma delta T cell, or NK-T cell, preferable the immune cell is a CAR-T cell.

[40] The VLP for use according to any one of [28] to [39], wherein said cancer is an EBV-associated cancer, selected from a lymphoproliferative disorder (LPD), such as B-cell lymphoma, including Burkitt lymphoma (BL), Hodgkin lymphoma (HL), a diffuse large B cell lymphoma (DLBCL), T-cell lymphoma, NK/T-cell lymphoma, or a post-transplant lymphoproliferative disorder (PTLD), or an epithelial carcinoma (nasopharyngeal, lung, breast), a lymphoepithelioma, a carcinoma with lymphoid stroma (GCLS, e.g. gastric carcinoma) or a glioma.

[41] The VLP for use according to any one of [28] to [40], wherein said antigen that is recognized by the CAR is an Epstein-Barr virus antigen (EBV antigen), wherein preferably said EBV antigen is EBV glycoprotein 350/220 (gp350/220).

[42] The VLP for use according to any one of [28] to [41], wherein the cell comprises CAR that comprises an antigen-binding domain that binds EBV gp350/220 comprising a heavy chain variable region (VH) and/or light chain variable region (VL) as provided in Table 1 and Table 2, or a specific VH and VL combination as provided in Table 1 and Table 2.

[43]. The VLP for use according to any one of [28] to [42], wherein the cell comprises a CAR that comprises an antigen-binding domain that binds EBV gp350/220 comprising one, two, three or more HCDRs and/or one, two, three or more LCDR as provided in Table 3 and Table 4, or a specific HCDR1-3 and LCDR1-3 combination as provided in Table 3 and Table 4.

[44] The VLP for use according to any one of [28] to [43], wherein the cell comprises a CAR comprising any one of the CAR amino acid sequence as provided in Table 5.

[45] The VLP for use according to any one of [28] to [43], wherein the cell comprises a CAR comprising an antigen binding domain that binds EBV gp350/220 protein which comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively;
(5) SEQ ID NOs: 23, 24, 25, 38, 39 and 40, respectively;
(6) SEQ ID NOs: 118, 119, 120, 121, 122, 123, respectively; or
(7) SEQ ID NOs: 124, 125, 126, 127, 128, 129, respectively.

[46] The VLP for use according to any one of [28] to [43], wherein the cell comprises a CAR comprising an antigen binding domain that binds EBV gp350/220, wherein the antigen binding domain is an antigen binding domain which competes therewith or binds to the same epitope as that in the Epstein-Barr virus (EBV) antigen glycoprotein 350/220 to which any one of the antigen binding domain (1) to (5) of the CAR described in [45] bind.

[47] The VLP for use according to any one of [28] to [43], wherein the cell comprises a CAR comprising an antigen binding domain that binds EBV gp350/220, wherein the antigen binding domain comprises a heavy chain variable region having a polypeptide sequence at least 80%, at least 90%, at least 95%, at least 96%, at least 97%, at least 98%, or at least 99% identical to any one of SEQ ID NOs: 1, 2, 3, 4, 5, 138, 139 or 140 and/or a light chain variable region having a polypeptide sequence at least 80%, at least 90%, at least 95%, at least 96%, at least 97%, at least 98%, or at least 99% identical to any one of SEQ ID NOs: 6, 7, 8, 9, 10, 141, 142 or 143.

[48] The VLP for use according to any one of [28] to [43], wherein the cell comprises a CAR comprising an antigen binding domain that binds EBV gp350/220 protein, wherein the antigen binding domain comprises any one of:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO: 4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO: 5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence;
(6) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(15) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(16) a heavy chain variable region (VH) having the polypeptide sequence of SEQ ID NO: 130 or a variant in which 1, 2 or 3 amino acids are substituted related to the sequence, and a light chain variable region (VL) having the polypeptide sequence of SEQ ID NO: 131 or a variant in which 1, 2 or 3 amino acids are substituted related to the sequence; or
(17) a heavy chain variable region (VH) having the polypeptide sequence of SEQ ID NO: 132 or a variant in which 1, 2 or 3 amino acids are substituted related to the sequence, and a light chain variable region (VL) having the polypeptide sequence of SEQ ID NO: 133 or a variant in which 1, 2 or 3 amino acids are substituted related to the sequence.

In some further aspects, the present disclosure provides a CAR, or an immune cell (preferably CAR-T cell) which comprises a CAR, wherein the CAR comprises an EBV glycoprotein 350/220 antigen-binding domain comprising a heavy chain variable region (VH) and/or light chain variable region (VL) as provided in Table 1 and Table 2, or a specific VH and VL combination as provided in Table 1 and Table 2.

In some further aspects, the present disclosure provides a CAR, or a CAR-T cell which comprises a CAR, wherein the CAR comprises an EBV glycoprotein 350/220 antigen-binding domain comprising one, two, three or more HCDRs and/or one, two, three or more LCDR as provided in Table 3 and Table 4, or a specific HCDR1-3 and LCDR1-3 combination as provided in Table 3 and Table 4.

In some further aspects, the present disclosure provides a CAR, or an immune cell (preferably CAR-T cell) which comprises a CAR comprising any one of a CAR amino acid sequence as provided in Table 5.

In some further aspects, the present disclosure provides a CAR, or an immune cell (preferably CAR-T cell) which comprises a CAR, wherein the CAR comprises an EBV glycoprotein 350/220 binding domain which comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively;
(5) SEQ ID NOs: 23, 24, 25, 38, 39 and 40, respectively;
(6) SEQ ID NOs: 118, 119, 120, 121, 122, 123, respectively; or
(7) SEQ ID NOs: 124, 125, 126, 127, 128, 129, respectively;
   or
   an EBV glycoprotein 350/220 antigen-binding domain which competes therewith or binds to the same epitope as that in the EBV glycoprotein 350/220 to which any one of the antigen binding domain (1) to (7) of the CAR as described herein bind.

In some further aspects, the present disclosure provides a CAR, or an immune cell (preferably CAR-T cell) which comprises a CAR, wherein the CAR comprises an EBV glycoprotein 350/220 antigen binding domain, wherein the antigen binding domain comprises a heavy chain variable region having a polypeptide sequence at least 80%, at least 90%, at least 95%, at least 96%, at least 97%, at least 98%, or at least 99% identical to any one of SEQ ID NOs: 1, 2, 3, 4, 5, 138, 139 or 140, and/or a light chain variable region having a polypeptide sequence at least 80%, at least 90%, at least 95%, at least 96%, at least 97%, at least 98%, or at least 99% identical to any one of SEQ ID NOs: 6, 7, 8, 9, 10, 141, 142 or 143.

In some further aspects, the present disclosure provides a CAR, or an immune cell (preferably CAR-T cell) which comprises a CAR, wherein the CAR comprises an EBV glycoprotein 350/220 antigen binding domain, wherein the antigen binding domain comprises any one of:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO: 4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO: 5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence;
(6) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(15) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(16) a heavy chain variable region (VH) having the polypeptide sequence of SEQ ID NO: 130 or a variant in which 1, 2 or 3 amino acids are substituted related to the sequence, and a light chain variable region (VL) having the polypeptide sequence of SEQ ID NO: 131 or a variant in which 1, 2 or 3 amino acids are substituted related to the sequence; or
(17) a heavy chain variable region (VH) having the polypeptide sequence of SEQ ID NO: 132 or a variant in which 1, 2 or 3 amino acids are substituted related to the sequence, and a light chain variable region (VL) having the polypeptide sequence of SEQ ID NO: 133 or a variant in which 1, 2 or 3 amino acids are substituted related to the sequence;
or
an EBV glycoprotein 350/220 antigen-binding domain which competes therewith or binds to the same epitope as that in the EBV glycoprotein 350/220 to which any one of the antigen binding domain (1) to (17) of the CAR as described herein bind.

In some further aspects, the present disclosure provides the immune cell or CAR-T cell as described herein for use in cancer treatment, wherein preferably said cancer is an EBV-associated cancer, selected from a lymphoproliferative disorder (LPD), such as B-cell lymphoma, including Burkitt lymphoma (BL), Hodgkin lymphoma (HL), a diffuse large B cell lymphoma (DLBCL), T-cell lymphoma, NK/T-cell lymphoma, or a post-transplant lymphoproliferative disorder (PTLD), or an epithelial carcinoma (nasopharyngeal, lung, breast), a lymphoepithelioma, a carcinoma with lymphoid stroma (GCLS, e.g. gastric carcinoma) or a glioma.

In some embodiments, in relation to the composition, the use, or the method as described in the disclosure, said cancer is selected from the group consisting of B-cell lymphoma, T-cell lymphoma, multiple myeloma, chronic myeloid leukemia (CML), acute myeloma leukemia (AML), myelodysplastic syndromes (MDS), chronic myeloproliferative neoplasms (MPN), B-cell acute lymphoblastic leukemia (B-ALL), solid tumor, carcinoma, or sarcoma; and preferably said cancer is a solid tumor. In another embodiments, said cancer is a virus-specific cancers.

In some embodiments, said cancer is an EBV-associated cancer, selected from a lymphoproliferative disorder (LPD), such as B-cell lymphoma, including Burkitt lymphoma (BL), Hodgkin lymphoma (HL), a diffuse large B cell lymphoma (DLBCL), T-cell lymphoma, NK/T-cell lymphoma, or a post-transplant lymphoproliferative disorder (PTLD), or an epithelial carcinoma (nasopharyngeal, lung, breast), a lymphoepithelioma, a carcinoma with lymphoid stroma (GCLS, e.g. gastric carcinoma) or a glioma.

In some embodiments, in relation to the composition, the use, or the method as described in any one of [10] to [28] above, said antigen that is recognized by the CAR is selected from the group consisting of CD38, GD2, CD123, CLL-1, CD19, CD33, BCMA, CS1, CD4, CD5, CD7, CD20, DLL3, GPC3, GPC2, EpCAM, NY-ESO-1, alpha fetoprotein (AFP), Flt3 receptor, Transmembrane activator and calcium-modulator and cyclophilin ligand interactor (TACI), CEA, ERBB2, EGFR, GD2, MSCA, mesothelin, MUC1, PSMA, HER2, Claudin-6, Trop2, MUC3A, Claudin18.2, gp100, MAGE-A1/3/4, LMP1, Nectin4/FAP, CD171, MUC16, CD20, CD80/86, c-MET, DR5, EpHA2, FR-α.

In some embodiments, said antigen that is recognized by the CAR is a virus antigen. In some embodiments, said antigen that is recognized by the CAR is a Epstein-Barr virus antigen (EBV antigen).

In some embodiments, said EBV antigen may be present on the surface of EBV-infected cells, preferably EBV-infected cancer cells, EBV-infected B cells or EBV-infected epithelial cells. The EBV antigen may be an EBV virion envelope protein or a protein of the EBV envelope complex (such as gB, gL, or gH). The EBV virus antigen preferably is the EBV glycoprotein 350/220 (gp350/220). In preferred embodiments the disclosure is focused on targeting EBV-antigens and treating EBV-associated medical conditions. An exemplary EBV gp350 protein is shown in the UniProt database, entry P03200-1, version 1 of 21 Jul. 1986. An exemplary EBV gp220 is shown in the same database entry but positions 502 to 750 are missing.

In some relevant embodiments, the immune cell or CAR-T cell comprises a CAR which comprises an EBV glycoprotein 350/220 antigen-binding domain comprising a heavy chain variable region (VH) and/or light chain variable region (VL) as provided in Table 1 and Table 2, or a specific VH and VL combination as provided in Table 1 and Table 2. In another embodiments, the CAR comprises an EBV glycoprotein 350/220 antigen-binding domain comprising a heavy chain variable region (VH) and/or light chain variable region (VL) which have at least 60%, 70%, 80%, 85%, 90%, 95%, 99% identity of the VH and/or VL amino acid sequences as provided in Table 1 and Table 2.

In some relevant embodiments, the CAR comprises an EBV glycoprotein 350/220 antigen-binding domain comprising one, two, three or more HCDRs and/or one, two, three or more LCDRs as provided in Table 3 and Table 4, or a specific HCDR1-3 and LCDR1-3 combination as provided in Table 3 and Table 4.

In some relevant embodiments, the cell comprises a EBV glycoprotein 350/220-binding CAR that comprises an amino acid sequence as provided in Table 5, preferably the CAR comprises an amino acid sequence selected from the group consisting of SEQ ID NO: 144 to 161. In another embodiments, the CAR comprises an amino acid sequence of at least 60%, 70%, 80%, 85%, 90%, 95%, 99% identity of any one of the CAR sequences as provided in Table 5, preferably the CAR is selected from the group consisting of SEQ ID NO: 144 to 161.

In some embodiments, in relation to the composition, the use, or the method described in to any one of [2] to [29] above, the composition and the cell are (to be) administered simultaneously or sequentially, e.g., the composition is (to be) administered prior to or subsequent to the administration of the cell.

In some aspects, the present application surprisingly and unexpectedly found that the combined therapy of the immunogenic composition comprising PIC and optionally an immunogen with immune cell therapy such as CAR-T cells is particularly effective in treating solid tumours. The immunogenic composition described herein is envisioned to be capable of improving, enhancing or increasing the immune response and anti-cancer activity of administered immune cells (e.g. CAR-T cells) in a subject having a solid tumor. Without wishing to be bound by the theory, it is envisioned that PIC can promote activation and proliferation of T cells (see Example 5). It is also envisioned that PIC may stimulate toll-like receptor 3 (TLR-3) and other cellular pathways, enhancing antigen presentation by antigen-presenting cells (APCs) and inducing production of pro-inflammatory cytokines. Previous studies have demonstrated PIC can enhance immune response against non-cancer indications such as rabies virus, hepatitis viruses and SAR-Cov (Lau et al., 2009, Lau et al., 2010 & Cell Mol Immunol. 2007 April; 4(2):113-20); VIROLOGICA SINICA, April 2011, 26(2):81-94). Secondly, PIC may induce the activation and proliferation of both B cells and NK cells as well as secretion of cytokines such as IFN γ and IL-2, which can increase the tumor infiltration, as well as stimulation and expansion of CAR-T cell. In addition, the presence of immunogen acting as a vaccine may further enhance the presentation of the target antigens on antigen-presenting cells available for enhancing said CAR-T therapy. Further, the immunogenic composition described herein may contribute to overcoming the immunosuppressive tumor microenvironment, a major issue for the limited success of CAR-T therapy on solid tumors.

In another aspects, the present application has successfully designed specific CARs and generated CAR-T cells comprising said CARs that targets EBV, which is particularly effective in treating cancers associated with EBV infections. Furthermore, the present inventors have unexpectedly found that the combined therapy of the immunogenic composition comprising VLP of EBV with immune cell therapy using said CAR-T cells that targets EBV is particularly effective in treating cancers associated with EBV infections. The immunogenic composition comprising the VLP of EBV as described herein is capable of improving, enhancing or increasing the immune response and anti-cancer activity of administered immune cells (e.g. CAR-T cells) in a subject having such cancers. Without wishing to be bound by the theory, it is envisioned that EBV VLP can promotes activation, proliferation and killing activities of T cells (see Example 7). In addition, the presence of said EBV-VLP acting as a vaccine may further enhance the presentation of the EBV antigens on antigen-presenting cells available for enhancing said CAR-T therapy. Accordingly, the immunogenic composition described herein may contribute to overcoming the immunosuppressive tumor microenvironment, a major issue for the limited success of CAR-T therapy on solid tumors.

### BRIEF DESCRIPTION OF THE FIGURES

FIG. 1 shows representative anti-EBV Gp350 CAR-T cells demonstrated potent killing on PCI-gp350 cells but did not show detectable activity in PCI cells without gp350 expression.

FIG. 2 shows co-incubation of representative Polyinosinic Polycytidylic Acid Based Adjuvant composition (named as ZNP) prepared in Example 1 with T cells and PBMCs promotes activation and proliferation of T cells in a ZNP concentration dependent manner, suggesting the ZNP composition (or ZNP-EBV-VLP) can be useful in enhancing efficacy of T cell therapy including CAR-T cells.

FIG. 3 shows co-incubating of VLP of EBV with Raji cells significantly enhances the cytotoxicity (% cytolysis) of anti-EBV Gp350 CAR-T cells to target cells PCI-gp350 cell line (PCI-g) in the VLP-concentration manner (PCI-g+1.25×10⁷ VLP, PCI-g+1.25×10⁸ VLP compared to PCI-g+0 VLP). In contrast, VLP of EBV does not increase cytotoxicity (% cytolysis) of anti-EBV Gp350 CAR-T cells to control PCI cells.

FIG. 4 shows that the enhanced cytotoxicity of anti-EBV Gp350 CAR-T cells to target cells PCI-gp350 cell line in the presence of VLP of EBV is dependent on the co-incubation with VLP-targeting cell Raji cells. Cytotoxicity of the sample added with 1.25×10⁸ VLP co-incubated with Raji cells (Raji+1.25×10⁸ VLP) is clearly higher than that of the sample added with 1.25×10⁸ VLP without co-incubated with Raji cells (Raji+0 VLP).

FIG. 5 shows that anti-EBV Gp350 CAR-T cells show little or low cytotoxicity to PCI cell line which does not express gp350 antigen, in the absence or in the presence of VLP of EBV.

FIG. 6 shows co-incubating of VLP of EBV with Raji cells significantly enhances the cytotoxicity (% cytolysis) of anti-EBV Gp350 CAR-T cells to target cells PCI-gp350 cell line (PCI-g) in the VLP-concentration manner (CAR+VLPe7 or CAR+VLPe8 compared to CAR only). In contrast, VLP of EBV does not increase cytotoxicity (% cytolysis) of anti-EBV Gp350 CAR-T cells to control PCI cells. VLP of EBV alone does not increase cytotoxicity (% cytolysis) of mock T cells or PCI cell without gp350 expression.

FIG. 7 shows that Gp350 CAR-T cells inhibited nasopharyngeal carcinoma cells C666-1 growth and suppressed tumor formation in vivo. C666-1 (engineered with gp350 and luciferase expression) cells were subcutaneously injected into mice followed by 4×106 (4E6) or 2×106 (4E6) gp350 CAR-T or Mock T cells administration.

FIG. 7A shows results of tumor cells growth luminescence imaging at Day 0, 7, 14, 18 (4×106 groups) or 21 (2×106 and PBS groups) time points were recorded and compared, showing the specific tumor formation suppression by gp350 CAR-T cells. FIG. 7B shows average tumor cell luminescence intensity change at Day 0, 7, 14, 18 or 21 post CAR-T injection for 4×106 (4E6) CAR-T cell or Mock T cells treated groups. FIG. 7C shows average tumor cell luminescence intensity change at Day 0, 7, 14, 18 or 21 post CAR-T injection for 2×106 (2E6) of CAR-T cell, Mock T cells, or PBS treated groups. FIG. 7D shows the average tumor volume change pattern post CAR-T injection on Day 0, 7, 14, 18 or 21. FIG. 7E shows dissection and evaluation of tumor tissue weight in mice across different experimental groups, showing reduction of tumor growth in CAR-T treat group.

FIG. 8 shows cell percentage and counts comparison of human CD45+, CD8+, CAR+, and CAR+/CD8+ cells in blood or spleen samples from of 4×106 (4E6) CAR T and Mock T treatment groups at Day 18 (FIG. 8A), and 2×106 (2E6) CAR T cells and Mock T cells treatment groups at Day 24 (FIG. 8B). The results shows that CAR-T cells was found existence in blood and spleen while suppressing tumor formation.

FIG. 9 shows gp350-targeting CAR-T cells inhibit T-cell lymphoma cell growth and suppress tumor formation in vivo. FIG. 9A shows tumor profile of T-cell lymphoma mice model which were injected with Jurkat-gp350-luc cells 5 or 7 days for tumor formation. The mice were examined by IVIS imaging and being administrated with 2×10⁶ (2E6) or 1×10⁶ (1E6) of CAR-T cells or Mock T cells respectively. Tumor cells growth luminescence imaging at Day 0, 7, 14, 21, 28, 32 time points were recorded and compared, showing the specific tumor formation suppression by gp350 CAR-T cells. FIGS. 9B and 9C respectively shows the average tumor cell luminescence intensity change along the time course in 2×10⁶ (2E6) or 1×10⁶ (1E6) of CAR-T, or Mock T groups post CAR-T injection.

FIG. 10 shows tumor shrinkage induced by CAR T-cells comprising anti-EBV Gp350 in a patient diagnosed with B-cell acute lymphoblastic leukemia with central nervous system involvement. FIG. 10 (A) shows MRI imaging result which showed the lesion in the brain before treatment (top panel) and after treatment (bottom panel), which almost disappeared after treatment; FIG. 10 (B) shows that the percentages of aberrant blasts in blood dropped after treatment. After the treatment, the subject was discharged from hospital without CRS, ICANS and significant abnormality of heart, liver and kidney function tests.

### DETAILED DESCRIPTION

Unless defined otherwise, all technical and scientific terms used herein have the same meaning as commonly understood by one of ordinary skill in the art to which the disclosure pertains.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances 10%, or in some instances ±5%, or in some instances ±1%, or in some instances ±0.1% from the specified value, as such variations are appropriate to perform the disclosed methods.

An “immunogenic composition” as used here in refers to a substance that elicits an immune response when administered to a host.

The term “Poly I:C” or “PIC” refers to a composition comprising polyriboinosinic and polyribocytidylic nucleic acids, which may also be referred to as polyinosinic acid-polycytidylic acid or polyinosinic acid-polycytidilic, respectively.

The term “polypeptide,” “peptide,” “oligopeptide,” and “protein,” are used interchangeably herein, and refer to a polymeric form of amino acids of any length, which can include coded and non-coded amino acids, chemically or biochemically modified or derivatized amino acids, and polypeptides having modified peptide backbones.

The term “immunogen” refers to an antigen that is able to elicit an immune response, such as B-cell (humoral/antibody) and/or T-cell (cellular) adaptive immune responses upon exposure to a host organism. The term “antigen” includes but is not limited to cells; cell extracts; proteins; lipoproteins; glycoproteins; nucleoproteins; polypeptides; peptides; polysaccharides; polysaccharide conjugates; peptide mimics of polysaccharides; lipids; glycolipids; carbohydrates; viruses; viral extracts; bacteria; bacterial extracts; fungi; fungal extracts; multicellular organisms such as parasites; and allergens. Antigens may be exogenous (e.g., from a source other than the individual to whom the antigen is administered, e.g., from a different species) or endogenous (e.g., originating from within the host, e.g., a diseased element of body, a cancer antigen, a virus infected cell producing antigen, and the like). Antigens may be native (e.g., naturally-occurring); synthetic; or recombinant. Antigens include crude extracts; whole cells; and purified antigens, where “purified” indicates that the antigen is in a form that is enriched relative to the environment in which the antigen normally occurs and/or relative to the crude extract, for example, a cultured form of the antigen.

In some embodiments, the immunogen is a recombinant protein, virus-like particle (VLP), peptide, mRNA, DNA, vaccine or dendritic cell vaccine. In some other embodiments, the immunogen can be one or more polypeptides/peptides derived from cancer cells or antigenic fragments or variants thereof. It will be understood that the immunogen described herein may further comprise additional components. For example, one or more immunogen may be comprised in a lipid or liposome. In some embodiments, a peptide or polypeptide corresponding to a cancer antigen may generally be 10-20 amino acid residues in length, and may contain more than one peptide determinants or up to about 30-50 residues or so. In some embodiments, the polypeptide is between 10 and about 150 residues or more in length. In some embodiments, longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present disclosure. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

In some embodiments, the immunogen is a “tumor-associated antigen” or “cancer antigen” and may be selected from CTA, NY-ESO-1, LAGE-1, MAGE-A1, MAGE-A3, MAGE-A4, MAGE-A10, CT7, CT10, GAGE, PRAME; BAGE; RAGE, SAGE, HAGE, MPHOSPH1, DEPDC1, IMP3 and MAGE-A, and T-antigen BK, p53, Ras, c-Myc, A-Raf, B-Raf, C-Raf, cyclin-dependent kinases, MAGE-A2, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, tyrosinases, TRP-1, TRP-2, ART-4, CAMEL, CEA, Cyp-B, hTERT, hTRT, ICE, MUC1, MUC2, phosphoinositide 3-kinases (PI3K), TRK, PRAME, P15, RU1, RU2, SART-1, SART-3 receptors, Wilms tumor antigen (WT1), AFP, β-catenin/ω, caspases-8/m, CEA, CDK-4/m, ELF2M, GnT-V, G250, HSP70-2M, HST-2, KIAA0205, MUM-1, MUM-2, MUM-3, myosin/m, RAGE, SART-2, TRP-2/INT2, 707-AP, annexin II, CDC27/m, TPI/mber-abl, BCRABL, interferon regulatory factor 4 (IRF4), ETV6/AML, LDLR/FUT, Pml/RARa, tumor-associated calcium signal transducer 1 (TACSTD1) TACSTD2 receptor tyrosine kinases, epidermal growth factor receptor (EGFR), EGFRvIII, platelet growth factor receptor (PDGFR), vascular endothelial growth factor receptor ia (VEGFR), cytoplasmic tyrosine kinases, src family, syk-ZAP70, integrin-linked kinase (ILK), signal transducers and transcription activators STAT3, STAT5 and STAT6, factors induced by hypoxia, HIF-1a and HIF-2a, nuclear factor kappa in (NF-kB), Notch, Notch1-4, c-Met receptors, mammalian rapamycin targets (mTOR), WNT, extracellular signal-regulated kinases (ERK), PMSA, PR-3, MDM2, mesothelin, cancer kidney cells-5T4, SM22-alpha, carbonic anhydrase I (CAI) and IX (CAIX), STEAD, TEL/AML1, GD2, proteinase 3, hTERT, sarcoma translocation control points, EphA2, ML-IAP, EpCAM, ERG (fusion gene TMPRSS2 ETS), NA17, PAX3, ALK, androgen receptor, zclin B1, polysialic acid, MYCN, RhoC, GD3, fucosyl GM1, mesothelin, PSCA, sLe, PLAC1, GM3, BORIS, Tn, GLoboH, NYBR-1, RG SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2 and fos-related antigen 1.

In yet another embodiment, the immunogen is a viral antigen from an oncogenic virus. Typical oncogenic viruses include, but are not limited to, EBV, HPV, HBV, HCV, HTLV, and KSHV. Typical viral antigens from oncogenic viruses that can be used in this disclosure include, but are not limited to, EBV: EBNA-1, LMP-1, LMP-2A; HPV: E6, E7, E5; HBV: HBx; HCV: Core, NS3, Ns5A; HTLV: Tax, HBZ; KSHV: vFLIP, LANA, vGPCR, vIRF-1.

In some embodiments, said immunogen is an Epstein-Barr virus antigen (EBV antigen). The EBV antigen may be an EBV VLP. In preferred embodiment, the EBV virus antigen preferably is the EBV glycoprotein 350/220 (gp350/220). An exemplary EBV gp350 protein is shown in the UniProt database, entry P03200-1, version 1 of 21 Jul. 1986. An exemplary EBV gp220 is shown in the same database entry but positions 502 to 750 are missing. In some embodiments, said immunogen is an EBV VLP.

### EBV VLP and Immunogenic Composition Comprising EBV VLP

In some embodiments, the immunogenic composition of the present disclosure comprises a VLP of Epstein Barr virus (EBV), i.e. EBV-VLP.

In some embodiments of the disclosure, said EBV-VLP comprises (i) at least one EBV structural polypeptide, (ii) at least one EBV lytic polypeptide, (iii) membrane lipids, wherein preferably said VLP has one of more of the following properties:
(a) being devoid of EBV DNA;
(b) the B-cell transformation capacity of one of more EBV required for B-cell transformation is disabled or removed while their immunogenicity is maintained; and/or
(c) said EBV does not comprise one or more EBV polypeptides required for B-cell transformation which is selected from the group consisting of LMP1, EBNA2, EBNA3a, EBNA3b and EBNA3c.

In some embodiments of the EBV VLP of the disclosure, said VLP comprises at least one EBV polypeptide of gp350 and/or further comprises at least one EBV latent polypeptide.

The EBV polypeptide gp350 (glycoprotein 350) is a membrane bound glycoprotein. Said gp350 is responsible for the specificity (tropism) for B cells by binding to CD21 on the cell surface of B-cells. Additional accessory viral polypeptides may contribute to a fully efficient infection (Chesnokova et al., 2009; Omerovic et al., 2005; Silva et al., 2004; Sorem and Longnecker, 2009). Also, infection at low efficiency has also been demonstrated with recombinant EBV particles devoid of gp350 (Janz et al., 2000). Recent research also postulates an implication of gp350 after the internalization step and presumably during release of the viral capsid from the endosomal compartment (Busse et al., 2010). While not crucial for the VLP according to the invention, it is preferred that gp350 is comprised in the VLP particle's membrane since upon administration of the vaccine the immune response generated more closely resembles the immune response elicited upon infection by a wildtype EBV,

The term “EBV” as used herein relates to any wildtype, i.e. naturally occurring, EBV strain and is not restricted to one particular strain, Specifically, EBV type 1 and EBV type 2 strains are well-known in the art and have been extensively characterised. These two EBV-types differ largely in nuclear polypeptide genes that encode EBNA-LP, EBNA-2, EBNA-3A, EBNA-3B and EBNA-3C. Beyond differences relating to genes encoding polypeptides of the EBNA-family, the genomes of type 1 and 2 differ little. Type 1 is dominantly prevalent in developed world populations, whereas type 2 is also prevalent in equatorial Africa and New Guinea (Kieff and Rickinson, 2007, for review). Further, the term EBV polypeptide comprises also polypeptides that are not identical to wildtype EBV strains as regards the sequence, but comprise proteins which share at least (for each value) 99%, 98%, 97%, 96%, 95%, 90%, 85%, 80% and at least 75% identity in sequence to a wildtype EBV polypeptide. The degree of identity of polypeptide sequences can be calculated by well-known methods by the person skilled in the art and may comprise the automatic execution of algorithms effecting the alignment of sequence data and calculation of sequence homologies. The EBV polypeptides of the particle may originate from different EBV strains; preferably they originate from one strain. As mentioned above, EBV polypeptides required to be comprised in the particle belong to the groups of EBV structural polypeptides and EBV lytic polypeptides. As will be understood by the skilled person, a particular polypeptide of EBV may belong to more than one of the above mentioned groups of polypeptides. In other words, an EBV polypeptide may represent a structural polypeptide as well as a lytic polypeptide as will be apparent from the specific EBV polypeptides mentioned in the following paragraphs. In the latter case, the particle need not comprise a further EBV polypeptide that is either a structural or a lytic polypeptide. Preferably, the particle comprises at least one separate EBV polypeptide for each of the above-mentioned groups of EBV polypeptides as this typically increases the antigenic potential of the vaccine. More preferred is that at least (for each value) 2, 3, 4, 5, 6, 7, 8, 9, 10, 11, or at least 12 separate polypeptides are independently part of each of said polypeptide groups comprised by the particle of the vaccine.

The term “latent polypeptides” relates to EBV polypeptides that are involved in the induction and maintenance of the EBV latent cycle and/or are expressed as a consequence of the induction of the latent cycle. Preferably, the at least one latent polypeptide is LMP-1 (also termed BNLF1) and/or L P-2.

The term “structural polypeptide” of EBV relates to polypeptides involved in the structural setup of the EBV. Said polypeptides are preferably selected from the group consisting of membrane polypeptides, tegument polypeptides and capsid polypeptides. EBV membrane polypeptides comprise the polypeptides selected from the group consisting of BALF4, BLLF1 (also termed gp350), BDLF2, BDLF3, BKRF2, BLRF1, BNLF1 (also termed LMP-1), TP (also termed LMP-2a), BXLF2, BZLF2 and any combination thereof. EBV tegument polypeptides comprise the polypeptides selected from the group consisting of BBRF2, BGLF2, B LF1, BNRF1, BOLF1, BPLF1, BTRF1, BVRF1 and any combination thereof. EBV capsid polypeptides comprise the polypeptides selected from the group consisting of BBRF1, BcLF1, BDLF1, BFRF3 and any combination thereof. Preferably, the at least one structural polypeptide is selected from the group consisting of BLLF1, BMLF1, BNRF1 or any combination thereof such as BLLF1 and BMLF1, BLLF1 and BNRF1, or BMLF1 and BNRF1.

The term “lytic polypeptides” relates to EBV polypeptides that are involved in the induction and maintenance of the EBV lytic cycle (herein also referred to as replicative phase) and/or are expressed as a consequence of the induction of the lytic cycle. Said lytic polypeptides are preferably selected from the group comprising the immediate early genes, the early genes and the late lytic genes (Kieff and Rickinson, 2007). The lytic cycle is initiated by the expression of BZLF1 and BRLF1, both immediate early proteins, followed by the expression of the early and late proteins. Following induction, cells that have become permissive for virus replication undergo cytopathic changes characteristic of herpesviruses (Kieff and Rickinson, 2007). Exemplary lytic polypeptides to be used in accordance with the invention are selected from the group comprising BZLF1, BRLF1, BMRF1, BMLF1, BALF2, BALF5, BGL2, BHRF1, BALF4, BDLF3 and any combination thereof. Preferably, the at least one lytic polypeptide is BLLF1 (also termed gp350) or any combination thereof.

The term “membrane lipids” as used in accordance with the present invention relates to lipids that are capable of spontaneously arranging to form a lipid bilayer. Such membrane lipids are lipids that comprise a hydrophobic and a hydrophilic region, wherein after self-assembly the hydrophobic regions of the membrane lipids form the inner part of the bilayer whereas the hydrophilic regions form the outer face of the membrane. Preferably, the membrane lipids are lipids that naturally form cell membranes such as amphipathic phospholipids. Also preferred is that said membrane lipids originate from a host cell where wildtype EBV is capable of replicating. More preferred, said membrane lipids originate from a cell according to the present invention. In accordance with the invention, the membranes comprised in the particle are present in an amount sufficient to form a membrane which constitutes the outer shell of the particle. The particle must possess said membrane outer shell, which preferably comprises at least one EBV structural polypeptide. As outlined below, preferred examples of membrane-bound EBV structural polypeptides are the gp350 polypeptide and the L P-1 polypeptide. As further detailed below, the B-cell transformation capacity of LMP-1 may be disabled. Also preferred is that the particle's membrane comprises further membrane constituents also found naturally in an EBV membrane such as, e.g. further membrane polypeptides which may be found on the inside, on the outside of the membrane or spanning the membrane.

In some embodiments of the immunogenic composition of the invention, said EBV VLP comprises glycoprotein 350/220 (gp350) protein or fragment thereof, optionally wherein the VLP comprises one, two, three or more polypeptide sequences that is/are at least 80%, at least 90%, at least 95%, at least 96%, at least 97%, at least 98%, at least 99% identical or 100% identical to the polypeptide sequences selected from the group consisting of SEQ ID NOs: 102 to 117.

In some embodiments, said EBV VLP comprises a protein sequence of gp350 of Epstein-Barr virus as represented by:

MEAALLVCQYTIQSLIHLTGEDPGFFNVEIPEFPFYPTCNVCTADVNVTINFDVGGKK HQLDLDFGQLTPHTKAVYQPRGAFGGSENATNLFLLELLGAGELALTMRSKKLPIN VTTGEEQQVSLESVDVYFQDVFGTMWCHHAEMQNPVYLIPETVPYIKWDNONSTN ITAVVRAQGLDVTLPLSLPTSAQDSNFSVKTEMLGNEIDIECIMEDGEISQVLPGDNK FNITCSGYESHVPSGGILTSTSPVATPIPGTGYAYSLRLTPRPVSRFLGNNSILYVFY SGNGPKASGGDYCIQSNIVFSDEIPASQDMPTNTTDITYVGDNATYSVPMVTSEDA NSPNVTVTAFWAWPNNTETDFKCKWTLTSGTPSGCENISGAFASNRTFDITVSGL GTAPKTLIITRTATNATTTTHKVIFSKAPESTTTSPTLNTTGFADPNTTTGLPSSTHVP TNLTAPASTGPTVSTADVTSPTPAGTTSGASPVTPSPSPWDNGTESKAPDMTSST SPVTTPTPNATSPTPAVTTPTPNATSPTPAVTTPTPNATSPTLGKTSPTSAVTTPTP NATSPTLGKTSPTSAVTTPTPNATSPTLGKTSPTSAVTTPTPNATGPTVGETSPQA NATNHTLGGTSPTPVVTSQPKNATSAVTTGQHNITSSSTSSMSLRPSSNPETLSPS TSDNSTSHMPLLTSAHPTGGENITQVTPASISTHHVSTSSPAPRPGTTSQASGPGN SSTSTKPGEVNVTKGTPPQNATSPQAPSGQKTAVPTVTSTGGKANSTTGGKHTTG HGARTSTEPTTDYGGDSTTPRPRYNATTYLPPSTSSKLRPRWTFTSPPVTTAQATV PVPPTSQPRFSNLSMLVLQWASLAVLTLLLLLVMADCAFRRNLSTSHTYTTPPYDD AETYV (SEQ ID NO: 102); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a truncated protein sequence of gp350 of Epstein-Barr virus as represented by:

MEAALLVCQYTIQSLIHLTGEDPGFFNVEIPEFPFYPTCNVCTADVNVTINFDVGGKK HQLDLDFGQLTPHTKAVYQPRGAFGGSENATNLFLLELLGAGELALTMRSKKLPIN VTTGEEQQVSLESVDVYFQDVFGTMWCHHAEMQNPVYLIPETVPYIKWDNCNSTN ITAVVRAQGLDVTLPLSLPTSAQDSNFSVKTEMLGNEIDIECIMEDGEISQVLPGDNK FNITCSGYESHVPSGGILTSTSPVATPIPGTGYAYSLRLTPRPVSRFLGNNSILYVFY SGNGPKASGGDYCIQSNIVFSDEIPASQDMPTNTTDITYVGDNATYSVPMVTSEDA NSPNVTVTAFWAWPNNTETDFKCKWTLTSGTPSGCENISGAFASNRTFDITVSGL GTAPKTLIITRTATNATTTTHKVIFSKAPESTTTSPTLNTTGFADPNTTTGLPSSTHVP TNLTAPASTGPTVSTADVTSPTPAGTTSGASPVTPSPSPWDNGTES (SEQ ID NO: 103); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of LMP1 of Epstein-Barr virus as represented by:

MEHDLERGPPGPRRPPRGPPLSSSLGLALLLLLLALLFWLYIVMSDWTGGALLVLY SFALMLIIIILIIFIFRRDLLCPLGALCILLLMITLLLIALWNLHGQALFLGIVLFIFGCLLVL GIWIYLLEMLWRLGATIWQLLAFFLAFFLDLILLIIALYLQQNWWTLLVDLLWLLLFLAI LIWMYYHGQRHSDEHHHDDSLPHPQQATDDSGHESDSNSNEGRHHLLVSGAGD GPPLCSQNLGAPGGGPDNGPQDPDNTDDNGPQDPDNTDDNGPHDPLPQDPDNT DDNGPQDPDNTDDNGPHDPLPHSPSDSAGNDGGPPQLTEEVENKGGDQGPPLM TDGGGGHSHDSGHGGGDPHLPTLLLGSSGSGGDDDDPHGPVQLSYYD (SEQ ID NO: 104); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of LMP2A of Epstein-Barr virus as represented by:

MGSLEMVPMGAGPPSPGGDPDGYDGGNNSQYPSASGSSGNTPTPPNDEERESN EEPPPPYEDPYWGNGDRHSDYQPLGTQDQSLYLGLQHDGNDGLPPPPYSPRDDS SQHIYEEAGRGSMNPVCLPVIVAPYLFWLAAIAASCFTASVSTVVTATGLALSLLLLA AVASSYAAAQRKLLTPVTVLTAVVTFFAICLTWRIEDPPFNSLLFALLAAAGGLQGIY VLVMLVLLILAYRRRWRRLTVCGGIMFLACVLVLIVDAVLQLSPLLGAVTVVSMTLLL LAFVLWLSSPGGLGTLGAALLTLAAALALLASLILGTLNLTTMFLLMLLWTLVVLLICS SCSSCPLSKILLARLFLYALALLLLASALIAGGSILQTNFKSLSSTEFIPNLFCMLLLIVA GILFILAILTEWGSGNRTYGPVFMCLGGLLTMVAGAVWLTVMSNTLLSAWILTAGFLI FLIGFALFGVIRCCRYCCYYCLTLESEERPPTPYRNTV (SEQ ID NO: 105); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of EBNA-1 of Epstein-Barr virus as represented by:

MSDEGPGTGPGNGLGEKGDTSGPEGSGGSGPQRRGGDNHGRGRGRGRGRGG GRPGAPGGSGSGPRHRDGVRRPQKRPSCIGCKGTHGGTGAGAGAGGAGAGGA GAGGGAGAGGGAGGAGGAGGAGAGGGAGAGGGAGGAGGAGAGGGAGAGGG AGGAGAGGGAGGAGGAGAGGGAGAGGGAGGAGAGGGAGGAGGAGAGGGAG AGGAGGAGGAGAGGAGAGGGAGGAGGAGAGGAGAGGAGAGGAGAGGAGGAG AGGAGGAGAGGAGGAGAGGGAGGAGAGGGAGGAGAGGAGGAGAGGAGGAGA GGAGGAGAGGGAGAGGAGAGGGGRGRGGSGGRGRGGSGGRGRGGSGGRRG RGRERARGGSRERARGRGRGRGEKRPRSPSSQSSSSGSPPRRPPPGRRPFFHP VGEADYFEYHQEGGPDGEPDVPPGAIEQGPADDPGEGPSTGPRGQGDGGRRKK GGWFGKHRGQGGSNPKFENIAEGLRALLARSHVERTTDEGTWVAGVFVYGGSKT SLYNLRRGTALAIPQCRLTPLSRLPFGMAPGPGPQPGPLRESIVCYFMVFLQTHIFA EVLKDAIKDLVMTKPAPTCNIRVTVCSFDDGVDLPPWFPPMVEGAAAEGDDGDDG DEGGDGDEGEEGQE (SEQ ID NO: 106); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of truncated EBNA-1 (326-641) of Epstein-Barr virus as represented by:

GGGRGRGGSGGRGRGGSGGRGRGGSGGRRGRGRERARGGSRERARGRGRG RGEKRPRSPSSQSSSSGSPPRRPPPGRRPFFHPVGEADYFEYHQEGGPDGEPDV PPGAIEQGPADDPGEGPSTGPRGQGDGGRRKKGGWFGKHRGQGGSNPKFENIA EGLRALLARSHVERTTDEGTWVAGVFVYGGSKTSLYNLRRGTALAIPQCRLTPLSR LPFGMAPGPGPQPGPLRESIVCYFMVFLQTHIFAEVLKDAIKDLVMTKPAPTCNIRV TVCSFDDGVDLPPWFPPMVEGAAAEGDDGDDGDEGGDGDEGEEGQE (SEQ ID NO: 107); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of truncated EBNA-1 (326-641)-LMP2 fusion protein of Epstein-Barr virus as represented by:

GGGRGRGGSGGRGRGGSGGRGRGGSGGRRGRGRERARGGSRERARGRGRG RGEKRPRSPSSQSSSSGSPPRRPPPGRRPFFHPVGEADYFEYHQEGGPDGEPDV PPGAIEQGPADDPGEGPSTGPRGQGDGGRRKKGGWFGKHRGQGGSNPKFENIA EGLRALLARSHVERTTDEGTWVAGVFVYGGSKTSLYNLRRGTALAIPQCRLTPLSR LPFGMAPGPGPQPGPLRESIVCYFMVFLQTHIFAEVLKDAIKDLVMTKPAPTCNIRV TVCSFDDGVDLPPWFPPMVEGAAAEGDDGDDGDEGGDGDEGEEGQEMGSLEM VPMGAGPPSPGGDPDGYDGGNNSQYPSASGSSGNTPTPPNDEERESNEEPPPP YEDPYWGNGDRHSDYQPLGTQDQSLYLGLQHDGNDGLPPPPYSPRDDSSQHIYE EAGRGSMNPVCLPVIVAPYLFWLAAIAASCFTASVSTVVTATGLALSLLLLAAVASSY AAAQRKLLTPVTVLTAVVTFFAICLTWRIEDPPFNSLLFALLAAAGGLQGIYVLVMLV LLILAYRRRWRRLTVCGGIMFLACVLVLIVDAVLQLSPLLGAVTVVSMTLLLLAFVLW LSSPGGLGTLGAALLTLAAALALLASLILGTLNLTTMFLLMLLWTLVVLLICSSCSSCP LSKILLARLFLYALALLLLASALIAGGSILQTNFKSLSSTEFIPNLFCMLLLIVAGILFILAI LTEWGSGNRTYGPVFMCLGGLLTMVAGAVWLTVMSNTLLSAWILTAGFLIFLIGFAL FGVIRCCRYCCYYCLTLESEERPPTPYRNTV (SEQ ID NO: 108); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of glycoprotein H (or BXLF2) of Epstein-Barr virus as represented by:

MQLLCVFCLVLLWEVGAASLSEVKLHLDIEGHASHYTIPWTELMAKVPGLSPEALW REANVTEDLASMLNRYKLIYKTSGTLGIALAEPVDIPAVSEGSMQVDASKVHPGVIS GLNSPACMLSAPLEKQLFYYIGTMLPNTRPHSYVFYQLRCHLSYVALSINGDKFQYT GAMTSKFLMGTYKRVTEKGDEHVLSLVFGKTKDLPDLRGPFSYPSLTSAQSGDYS LVIVTTFVHYANFHNYFVPNLKDMFSRAVTMTAASYARYVLQKLVLLEMKGGCREP ELDTETLTTMFEVSVAFFKVGHAVGETGNGCVDLRWLAKSFFELTVLKDIIGICYGA TVKGMQSYGLERLAAMLMATVKMEELGHLTTEKQEYALRLATVGYPKAGVYSGLI GGATSVLLSAYNRHPLFQPLHTVMRETLFIGSHVVLRELRLNVTTQGPNLALYQLLS TALCSALEIGEVLRGLALGTESGLFSPCYLSLRFDLTRDKLLSMAPQEATLDQAAVS NAVDGFLGRLSLEREDRDAWHLPAYKCVDRLDKVLMIIPLINVTFIISSDREVRGSAL YEASTTYLSSSLFLSPVIMNKCSQGAVAGEPRQIPKIQNFTRTQKSCIFCGFALLSYD EKEGLETTTYITSQEVQNSILSSNYFDFDNLHVHYLLLTTNGTVMEIAGLYEERAHVV LAIILYFIAFALGIFLVHKIVMFFL (SEQ ID NO: 109); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of truncated glycoprotein H of Epstein-Barr virus as represented by:

MQLLCVFCLVLLWEVGAASLSEVKLHLDIEGHASHYTIPWTELMAKVPGLSPEALW REANVTEDLASMLNRYKLIYKTSGTLGIALAEPVDIPAVSEGSMQVDASKVHPGVIS GLNSPACMLSAPLEKQLFYYIGTMLPNTRPHSYVFYQLRCHLSYVALSINGDKFQYT GAMTSKFLMGTYKRVTEKGDEHVLSLVFGKTKDLPDLRGPFSYPSLTSAQSGDYS LVIVTTFVHYANFHNYFVPNLKDMFSRAVTMTAASYARYVLQKLVLLEMKGGCREP ELDTETLTTMFEVSVAFFKVGHAVGETGNGCVDLRWLAKSFFELTVLKDIIGICYGA TVKGMQSYGLERLAAMLMATVKMEELGHLTTEKQEYALRLATVGYPKAGVYSGLI GGATSVLLSAYNRHPLFQPLHTVMRETLFIGSHVVLRELRLNVTTQGPNLALYQLLS TALCSALEIGEVLRGLALGTESGLFSPCYLSLRFDLTRDKLLSMAPQEATLDQAAVS NAVDGFLGRLSLEREDRDAWHLPAYKCVDRLDKVLMIIPLINVTFIISSDREVRGSAL YEASTTYLSSSLFLSPVIMNKCSQGAVAGEPRQIPKIQNFTRTQKSCIFCGFALLSYD EKEGLETTTYITSQEVQNSILSSNYFDFDNLHVHYLLLTTNGTVMEIAGLYEERAH (SEQ ID NO: 110); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of glycoprotein L precursor of Epstein-Barr virus as represented by:

MRAVGVFLAICLVTIFVLPTWGNWAYPCCHVTQLRAQHLLALENISDIYLVSNQTCD GFSLASLNSPKNGSNQLVISRCANGLNVVSFFISILKRSSSALTGHLRELLTTLETLY GSFSVEDLFGANLNRYAWHRGG (SEQ ID NO: 111); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of truncated glycoprotein L precursor of Epstein-Barr virus as represented by:

YPCCHVTQLRAQHLLALENISDIYLVSNQTCDGFSLASLNSPKNGSNQLVISRCANG LNVVSFFISILKRSSSALTGHLRELLTTLETLYGSFSVEDLFGANLNRYAWHRGG (SEQ ID NO: 112); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of glycoprotein B precursor of Epstein-Barr virus as represented by:

MTRRRVLSVVVLLAALACRLGAQTPEQPAPPATTVQPTATRQQTSFPFRVCELSSH GDLFRFSSDIQCPSFGTRENHTEGLLMVFKDNIIPYSFKVRSYTKIVTNILIYNGWYA DSVTNRHEEKFSVDSYETDQMDTIYQCYNAVKMTKDGLTRVYVDRDGVNITVNLK PTGGLANGVRRYASQTELYDAPGWLIWTYRTRTTVNCLITDMMAKSNSPFDFFVTT TGQTVEMSPFYDGKNKETFHERADSFHVRTNYKIVDYDNRGTNPQGERRAFLDKG TYTLSWKLENRTAYCPLQHWQTFDSTIATETGKSIHFVTDEGTSSFVTNTTVGIELP DAFKCIEEQVNKTMHEKYEAVQDRYTKGQEAITYFITSGGLLLAWLPLTPRSLATVK NLTELTTPTSSPPSSPSPPAPSAARGSTPAAVLRRRRRDAGNATTPVPPTAPGKSL GTLNNPATVQIQFAYDSLRRQINRMLGDLARAWCLEQKRQNMVLRELTKINPTTVM SSIYGKAVAAKRLGDVISVSQCVPVNQATVTLRKSMRVPGSETMCYSRPLVSFSFI NDTKTYEGQLGTDNEIFLTKKMTEVCQATSQYYFQSGNEIHVYNDYHHFKTIELDGI ATLQTFISLNTSLIENIDFASLELYSRDEQRASNVFDLEGIFREYNFQAQNIAGLRKDL DNAVSNGRNQFVDGLGELMDSLGSVGQSITNLVSTVGGLFSSLVSGFISFFKNPFG GMLILVLVAGVVILVISLTRRTRQMSQQPVQMLYPGIDELAQQHASGEGPGINPISKT ELQAIMLALHEQNQEQKRAAQRAAGPSVASRALQAARDRFPGLRRRRYHDPETAA ALLGEAETEF (SEQ ID NO: 113); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of BNRF1 of Epstein-Barr virus as represented by:

MEERGRETQMPVARYGGPFIMVRLFGQDGEANIQEERLYELLSDPRSALGLDPGP LIAENLLLVALRGTNNDPRPQRQERARELALVGILLGNGEQGEHLGTESALEASGN NYVYAYGPDWMARPSTWSAEIQQFLRLLGATYVLRVEMGRQFGFEVHRSRPSFR QFQAINHLVLFDNALRKYDSGQVAAGFQRALLVAGPETADTRPDLRKLNEWVFGG RAAGGROLADELKIVSALRDTYSGHLVLQPTETLDTWKVLSRDTRTAHSLEHGFIH AAGTIQANCPQLFMRRQHPGLFPFVNAIASSLGWYYQTATGPGADARAAARRQQA FQTRAAAECHAKSGVPVVAGFYRTINATLKGGEGLQPTMFNGELGAIKHQALDTVR YDYGHYLIMLGPFQPWSGLTAPPCPYAESSWAQAAVQTALELFSALYPAPCISGYA RPPGPSAVIEHLGSLVPKGGLLLFLSHLPDDVKDGLGEMGPARATGPGMQQFVSS YFLNPACSNVFITVRQRGEKINGRTVLQALGRACDMAGCQHYVLGSTVPLGGLNFV NDLASPVSTAEMMDDFSPFFTVEFPPIQEEGASSPVPLDVDESMDISPSYELPWLS LESCLTSILSHPTVGSKEHLVRHTDRVSGGRVAQQPGVGPLDLPLADYAFVAHSQV WTRPGGAPPLPYRTWDRMTEKLLVSAKPGGENVKVSGTVITLGEQGYKVSLDLRE GTRLAMAEALLNAACAPILDPEDVLLTLHLHLDPRRADNSAVMEAMTAASDYARGL GVKLTFGSASCPETGSSASNFMTVVASVSAPGEFSGPLITPVLQKTGSLLIAVRCGD GKIQGGSLFEQLFSDVATTPRAPEALSLKNLFRAVQQLVKSGIVLSGHDISDGGLVT CLVEMALAGQRGVTITMPVASDYLPEMFAEHPGLVFEVEERSVGEVLQTLRSMNM YPAVLGRVGEQGPDQMFEVQHGPETVLRQSLRLLLGTWSSFASEQYECLRPDRIN RSMHVSDYGYNEALAVSPLTGKNLSPRRLVTEPDPRCQVAVLCAPGTRGHESLLA AFTNAGCLCRRVFFREVRDNTFLDKYVGLAIGGVHGARDSALAGRATVALINRFPA LRDAILKFLNRPDTFSVALGELGVQVLAGLGAVGSTDNPPAPGVEVNVQRSPLILAP NASGMFESRWLNISIPATTSSVMLRGLRGCVLPCWVQGSCLGLQFTNLGMPYVLQ NAHQIACHFHSNGTDAWRFAMNYPRNPTEQGNIAGLCSRDGRHLALLCDPSLCTD FWQWEHIPPAFGHPTGCSPWTLMFQAAHLWSLRHGRPSE (SEQ ID NO: 114); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of BNRF1-EBNA1 (387-513aa) fusion protein as represented by:

MEERGRETQMPVARYGGPFIMVRLFGQDGEANIQEERLYELLSDPRSALGLDPGP LIAENLLLVALRGTNNDPRPQRQERARELALVGILLGNGEQGEHLGTESALEASGQ SSSSGSPPRRPPPGRRPFFHPVGEADYFEYHQEGGPDGEPDVPPGAIEQGPADD PGEGPSTGPRGQGDGGRRKKGGWFGKHRGQGGSNPKFENIAEGLRALLARSHV ERTTDEGTWVAGVFVYGGSASGNNYVYAYGPDWMARPSTWSAEIQQFLRLLGAT YVLRVEMGRQFGFEVHRSRPSFRQFQAINHLVLFDNALRKYDSGQVAAGFORALL VAGPETADTRPDLRKLNEWVFGGRAAGGRQLADELKIVSALRDTYSGHLVLQPTE TLDTWKVLSRDTRTAHSLEHGFIHAAGTIQANCPQLFMRRQHPGLFPFVNAIASSL GWYYQTATGPGADARAAARRQQAFQTRAAAECHAKSGVPVVAGFYRTINATLKG GEGLQPTMFNGELGAIKHQALDTVRYDYGHYLIMLGPFQPWSGLTAPPCPYAESS WAQAAVQTALELFSALYPAPCISGYARPPGPSAVIEHLGSLVPKGGLLLFLSHLPDD VKDGLGEMGPARATGPGMQQFVSSYFLNPACSNVFITVRQRGEKINGRTVLQALG RACDMAGCQHYVLGSTVPLGGLNFVNDLASPVSTAEMMDDFSPFFTVEFPPIQEE GASSPVPLDVDESMDISPSYELPWLSLESCLTSILSHPTVGSKEHLVRHTDRVSGG RVAQQPGVGPLDLPLADYAFVAHSQVWTRPGGAPPLPYRTWDRMTEKLLVSAKP GGENVKVSGTVITLGEQGYKVSLDLREGTRLAMAEALLNAACAPILDPEDVLLTLHL HLDPRRADNSAVMEAMTAASDYARGLGVKLTFGSASCPETGSSASNFMTVVASVS APGEFSGPLITPVLQKTGSLLIAVRCGDGKIQGGSLFEQLFSDVATTPRAPEALSLK NLFRAVQQLVKSGIVLSGHDISDGGLVTCLVEMALAGQRGVTITMPVASDYLPEMF AEHPGLVFEVEERSVGEVLQTLRSMNMYPAVLGRVGEQGPDQMFEVQHGPETVL RQSLRLLLGTWSSFASEQYECLRPDRINRSMHVSDYGYNEALAVSPLTGKNLSPR RLVTEPDPRCQVAVLCAPGTRGHESLLAAFTNAGCLCRRVFFREVRDNTFLDKYV GLAIGGVHGARDSALAGRATVALINRFPALRDAILKFLNRPDTFSVALGELGVQVLA GLGAVGSTDNPPAPGVEVNVQRSPLILAPNASGMFESRWLNISIPATTSSVMLRGL RGCVLPCWVQGSCLGLQFTNLGMPYVLQNAHQIACHFHSNGTDAWRFAMNYPRN PTEQGNIAGLCSRDGRHLALLCDPSLCTDFWQWEHIPPAFGHPTGCSPWTLMFQA AHLWSLRHGRPSE (SEQ ID NO: 115); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of BNRF1-EBNA1 (497-619aa) fusion protein as represented by:

MEERGRETQMPVARYGGPFIMVRLFGQDGEANIQEERLYELLSDPRSALGLDPGP LIAENLLLVALRGTNNDPRPQRQERARELALVGILLGNGEQGEHLGTESALEASGTT DEGTWVAGVFVYGGSKTSLYNLRRGTALAIPQCRLTPLSRLPFGMAPGPGPQPGP LRESIVCYFMVFLQTHIFAEVLKDAIKDLVMTKPAPTCNIRVTVCSFDDGVDLPPWFP PMVEGAAAASGNNYVYAYGPDWMARPSTWSAEIQQFLRLLGATYVLRVEMGRQF GFEVHRSRPSFRQFQAINHLVLFDNALRKYDSGQVAAGFQRALLVAGPETADTRP DLRKLNEWVFGGRAAGGRQLADELKIVSALRDTYSGHLVLQPTETLDTWKVLSRD TRTAHSLEHGFIHAAGTIQANCPQLFMRRQHPGLFPFVNAIASSLGWYYQTATGPG ADARAAARRQQAFQTRAAAECHAKSGVPVVAGFYRTINATLKGGEGLQPTMENGE LGAIKHQALDTVRYDYGHYLIMLGPFQPWSGLTAPPCPYAESSWAQAAVQTALELF SALYPAPCISGYARPPGPSAVIEHLGSLVPKGGLLLFLSHLPDDVKDGLGEMGPAR ATGPGMQQFVSSYFLNPACSNVFITVRQRGEKINGRTVLQALGRACDMAGCQHYV LGSTVPLGGLNFVNDLASPVSTAEMMDDFSPFFTVEFPPIQEEGASSPVPLDVDES MDISPSYELPWLSLESCLTSILSHPTVGSKEHLVRHTDRVSGGRVAQQPGVGPLDL PLADYAFVAHSQVWTRPGGAPPLPYRTWDRMTEKLLVSAKPGGENVKVSGTVITL GEQGYKVSLDLREGTRLAMAEALLNAACAPILDPEDVLLTLHLHLDPRRADNSAVM EAMTAASDYARGLGVKLTFGSASCPETGSSASNFMTVVASVSAPGEFSGPLITPVL QKTGSLLIAVRCGDGKIQGGSLFEQLFSDVATTPRAPEALSLKNLFRAVQQLVKSGI VLSGHDISDGGLVTCLVEMALAGQRGVTITMPVASDYLPEMFAEHPGLVFEVEERS VGEVLQTLRSMNMYPAVLGRVGEQGPDQMFEVQHGPETVLRQSLRLLLGTWSSF ASEQYECLRPDRINRSMHVSDYGYNEALAVSPLTGKNLSPRRLVTEPDPRCQVAV LCAPGTRGHESLLAAFTNAGCLCRRVFFREVRDNTFLDKYVGLAIGGVHGARDSAL AGRATVALINRFPALRDAILKFLNRPDTFSVALGELGVQVLAGLGAVGSTDNPPAPG VEVNVQRSPLILAPNASGMFESRWLNISIPATTSSVMLRGLRGCVLPCWVQGSCLG LQFTNLGMPYVLQNAHQIACHFHSNGTDAWRFAMNYPRNPTEQGNIAGLCSRDG RHLALLCDPSLCTDFWQWEHIPPAFGHPTGCSPWTLMFQAAHLWSLRHGRPSE (SEQ ID NO: 116); or a functional variant thereof that is at least 80%, at least 90%, at least 95%, at least 96%, at least 97%, at least 98%, at least 99% identical or 100% identical to the polypeptide sequence described herein.

In some embodiments, said EBV VLP comprises a protein sequence of BNRF1-EBNA3C-EBNA1 fusion protein as represented by:

MEERGRETQMPVARYGGPFIMVRLFGQDGEANIQEERLYELLSDPRSALGLDPGP LIAENLLLVALRGTNNDPRPQRQERARELALVGILLGNGEQGEHLGTESALEASGN NAPPNENPYHARRGIKEHVIQNAFRASGIMAPPVVRMFMRERQLPQSTGRKPASG PKFENIAEGLRALLARSHVERTTDEGTWVARGTALAIPQCRLTPLSRLPFGMAPGN YVYAYGPDWMARPSTWSAEIQQFLRLLGATYVLRVEMGRQFGFEVHRSRPSFRQ FQAINHLVLFDNALRKYDSGQVAAGFQRALLVAGPETADTRPDLRKLNEWVFGGR AAGGRQLADELKIVSALRDTYSGHLVLQPTETLDTWKVLSRDTRTAHSLEHGFIHAA GTIQANCPQLFMRRQHPGLFPFVNAIASSLGWYYQTATGPGADARAAARRQQAFQ TRAAAECHAKSGVPVVAGFYRTINATLKGGEGLQPTMFNGELGAIKHQALDTVRYD YGHYLIMLGPFQPWSGLTAPPCPYAESSWAQAAVQTALELFSALYPAPCISGYARP PGPSAVIEHLGSLVPKGGLLLFLSHLPDDVKDGLGEMGPARATGPGMQQFVSSYF LNPACSNVFITVRQRGEKINGRTVLQALGRACDMAGCQHYVLGSTVPLGGLNFVN DLASPVSTAEMMDDFSPFFTVEFPPIQEEGASSPVPLDVDESMDISPSYELPWLSL ESCLTSILSHPTVGSKEHLVRHTDRVSGGRVAQQPGVGPLDLPLADYAFVAHSQV WTRPGGAPPLPYRTWDRMTEKLLVSAKPGGENVKVSGTVITLGEQGYKVSLDLRE GTRLAMAEALLNAACAPILDPEDVLLTLHLHLDPRRADNSAVMEAMTAASDYARGL GVKLTFGSASCPETGSSASNFMTVVASVSAPGEFSGPLITPVLQKTGSLLIAVRCGD GKIQGGSLFEQLFSDVATTPRAPEALSLKNLFRAVQQLVKSGIVLSGHDISDGGLVT CLVEMALAGQRGVTITMPVASDYLPEMFAEHPGLVFEVEERSVGEVLQTLRSMNM YPAVLGRVGEQGPDQMFEVQHGPETVLRQSLRLLLGTWSSFASEQYECLRPDRIN RSMHVSDYGYNEALAVSPLTGKNLSPRRLVTEPDPRCQVAVLCAPGTRGHESLLA AFTNAGCLCRRVFFREVRDNTFLDKYVGLAIGGVHGARDSALAGRATVALINRFPA LRDAILKFLNRPDTFSVALGELGVQVLAGLGAVGSTDNPPAPGVEVNVQRSPLILAP NASGMFESRWLNISIPATTSSVMLRGLRGCVLPCWVQGSCLGLQFTNLGMPYVLQ NAHQIACHFHSNGTDAWRFAMNYPRNPTEQGNIAGLCSRDGRHLALLCDPSLCTD FWQWEHIPPAFGHPTGCSPWTLMFQAAHLWSLRHGRPSE (SEQ ID NO: 117); or a functional variant thereof that is at least 80%, at least 90%, at least 95%, at least 96%, at least 97%, at least 98%, at least 99% identical or 100% identical to the polypeptide sequence described herein.

In some preferred embodiments of the immunogenic composition of the invention, the EBV-VLP comprises one, two, three, four, or more EBV proteins selected from the group consisting of gp350 (UniProt Entry P03200), BKRF4 (UniProt Entry P30117), BVRF1 (UniProt Entry P03233), BDLF3 (UniProt Entry P03224), BZLF2 (UniProt Entry P03205), BXLF2 (UniProt Entry P03231), BNRF1 (UniProt Entry P03179), BALF4 (UniProt Entry P03188) and BZLF1 (UniProt Entry P03206); or functional variants thereof that is at least 80%, at least 90%, at least 95%, at least 96%, at least 97%, at least 98%, at least 99% identical or 100% identical to the polypeptide sequence described herein.

In some preferred embodiments of the immunogenic composition of the invention, the EBV-VLP comprises the EBV proteins of gp350 (UniProt Entry P03200), BKRF4 (UniProt Entry P30117), BVRF1 (UniProt Entry P03233), BDLF3 (UniProt Entry P03224), BZLF2 (UniProt Entry P03205), BXLF2 (UniProt Entry P03231), BNRF1 (UniProt Entry P03179), BALF4 (UniProt Entry P03188) and BZLF1 (UniProt Entry P03206), or their functional variants thereof that are at least 80%, at least 90%, at least 95%, at least 96%, at least 97%, at least 98%, at least 99% identical or 100% identical to the polypeptide sequences described herein, wherein the presence of more than one the said EBV proteins contributes to enhanced immunogenicity of the VLP vaccine.

In some preferred embodiments of the immunogenic composition of the invention, the EBV-VLP does not comprise LMP1 (UniProt Entry P03230), EBNA2 (UniProt Entry P12978), EBNA3a (UniProt Entry P12977), EBNA3b (UniProt Entry P03203) and EBNA3c (UniProt Entry P03204), wherein the absence of these B-cell transformation EBV proteins contributes to enhanced safety of the VLP vaccine.

TABLE A
summarizes some of the EBV proteins that comprised in the one preferred
EBV VLP of the invention:

| | Amino acid sequence |
|---|---|
| gp350 | SEQ ID NO: 102 |
| (UniProt | |
| Entry | |
| P03200) | |
| BKRF4 | MAMFLKSRGVRSCRDRRLLSDEEEETSQSSSYTLGSQASQSIQEEDVS |
| (UniProt | DTDESDYSDEDEEIDLEEEYPSDEDPSEGSDSDPSWHPSDSDESDYSE |
| Entry | SDEDEATPGSQASRSSRVSPSTQQSSGLTPTPSFSRPRTRAPPRPPAP |
| P30117) | APVRGRASAPPRPPAPVQQSTKDKGPHRPTRPVLRGPAPRRPPPPSSP |
| | NTYNKHMMETTPPIKGNNNYNWPWL |
| BVRF1 | MALSGHVLIDPARLPRDTGPELMWAPSLRNSLRVSPEALELAEREAERA |
| (UniProt | RSERWDRCAQVLKNRLLRVELDGIMRDHLARAEEIRQDLDAVVAFSDGL |
| Entry | ESMQVRSPSTGGRSAPAPPSPSPAQPFTRLTGNAQYAVSISPTDPPLMV |
| P03233) | AGSLAQTLLGNLYGNINQWVPSFGPWYRTMSANAMQRRVFPKQLRGNL |
| | NFTNSVSLKLMTEVVAVLEGTTQDFFSDVRHLPDLQAALILSVAYLLLQG |
| | GSSHQQRPLPASREELLELGPESLEKIIADLKAKSPGGNFMILTSGNKEA |
| | RQSIAPLNRQAAYPPGTFADNKIYNLFVGAGLLPTTAALNVPGAAGRDR |
| | DLVYRIANQIFGEDVPPFSSHQWNLRVGLAALEALMLVYTLCETANLAEA |
| | ATRRLHLSSLLPQAMQRRKPAMASAGMPGAYPVQTLFRHGELFRFIWA |
| | HYVRPTVAADPQASISSLFPGLVLLALELKLMDGQAPSHYAINLTGQKF |
| | DTLFEIINQKLLFHDPAAMLAARTQLRLAFEDGVGVALGRPSPMLAARE |
| | ILERQFSASDDYDRLYFLTLGYLASPVAPS |
| BDLF3 | MAHARDKAGAVMAMILICETSLIWTSSGSSTASAGNVTGTTAVTTPSPS |
| (UniProt | ASGPSTNQSTTLTTTSAPITTTAILSTNTTTVTFTGTTVTPVPTTSNASTIN |
| Entry | VTTKVTAQNITATEAGTGTSTGVTSNVTTRSSSTTSATTRITNATTLAPTL |
| P03224) | SSKGTSNATKTTAELPTVPDERQPSLSYGLPLWTLVFVGLTFLMLILIFAA |
| | GLMMSAKNKPLDEALLTNAVTRDPSLYKGLV |
| BZLF2 | MVSFKQVRVPLFTAIALVIVLLLAYFLPPRVRGGGRVAAAAITWVPKPNVE |
| (UniProt | VWPVDPPPPVNFNKTAEQEYGDKEVKLPHWTPTLHTFQVPQNYTKANC |
| Entry | TYCNTREYTFSYKGCCFYFTKKKHTWNGCFQACAELYPCTYFYGPTPDIL |
| P03205) | PVVTRNLNAIESLWVGVYRVGEGNWTSLDGGTFKVYQIFGSHCTYVSKF |
| | STVPVSHHECSFLKPCLCVSQRSNS |
| BXLF2 | SEQ ID NO: 109 |
| (UniProt | |
| Entry | |
| P03231) | |
| or | |
| BNRF1 | SEQ ID NO: 114 |
| (UniProt | |
| Entry | |
| P03179) | |
| BALF4 | MTRRRVLSVVVLLAALACRLGAQTPEQPAPPATTVQPTATRQQTSFPFRVC |
| (UniProt | ELSSHGDLFRFSSDIQCPSFGTRENHTEGLLMVFKDNIIPYSFKVRSYTKIVT |
| Entry | NILIYNGWYADSVTNRHEEKFSVDSYETDQMDTIYQCYNAVKMTKDGLTRV |
| P03188) | YVDRDGVNITVNLKPTGGLANGVRRYASQTELYDAPGWLIWTYRTRTTVNC |
| | LITDMMAKSNSPFDFFVTTTGQTVEMSPFYDGKNKETFHERADSFHVRTNY |
| | KIVDYDNRGTNPQGERRAFLDKGTYTLSWKLENRTAYCPLQHWQTFDSTIA |
| | TETGKSIHFVTDEGTSSFVTNTTVGIELPDAFKCIEEQVNKTMHEKYEAVQDR |
| | YTKGQEAITYFITSGGLLLAWLPLTPRSLATVKNLTELTTPTSSPPSSPSPPAP |
| | SAARGSTPAAVLRRRRRDAGNATTPVPPTAPGKSLGTLNNPATVQIQFAYD |
| | SLRRQINRMLGDLARAWCLEQKRQNMVLRELTKINPTTVMSSIYGKAVAAKR |
| | LGDVISVSQCVPVNQATVTLRKSMRVPGSETMCYSRPLVSFSFINDTKTYEG |
| | QLGTDNEIFLTKKMTEVCQATSQYYFQSGNEIHVYNDYHHFKTIELDGIATLQ |
| | TFISLNTSLIENIDFASLELYSRDEQRASNVFDLEGIFREYNFQAQNIAGLRKD |
| | LDNAVSNGRNQFVDGLGELMDSLGSVGQSITNLVSTVGGLFSSLVSGFISFF |
| | KNPFGGMLILVLVAGVVILVISLTRRTRQMSQQPVQMLYPGIDELAQQHASG |
| | EGPGINPISKTELQAIMLALHEQNQEQKRAAQRAAGPSVASRALQAARDRFP |
| | GLRRRRYHDPETAAALLGEAETEF |
| BZLF1 | MMDPNSTSEDVKFTPDPYQVPFVQAFDQATRVYQDLGGPSQAPLPCVLWP |
| (UniProt | VLPEPLPQGQLTAYHVSTAPTGSWFSAPQPAPENAYQAYAAPQLFPVSDITQ |
| Entry | NQQTNQAGGEAPQPGDNSTVQTAAAVVFACPGANQGQQLADIGVPQPAPV |
| P03206) | AAPARRTRKPQQPESLEECDSELEIKRYKNRVASRKCRAKFKQLLQHYREVA |
| | AAKSSENDRLRLLLKQMCPSLDVDSIIPRTPDVLHEDLLNF |

The term “individual,” used interchangeably herein with “host,” “subject,” and includes humans, non-human primates, all domestic livestock and pets, wild mammals and fowl, including, without limitation, cattle, horses, cows, swine, sheep, goats, dogs, cats, rabbits, deer, mink, chickens, ducks, geese, turkeys, game hens, and the like.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some examples, said cancer is selected from the group consisting of B-cell lymphoma, T-cell lymphoma, multiple myeloma, chronic myeloid leukemia (CML), acute myeloma leukemia (AML), myelodysplastic syndromes (MDS), chronic myeloproliferative neoplasms (MPN), B-cell acute lymphoblastic leukemia (B-ALL), solid tumor, carcinoma, or sarcoma; and preferably said cancer is a solid tumor. In another examples, said cancer is a virus-specific cancers. In some examples, said cancer is an EBV-associated cancer, selected from a lymphoproliferative disorder (LPD), such as B-cell lymphoma, including Burkitt lymphoma (BL), Hodgkin lymphoma (HL), a diffuse large B cell lymphoma (DLBCL), T-cell lymphoma, NKT-cell lymphoma, NK-cell lymphoma, or a post-transplant lymphoproliferative disorder (PTLD), or an epithelial carcinoma (nasopharyngeal, lung, breast), a lymphoepithelioma, a carcinoma with lymphoid stroma (GCLS, e.g. gastric carcinoma) or a glioma.

DLBCL is a cancer of B cells. Typically, DLBCL arises from normal B cells, but it can also represent a malignant transformation of other types of lymphoma or leukemia. An underlying immunodeficiency is a significant risk factor and infection with Epstein-Barr virus has also been found to contribute to the development of DLBCL. In one embodiment, the herpes virus-associated cancer is DLBCL.

Burkitt lymphoma is a cancer of the lymphatic system, particularly B lymphocytes found in the germinal center. Burkitt lymphoma can be divided into three main clinical variants: the endemic, the sporadic, and the immunodeficiency-associated variants. EBV infection is found in nearly all endemic variants. In one embodiment, the herpes virus-associated cancer is Burkitt lymphoma.

Also relevant within the context of the disclosure is the treatment or prevention of a post-transplant lymphoproliferative disorder (PTLD), an epithelial carcinoma (nasopharyngeal, lung, breast), a lymphoepithelioma, a carcinoma with lymphoid stroma (GCLS, e.g. gastric carcinoma) or a glioma. PTLD is the name given to a B-cell proliferation due to therapeutic immunosuppression after organ transplantation. These patients may develop infectious mononucleosis-like lesions or polyclonal polymorphic B-cell hyperplasia. The disclosure therefore also relates to the treatment of immune deficient or immune compromised patients after chemotherapy, radiation, immune suppression or transplantation. In one embodiment, the herpes virus-associated cancer is PTLD.

Nasopharynx cancer or nasopharyngeal carcinoma (NPC) is the most common cancer originating in the nasopharynx, most commonly in the postero-lateral nasopharynx or pharyngeal recess, accounting for 50% cases. NPC occurs in children and adults. The association between Epstein-Barr virus and nasopharyngeal carcinoma is unequivocal in World Health Organization (WHO) types II and III tumors. In one embodiment, the herpes virus-associated cancer is NPC.

Lymphoepithelioma is a type of poorly differentiated nasopharyngeal carcinoma characterized by prominent infiltration of lymphocytes in the area involved by tumor. Lymphoepithelioma is also known as “class III nasopharyngeal carcinoma” in the WHO classification system. In one embodiment, the herpes virus-associated cancer is lymphoepithelioma.

Gastric carcinoma with lymphoid stroma (GCLS) is a distinct histologic subtype of gastric cancer that is characterized by undifferentiated carcinoma mixed with prominent lymphoid infiltration. More than 80% of GCLS cases are associated with EBV infection, but it is unclear if the virus affects disease progression. In one embodiment, the herpes virus-associated cancer is GCLS.

A glioma is a type of tumor that starts in the glial cells of the brain or the spine. Gliomas comprise about 30 percent of all brain tumors and central nervous system tumors, and 80 percent of all malignant brain tumors. Studies have revealed that EBV is present in elevated frequencies in glioma patients, indicating potential targeting of EBV associated glioma using the present disclosure. In one embodiment, the herpes virus-associated cancer is glioma.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the compositions, peptides, and/or cells in prevention of the occurrence of cancer in the first place.

The term “specifically binds,” refers to an antigen-binding domain (antibody), or a ligand, which recognizes and binds with a cognate binding partner (e.g., a stimulatory and/or costimulatory molecule present on a T cell) protein present in a sample, but which antigen-binding domain (antibody), or ligand does not substantially recognize or bind other molecules in the sample.

The term “Chimeric Antigen Receptor” or “CAR” refers to a recombinant polypeptide construct comprising at least an extracellular domain which comprises an antigen binding domain, a transmembrane domain and a cytoplasmic signaling domain (also referred to herein as “an intracellular signaling domain” or “intracellular domain”) comprising a functional signaling domain derived from a stimulatory molecule as defined below. In some embodiments, the domains in the CAR polypeptide construct are in the same polypeptide chain, e.g., comprise a chimeric fusion protein.

In one aspect, the intracellular domain comprises at least one activating domain (e.g., an activating domain of CD3-zeta). In one aspect, the intracellular domain further comprises one or more costimulatory derived from at least one costimulatory molecule as defined below. In one aspect, the costimulatory molecule is chosen from 41BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., an scFv) during cellular processing and localization of the CAR to the cellular membrane.

A CAR that comprises an antigen binding domain (e.g., an scFv, a single domain antibody, or TCR (e.g., a TCR alpha binding domain or TCR beta binding domain)) that targets a specific tumor marker X, wherein X can be a tumor marker as described herein, is also referred to as XCAR. For example, a CAR that comprises an antigen binding domain that targets CD 19 is referred to as CD19CAR. The CAR can be expressed in any cell, e.g., an immune effector cell as described herein (e.g., a T cell or an NK cell).

The term “signaling domain” refers to the functional portion of a protein which acts by transmitting information within the cell to regulate cellular activity via defined signaling pathways by generating second messengers or functioning as effectors by responding to such messengers. The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VF or VH), camelid VHH domains, and multi-specific molecules formed from antibody fragments such as a bivalent fragment comprising two or more, e.g., two, Fab fragments linked by a disulfide bond at the hinge region, or two or more, e.g., two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VF and VH variable regions in either order, e.g., with respect to the N-terminal and C-terminal ends of the polypeptide, the scFv may comprise VF-linker-VH or may comprise VH-linker-VF.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (FCDR1, FCDR2, and FCDR3). The precise amino acid sequence boundaries of a given CDR can be determined using any of a number of well-known schemes, including those described by Rabat et al. (1991), “Sequences of Proteins of Immunological Interest,” 5th Ed. Public Health Service, National Institutes of Health, Bethesda, MD (“Rabat” numbering scheme), Al-Fazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3).

Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the disclosure comprising an antibody or antibody fragment thereof may exist in a variety of forms, for example, where the antigen binding domain is expressed as part of a polypeptide chain including, for example, a single domain antibody fragment (sdAb), a single chain antibody (scFv), or e.g., a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR composition of the disclosure comprises an antibody fragment. In a further aspect, the CAR comprises an antibody fragment that comprises an scFv.

As used herein, the term “binding domain” or “antibody molecule” (also referred to herein as “anti-target binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, said antigen-binding domain is a scFv domain.

The term “autologous” refer to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In one aspect, the primary signal is initiated by, for instance, binding of a TCR/CD3 complex with an MF1C molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FceRI and CD66d, DAP10 and DAP12. In a specific CAR of the disclosure, the intracellular signaling domain in any one or more CARS of the disclosure comprises an intracellular signaling sequence, e.g., a primary signaling sequence of CD3-zeta. The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain” or “intracellular domain” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FceRI, CD66d, DAP 10 and DAP12. The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (e.g., a molecule having mutations, e.g., point mutations, fragments, insertions, or deletions). In one embodiment, the cytoplasmic domain of zeta comprises residues 52 through 164 of GenBank Ace. No. BAG36664.1 or a variant thereof (e.g., a molecule having mutations, e.g., point mutations, fragments, insertions, or deletions). In one embodiment, the “zeta stimulatory domain” or a “CD3-zeta stimulatory domain” is the sequence provided as SEQ ID NO: 641 or 643 or a variant thereof (e.g., a molecule having mutations, e.g., point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof. The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (e.g., a molecule having mutations, e.g., point mutations, fragments, insertions, or deletions).

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “expression vector” refers to a vector comprising a recombinant polynucleotide comprising expression control sequences operatively linked to a nucleotide sequence to be expressed.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions, e.g., conservative substitutions), alleles, orthologs, SNPs, and complementary sequences as well as the sequence explicitly indicated. Specifically, degenerate codon substitutions, e.g., conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present disclosure includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by ah nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-Al or HLA-A2 have been described (see, e.g., Sastry et al., J Virol. 2011 85(5): 1935-1942; Sergeeva et al., Blood, 2011 117(16):4262-4272; Verma et al., J Immunol 2010 184(4):2156-2165; Willemsen et al., Gene Ther 2001 8(21): 1601-1608; Dao et al., Sci Transl Med 2013 5(176):l76ra33; Tassev et al., Cancer Gene Ther 2012 19(2):84-100). For example, TCR-like antibody can be identified from screening a library, such as a human scFv phage displayed library.

The term “subject” is intended to include living organisms in which an immune response can be elicited (e.g., mammals, human)

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

### Composition and Medical Use

In some aspects, the present disclosure provides compositions for improving immune cell therapy, wherein said compositions comprising: (a) a polyinosinic-polycytidylic acid (PIC), (b) a stabilizer, (c) at least one cation. In some embodiments, said compositions are useful for the eliciting, inducing, enhancing and/or potentiating an immune response, which may be an innate and/or adaptive immune response mediated by an immune cell therapy. In some embodiments, said immune cell is immune effector cells (e.g., T cells or NK cells) that express a chimeric antigen receptor (CAR) molecule, e.g., a CAR molecule that binds to a tumor antigen, e.g., an antigen expressed on the surface of a solid tumor or a hematological tumor.

In some embodiments, the present disclosure provides an immunogenic composition comprising: (a) a polyinosinic-polycytidylic acid (PIC), (b) a stabilizer which is an aminoglycoside antibiotic or non-aminoglycoside amine, (c) at least one cation which is preferably calcium. In some embodiments, said composition further comprises at least one immunogen or antigen. In some embodiments, said immunogen or antigen is a recombinant protein, virus-like particle (VLP), peptide, mRNA or vaccine.

In some embodiments, the present disclosure relates to an immunogenic composition comprising a VLP of Epstein Barr virus (EBV). Said immunogenic composition may be used for inducing activation and/or increasing of an immune response in an individual, such as immune response of an immune cell therapy (preferably CAR-T cell therapy) for cancer treatment. Said immunogenic composition may be used in combination with an engineered immune cell (e.g. CAR-T cell or engineered TCR-T cell), in the treatment of cancer. In some examples, said immunogenic composition is to be administered in combination (simultaneously or sequentially) with an engineered immune cell, preferably CAR-T cell, more preferably a CAR-T cell that targets EBV-associated cancers.

In some embodiments, said immunogenic composition may be for use in inducing activation and/or increasing of an immune response in an individual, such as immune response of an immune cell therapy (preferably CAR-T cell therapy) for cancer treatment. In some further embodiments, said immunogenic composition may be also useful for use in combination with an engineered immune cell (preferably CAR-T cell or engineered TCR-T cell), in the treatment of cancer. In one embodiment, said composition immunogenic increases anti-cancer effect of the cell therapy.

PIC is typically a double stranded polymer comprising one strand of inosinic acid polymer (polyinosinic acid; polyI) and one strand of cytidylic acid polymer (polycytidylic acidpolyC). The polymer backbone may be a deoxyribonucleic acid backbone or ribonucleic acid backbone. The PIC may be an oligonucleotide analog. In a preferred embodiment, the polyI is preferably polyriboinosinic acid. In a preferred embodiment, the PolyC is preferably polyribocytidylic acid. In preferred embodiments the PIC is polyriboinosinic: polyribocytidylic acid, i.e. a double stranded RNA (dsRNA)-like polymer.

In some embodiments, the concentration of the PIC is 0.5 mg/ml to 10 mg/ml.

In some embodiments, the PIC are heterogeneous for molecular weight, where the average molecular weight is equal to or greater than 66,000 Daltons. The value of 66,000 Daltons corresponds to the molecular size of 6.4 sedimentation unit (Svedbergs). In some embodiments, the PIC has an average molecular weight equal to or greater than 150,000 Daltons or average molecular size equal to or greater than 9.3 Svedbergs. In some embodiments, the PIC has an average molecular weight equal to or greater than 250,000 Daltons or average molecular size equal to or greater than 11.8 Svedbergs. In some embodiments, the PIC has an average molecular weight equal to or greater than 350,000 Daltons or average molecular size equal to or greater than 15.3 Svedbergs.

In some embodiments, the molecular weight of PIC is from 66,000 to 2,000,000 Daltons. In some embodiments, the molecular weight of PIC is from 66,000 to 1,200,000 Daltons (equivalent to 6.4 to 24.0 sedimentation unit). In some embodiments, the molecular weight of PIC is from 66,000 to 660,000 Daltons or molecular size range from about 6.4 to 18.3 Svedbergs. In some embodiments, the PIC has a molecular weight range from about 300,000 to 1,200,000 Daltons or size from about 6.4 to 24.0 Svedbergs. In some embodiments, the PIC has a molecular weight range from about 300,000 to 660,000 Daltons or molecular size range from about 12.8 to 18.3 Svedbergs. In some other embodiments, the molecular weight of PIC is from 100,000 to 200,000 Daltons, or from 300,000 to 4,000,000 Daltons, or from 500,000 to 1,000,000 Daltons, or from 1,000,000 to 1,500,000 Daltons, or from 1,500,000 to 2,000,000 Daltons, or from 2,000,000 to 2,500,000 Daltons, or from 2,500,000 to 3,000,000 Daltons, or from 3,000,000 to 3,500,000 Daltons, or from 3,500,000 to 4,000,000 Daltons, or from 4,000,000 to 4,500,000 Daltons, or from 4,500,000 to 5,000,000 Daltons.

In some embodiments, PIC compositions is obtained by mixing the polyinosinic and polycytidylic acid in a certain ratio. Preferably, the ratio may be 0.5:1.0, 0.6:1.0, 0.7:1.0, 0.8:1.0, 0.9:1.0, 1.0:1.0, 1.0:1.1, 1.0:1.2, 1.0:1.3, 1.0:1.4, 1.0:1.5, 1.5:1.0, 1.4:1.0, 1.3:1.0, 1.2:1.0, 1.1:1.0, 1.0:0.9, 1.0:0.8, 1.0:0.7, 1.0:0.6, or 1.0:0.5. In a preferred embodiment, the ratio is 1:1. Moreover, the mixture is further mixed with 200 to 2000 IU of kanamycin, and 0.02 to 10 mM CaCl₂).

In some particular embodiments, the stabilizer is selected from the group consisting of tacrolamycin, anthracycline, butyrin sulphate, gentamicin, hygromycin, amikacin, dideoxy kanamycin, nebramycin, β-lactam, neomycin, puromycin, streptomycin, streptozocin, and any combination thereof. The polyamine compound is selected from the group consisting of arginine salt, spermidine, N-(3-aminopropyl), N-(3-aminopropyl)-1,4-butanediamine, spermine, OS-dimethylaminothiophosphate, poly-lysine, aminoglycoside, and any combination thereof. In some particular embodiments, the stabilizer is kanamycin. In another particular embodiments, the stabilizer is ε-polylysine, hexylglucosamine a polyethylene glycol monomethyl ether, polyethylene glycol, polyethyleneimine, folic acid, and galactose or acetylglucosamine. In some embodiments, the concentration of the stabilizer in the composition is from 10 unit/ml to 100,000 unit/ml, preferably from 100 unit/ml to 10,000 unit/ml, more preferably from 500 unit/ml to 5,000 unit/ml.

In some embodiments, the positive ion is a cation and is selected from the group consisting of calcium, cadmium, lithium, magnesium, cerium, cesium, chromium, cobalt, deuterium, gallium, iodine, iron, zinc, and any combination thereof. In some particular embodiments, the positive ion is calcium. The positive ion may be in the form of any suitable salt or organic complex including, but not limited to, chloride, fluoride, hydroxide, phosphate or sulfate. For example, when the positive ion is calcium, the calcium ion may be in the form of calcium carbonate, calcium chloride, calcium fluoride, calcium hydroxide, calcium phosphate or calcium sulfate. In some embodiments, the concentration of the positive ion in the composition is from 0.01 pmol to 10 mmol/ml, preferably from 0.02 pmol to 5 mmol/ml, more preferably from 0.1 pmol to 1 mmol/ml, most preferably from 0.1 pmol to 100 pmol/ml.

In some embodiments, the immunogen is a recombinant protein. In some embodiments, the immunogen is a virus-like particle (VLP). In some embodiments, the immunogen is a peptide vaccine. In some embodiments, the immunogen is a mRNA vaccine. In some other embodiments, the immunogen can be one or more polypeptides/peptides derived from cancer cells or antigenic fragments or variants thereof. It will be understood that the immunogen described herein may further comprise additional components. For example, one or more immunogen may be comprised in a lipid or liposome. In some embodiments, a peptide or polypeptide corresponding to a cancer antigen may generally be 10-20 amino acid residues in length, and may contain more than one peptide determinants or up to about 30-50 residues or so. In some embodiments, the polypeptide is between 10 and about 150 residues or more in length. In some embodiments, longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present disclosure. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell. In some embodiments, the immunogen is identical or derived from the same antigen recognized by the CAR.

In some embodiments, the immunogen is a “tumor-associated antigen” or “cancer antigen” and may be selected from CTA, NY-ESO-1, LAGE-1, MAGE-A1, MAGE-A3, MAGE-A4, MAGE-A10, CT7, CT10, GAGE, PRAME; BAGE; RAGE, SAGE, HAGE, MPHOSPH1, DEPDC1, IMP3 and MAGE-A, and T-antigen BK, p53, Ras, c-Myc, A-Raf, B-Raf, C-Raf, cyclin-dependent kinases, MAGE-A2, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, tyrosinases, TRP-1, TRP-2, ART-4, CAMEL, CEA, Cyp-B, hTERT, hTRT, ICE, MUC1, MUC2, phosphoinositide 3-kinases (PI3K), TRK, PRAME, P15, RU1, RU2, SART-1, SART-3 receptors, Wilms tumor antigen (WT1), AFP, β-catenin/ω, caspases-8/m, CEA, CDK-4/m, ELF2M, GnT-V, G250, HSP70-2M, HST-2, KIAA0205, MUM-1, MUM-2, MUM-3, myosin/m, RAGE, SART-2, TRP-2/INT2, 707-AP, annexin II, CDC27/m, TPI/mbcr-abl, BCRABL, interferon regulatory factor 4 (IRF4), ETV6/AML, LDLR/FUT, Pml/RARa, tumor-associated calcium signal transducer 1 (TACSTD1) TACSTD2 receptor tyrosine kinases, epidermal growth factor receptor (EGFR), EGFRvIII, platelet growth factor receptor (PDGFR), vascular endothelial growth factor receptor ia (VEGFR), cytoplasmic tyrosine kinases, src family, syk-ZAP70, integrin-linked kinase (ILK), signal transducers and transcription activators STAT3, STAT5 and STAT6, factors induced by hypoxia, HIF-1a and HIF-2a, nuclear factor kappa in (NF-kB), Notch, Notch1-4, c-Met receptors, mammalian rapamycin targets (mTOR), WNT, extracellular signal-regulated kinases (ERK), PMSA, PR-3, MDM2, mesothelin, cancer kidney cells-5T4, SM22-alpha, carbonic anhydrase I (CAI) and IX (CAIX), STEAD, TEL/AML1, GD2, proteinase 3, hTERT, sarcoma translocation control points, EphA2, ML-IAP, EpCAM, ERG (fusion gene TMPRSS2 ETS), NA17, PAX3, ALK, androgen receptor, zclin B1, polysialic acid, MYCN, RhoC, GD3, fucosyl GM1, mesotheliana, PSCA, sLe, PLAC1, GM3, BORIS, Tn, GLoboH, NYBR-1, RG SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2 and fos-related antigen 1.

In yet another embodiment, the immunogen is a viral antigen from an oncogenic virus. Typical oncogenic viruses include, but are not limited to, EBV, HPV, HBV, HCV, HTLV, and KSHV. Typical viral antigens from oncogenic viruses that can be used in the disclosure include, but are not limited to, EBV: EBNA-1, LMP-1, LMP-2A; HPV: E6, E7, E5; HBV: HBx; HCV: Core, NS3, Ns5A; HTLV: Tax, HBZ; KSHV: vFLIP, LANA, VGPCR, vIRF-1.

In some embodiments, the immunogen is a virus-like particle (VLP). In another embodiments, the immunogen is a VLP of Epstein Barr virus (EBV), preferably said VLP comprises glycoprotein 350/220 (gp350) protein or fragment thereof.

In some embodiments, the composition is administered by parenteral administration, intramuscular injection, intraperitoneal perfusion, intrapleural perfusion, intravenous perfusion, subcutaneous injection, intrapericardial injection, inhalation, endorectal perfusion, suppository, intra nasal, ophthalmic, transdermal or oral administration.

In some embodiments, PIC is preferably synthetic and is preferably obtained by de novo chemical synthesis. Synthetic PIC molecules may be obtained from commercial suppliers (e.g. Sigma, or Midland Certified). PIC molecules may be synthesised with or chemically modified to contain 2′-position modifications such as 2′ O-methyl, 2′-Fluoro or 2′-NH₂.

In some embodiments, the immune cell described herein may be selected from the group consisting of a T lymphocyte, an NK cell, a macrophage and a dendritic cell, wherein the T lymphocyte preferably is a cytotoxic T lymphocyte or a T helper cell, more preferably a cytotoxic T lymphocyte. The immune cell may be a T lymphocyte. The T lymphocyte may be a cytotoxic T lymphocyte. The T lymphocyte may be a T helper cell. The immune cell may be an NK cell. The immune cell may be a macrophage. The immune cell may be dendritic cell. These immune cells are known in the field to exhibit cytotoxic and/or other beneficial activity in response to unwanted agents, cells or pathogens such as cells infected by a herpes virus. By directing the activity of these cells to particular immunogenic targets, namely the herpes viral antigens described herein, infected pathogenic cells can be eliminated by the corresponding activity of the immune cell described herein.

### Chimeric Antigen Receptor (CAR)

In one aspect, disclosed herein are methods using a cell (e.g., a population of cells) that expresses a CAR molecule. In one aspect, an exemplary CAR construct comprises an optional leader sequence (e.g., a leader sequence described herein), an antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

### CAR Antigen Binding Domain

In one aspect, the portion of the CAR comprising the antigen binding domain comprises an antigen binding domain that targets a tumor antigen, e.g., a tumor antigen described herein. In some embodiments, the antigen binding domain binds to: CD19; CD123; CD22; CD30; CD171; CS-1; C-type lectin-like molecule-1, CD33; epidermal growth factor receptor variant III (EGFRvIII); ganglioside G2 (GD2); ganglioside GD3; TNF receptor family member; B-cell maturation antigen (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Fike Tyrosine Kinase 3 (FFT3); Tumor-associated glycoprotein 72 (TAG72); CD38; CD44v6; Carcinoembryonic antigen (CEA); Epithelial cell adhesion molecule (EPCAM); B7H3 (CD276); KIT (CD 117); Interleukin-13 receptor subunit alpha-2; Mesothelin; Interleukin 11 receptor alpha (IL-11Ra); prostate stem cell antigen (PSCA); Protease Serine 21; vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1; tyrosinase; ephrin type-A receptor 2 (EphA2); Fucosyl GM1; sialyl Lewis adhesion molecule (sLe); ganglioside GM3; transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA 17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1, melanoma antigen recognized by T cells 1; Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Fike, Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (FCK); A kinase anchor protein 4 (AKAP-4); synovial sarcoma, X breakpoint 2 (SSX2); Receptor for Advanced Glycation Endproducts (RAGE-I); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In some embodiments, said antigen that is recognized by the CAR is a virus antigen. In some embodiments, said antigen that is recognized by the CAR is an Epstein-Barr virus antigen (EBV antigen). In some embodiments, said EBV antigen may be present on the surface of EBV-infected cells, preferably EBV-infected cancer cells, EBV-infected B cells or EBV-infected epithelial cells. The EBV antigen may be an EBV virion envelope protein or a protein of the EBV envelope complex (such as gB, gL, or gH). The EBV virus antigen preferably is the EBV glycoprotein 350/220 (gp350/220). In preferred embodiments the disclosure is focused on targeting EBV-antigens and treating EBV-associated medical conditions. An exemplary EBV gp350 protein is shown in the UniProt database, entry P03200-1, version 1 of 21 Jul. 1986. An exemplary EBV gp220 is shown in the same database entry but positions 502 to 750 are missing.

In some embodiments, the CAR of the disclosure comprises an antigen-binding domain comprising a heavy chain variable region (VH) and/or light chain variable region (VL) as provided in Table 1 and Table 2, or a specific VH and VL combination as provided in Table 1 and Table 2. In another embodiments, the CAR comprises an antigen-binding domain comprising a heavy chain variable region (VH) and/or light chain variable region (VL) which have at least 60%, 70%, 80%, 85%, 90%, 95%, 99% identity of the VH and/or VL amino acid sequences as provided in Table 1 and Table 2. In another embodiments, the CAR comprises an antigen-binding domain that binds to the same epitope on EBV gp350 or competitively binds to EBV gp350 with comprises an antigen-binding domain comprising a VH and VL specific combination as provided in Table 1 and Table 2.

In some embodiments, the CAR of the disclosure comprises an antigen-binding domain which binds EBV glycoprotein 350/220 (gp350/220), wherein said antigen-binding domain comprises:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO: 4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO: 5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence.
(6) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO: 138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO: 139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence; or
(15) a heavy chain variable region (VH) having the polypeptide sequence of SEQ ID NO: 140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence.

In some embodiments, the CAR of the disclosure comprises an antigen-binding domain comprising one, two, three or more HCDRs and/or one, two, three or more LCDRs as provided in Table 3 and Table 4, or a specific HCDR1-3 and LCDR1-3 combination as provided in Table 3 and Table 4.

In some embodiments, the CAR comprises an antigen-binding domain which binds EBV glycoprotein 350/220 (gp350/220), wherein said antigen-binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively; or
(5) SEQ ID NOs: 23, 24, 25, 38, 39 and 40, respectively; or
   an EBV glycoprotein 350/220 antigen-binding domain which competes therewith or binds to the same epitope as that in the EBV glycoprotein 350/220 to which any one of the antigen binding domain (1) to (5) of the CAR as described herein bind.

In some embodiments, the CAR of the disclosure comprising amino acid sequence as provided in Table 5, preferably the CAR comprises an amino acid sequence selected from the group consisting of SEQ ID NO: 144 to 161. In another embodiments, the CAR comprises at least 60%, 70%, 80%, 85%, 90%, 95%, 99% or 100% identity to the CAR sequences as provided in Table 5, preferably the CAR is selected from the group consisting of SEQ ID NO: 144 to 161

In yet another embodiments, the CAR of the disclosure is characterized in that the antigen-binding domain comprises a variable heavy chain (VH), said VH comprising:
heavy chain complementary determining regions H-CDR1 according to SEQ ID NO: 118 (GLSLTSN), H-CDR2 according to SEQ ID NO: 119 (WSNGG), and H-CDR3 according to SEQ ID NO: 120 (PRYNSGYFFDY), or one or more corresponding CDR sequences of at least 80% sequence identity to SEQ ID NOs 118 to 120;
and a variable light chain (VL), said VL comprising:
light chain complementary determining regions L-CDR1 according to SEQ ID NO: 121 (KASESVSTRMH), L-CDR2 according to SEQ ID NO: 122 (KTSNLAS), and L-CDR3 according to SEQ ID NO: 123 (QQSWNGPLT), or one or more corresponding CDR sequences of at least 80% sequence identity to SEQ ID NOs 121 to 123.

In yet another embodiments, the CAR of the disclosure is characterized in that the antigen-binding domain comprises a variable heavy chain (VH), said VH comprising:
heavy chain complementary determining regions H-CDR1 according to SEQ ID NO: 124 (GFSLTSY), H-CDR2 according to SEQ ID NO: 125 (WSDGD), and H-CDR3 according to SEQ ID NO: 126 (LQSEDTATYYCARLQVFGYPGIRDYVMDA), or one or more corresponding CDR sequences of at least 80% sequence identity to SEQ ID NOs 124 to 126;
and a variable light chain (VL), said VL comprising:
light chain complementary determining regions L-CDR1 according to SEQ ID NO: 127 (KSSQSLLSSRHQKNFLA), L-CDR2 according to SEQ ID NO: 128 (HASTRQS), and L-CDR3 according to SEQ ID NO: 129 (LQHYTSPYT), or a sequence of at least 80% sequence identity to SEQ ID NOs 127 to 129.

In yet another embodiments, the CAR of the disclosure comprises a VH domain
according to: SEQ ID NO 130:
(QVQLKESGPGLVQPSQTLSLTCTVSGLSLTSNGVSWIRQPPGKGLEWLGVIWSN
GGTDYNSAIKSRLSFSRDTSKSQVFLKMNSLQTEDTAMYFCARPRYNSGYFFDYW
GQGVMVIVSS)
and
a VL domain according to: (7A1) SEQ ID NO: 131
(DTVLTQSPALAVSPGERVTISCKASESVSTRMHWYRQKPGQQPKLLIYKTSNLAS
GVPARFSGSGSGTDFTLTIDPVEADDTATYFCQQSWNGPLTFGSGTKLEIKR),
or
a VH domain according to: (6G4) SEQ ID NO: 132
(QVQLKESGPGLVQPSQTLSLTCTVSGFSLTSYHVHWVRQPPGKGLEWMGVMWS
DGDTLYNSALKSRLSISRDTSKSQVLLQMDSLQSEDTATYYCARLQVFGYPGIRDY
VMDAWGQGASVTVSS)
and
a VL domain according to: (6G4) SEQ ID NO: 133
(DLVMTQSPFSLAVSEGEMVTIKCKSSQSLLSSRHQKNFLAWYRQKPGQSPKLLIY
HASTRQSGVPDRFIGSGSGTDFTLTI
SDVQAEDLADYYCLQHYTSPYTFGAGTKLELKR).
In yet another embodiments, the CAR of the disclosure comprising or
consisting of a sequence according to SEQ ID NO: 134:
MEFGLSWLFLVAILKGVQCQVQLKESGPGLVQPSQTLSLTCTVSGLSLTSNGVSWI
RQPPGKGLEWLGVIWSNGGTDYNSAIKSRLSFSRDTSKSQVFLKMNSLQTEDTAM
YFCARPRYNSGYFFDYWGQGVMVTVSSSGGGGSGGGGSGGGGSDTVLTQSPAL
AVSPGERVTISCKASESVSTRMHWYRQKPGQQPKLLIYKTSNLASGVPARFSGSG
SGTDFTLTIDPVEADDTATYFCQQSWNGPLTFGSGTKLEIKGDPAESKYGPPCPPC
PGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGKKD
PKFWVLVVVGGVLACYSLLVTVAFIIFWVRSKRSRLLHSDYMNMTPRRPGPTRKHY
QPYAPPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLYQGL
STATKDTYDALHMQALPPR,
or
SEQ ID NO: 135
MEFGLSWLFLVAILKGVQCQVQLKESGPGLVQPSQTLSLTCTVSGFSLTSYHVHW
VRQPPGKGLEWMGVMWSDGDTLYNSALKSRLSISRDTSKSQVLLQMDSLQSEDT
ATYYCARLQVFGYPGIRDYVMDAWGQGASVTVSSSGGGGSGGGGSGGGGSDLV
MTQSPFSLAVSEGEMVTIKCKSSQSLLSSRHQKNFLAWYRQKPGQSPKLLIYHAST
RQSGVPDRFIGSGSGTDFTLTISDVQAEDLADYYCLQHYTSPYTFGAGTKLELKGD
PAESKYGPPCPPCPGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNH
YTQKSLSLSPGKKDPKFWVLVVVGGVLACYSLLVTVAFIIFWVRSKRSRLLHSDYM
NMTPRRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGMKGER
RRGKGHDGLYQGLSTATKDPTTPFTCRPCPL,
or
SEQ ID NO: 136
MEFGLSWLFLVAILKGVQCQVQLKESGPGLVQPSQTLSLTCTVSGLSLTSNGVSWI
RQPPGKGLEWLGVIWSNGGTDYNSAIKSRLSFSRDTSKSQVFLKMNSLQTEDTAM
YFCARPRYNSGYFFDYWGQGVMVTVSSSGGGGSGGGGSGGGGSDTVLTQSPAL
AVSPGERVTISCKASESVSTRMHWYRQKPGQQPKLLIYKTSNLASGVPARFSGSG
SGTDFTLTIDPVEADDTATYFCQQSWNGPLTFGSGTKLEIKGDPAEPKSPDKTHTC
PPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGKK
DPKFWVISTSGRPWPGLVGSFSCHWLSPFTATTGKRGRKKLLYIFKQPFMRPVQT
TQEEDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGKGH
DGLYQGLSTATKDTYDALHMQALPPR,
or
SEQ ID NO: 137:
MEFGLSWLFLVAILKGVQCQVQLKESGPGLVQPSQTLSLTCTVSGFSLTSYHVHW
VRQPPGKGLEWMGVMWSDGDTLYNSALKSRLSISRDTSKSQVLLQMDSLQSEDT
ATYYCARLQVFGYPGIRDYVMDAWGQGASVTVSSSGGGGSGGGGSGGGGSDLV
MTQSPFSLAVSEGEMVTIKCKSSQSLLSSRHQKNFLAWYRQKPGQSPKLLIYHAST
RQSGVPDRFIGSGSGTDFTLTISDVQAEDLADYYCLQHYTSPYTFGAGTKLELKGD
PAEPKSPDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHN
HYTQKSLSLSPGKKDPKFWVISTSGRPWPGLVGSFSCHWLSPFTATTGKRGRKKL
LYIFKQPFMRPVQTTQEEDGCSCRFPEEEEGGCERVKFSRSADAPAYQQGQNQL
YNELNLGRREEYDVLDKRRGRDPEMGGKPRRKNPQEGLYNELQKDKMAEAYSEI
GMKGERRRGKGHDGLY.

The antigen binding domain can be any domain that binds to an antigen, including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CAR Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this disclosure may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD 134, CD 137, CD 154. In some embodiments, a transmembrane domain may include at least the transmembrane region(s) of, e.g., KIR2DS2, OX40, CD2, CD27, LFA-I (CDI la, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge.

In one aspect, the hinge or spacer comprises an IgG4 hinge. In one aspect, the hinge or spacer comprises an IgD hinge. In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain. Optionally, a short oligo- or polypeptide linker, between 2 and 10 amino acids in length may form the linkage between the transmembrane domain and the cytoplasmic region of the CAR. A glycine-serine doublet provides a particularly suitable linker. In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the disclosure include those of TCR zeta, FcR gamma, FcR beta, CD3 gamma, CD3 delta, CD3 epsilon, CD5, CD22, CD79a, CD79b, CD278 (also known as “ICOS”), FceRI, DAP 10, DAP12, and CD66d. In one embodiment, a CAR of the disclosure comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

### Costimulatory Signaling Domain

The intracellular signalling domain of the CAR can comprise the CD3-zeta signaling domain by itself or it can be combined with any other desired intracellular signaling domain(s) useful in the context of a CAR of the disclosure. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, HVEM (LIGHTR), KIRDS2, SLAMF7, NKp80 (KLRF1), NKp44, NKp30, NKp46, CD19, CD4, CD8alpha., CD8beta, IL-2R beta, IL-2R gamma, IL-7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. For example, CD27 costimulation has been demonstrated to enhance expansion, effector function, and survival of human CART cells in vitro and augments human T cell persistence and antitumor activity in vivo (Song et al. Blood. 2012; H9 (3): 696-706). Further examples of such costimulatory molecules include CDS, ICAM-1, GITR, BAFFR, F1VEM (LIGF1TR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, NKG2D, NKG2C and PAG/Cbp. The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB.

In one aspect, the intracellular signaling domain is designed to comprise the signaling domain of CD3-zeta and the signaling domain of CD27.

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs. For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell. As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGFbeta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

### Nucleic Acid Constructs Encoding a CAR

The present disclosure also provides nucleic acid molecules encoding one or more CAR constructs described herein. In one aspect, the nucleic acid molecule is provided as a messenger RNA transcript. In one aspect, the nucleic acid molecule is provided as a DNA construct.

In some embodiments, the disclosure pertains to an isolated nucleic acid molecule encoding a chimeric antigen receptor (CAR), wherein the CAR comprises an antigen binding domain, a transmembrane domain, and an intracellular signaling domain comprising a stimulatory domain, e.g., a costimulatory signaling domain and/or a primary signaling domain, e.g., zeta chain.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (y), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the disclosure is an adenoviral vector (A5/35). In another embodiment, the expression of nucleic acids encoding CARs can be accomplished using of transposons such as sleeping beauty, CRISPR, CAS9, and zinc finger nucleases. See below June et al. 2009 Nature Reviews Immunology 9.10:704-716, is incorporated herein by reference. In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used.

A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009).

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to a target expressed on acute myeloid leukemia cells, such as, e.g., CD123, CD34, CLL-1, folate receptor beta, or FLT3; or a target expressed on a B cell, e.g., CD10, CD19, CD20, CD22, CD34, CD123, FLT-3, ROR1, CD79b, or CD79a. In one embodiment, the vector comprises a nucleic acid sequence encoding a first CAR that specifically binds a first antigen and includes an intracellular signaling domain having a costimulatory signaling domain but not a primary signaling domain, and a nucleic acid encoding a second CAR that specifically binds a second, different, antigen and includes an intracellular signaling domain having a primary signaling domain but not a costimulatory signaling domain.

In one embodiment, the vector comprises a nucleic acid encoding a CAR described herein and a nucleic acid encoding an inhibitory CAR. In one embodiment, the inhibitory CAR comprises an antigen binding domain that binds an antigen found on normal cells but not cancer cells. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta.

In embodiments, the vector may comprise two or more nucleic acid sequences encoding a CAR, e.g., a CAR described herein and a second CAR, e.g., an inhibitory CAR or a CAR that specifically binds to a different antigen. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites (e.g., an auto-cleavage site or a substrate for an intracellular protease).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al, 2012, MOLECULAR CLONING: A LABORATORY MANUAL, volumes 1-4, Cold Spring Harbor Press, NY). A preferred method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates.

Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELIS As and Western blots) or by assays described herein to identify agents falling within the scope of the disclosure.

The present disclosure further provides a vector comprising a CAR encoding nucleic acid molecule. In one aspect, a CAR vector can be directly transduced into a cell, e.g., a T cell or NK cell.

In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells or NK cells. In one aspect, the mammalian T cell is a human T cell. In one aspect, the mammalian NK cell is a human NK cell. In one embodiment, the vector is selected from the group consisting of a DNA vector, an RNA vector, a plasmid vector, a lentivirus vector, an adenoviral vector, or a retrovirus vector.

Further disclosed is a vector comprising a nucleic acid molecule encoding an RNA molecule disclosed herein, e.g., an immune stimulatory RNA molecule disclosed herein. In one embodiment, the vector can be directly transduced into a cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the RNA molecule in mammalian T cells or NK cells. In one aspect, the mammalian T cell is a human T cell. In one aspect, the mammalian NK cell is a human NK cell. In one embodiment, the vector is selected from the group consisting of a DNA vector, an RNA vector, a plasmid vector, a lentivirus vector, an adenoviral vector, or a retrovirus vector. In some embodiments, the nucleic acid molecule encoding the CAR and the nucleic acid molecule encoding the RNA molecule, e.g., the immune stimulatory RNA molecule, are disposed on a single vector. In some embodiments, the nucleic acid molecule encoding the CAR and the nucleic acid molecule encoding the RNA molecule, e.g., the immune stimulatory RNA molecule, are disposed on separate vectors.

In some embodiments, the present disclosure provides nucleic acid molecules (or vector) encoding a polypeptide which comprises a heavy chain variable region (VH) and/or light chain variable region (VL) as provided in Table 1 and Table 2, or a specific VH and VL combination as provided in Table 1 and Table 2. In another embodiments, the present disclosure provides nucleic acid molecules encoding a polypeptide which comprises a heavy chain variable region (VH) and/or light chain variable region (VL) which have at least 60%, 70%, 80%, 85%, 90%, 95%, 99% identity of the VH and/or VL amino acid sequences as provided in Table 1 and Table 2.

In some embodiments, the present disclosure provides nucleic acid molecules (or vector) encoding a polypeptide which comprises one, two, three or more HCDRs and/or one, two, three or more LCDRs as provided in Table 3 and Table 4, or a specific HCDR1-3 and LCDR1-3 combination as provided in Table 3 and Table 4.

In some embodiments, the present disclosure provides nucleic acid molecules (or vector) encoding a polypeptide comprising amino acid sequence as provided in Table 5. In another embodiments, the polypeptide comprises a at least 60%, 70%, 80%, 85%, 90%, 95%, 99% identity of the CAR sequences as provided in Table 5. In another embodiments, said polypeptide is a CAR.

In some embodiments, the present disclosure provides nucleic acid molecules (or vector) comprising a nucleic acid sequence as provided in Table 6. In another embodiments, the present disclosure provides nucleic acid molecules (or vector) comprising a nucleic acid sequence having a at least 60%, 70%, 80%, 85%, 90%, 95%, 99% identity of a nucleic acid sequence as provided in Table 6.

### Combination Therapies

The compositions or immunogenic compositions comprising PIC as described herein may be used in combination with a CAR-expressing cell (CAR therapy). In some embodiments, besides in combination with a CAR-expressing cell, the compositions or immunogenic compositions may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The compositions or immunogenic compositions combined with a CAR-expressing cell as described herein and the at least one additional therapeutic agent can be administered simultaneously, in the same or in separate compositions, or sequentially. For sequential administration, the compositions or immunogenic compositions combined with a CAR-expressing cell as described herein can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, compositions or immunogenic compositions, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the compositions or immunogenic compositions, the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the compositions or immunogenic compositions, the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In some embodiments, the disclosure discloses a combination therapy including a composition or immunogenic composition described herein, a CAR-expressing cell therapy described herein, and an additional therapeutic agent.

### PD-1 Inhibitor

In some embodiments, the additional therapeutic agent is a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In one embodiment, the PD-I inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or B AP049-Clone-B disclosed in US 2015/0210769. The antibody molecules described herein can be made by vectors, host cells, and methods described in US 2015/0210769, incorporated by reference in its entirety.

In one embodiment, the anti-PD-1 antibody molecule is Nivolumab (Bristol-Myers Squibb), also known as MDX-1106, MDX-I 106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34 (5): 409-18, U.S. Pat. Nos. 7,695,715, 7,332,582, and 8,686,119, incorporated by reference in their entirety. In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule is REGN2810 (Regeneron). In one embodiment, the anti-PD-1 antibody molecule is PF-06801591 (Pfizer). In one embodiment, the anti-PD-1 antibody molecule is BGB-A317 or BGB-108 (Beigene). In one embodiment, the anti-PD-1 antibody molecule is INCSHR1210 (Incyte), also known as INCSHR01210 or SHR-1210. In one embodiment, the anti-PD-1 antibody molecule is TSR-042 (Tesaro), also known as ANB011. Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

### PD-L1 Inhibitors

In some embodiments, the additional therapeutic agent is a PD-L1 inhibitor. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone O or BAP058-Clone N disclosed in US 2016/0108123.

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment MEDI4736, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

### LAG-3 Inhibitors

In some embodiments, the additional therapeutic agent is a LAG-3 inhibitor. In some embodiments, the LAG-3 inhibitor is chosen from LAG525 (Novartis), BMS-986016 (Bristol-Myers Squibb), or TSR-033 (Tesaro).

In one embodiment, the LAG-3 inhibitor is an anti-LAG-3 antibody molecule. In one embodiment, the LAG-3 inhibitor is an anti-LAG-3 antibody molecule as disclosed in US2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420.

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule is TSR-033 (Tesaro). In one embodiment, the anti-LAG-3 antibody molecule is IMP731 or GSK2831781 (GSK and Prim a BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule is IMP761 (Prima BioMed). Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety. In one embodiment, the anti-LAG-3 inhibitor is a soluble LAG-3 protein, e.g., IMP321 (Prima BioMed), e.g., as disclosed in WO 2009/044273, incorporated by reference in its entirety.

### TIM-3 Inhibitors

In some embodiments, the additional therapeutic agent is a TIM-3 inhibitor. In some embodiments, the TIM-3 inhibitor is MGB453 (Novartis) or TSR-022 (Tesaro).

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-huml I or ABTIM3-hum03 disclosed in US 2015/0218274.

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2. Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

### Inhibitors of a Pro-M2 Macrophage Molecule

In some embodiments, the additional therapeutic agent is an inhibitor of a pro-M2 macrophage molecule. Macrophages with the M2 phenotype are known to play a role in inhibiting T cell function, including cytotoxic function. Certain cytokines, such as IL-13, IL-4, IL-10, CSF-1, TGF-beta and GM-CSF are known to polarize macrophages to the M2 phenotype, for example (in the case of IL-13 and/or IL-4), by interaction with the IL-13Ra1 chain and/or IL-4Ra chain expressed on macrophages. Molecules that block such molecules are useful in the methods and compositions described herein. Exemplary inhibitors of a pro-M2 macrophage molecule include inhibitors of IL-13, inhibitors of IL-4, inhibitors of IL-13Ra1, and/or inhibitors of IL-4Ra, e.g., as described herein.

Inhibitors of a pro-M2 macrophage molecule include, for example, small molecules. An example of a small molecule inhibitor that can be administered with a CAR-expressing cell disclosed herein and an RNA molecule disclosed herein is pterostilbene (see, e.g., Huang et al., Oncotarget. 2016 Jun. 28; 7(26): 39363-39375), which is hereby incorporated by reference in its entirety.

Inhibitors of a pro-M2 macrophage molecule include, for example, an antibody molecule, a polypeptide, e.g., a fusion protein, or an inhibitory nucleic acid, e.g., a siRNA or shRNA, or a CAR-expressing cell which binds one or more surface antigens on MDSCs or TAMs.

In one aspect, the inhibitor of a pro-M2 macrophage molecule is an anti-IL-13 antibody. Generation of such antibodies may be undertaken by methods known in the art. An example of anti-IL-13 antibodies includes, for example, lebrikizumab (see CAS number 953400-68-5). Another example of an anti-IL-13 antibody is tralokinumab (CAS number 1044515-88-9). Another example of an anti-IL-13 antibody is or comprises the anti-IL-13 binding domain of GSK2434735. Another example of an anti-IL-13 antibody is QAX576 (see, e.g., Rothenberg et al., J. Allergy Clin. Immunol., 2015, 135(2), pp. 500-507, which is hereby incorporated by reference in its entirety).

In another aspect, the inhibitor of a pro-M2 macrophage molecule is an anti-IL-4 antibody or anti-IL-4Ra antibody. Generation of such antibodies may be undertaken by methods known in the art. An example of anti-IL-4 antibodies includes, for example, the anti-IL-4 binding domain of GSK2434735. Another example of an anti-IL-4 antibody is, for example, dupilumab (see CAS number 1190264-60-8).

In another embodiment, the inhibitor of a pro-M2 macrophage is an inhibitor of IL-13 and/or IL-4. An example of an inhibitor of IL-13 and IL-4 that can be administered with a CAR-expressing cell disclosed herein and a composition or immunogenic composition disclosed herein is the vitamin A derivative Fenretinide ((e.g., 4-HPR) see, e.g., Dong et al. Cancer Letters. Mar. 1, 2017. Volume 388, Pages 43-53, which is hereby incorporated by reference in its entirety).

In another aspect, the inhibitor of a pro-M2 macrophage molecule is an anti-CSF-1 antibody or small molecule inhibitor of CSF-1. Generation of such antibodies may be undertaken by methods known in the art. An example of an anti-CSF-I antibody is emactuzumab. Another example of a CSF-1 inhibitor is BLZ945 (see, e.g., Strachan, D C et al., Oncoimmunology, 2013 Dec. 1, 2(12): e26968, which is hereby incorporated by reference in its entirety). Another example of an inhibitor of CSF-1 that can be administered with a CAR-expressing cell disclosed herein and a composition or immunogenic composition disclosed herein is nintedanib (see, e.g., Tandon et al. American Journal of Respiratory and Critical Care Medicine 2017; 195: A2397, which is hereby incorporated by reference in its entirety). BLZ945 is a small molecule inhibitor of colony stimulating factor 1 receptor (CSF1R). See, e.g., Pyonteck et al. Nat. Med. 19(2013): 1264-72. The structure of BLZ945 is shown below.

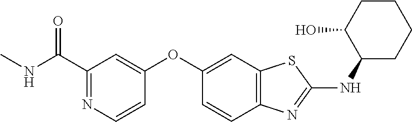 [SMILES: CNC(=O)c1cc(Oc2ccc3nc(N[C@@H]4CCCC[C@H]4O)sc3c2)ccn1]

In another aspect, the inhibitor of a pro-M2 macrophage molecule is a CAR-expressing cell which binds an antigen expressed on the surface of a MDSC or TAM (i.e., a TAM antigen), e.g., an antigen that is upregulated on the surface of a MDSCs or TAM, relative to other macrophages. In embodiments, the CAR-expressing cell which binds a MDSCs or TAM antigen binds to CD123. In embodiments, the CAR-expressing cell which binds a MDSCs or TAM antigen binds to CSF1R. In embodiments, the CAR-expressing cell which binds a MDSCs or TAM antigen binds to CD68. In embodiments, the CAR-expressing cell which binds a MDSCs or TAM antigen binds to CD206.

In another embodiment, the inhibitor of a pro-M2 macrophage is a JAK2 inhibitor. An example of a JAK2 inhibitor that can be administered with a CAR-expressing cell disclosed herein and a composition or immunogenic composition disclosed herein is Ruxolitinib (see, e.g., Chen et al. Clinical Lymphoma, Myeloma and Leukemia, Volume 17, Issue 1, e93, 2017, which is hereby incorporated by reference in its entirety). In another embodiment, the inhibitor of a pro-M2 macrophage molecule is a cell surface molecule. An example of a cell surface molecule that can be administered with a CAR-expressing cell disclosed herein and a composition or immunogenic composition disclosed herein is Dipeptidyl peptidase 4 (DPP-4) or CD26 (see, e.g., Zhuge et al. Diabetes 2016 October; 65(10): 2966-2979, which is hereby incorporated by reference in its entirety).

In another embodiment, the inhibitor of a pro-M2 macrophage molecule is an HD AC inhibitor. An example of an HD AC inhibitor that can be administered with a CAR-expressing cell disclosed herein and a composition or immunogenic composition disclosed herein is suberanilohydroxamic acid (SAHA). In another embodiment, the inhibitor of a pro-M2 macrophage molecule is an inhibitor of the glycolytic pathway. An example of an inhibitor of the glycolytic pathway that can be administered with a CAR-expressing cell disclosed herein and a composition or immunogenic composition disclosed herein is 2-deoxy-d-glucose ((2-DG), see, e.g., Zanganeh, Nat Nanotechnol. 2016 November; 11(11): 986-994, which is hereby incorporated by reference in its entirety).

In another embodiment, the inhibitor of a pro-M2 macrophage molecule is a mitochondria-targeted antioxidant. An example of a mitochondria-targeted antioxidant that can be administered with a CAR-expressing cell disclosed herein and a composition or immunogenic composition disclosed herein is MitoQ (Formentini et al., Cell Reports, Volume 19, Issue 6, 9 May 2017, Pages 1202-1213, which is hereby incorporated by reference in its entirety). In another embodiment, the inhibitor of a pro-M2 macrophage molecule is an iron oxide. An example of an iron oxide that can be administered with a CAR-expressing cell disclosed herein and a composition or immunogenic composition disclosed herein is ferumoxytol (see, e.g., Zanganeh, Nat Nanotechnol. 2016 November; 11(11): 986-994, which is hereby incorporated by reference in its entirety).

In embodiments, the disclosure includes a composition comprising an inhibitor of a pro-M2 macrophage molecule, and a pharmaceutically acceptable carrier.

### Flt3 Ligand Polypeptide

In some embodiments, the additional therapeutic agent is a Fms-like tyrosine kinase 3 ligand (Flt3 ligand) polypeptide. Flt3 ligand is a cytokine that affects growth, survival, and/or differentiation of cells in the hematopoietic lineage. In combination with other growth factors, Flt3 ligand can stimulate proliferation and development of various cell types, including stem cells, myeloid and lymphoid precursor cells, dendritic cells and NK cells. Exemplary Flt3 ligand polypeptides are disclosed in U.S. Pat. Nos. 5,554,512, 6,291,661, 7,294,331, 7,361,330, and 9,486,519, incorporated herein by reference in their entirety.

### Chemotherapeutic Agents

In some embodiments, the additional therapeutic agent is a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-UR), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide tezacitibine, (Eulexin®), Gemcitabine (difluorodeoxycitidine), hydroxyurea (Flydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel), tamoxifen citrate (Nolvadex®), teniposide (Vumon®), 6-thioguanine, thiotepa, tirapazamine (Tirazone), topotecan hydrochloride for injection (Hycamptin®), vinblastine (Velban®), vincristine (Oncovin®), and vinorelbine (Navelbine®).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Akeran®), Chlorambucil (Leukeran®), pipobroman (Amedel®, Vercyte®), triethylenemelamine (Hemel®, is Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol-AQ); Chlorambucil (Leukeran®); Cyclophosphamide (Cytoxan® and Neosar); Dacarbazine (also known as DTIC, DIC and imidazole carboxamide, DTIC-Dome); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (IR,2R,45)-4-[(2R)-2 [(1R,95,125,15R,16E,18R,19R,21R, 23S,24£,26£,28Z,30S,32S,35R)-1,18-dihydroxy-19,30-dimethoxy-15,17,21,23, 29,35-hexamethyl-2,3,10,14,20-pentaoxo-I I,36-dioxa-4-azatricyclo[30.3.1.0^{4.9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-i]pyrimidin-7-yl}-2-methoxyphenyl)methanol (AZD8055); 2-Amino-8-| s-4-(2-hydiOxycthoxy)cyclohexyl]-6-(6-methoxy-3-pyridinyl)-4-methyl-pyrido|2,3-r]pyrimidn-7(8//)-one (PF04691502, CAS 1013101-36-4); and N²-|1,4-dioxo-4-| |4-(4-oxo-8-phenyl-4//-1-benzopyran-2-yl)morpholinium-4-yl]methoxy]butyl]-L-arginylglycyl-L-a-aspartylL-serine- (SEQ ID NO: 846), inner salt (SF1126, CAS 936487-67-1), and XL765.

Exemplary immunomodulators include, e.g., afutuzumab (available from so Roche®); pegfilgrastim (Neulasta): lenalidomide (CC-5013, Revlimid®); thalidomide (Thalomid®), actimid (CC4047); and IRX-2 (mixture of human cytokines including interleukin 1, interleukin 2, and interferon g, CAS 951209-71-5, available from IRX Therapeutics).

Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-/V—((S)—I—(((S)-4-methyl-1-((R)-2-methyloxiran-2-yl)-I oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and 0-Methyl-/V-[(2-methyl-5-thiazolyl) carbonyl]-L-seryl-0-methyl-/V—[(IS)-2-[(2R)-2-methyl-2-oxiranyl]-2-oxo-I-(phenylmethyl)ethyl]-L-serinamide (ONX-0912).

### Pharmaceutical Compositions and Treatments

Pharmaceutical compositions of the present disclosure may comprise a CAR-expressing cell, e.g., a plurality of CAR-expressing cells, combined with a composition or immunogenic composition as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. Compositions of the present disclosure are in one aspect formulated for intravenous administration. Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the CAR-expressing cell (e.g., T cell or NK cell) compositions of the present disclosure are administered by i.v. injection. The compositions of CAR-expressing cells (e.g., T cells or NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the disclosure may be introduced, thereby creating a CAR-expressing cell (e.g., CAR T cell or CAR-expressing NK cell) of the disclosure. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells (e.g., CAR T cells or NK cells) combined with a composition or immunogenic composition of the present disclosure. In an additional aspect, expanded cells combined with an RNA molecule described herein are administered before or following surgery. In embodiments, lymphodepletion is performed on a subject, e.g., prior to administering one or more cells that express a CAR combined with a composition or immunogenic composition as described herein. In embodiments, the lymphodepletion comprises administering one or more of melphalan, cytoxan, cyclophosphamide, and fludarabine.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the CAR is introduced into immune effector cells (e.g., T cells or NK cells), e.g., using in vitro transcription, and the subject (e.g., human) receives an initial administration of CAR immune effector cells (e.g., T cells or NK cells) of the disclosure, and one or more subsequent administrations of the CAR immune effector cells (e.g., T cells or NK cells) of the disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR immune effector cells (e.g., T cells or NK cells) of the disclosure are administered to the subject (e.g., human) per week, e.g., 2, 3, or 4 administrations of the CAR immune effector cells (e.g., T cells or NK cells) of the disclosure are administered per week. In one embodiment, the subject (e.g., human subject) receives more than one administration of the CAR immune effector cells (e.g., T cells or NK cells) per week (e.g., 2, 3 or 4 administrations per week) (also referred to herein as a cycle), followed by a week of no CAR immune effector cells (e.g., T cells or NK cells) administrations, and then one or more additional administration of the CAR immune effector cells (e.g., T cells or NK cells) (e.g., more than one administration of the CAR immune effector cells (e.g., T cells or NK cells) per week) is administered to the subject. In another embodiment, the subject (e.g., human subject) receives more than one cycle of CAR immune effector cells (e.g., T cells or NK cells), and the time between each cycle is less than 10, 9, 8, 7, 6, 5, 4, or 3 days. In one embodiment, the CAR immune effector cells (e.g., T cells or NK cells) are administered every other day for 3 administrations per week. In one embodiment, the CAR immune effector cells (e.g., T cells or NK cells) of the disclosure are administered for at least two, three, four, five, six, seven, eight or more weeks.

In one aspect, CAR-expressing cells (e.g., CARTs or CAR-expressing NK cells) are generated using lentiviral viral vectors, such as lentivirus. CAR-expressing cells (e.g., CARTs or CAR-expressing NK cells) generated that way will have stable CAR expression. In one aspect, this disclosure features a cell expressing a stimulatory RNA molecule, e.g., an immune stimulatory RNA molecule, disclosed herein, wherein the cell is generated using lentiviral viral vectors, such as lentivirus.

In one aspect, CAR-expressing cells, e.g., CARTs, are generated using a viral vector such as a gammaretro viral vector, e.g., a gammaretro viral vector described herein. CARTs generated using these vectors can have stable CAR expression. In one aspect, a cell expressing a stimulatory RNA molecule, e.g., an immune stimulatory RNA molecule, disclosed herein is generated using a viral vector such as a gammaretroviral vector, e.g., a gammaretroviral vector described herein.

In one aspect, CAR-expressing cells (e.g., CARTs or CAR-expressing NK cells) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., T cell or NK cell, by electroporation. In one aspect, a cell expressing a stimulatory RNA molecule, e.g., an immune stimulatory RNA molecule, disclosed herein transiently expresses the RNA molecule. In one aspect, the stimulatory RNA molecule is delivered into the cell by electroporation. A potential issue that can arise in patients being treated using transiently expressing CAR-expressing cells (e.g., CARTs or CAR-expressing NK cells) (particularly with murine scFv bearing CAR-expressing cells (e.g., CARTs or CAR-expressing NK cells)) is anaphylaxis after multiple treatments. Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen-day break in exposure to antigen.

If a patient is at high risk of generating an anti-CAR antibody response during the course of transient CAR therapy (such as those generated by RNA transductions), CAR-expressing cell (e.g., CART or CAR-expressing NK cell) infusion breaks should not last more than ten to fourteen days.

The disclosures of each and every patent, patent application, and publication cited herein are hereby incorporated herein by reference in their entirety. While this disclosure has been disclosed with reference to specific aspects, it is apparent that other aspects and variations of this disclosure may be devised by others skilled in the art without departing from the true spirit and scope of the disclosure. The appended claims are intended to be construed to include all such aspects and equivalent variations.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present disclosure and practice the claimed methods. The following working examples specifically point out various aspects of the present disclosure, and are not to be construed as limiting in any way the remainder of the disclosure.

### Example 1: Preparation of Immunogenic Composition Comprising Polyinosinic-Polycytidylic Acid (PIC) Kanamycin and Calcium Chloride or ZNP (Polyinosinic Polycytidylic Acid Based Adjuvant)

Polyinosinic Polycytidylic Acid Based Adjuvant (ZNP), an immunogenic composition comprising polyinosinic-polycytidylic acid (PIC), kanamycin and calcium chloride was produced as previously reported (Cell Mol Immunol. 2007 April; 4(2):113-20). Under sterile condition, the ZNP composition is to be formulated in a physiologically acceptable buffer. The concentration and volume of the composition to be formulated are allowed to be adjusted according to the factors including subject to be administered (including but not limited to age, sex, body weight, health condition), cancer condition (including but not limited to cancer type, severity), administration route, and administration frequency. For the same therapeutically effective amount, when the concentration of the composition is high, the administration volume is small; when the concentration of the composition is low, the administration volume is large.

PIC is unstable in human body, and can be quickly broken down by nuclease, limiting their effective use in human body. In the present disclosure, the inventors envisioned that the presence of antibiotic (or polyamine compound) and positive ion (calcium) in the ZNP composition can form a stable three-dimensional structure with PIC, thereby increasing PIC stability of the ZNP composition, allowing effective therapeutic use in human body.

In the following illustrative examples, representative ZNP composition was prepared in the concentration of 0.5 mg/ml to 10 mg/ml, wherein said PIC has a molecular weight range of 66,000 to 2,000,000 Daltons.

### Example 2A: Preparation of Immunogenic Composition Comprising a EBV VLP

Epstein-Barr virus (EBV), a widespread human y-herpesvirus, causes persistent infection in more than 95% of the world population. Primary EBV infection is usually asymptomatic and often occurs during childhood. Epstein-Barr virus (EBV) is the causative pathogen for infectious mononucleosis and many kinds of malignancies including several lymphomas such as Hodgkin's lymphoma, Burkitt's lymphoma and malignant B-cell lymphoma, age-related EBV-positive B-lymphoproliferative diseases (LPDs), T-cell and natural killer (NK)-cell LPDs, NK/T cell lymphoma, Leiomyosarcoma as well as carcinomas such as nasopharyngeal cancer (NPC) and EBV-associated gastric carcinoma (EBV-GC), and breast, lung, colon, renal carcinoma. EBV encodes many envelope glycoproteins. The most abundant glycoprotein on the virion surface, gp350 has been one of the most studied targets for development of a prophylactic subunit vaccine to neutralize infection of B cells.

An immunogenic composition comprising EBV VLP was prepared. Virus-like particle of Epstein-Barr virus (EB-VLP) was prepared as described in WO2022/084373. In short, EB-VLP producer cells 87H7 were plated in RPMI 1640 cell culture medium supplemented with 10% FBS, penicillin (100 U/ml), and streptomycin (100 mg/ml) and puromycin (0.5 pg/ml) for 24 hours. After which the cells were changed into supplement-free RPMI 1640 cell culture medium with 1 pM 4-hydroxy-tamoxifen to induce EB-VLP production for 4 days. Then the supernatant containing secreted EB-VLPs was collected and centrifuged to remove any cells and debris at 300 g for 10 min and 2,000 g for 20 min in tandem. After filtration through 0.45-μm low protein binding filter, EB-VLP in the conditioned medium was purified after ultracentrifugation at 100,000 g for 2 h and at 160,000 g for 1.5 h respectively, then resuspended to be stored in filtered PBS.

In an illustrative example, the EB-VLP particles produced comprise most of the EBV encoded proteins including gp350, BKRF4, BVRF1, BDLF3, BZLF2, BXLF2, BNRF1, BALF4 and BZLF1, except for LMP1, EBNA2, EBNA3a, EBNA3b, EBNA3c. In another examples, full length or truncated proteins were expressed in fusion with NDV-M, NP, F, HN proteins.

### Example 2B: Preparation of Immunogenic Composition Comprising Polyinosinic-Polycytidylic Acid (PIC) Kanamycin and Calcium Chloride (i.e. ZNP) and EBV VLP, Forming ZNP-EBV VLP Vaccine

ZNP composition (ZNP) described in Example 1 will be mixed with virus-like particle (VLP) of Epstein-Barr virus (EBV) described in Example 2A to form an EBV vaccine composition comprising polyinosinic-polycytidylic acid (PIC) kanamycin and calcium chloride (i.e. ZNP) and EB-VLP (referred as “ZNP-EB-VLP vaccine”).

### Example 3: Construction of Chimeric CAR Constructs Comprising Anti-EBV Gp350 scFv

Chimeric CAR constructs comprising anti-EBV Gp350 scFvs which specifically binds to gp350 protein were designed and generated (Tables 5 and 6). The amino acid sequences of the heavy chain variable regions (VH), light chain variable regions (VL) and respective CDRs of anti-EBV Gp350 scFvs having the general structure of VH-linker-VL or VL-linker-VH are shown in Tables 1 to 4. The linker used is S(G₄S)₃ (SEQ ID NO: 101). Each of V01, V02 and V04 is sequence comprising/encoding the hinge, transmembrane region and cytoplasmic region of the CAR (underlined sequence in Tables 5 and 6). The hinge region of V01 sequence comprises TTTPAPRPPTPAPTIASQPLSLRPEACRPAAGGAVHTRGLDFACD, the hinge region of V02 comprises ESKYGPPCPPCP, and the hinge region of V04 sequence comprises ESKYGPPCPPCPAPPVAGPSVFLFPPKPKDTLMISRTPEVTCVVVDVSQEDPEVQF NWYVDGVEVHNAKTKPREEQFQSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSPGK.

TABLE 1
Heavy chain variable regions (VH) of exemplary anti-EBV Gp350 scFvs

| | | SEQ ID |
|---|---|---|
| Name | VH | NO: |
| EBV-Gp350- | QIQLVQSGPDLKKTGETVKISCKASGYTFTTYGVSW | 1 |
| 001-VH | VKQAPGKVLKWMGWINTYSGVPRYADDFKGRFAFS | |
| | LETSASTAYLQINNLKNEDTATYFCARFFYGYDHTMD | |
| | YWGQGTSVTVSS | |
| EBV-Gp350- | QIQLVQSGPELKKPGETVKISCKASGYTFTIYGMSWV | 2 |
| 002-VH | KQAPGKGLRWMGWINTYSGVPKYADDFKGHFAFSL | |
| | ETSASTAYLRINNLKDEDTATYFCARFYYGYDHAMD | |
| | YWGQGTSVIVSS | |
| EBV-Gp350- | EFQLQQSGPDLVKPGASVKISCKASGYSFTDYNMN | 3 |
| 003-VH | WVKQSNGKNLEWIGVINPNFGTTSYNQKFKGKATLT | |
| | VDQSSSTAYMQLNSLTSEDSAVYYCARSGYYGSNR | |
| | YWYFDVWGTGTTVTVSS | |
| EBV-Gp350- | EVQLQQSGPVLEKPGASVKMSCKASGYTFTDYYMN | 4 |
| 004-VH | WVKQSHGKSLEWIGVINPYNGGTSHNEKLKGKATLT | |
| | VDKSSSTAYMELNSLTSEDSAVYYCARGNYSAWFA | |
| | YWGQGTLVTVSA | |
| EBV-Gp350- | QVHLQQSGPELVKPGASVKISCKASGYNFNDYYINW | 5 |
| 005-VH | VKQRPGQGLEWIGWIFPGRGRSNYSEKFKGKATLTV | |
| | DKSSSTAYVLLSSLTSEDSAVYFCARGTAQAGFPYW | |
| | GQGTLVTVSA | |
| EBV-Gp350- | QVQLVQSGAEVKKPGASVKVSCKASGYSFTDYNMN | 138 |
| 003-VH1 | WVRQAPGQGLEWIGVINPNFGTTSYNQKFKGRVTM | |
| | TVDTSTSTVYMELSSLRSEDTAVYYCARSGYYGSNR | |
| | YWYFDVWGQGTTVTVSS | |
| EBV-Gp350- | QVQLVQSGAEVKKPGASVKVSCKASGYSFTDYNMN | 139 |
| 003-VH2 | WVKQAPGQGLEWIGVINPNFGTTSYNQKFKGRATM | |
| | TVDTSTSTVYMELSSLRSEDTAVYYCARSGYYGSNR | |
| | YWYFDVWGQGTTVTVSS | |
| EBV-Gp350- | QVQLVQSGAEVKKPGASVKVSCKASGYSFTDYNMN | 140 |
| 003-VH3 | WVKQAPGQGLEWIGVINPNFGTTSYNQKFKGRATLT | |
| | VDQSTSTVYMELSSLRSEDTAVYYCARSGYYGSNR | |
| | YWYFDVWGQGTTVTVSS | |

TABLE 2
Light chain variable regions (VL) of exemplary anti-EBV Gp350 scFvs

| | | SEQ ID |
|---|---|---|
| Name | VL | NO: |
| EBV-Gp350- | DVVMTQTPLSLPVSLGDQASISCRSSQSLAHSNGNT | 6 |
| 001-VL | YLHWYLQKPGQSPKLLIYKVSNRFSGVPDRENGSGS | |
| | GTDFTLKISRVEAEDLGVYFCSQSTHFPWTFGGGTK | |
| | LEIK | |
| EBV-Gp350- | DVVMTQTPLSLPVSLGDQASISCSSSQSLVHSNGNT | 7 |
| 002-VL | YLHWYLQRPGQSPKLLIYKVSNRFSGVPDRFSGSGS | |
| | GTDFTLKISRVEAEDLGVYFCSQSTHFPWTIGGGIKL | |
| | EIK | |
| EBV-Gp350- | DIVMTQSQKFMSTSVGDRVSVTCKASQSVGTNVAW | 8 |
| 003-VL | YQQKPGQSPKALIYSASYRYSGVPDRFTGSGSGTDF | |
| | TLTISNVQSEDLAEYFCQQYNSYPSTFGGGTKLEIK | |
| EBV-Gp350- | QAVVTQESALTTSPGETVTLTCRSSTGAVTTSNYAN | 9 |
| 004-VL | WVQEKPDHLFTGLIGGTNNRAPGVPARFSGSLIGDK | |
| | AALTITGAQTEDEAIYFCVLWYSNLWVFGGGTKLTVL | |
| EBV-Gp350- | QIVLTQSPAIMSASLGERVTMTCTASSSVSSSYLHWY | 10 |
| 005-VL | QQKPGSSPKLWIYSASNLASGVPPRFSGSGSGTSYS | |
| | LTISSMEAEDAATYYCHQYHRSPPWTFGGGTKLEIK | |
| EBV-Gp350- | DIQMTQSPSSLSASVGDRVTITCKASQSVGTNVAWF | 141 |
| 003-VL1 | QQKPGKAPKALIYSASYRYSGVPSRFSGSGSGTDFT | |
| | LTISSLQPEDFATYFCQQYNSYPSTFGGGTKLEIK | |
| EBV-Gp350- | DIQMTQSPSSLSASVGDRVTITCKASQSVGTNVAWY | 142 |
| 003-VL2 | QQKPGKAPKALIYSASYRYSGVPDRFSGSGSGTDFT | |
| | LTISSLQPEDFATYFCQQYNSYPSTFGGGTKLEIK | |
| EBV-Gp350- | DIQMTQSPSSLSASVGDRVTITCKASQSVGTNVAWY | 143 |
| 003-VL3 | QQKPGKAPKALIYSASYRYSGVPDRFTGSGSGTDFT | |
| | LTISSLQPEDFATYFCQQYNSYPSTFGGGTKLEIK | |

TABLE 3
Heavy chain CDR regions 1-3 of anti-EBV Gp350 scFvs

| | | SEQ | | SEQ | | SEQ |
|---|---|---|---|---|---|---|
| Name | HCDR1 | ID NO: | HCDR2 | ID NO: | HCDR3 | ID NO: |
| EBV- | TYGVS | 11 | WINTYSGVPR | 12 | FFYGYDHTMD | 13 |
| Gp350- | | | YADDFKG | | Y | |
| 001 | | | | | | |
| EBV- | IYGMS | 14 | WINTYSGVPKY | 15 | FYYGYDHAMD | 16 |
| Gp350- | | | ADDFKG | | Y | |
| 002 | | | | | | |
| EBV- | DYNMN | 17 | VINPNFGTTSY | 18 | SGYYGSNRYW | 19 |
| Gp350- | | | NQKFKG | | YFDV | |
| 003 | | | | | | |
| EBV- | DYYMN | 20 | VINPYNGGTSH | 21 | GNYSAWFAY | 22 |
| Gp350- | | | NEKLKG | | | |
| 004 | | | | | | |
| EBV- | DYYIN | 23 | WIFPGRGRSN | 24 | GTAQAGFPY | 25 |
| Gp350- | | | YSEKFKG | | | |
| 005 | | | | | | |

TABLE 4
Light chain CDR regions 1-3 of anti-EBV Gp350 scFvs

| | | SEQ | | SEQ | | SEQ |
|---|---|---|---|---|---|---|
| Name | LCDR1 | ID NO: | LCDR2 | ID NO: | LCDR3 | ID NO: |
| EBV- | RSSQSLAH | 26 | KVSNRFS | 27 | SQSTHFPWT | 28 |
| Gp350- | SNGNTYLH | | | | | |
| 001 | | | | | | |
| EBV- | SSSQSLVH | 29 | KVSNRFS | 30 | SQSTHFPWT | 31 |
| Gp350- | SNGNTYLH | | | | | |
| 002 | | | | | | |
| EBV- | KASQSVGT | 32 | SASYRYS | 33 | QQYNSYPST | 34 |
| Gp350- | NVA | | | | | |
| 003 | | | | | | |
| EBV- | RSSTGAVT | 35 | GTNNRAP | 36 | VLWYSNLWV | 37 |
| Gp350- | TSNYAN | | | | | |
| 004 | | | | | | |
| EBV- | TASSSVSS | 38 | SASNLAS | 39 | HQYHRSPPW | 40 |
| Gp350- | SYLH | | | | T | |
| 005 | | | | | | |

TABLE 5
CAR amino acid sequences.

| | | SEQ |
|---|---|---|
| Name | Amino acid Sequences | ID NO: |
| CAR_EBV- | MEFGLSWLFLVAILKGVQCDVVMTQTPLSLPVSLGDQ | 41 |
| Gp350- | ASISCRSSQSLAHSNGNTYLHWYLQKPGQSPKLLIYK | |
| 001_VL- | VSNRFSGVPDRFNGSGSGTDFTLKISRVEAEDLGVYF | |
| linker-VH_V01 | CSQSTHFPWTFGGGTKLEIKSGGGGSGGGGSGGGG | |
| (CAR | SQIQLVQSGPDLKKTGETVKISCKASGYTFTTYGVSW | |
| comprising | VKQAPGKVLKWMGWINTYSGVPRYADDFKGRFAFSL | |
| EBV-Gp350- | ETSASTAYLQINNLKNEDTATYFCARFFYGYDHTMDY | |
| 001 in VL- | WGQGTSVTVSSGSTTTPAPRPPTPAPTIASQPLSLRP | |
| linker-VH | EACRPAAGGAVHTRGLDFACDIYIWAPLAGTCGVLLL | |
| orientation, | SLVITLYCKRGRKKLLYIFKQPFMRPVQTTQEEDGCSC | |
| and V01 | RFPEEEEGGCELRVKFSRSADAPAYKQGQNQLYNEL | |
| sequence | NLGRREEYDVLDKRRGRDPEMGGKPRRKNPQEGLY | |
| which is | NELQKDKMAEAYSEIGMKGERRRGKGHDGLYQGLST | |
| underlined) | ATKDTYDALHMQALPPR | |
| CAR_EBV- | MEFGLSWLFLVAILKGVQCDVVMTQTPLSLPVSLGDQ | 42 |
| Gp350- | ASISCRSSQSLAHSNGNTYLHWYLQKPGQSPKLLIYK | |
| 001_VL- | VSNRFSGVPDRFNGSGSGTDFTLKISRVEAEDLGVYF | |
| linker-VH_V02 | CSQSTHFPWTFGGGTKLEIKSGGGGSGGGGSGGGG | |
| (CAR | SQIQLVQSGPDLKKTGETVKISCKASGYTFTTYGVSW | |
| comprising | VKQAPGKVLKWMGWINTYSGVPRYADDFKGRFAFSL | |
| EBV-Gp350- | ETSASTAYLQINNLKNEDTATYFCARFFYGYDHTMDY | |
| 001 in VL- | WGQGTSVTVSSGSESKYGPPCPPCPIYIWAPLAGTC | |
| linker-VH | GVLLLSLVITLYCKRGRKKLLYIFKQPFMRPVQTTQEE | |
| orientation, | DGCSCRFPEEEEGGCELRVKFSRSADAPAYKQGQNQ | |
| and V02 | LYNELNLGRREEYDVLDKRRGRDPEMGGKPRRKNPQ | |
| sequence | EGLYNELQKDKMAEAYSEIGMKGERRRGKGHDGLYQ | |
| which is | GLSTATKDTYDALHMQALPPR | |
| underlined) | | |
| CAR_EBV- | MEFGLSWLFLVAILKGVQCDVVMTQTPLSLPVSLGDQ | 43 |
| Gp350- | ASISCRSSQSLAHSNGNTYLHWYLQKPGQSPKLLIYK | |
| 001_VL- | VSNRFSGVPDRFNGSGSGTDFTLKISRVEAEDLGVYF | |
| linker-VH_V04 | CSQSTHFPWTFGGGTKLEIKSGGGGSGGGGSGGGG | |
| (CAR | SQIQLVQSGPDLKKTGETVKISCKASGYTFTTYGVSW | |
| comprising | VKQAPGKVLKWMGWINTYSGVPRYADDFKGRFAFSL | |
| EBV-Gp350- | ETSASTAYLQINNLKNEDTATYFCARFFYGYDHTMDY | |
| 001 in VL- | WGQGTSVTVSSGSESKYGPPCPPCPAPPVAGPSVFL | |
| linker-VH | FPPKPKDTLMISRTPEVTCVVVDVSQEDPEVQFNWYV | |
| orientation, | DGVEVHNAKTKPREEQFQSTYRVVSVLTVLHQDWLN | |
| and V04 | GKEYKCKVSNKGLPSSIEKTISKAKGQPREPQVYTLPP | |
| sequence | SQEEMTKNQVSLTCLVKGFYPSDIAVEWESNGQPEN | |
| which is | NYKTTPPVLDSDGSFFLYSRLTVDKSRWQEGNVFSCS | |
| underlined) | VMHEALHNHYTQKSLSLSPGKIYIWAPLAGTCGVLLLS | |
| | LVITLYCKRGRKKLLYIFKQPFMRPVQTTQEEDGCSCR | |
| | FPEEEEGGCELRVKFSRSADAPAYKQGQNQLYNELN | |
| | LGRREEYDVLDKRRGRDPEMGGKPRRKNPQEGLYN | |
| | ELQKDKMAEAYSEIGMKGERRRGKGHDGLYQGLSTA | |
| | TKDTYDALHMQALPPR | |
| CAR_EBV- | MEFGLSWLFLVAILKGVQCQIQLVQSGPDLKKTGETV | 44 |
| Gp350- | KISCKASGYTFTTYGVSWVKQAPGKVLKWMGWINTY | |
| 001_VH- | SGVPRYADDFKGRFAFSLETSASTAYLQINNLKNEDTA | |
| linker-VL_V01 | TYFCARFFYGYDHTMDYWGQGTSVTVSSSGGGGSG | |
| (CAR | GGGSGGGGSDVVMTQTPLSLPVSLGDQASISCRSSQ | |
| comprising | SLAHSNGNTYLHWYLQKPGQSPKLLIYKVSNRFSGVP | |
| EBV-Gp350- | DRFNGSGSGTDFTLKISRVEAEDLGVYFCSQSTHFPW | |
| 001 in VH- | TFGGGTKLEIKGSTTTPAPRPPTPAPTIASQPLSLRPE | |
| linker-VL | ACRPAAGGAVHTRGLDFACDIYIWAPLAGTCGVLLLSL | |
| orientation, | VITLYCKRGRKKLLYIFKQPFMRPVQTTQEEDGCSCRF | |
| and V01 | PEEEEGGCELRVKFSRSADAPAYKQGQNQLYNELNL | |
| sequence | GRREEYDVLDKRRGRDPEMGGKPRRKNPQEGLYNE | |
| which is | LQKDKMAEAYSEIGMKGERRRGKGHDGLYQGLSTAT | |
| underlined) | KDTYDALHMQALPPR | |
| CAR_EBV- | MEFGLSWLFLVAILKGVQCQIQLVQSGPDLKKTGETV | 45 |
| Gp350- | KISCKASGYTFTTYGVSWVKQAPGKVLKWMGWINTY | |
| 001_VH- | SGVPRYADDFKGRFAFSLETSASTAYLQINNLKNEDTA | |
| linker-VL_V02 | TYFCARFFYGYDHTMDYWGQGTSVTVSSSGGGGSG | |
| (CAR | GGGSGGGGSDVVMTQTPLSLPVSLGDQASISCRSSQ | |
| comprising | SLAHSNGNTYLHWYLQKPGQSPKLLIYKVSNRFSGVP | |
| EBV-Gp350- | DRFNGSGSGTDFTLKISRVEAEDLGVYFCSQSTHFPW | |
| 001 in VH- | TFGGGTKLEIKGSESKYGPPCPPCPIYIWAPLAGTCGV | |
| linker-VL | LLLSLVITLYCKRGRKKLLYIFKQPFMRPVQTTQEEDG | |
| orientation, | CSCRFPEEEEGGCELRVKFSRSADAPAYKQGQNQLY | |
| and V02 | NELNLGRREEYDVLDKRRGRDPEMGGKPRRKNPQE | |
| sequence | GLYNELQKDKMAEAYSEIGMKGERRRGKGHDGLYQG | |
| which is | LSTATKDTYDALHMQALPPR | |
| underlined) | | |
| CAR_EBV- | MEFGLSWLFLVAILKGVQCQIQLVQSGPDLKKTGETV | 46 |
| Gp350- | KISCKASGYTFTTYGVSWVKQAPGKVLKWMGWINTY | |
| 001_VH- | SGVPRYADDFKGRFAFSLETSASTAYLQINNLKNEDTA | |
| linker-VL_V04 | TYFCARFFYGYDHTMDYWGQGTSVTVSSSGGGGSG | |
| (CAR | GGGSGGGGSDVVMTQTPLSLPVSLGDQASISCRSSQ | |
| comprising | SLAHSNGNTYLHWYLQKPGQSPKLLIYKVSNRFSGVP | |
| EBV-Gp350- | DRFNGSGSGTDFTLKISRVEAEDLGVYFCSQSTHFPW | |
| 001 in VH- | TFGGGTKLEIKGSESKYGPPCPPCPAPPVAGPSVFLF | |
| linker-VL | PPKPKDTLMISRTPEVTCVVVDVSQEDPEVQFNWYVD | |
| orientation, | GVEVHNAKTKPREEQFQSTYRVVSVLTVLHQDWLNG | |
| and V04 | KEYKCKVSNKGLPSSIEKTISKAKGQPREPQVYTLPPS | |
| sequence | QEEMTKNQVSLTCLVKGFYPSDIAVEWESNGQPENN | |
| which is | YKTTPPVLDSDGSFFLYSRLTVDKSRWQEGNVFSCSV | |
| underlined) | MHEALHNHYTQKSLSLSPGKIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDVVMTQTPLSLPVSLGDQ | 47 |
| Gp350- | ASISCSSSQSLVHSNGNTYLHWYLQRPGQSPKLLIYK | |
| 002_VL- | VSNRFSGVPDRFSGSGSGTDFTLKISRVEAEDLGVYF | |
| linker-VH_V01 | CSQSTHFPWTIGGGIKLEIKSGGGGSGGGGSGGGGS | |
| (CAR | QIQLVQSGPELKKPGETVKISCKASGYTFTIYGMSWVK | |
| comprising | QAPGKGLRWMGWINTYSGVPKYADDFKGHFAFSLET | |
| EBV-Gp350- | SASTAYLRINNLKDEDTATYFCARFYYGYDHAMDYWG | |
| 002 in VL- | QGTSVIVSSGSTTTPAPRPPTPAPTIASQPLSLRPEAC | |
| linker-VH | RPAAGGAVHTRGLDFACDIYIWAPLAGTCGVLLLSLVI | |
| orientation, | TLYCKRGRKKLLYIFKQPFMRPVQTTQEEDGCSCRFP | |
| and V01 | EEEEGGCELRVKFSRSADAPAYKQGQNQLYNELNLG | |
| sequence | RREEYDVLDKRRGRDPEMGGKPRRKNPQEGLYNEL | |
| which is | QKDKMAEAYSEIGMKGERRRGKGHDGLYQGLSTATK | |
| underlined) | DTYDALHMQALPPR | |
| CAR_EBV- | MEFGLSWLFLVAILKGVQCDVVMTQTPLSLPVSLGDQ | 48 |
| Gp350- | ASISCSSSQSLVHSNGNTYLHWYLQRPGQSPKLLIYK | |
| 002_VL- | VSNRFSGVPDRFSGSGSGTDFTLKISRVEAEDLGVYF | |
| linker-VH_V02 | CSQSTHFPWTIGGGIKLEIKSGGGGSGGGGSGGGGS | |
| (CAR | QIQLVQSGPELKKPGETVKISCKASGYTFTIYGMSWVK | |
| comprising | QAPGKGLRWMGWINTYSGVPKYADDFKGHFAFSLET | |
| EBV-Gp350- | SASTAYLRINNLKDEDTATYFCARFYYGYDHAMDYWG | |
| 002 in VL- | QGTSVIVSSGSESKYGPPCPPCPIYIWAPLAGTCGVLL | |
| linker-VH | LSLVITLYCKRGRKKLLYIFKQPFMRPVQTTQEEDGCS | |
| orientation, | CRFPEEEEGGCELRVKFSRSADAPAYKQGQNQLYNE | |
| and V02 | LNLGRREEYDVLDKRRGRDPEMGGKPRRKNPQEGLY | |
| sequence | NELQKDKMAEAYSEIGMKGERRRGKGHDGLYQGLST | |
| which is | ATKDTYDALHMQALPPR | |
| underlined) | | |
| CAR_EBV- | MEFGLSWLFLVAILKGVQCDVVMTQTPLSLPVSLGDQ | 49 |
| Gp350- | ASISCSSSQSLVHSNGNTYLHWYLQRPGQSPKLLIYK | |
| 002_VL- | VSNRFSGVPDRFSGSGSGTDFTLKISRVEAEDLGVYF | |
| linker-VH_V04 | CSQSTHFPWTIGGGIKLEIKSGGGGSGGGGSGGGGS | |
| (CAR | QIQLVQSGPELKKPGETVKISCKASGYTFTIYGMSWVK | |
| comprising | QAPGKGLRWMGWINTYSGVPKYADDFKGHFAFSLET | |
| EBV-Gp350- | SASTAYLRINNLKDEDTATYFCARFYYGYDHAMDYWG | |
| 002 in VL- | QGTSVIVSSGSESKYGPPCPPCPAPPVAGPSVFLFPP | |
| linker-VH | KPKDTLMISRTPEVTCVVVDVSQEDPEVQFNWYVDG | |
| orientation, | VEVHNAKTKPREEQFQSTYRVVSVLTVLHQDWLNGK | |
| and V04 | EYKCKVSNKGLPSSIEKTISKAKGQPREPQVYTLPPSQ | |
| sequence | EEMTKNQVSLTCLVKGFYPSDIAVEWESNGQPENNY | |
| which is | KTTPPVLDSDGSFFLYSRLTVDKSRWQEGNVFSCSV | |
| underlined) | MHEALHNHYTQKSLSLSPGKIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCQIQLVQSGPELKKPGETV | 50 |
| Gp350- | KISCKASGYTFTIYGMSWVKQAPGKGLRWMGWINTY | |
| 002_VH- | SGVPKYADDFKGHFAFSLETSASTAYLRINNLKDEDTA | |
| linker-VL_V01 | TYFCARFYYGYDHAMDYWGQGTSVIVSSSGGGGSG | |
| (CAR | GGGSGGGGSDVVMTQTPLSLPVSLGDQASISCSSSQ | |
| comprising | SLVHSNGNTYLHWYLQRPGQSPKLLIYKVSNRFSGVP | |
| EBV-Gp350- | DRFSGSGSGTDFTLKISRVEAEDLGVYFCSQSTHFPW | |
| 002 in VH- | TIGGGIKLEIKGSTTTPAPRPPTPAPTIASQPLSLRPEA | |
| linker-VL | CRPAAGGAVHTRGLDFACDIYIWAPLAGTCGVLLLSLV | |
| orientation, | ITLYCKRGRKKLLYIFKQPFMRPVQTTQEEDGCSCRFP | |
| and V01 | EEEEGGCELRVKFSRSADAPAYKQGQNQLYNELNLG | |
| sequence | RREEYDVLDKRRGRDPEMGGKPRRKNPQEGLYNEL | |
| which is | QKDKMAEAYSEIGMKGERRRGKGHDGLYQGLSTATK | |
| underlined) | DTYDALHMQALPPR | |
| CAR_EBV- | MEFGLSWLFLVAILKGVQCQIQLVQSGPELKKPGETV | 51 |
| Gp350- | KISCKASGYTFTIYGMSWVKQAPGKGLRWMGWINTY | |
| 002_VH- | SGVPKYADDFKGHFAFSLETSASTAYLRINNLKDEDTA | |
| linker-VL_V02 | TYFCARFYYGYDHAMDYWGQGTSVIVSSSGGGGSG | |
| (CAR | GGGSGGGGSDVVMTQTPLSLPVSLGDQASISCSSSQ | |
| comprising | SLVHSNGNTYLHWYLQRPGQSPKLLIYKVSNRFSGVP | |
| EBV-Gp350- | DRFSGSGSGTDFTLKISRVEAEDLGVYFCSQSTHFPW | |
| 002 in VH- | TIGGGIKLEIKGSESKYGPPCPPCPIYIWAPLAGTCGVL | |
| linker-VL | LLSLVITLYCKRGRKKLLYIFKQPFMRPVQTTQEEDGC | |
| orientation, | SCRFPEEEEGGCELRVKFSRSADAPAYKQGQNQLYN | |
| and V02 | ELNLGRREEYDVLDKRRGRDPEMGGKPRRKNPQEGL | |
| sequence | YNELQKDKMAEAYSEIGMKGERRRGKGHDGLYQGLS | |
| which is | TATKDTYDALHMQALPPR | |
| underlined) | | |
| CAR_EBV- | MEFGLSWLFLVAILKGVQCQIQLVQSGPELKKPGETV | 52 |
| Gp350- | KISCKASGYTFTIYGMSWVKQAPGKGLRWMGWINTY | |
| 002_VH- | SGVPKYADDFKGHFAFSLETSASTAYLRINNLKDEDTA | |
| linker-VL_V04 | TYFCARFYYGYDHAMDYWGQGTSVIVSSSGGGGSG | |
| (CAR | GGGSGGGGSDVVMTQTPLSLPVSLGDQASISCSSSQ | |
| comprising | SLVHSNGNTYLHWYLQRPGQSPKLLIYKVSNRFSGVP | |
| EBV-Gp350- | DRFSGSGSGTDFTLKISRVEAEDLGVYFCSQSTHFPW | |
| 002 in VH- | TIGGGIKLEIKGSESKYGPPCPPCPAPPVAGPSVFLFP | |
| linker-VL | PKPKDTLMISRTPEVTCVVVDVSQEDPEVQFNWYVD | |
| orientation, | GVEVHNAKTKPREEQFQSTYRVVSVLTVLHQDWLNG | |
| and V04 | KEYKCKVSNKGLPSSIEKTISKAKGQPREPQVYTLPPS | |
| sequence | QEEMTKNQVSLTCLVKGFYPSDIAVEWESNGQPENN | |
| which is | YKTTPPVLDSDGSFFLYSRLTVDKSRWQEGNVFSCSV | |
| underlined) | MHEALHNHYTQKSLSLSPGKIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIVMTQSQKFMSTSVGDR | 53 |
| Gp350- | VSVTCKASQSVGTNVAWYQQKPGQSPKALIYSASYR | |
| 003_VL- | YSGVPDRFTGSGSGTDFTLTISNVQSEDLAEYFCQQY | |
| linker-VH_V01 | NSYPSTFGGGTKLEIKSGGGGSGGGGSGGGGSEFQL | |
| (CAR | QQSGPDLVKPGASVKISCKASGYSFTDYNMNWVKQS | |
| comprising | NGKNLEWIGVINPNFGTTSYNQKFKGKATLTVDQSSS | |
| EBV-Gp350- | TAYMQLNSLTSEDSAVYYCARSGYYGSNRYWYFDVW | |
| 003 in VL- | GTGTTVTVSSGSTTTPAPRPPTPAPTIASQPLSLRPEA | |
| linker-VH | CRPAAGGAVHTRGLDFACDIYIWAPLAGTCGVLLLSLV | |
| orientation, | ITLYCKRGRKKLLYIFKQPFMRPVQTTQEEDGCSCRFP | |
| and V01 | EEEEGGCELRVKFSRSADAPAYKQGQNQLYNELNLG | |
| sequence | RREEYDVLDKRRGRDPEMGGKPRRKNPQEGLYNEL | |
| which is | QKDKMAEAYSEIGMKGERRRGKGHDGLYQGLSTATK | |
| underlined) | DTYDALHMQALPPR | |
| CAR_EBV- | MEFGLSWLFLVAILKGVQCDIVMTQSQKFMSTSVGDR | 54 |
| Gp350- | VSVTCKASQSVGTNVAWYQQKPGQSPKALIYSASYR | |
| 003_VL- | YSGVPDRFTGSGSGTDFTLTISNVQSEDLAEYFCQQY | |
| linker-VH_V02 | NSYPSTFGGGTKLEIKSGGGGSGGGGSGGGGSEFQL | |
| (CAR | QQSGPDLVKPGASVKISCKASGYSFTDYNMNWVKQS | |
| comprising | NGKNLEWIGVINPNFGTTSYNQKFKGKATLTVDQSSS | |
| EBV-Gp350- | TAYMQLNSLTSEDSAVYYCARSGYYGSNRYWYFDVW | |
| 003 in VL- | GTGTTVTVSSGSESKYGPPCPPCPIYIWAPLAGTCGV | |
| linker-VH | LLLSLVITLYCKRGRKKLLYIFKQPFMRPVQTTQEEDG | |
| orientation, | CSCRFPEEEEGGCELRVKFSRSADAPAYKQGQNQLY | |
| and V02 | NELNLGRREEYDVLDKRRGRDPEMGGKPRRKNPQE | |
| sequence | GLYNELQKDKMAEAYSEIGMKGERRRGKGHDGLYQG | |
| which is | LSTATKDTYDALHMQALPPR | |
| underlined) | | |
| CAR_EBV- | MEFGLSWLFLVAILKGVQCDIVMTQSQKFMSTSVGDR | 55 |
| Gp350- | VSVTCKASQSVGTNVAWYQQKPGQSPKALIYSASYR | |
| 003_VL- | YSGVPDRFTGSGSGTDFTLTISNVQSEDLAEYFCQQY | |
| linker-VH_V04 | NSYPSTFGGGTKLEIKSGGGGSGGGGSGGGGSEFQL | |
| (CAR | QQSGPDLVKPGASVKISCKASGYSFTDYNMNWVKQS | |
| comprising | NGKNLEWIGVINPNFGTTSYNQKFKGKATLTVDQSSS | |
| EBV-Gp350- | TAYMQLNSLTSEDSAVYYCARSGYYGSNRYWYFDVW | |
| 003 in VL- | GTGTTVTVSSGSESKYGPPCPPCPAPPVAGPSVFLFP | |
| linker-VH | PKPKDTLMISRTPEVTCVVVDVSQEDPEVQFNWYVD | |
| orientation, | GVEVHNAKTKPREEQFQSTYRVVSVLTVLHQDWLNG | |
| and V04 | KEYKCKVSNKGLPSSIEKTISKAKGQPREPQVYTLPPS | |
| sequence | QEEMTKNQVSLTCLVKGFYPSDIAVEWESNGQPENN | |
| which is | YKTTPPVLDSDGSFFLYSRLTVDKSRWQEGNVFSCSV | |
| underlined) | MHEALHNHYTQKSLSLSPGKIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCEFQLQQSGPDLVKPGASV | 56 |
| Gp350- | KISCKASGYSFTDYNMNWVKQSNGKNLEWIGVINPNF | |
| 003_VH- | GTTSYNQKFKGKATLTVDQSSSTAYMQLNSLTSEDSA | |
| linker-VL_V01 | VYYCARSGYYGSNRYWYFDVWGTGTTVTVSSSGGG | |
| (CAR | GSGGGGSGGGGSDIVMTQSQKFMSTSVGDRVSVTC | |
| comprising | KASQSVGTNVAWYQQKPGQSPKALIYSASYRYSGVP | |
| EBV-Gp350- | DRFTGSGSGTDFTLTISNVQSEDLAEYFCQQYNSYPS | |
| 003 in VH- | TFGGGTKLEIKGSTTTPAPRPPTPAPTIASQPLSLRPE | |
| linker-VL | ACRPAAGGAVHTRGLDFACDIYIWAPLAGTCGVLLLSL | |
| orientation, | VITLYCKRGRKKLLYIFKQPFMRPVQTTQEEDGCSCRF | |
| and V01 | PEEEEGGCELRVKFSRSADAPAYKQGQNQLYNELNL | |
| sequence | GRREEYDVLDKRRGRDPEMGGKPRRKNPQEGLYNE | |
| which is | LQKDKMAEAYSEIGMKGERRRGKGHDGLYQGLSTAT | |
| underlined) | KDTYDALHMQALPPR | |
| CAR_EBV- | MEFGLSWLFLVAILKGVQCEFQLQQSGPDLVKPGASV | 57 |
| Gp350- | KISCKASGYSFTDYNMNWVKQSNGKNLEWIGVINPNF | |
| 003_VH- | GTTSYNQKFKGKATLTVDQSSSTAYMQLNSLTSEDSA | |
| linker-VL_V02 | VYYCARSGYYGSNRYWYFDVWGTGTTVTVSSSGGG | |
| (CAR | GSGGGGSGGGGSDIVMTQSQKFMSTSVGDRVSVTC | |
| comprising | KASQSVGTNVAWYQQKPGQSPKALIYSASYRYSGVP | |
| EBV-Gp350- | DRFTGSGSGTDFTLTISNVQSEDLAEYFCQQYNSYPS | |
| 003 in VH- | TFGGGTKLEIKGSESKYGPPCPPCPIYIWAPLAGTCGV | |
| linker-VL | LLLSLVITLYCKRGRKKLLYIFKQPFMRPVQTTQEEDG | |
| orientation, | CSCRFPEEEEGGCELRVKFSRSADAPAYKQGQNQLY | |
| and V02 | NELNLGRREEYDVLDKRRGRDPEMGGKPRRKNPQE | |
| sequence | GLYNELQKDKMAEAYSEIGMKGERRRGKGHDGLYQG | |
| which is | LSTATKDTYDALHMQALPPR | |
| underlined) | | |
| CAR_EBV- | MEFGLSWLFLVAILKGVQCEFQLQQSGPDLVKPGASV | 58 |
| Gp350- | KISCKASGYSFTDYNMNWVKQSNGKNLEWIGVINPNF | |
| 003_VH- | GTTSYNQKFKGKATLTVDQSSSTAYMQLNSLTSEDSA | |
| linker-VL_V04 | VYYCARSGYYGSNRYWYFDVWGTGTTVTVSSSGGG | |
| (CAR | GSGGGGSGGGGSDIVMTQSQKFMSTSVGDRVSVTC | |
| comprising | KASQSVGTNVAWYQQKPGQSPKALIYSASYRYSGVP | |
| EBV-Gp350- | DRFTGSGSGTDFTLTISNVQSEDLAEYFCQQYNSYPS | |
| 003 in VH- | TFGGGTKLEIKGSESKYGPPCPPCPAPPVAGPSVFLF | |
| linker-VL | PPKPKDTLMISRTPEVTCVVVDVSQEDPEVQFNWYVD | |
| orientation, | GVEVHNAKTKPREEQFQSTYRVVSVLTVLHQDWLNG | |
| and V04 | KEYKCKVSNKGLPSSIEKTISKAKGQPREPQVYTLPPS | |
| sequence | QEEMTKNQVSLTCLVKGFYPSDIAVEWESNGQPENN | |
| which is | YKTTPPVLDSDGSFFLYSRLTVDKSRWQEGNVFSCSV | |
| underlined) | MHEALHNHYTQKSLSLSPGKIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCQAVVTQESALTTSPGETV | 59 |
| Gp350- | TLTCRSSTGAVTTSNYANWVQEKPDHLFTGLIGGTNN | |
| 004_VL- | RAPGVPARFSGSLIGDKAALTITGAQTEDEAIYFCVLW | |
| linker-VH_V01 | YSNLWVFGGGTKLTVLSGGGGSGGGGSGGGGSEVQ | |
| (CAR | LQQSGPVLEKPGASVKMSCKASGYTFTDYYMNWVKQ | |
| comprising | SHGKSLEWIGVINPYNGGTSHNEKLKGKATLTVDKSS | |
| EBV-Gp350- | STAYMELNSLTSEDSAVYYCARGNYSAWFAYWGQGT | |
| 004 in VL- | LVTVSAGSTTTPAPRPPTPAPTIASQPLSLRPEACRPA | |
| linker-VH | AGGAVHTRGLDFACDIYIWAPLAGTCGVLLLSLVITLYC | |
| orientation, | KRGRKKLLYIFKQPFMRPVQTTQEEDGCSCRFPEEEE | |
| and V01 | GGCELRVKFSRSADAPAYKQGQNQLYNELNLGRREE | |
| sequence | YDVLDKRRGRDPEMGGKPRRKNPQEGLYNELQKDK | |
| which is | MAEAYSEIGMKGERRRGKGHDGLYQGLSTATKDTYD | |
| underlined) | ALHMQALPPR | |
| CAR_EBV- | MEFGLSWLFLVAILKGVQCQAVVTQESALTTSPGETV | 60 |
| Gp350- | TLTCRSSTGAVTTSNYANWVQEKPDHLFTGLIGGTNN | |
| 004_VL- | RAPGVPARFSGSLIGDKAALTITGAQTEDEAIYFCVLW | |
| linker-VH_V02 | YSNLWVFGGGTKLTVLSGGGGSGGGGSGGGGSEVQ | |
| (CAR | LQQSGPVLEKPGASVKMSCKASGYTFTDYYMNWVKQ | |
| comprising | SHGKSLEWIGVINPYNGGTSHNEKLKGKATLTVDKSS | |
| EBV-Gp350- | STAYMELNSLTSEDSAVYYCARGNYSAWFAYWGQGT | |
| 004 in VL- | LVTVSAGSESKYGPPCPPCPIYIWAPLAGTCGVLLLSL | |
| linker-VH | VITLYCKRGRKKLLYIFKQPFMRPVQTTQEEDGCSCRF | |
| orientation, | PEEEEGGCELRVKFSRSADAPAYKQGQNQLYNELNL | |
| and V02 | GRREEYDVLDKRRGRDPEMGGKPRRKNPQEGLYNE | |
| sequence | LQKDKMAEAYSEIGMKGERRRGKGHDGLYQGLSTAT | |
| which is | KDTYDALHMQALPPR | |
| underlined) | | |
| CAR_EBV- | MEFGLSWLFLVAILKGVQCQAVVTQESALTTSPGETV | 61 |
| Gp350- | TLTCRSSTGAVTTSNYANWVQEKPDHLFTGLIGGTNN | |
| 004_VL- | RAPGVPARFSGSLIGDKAALTITGAQTEDEAIYFCVLW | |
| linker-VH_V04 | YSNLWVFGGGTKLTVLSGGGGSGGGGSGGGGSEVQ | |
| (CAR | LQQSGPVLEKPGASVKMSCKASGYTFTDYYMNWVKQ | |
| comprising | SHGKSLEWIGVINPYNGGTSHNEKLKGKATLTVDKSS | |
| EBV-Gp350- | STAYMELNSLTSEDSAVYYCARGNYSAWFAYWGQGT | |
| 004 in VL- | LVTVSAGSESKYGPPCPPCPAPPVAGPSVFLFPPKPK | |
| linker-VH | DTLMISRTPEVTCVVVDVSQEDPEVQFNWYVDGVEV | |
| orientation, | HNAKTKPREEQFQSTYRVVSVLTVLHQDWLNGKEYK | |
| and V04 | CKVSNKGLPSSIEKTISKAKGQPREPQVYTLPPSQEE | |
| sequence | MTKNQVSLTCLVKGFYPSDIAVEWESNGQPENNYKTT | |
| which is | PPVLDSDGSFFLYSRLTVDKSRWQEGNVFSCSVMHE | |
| underlined) | ALHNHYTQKSLSLSPGKIYIWAPLAGTCGVLLLSLVITL | |
| | YCKRGRKKLLYIFKQPFMRPVQTTQEEDGCSCRFPEE | |
| | EEGGCELRVKFSRSADAPAYKQGQNQLYNELNLGRR | |
| | EEYDVLDKRRGRDPEMGGKPRRKNPQEGLYNELQK | |
| | DKMAEAYSEIGMKGERRRGKGHDGLYQGLSTATKDT | |
| | YDALHMQALPPR | |
| CAR_EBV- | MEFGLSWLFLVAILKGVQCEVQLQQSGPVLEKPGASV | 62 |
| Gp350- | KMSCKASGYTFTDYYMNWVKQSHGKSLEWIGVINPY | |
| 004_VH- | NGGTSHNEKLKGKATLTVDKSSSTAYMELNSLTSEDS | |
| linker-VL_V01 | AVYYCARGNYSAWFAYWGQGTLVTVSASGGGGSGG | |
| (CAR | GGSGGGGSQAVVTQESALTTSPGETVTLTCRSSTGA | |
| comprising | VTTSNYANWVQEKPDHLFTGLIGGTNNRAPGVPARFS | |
| EBV-Gp350- | GSLIGDKAALTITGAQTEDEAIYFCVLWYSNLWVFGGG | |
| 004 in VH- | TKLTVLGSTTTPAPRPPTPAPTIASQPLSLRPEACRPA | |
| linker-VL | AGGAVHTRGLDFACDIYIWAPLAGTCGVLLLSLVITLYC | |
| orientation, | KRGRKKLLYIFKQPFMRPVQTTQEEDGCSCRFPEEEE | |
| and V01 | GGCELRVKFSRSADAPAYKQGQNQLYNELNLGRREE | |
| sequence | YDVLDKRRGRDPEMGGKPRRKNPQEGLYNELQKDK | |
| which is | MAEAYSEIGMKGERRRGKGHDGLYQGLSTATKDTYD | |
| underlined) | ALHMQALPPR | |
| CAR_EBV- | MEFGLSWLFLVAILKGVQCEVQLQQSGPVLEKPGASV | 63 |
| Gp350- | KMSCKASGYTFTDYYMNWVKQSHGKSLEWIGVINPY | |
| 004_VH- | NGGTSHNEKLKGKATLTVDKSSSTAYMELNSLTSEDS | |
| linker-VL_V02 | AVYYCARGNYSAWFAYWGQGTLVTVSASGGGGSGG | |
| (CAR | GGSGGGGSQAVVTQESALTTSPGETVTLTCRSSTGA | |
| comprising | VTTSNYANWVQEKPDHLFTGLIGGTNNRAPGVPARFS | |
| EBV-Gp350- | GSLIGDKAALTITGAQTEDEAIYFCVLWYSNLWVFGGG | |
| 004 in VH- | TKLTVLGSESKYGPPCPPCPIYIWAPLAGTCGVLLLSL | |
| linker-VL | VITLYCKRGRKKLLYIFKQPFMRPVQTTQEEDGCSCRE | |
| orientation, | PEEEEGGCELRVKFSRSADAPAYKQGQNQLYNELNL | |
| and V02 | GRREEYDVLDKRRGRDPEMGGKPRRKNPQEGLYNE | |
| sequence | LQKDKMAEAYSEIGMKGERRRGKGHDGLYQGLSTAT | |
| which is | KDTYDALHMQALPPR | |
| underlined) | | |
| CAR_EBV- | MEFGLSWLFLVAILKGVQCEVQLQQSGPVLEKPGASV | 64 |
| Gp350- | KMSCKASGYTFTDYYMNWVKQSHGKSLEWIGVINPY | |
| 004_VH- | NGGTSHNEKLKGKATLTVDKSSSTAYMELNSLTSEDS | |
| linker-VL_V04 | AVYYCARGNYSAWFAYWGQGTLVTVSASGGGGSGG | |
| (CAR | GGSGGGGSQAVVTQESALTTSPGETVTLTCRSSTGA | |
| comprising | VTTSNYANWVQEKPDHLFTGLIGGTNNRAPGVPARFS | |
| EBV-Gp350- | GSLIGDKAALTITGAQTEDEAIYFCVLWYSNLWVFGGG | |
| 004 in VH- | TKLTVLGSESKYGPPCPPCPAPPVAGPSVFLFPPKPK | |
| linker-VL | DTLMISRTPEVTCVVVDVSQEDPEVQFNWYVDGVEV | |
| orientation, | HNAKTKPREEQFQSTYRVVSVLTVLHQDWLNGKEYK | |
| and V04 | CKVSNKGLPSSIEKTISKAKGQPREPQVYTLPPSQEE | |
| sequence | MTKNQVSLTCLVKGFYPSDIAVEWESNGQPENNYKTT | |
| which is | PPVLDSDGSFFLYSRLTVDKSRWQEGNVFSCSVMHE | |
| underlined) | ALHNHYTQKSLSLSPGKIYIWAPLAGTCGVLLLSLVITL | |
| | YCKRGRKKLLYIFKQPFMRPVQTTQEEDGCSCRFPEE | |
| | EEGGCELRVKFSRSADAPAYKQGQNQLYNELNLGRR | |
| | EEYDVLDKRRGRDPEMGGKPRRKNPQEGLYNELQK | |
| | DKMAEAYSEIGMKGERRRGKGHDGLYQGLSTATKDT | |
| | YDALHMQALPPR | |
| CAR_EBV- | MEFGLSWLFLVAILKGVQCQIVLTQSPAIMSASLGERV | 65 |
| Gp350- | TMTCTASSSVSSSYLHWYQQKPGSSPKLWIYSASNLA | |
| 005_VL- | SGVPPRFSGSGSGTSYSLTISSMEAEDAATYYCHQYH | |
| linker-VH_V01 | RSPPWTFGGGTKLEIKSGGGGSGGGGSGGGGSQVH | |
| (CAR | LQQSGPELVKPGASVKISCKASGYNFNDYYINWVKQR | |
| comprising | PGQGLEWIGWIFPGRGRSNYSEKFKGKATLTVDKSSS | |
| EBV-Gp350- | TAYVLLSSLTSEDSAVYFCARGTAQAGFPYWGQGTLV | |
| 005 in VL- | TVSAGSTTTPAPRPPTPAPTIASQPLSLRPEACRPAAG | |
| linker-VH | GAVHTRGLDFACDIYIWAPLAGTCGVLLLSLVITLYCKR | |
| orientation, | GRKKLLYIFKQPFMRPVQTTQEEDGCSCRFPEEEEGG | |
| and V01 | CELRVKFSRSADAPAYKQGQNQLYNELNLGRREEYD | |
| sequence | VLDKRRGRDPEMGGKPRRKNPQEGLYNELQKDKMA | |
| which is | EAYSEIGMKGERRRGKGHDGLYQGLSTATKDTYDAL | |
| underlined) | HMQALPPR | |
| CAR_EBV- | MEFGLSWLFLVAILKGVQCQIVLTQSPAIMSASLGERV | 66 |
| Gp350- | TMTCTASSSVSSSYLHWYQQKPGSSPKLWIYSASNLA | |
| 005_VL- | SGVPPRFSGSGSGTSYSLTISSMEAEDAATYYCHQYH | |
| linker-VH_V02 | RSPPWTFGGGTKLEIKSGGGGSGGGGSGGGGSQVH | |
| (CAR | LQQSGPELVKPGASVKISCKASGYNFNDYYINWVKQR | |
| comprising | PGQGLEWIGWIFPGRGRSNYSEKFKGKATLTVDKSSS | |
| EBV-Gp350- | TAYVLLSSLTSEDSAVYFCARGTAQAGFPYWGQGTLV | |
| 005 in VL- | TVSAGSESKYGPPCPPCPIYIWAPLAGTCGVLLLSLVIT | |
| linker-VH | LYCKRGRKKLLYIFKQPFMRPVQTTQEEDGCSCRFPE | |
| orientation, | EEEGGCELRVKFSRSADAPAYKQGQNQLYNELNLGR | |
| and V02 | REEYDVLDKRRGRDPEMGGKPRRKNPQEGLYNELQ | |
| sequence | KDKMAEAYSEIGMKGERRRGKGHDGLYQGLSTATKD | |
| which is | TYDALHMQALPPR | |
| underlined) | | |
| CAR_EBV- | MEFGLSWLFLVAILKGVQCQIVLTQSPAIMSASLGERV | 67 |
| Gp350- | TMTCTASSSVSSSYLHWYQQKPGSSPKLWIYSASNLA | |
| 005_VL- | SGVPPRFSGSGSGTSYSLTISSMEAEDAATYYCHQYH | |
| linker-VH_V04 | RSPPWTFGGGTKLEIKSGGGGSGGGGSGGGGSQVH | |
| (CAR | LQQSGPELVKPGASVKISCKASGYNFNDYYINWVKQR | |
| comprising | PGQGLEWIGWIFPGRGRSNYSEKFKGKATLTVDKSSS | |
| EBV-Gp350- | TAYVLLSSLTSEDSAVYFCARGTAQAGFPYWGQGTLV | |
| 005 in VL- | TVSAGSESKYGPPCPPCPAPPVAGPSVFLFPPKPKDT | |
| linker-VH | LMISRTPEVTCVVVDVSQEDPEVQFNWYVDGVEVHN | |
| orientation, | AKTKPREEQFQSTYRVVSVLTVLHQDWLNGKEYKCK | |
| and V04 | VSNKGLPSSIEKTISKAKGQPREPQVYTLPPSQEEMTK | |
| sequence | NQVSLTCLVKGFYPSDIAVEWESNGQPENNYKTTPPV | |
| which is | LDSDGSFFLYSRLTVDKSRWQEGNVFSCSVMHEALH | |
| underlined) | NHYTQKSLSLSPGKIYIWAPLAGTCGVLLLSLVITLYCK | |
| | RGRKKLLYIFKQPFMRPVQTTQEEDGCSCRFPEEEEG | |
| | GCELRVKFSRSADAPAYKQGQNQLYNELNLGRREEY | |
| | DVLDKRRGRDPEMGGKPRRKNPQEGLYNELQKDKM | |
| | AEAYSEIGMKGERRRGKGHDGLYQGLSTATKDTYDA | |
| | LHMQALPPR | |
| CAR_EBV- | MEFGLSWLFLVAILKGVQCQVHLQQSGPELVKPGASV | 68 |
| Gp350- | KISCKASGYNFNDYYINWVKQRPGQGLEWIGWIFPGR | |
| 005_VH- | GRSNYSEKFKGKATLTVDKSSSTAYVLLSSLTSEDSAV | |
| linker-VL_V01 | YFCARGTAQAGFPYWGQGTLVTVSASGGGGSGGGG | |
| (CAR | SGGGGSQIVLTQSPAIMSASLGERVTMTCTASSSVSS | |
| comprising | SYLHWYQQKPGSSPKLWIYSASNLASGVPPRFSGSG | |
| EBV-Gp350- | SGTSYSLTISSMEAEDAATYYCHQYHRSPPWTFGGG | |
| 005 in VH- | TKLEIKGSTTTPAPRPPTPAPTIASQPLSLRPEACRPAA | |
| linker-VL | GGAVHTRGLDFACDIYIWAPLAGTCGVLLLSLVITLYCK | |
| orientation, | RGRKKLLYIFKQPFMRPVQTTQEEDGCSCRFPEEEEG | |
| and V01 | GCELRVKFSRSADAPAYKQGQNQLYNELNLGRREEY | |
| sequence | DVLDKRRGRDPEMGGKPRRKNPQEGLYNELQKDKM | |
| which is | AEAYSEIGMKGERRRGKGHDGLYQGLSTATKDTYDA | |
| underlined) | LHMQALPPR | |
| CAR_EBV- | MEFGLSWLFLVAILKGVQCQVHLQQSGPELVKPGASV | 69 |
| Gp350- | KISCKASGYNFNDYYINWVKQRPGQGLEWIGWIFPGR | |
| 005_VH- | GRSNYSEKFKGKATLTVDKSSSTAYVLLSSLTSEDSAV | |
| linker-VL_V02 | YFCARGTAQAGFPYWGQGTLVTVSASGGGGGGGG | |
| (CAR | SGGGGSQIVLTQSPAIMSASLGERVTMTCTASSSVSS | |
| comprising | SYLHWYQQKPGSSPKLWIYSASNLASGVPPRFSGSG | |
| EBV-Gp350- | SGTSYSLTISSMEAEDAATYYCHQYHRSPPWTFGGG | |
| 005 in VH- | TKLEIKGSESKYGPPCPPCPIYIWAPLAGTCGVLLLSLV | |
| linker-VL | ITLYCKRGRKKLLYIFKQPFMRPVQTTQEEDGCSCRFP | |
| orientation, | EEEEGGCELRVKFSRSADAPAYKQGQNQLYNELNLG | |
| and V02 | RREEYDVLDKRRGRDPEMGGKPRRKNPQEGLYNEL | |
| sequence | QKDKMAEAYSEIGMKGERRRGKGHDGLYQGLSTATK | |
| which is | DTYDALHMQALPPR | |
| underlined) | | |
| CAR_EBV- | MEFGLSWLFLVAILKGVQCQVHLQQSGPELVKPGASV | 70 |
| Gp350- | KISCKASGYNFNDYYINWVKQRPGQGLEWIGWIFPGR | |
| 005_VH- | GRSNYSEKFKGKATLTVDKSSSTAYVLLSSLTSEDSAV | |
| linker-VL_V04 | YFCARGTAQAGFPYWGQGTLVTVSASGGGGSGGGG | |
| (CAR | SGGGGSQIVLTQSPAIMSASLGERVTMTCTASSSVSS | |
| comprising | SYLHWYQQKPGSSPKLWIYSASNLASGVPPRFSGSG | |
| EBV-Gp350- | SGTSYSLTISSMEAEDAATYYCHQYHRSPPWTFGGG | |
| 005 in VH- | TKLEIKGSESKYGPPCPPCPAPPVAGPSVFLFPPKPKD | |
| linker-VL | TLMISRTPEVTCVVVDVSQEDPEVQFNWYVDGVEVH | |
| orientation, | NAKTKPREEQFQSTYRVVSVLTVLHQDWLNGKEYKC | |
| and V04 | KVSNKGLPSSIEKTISKAKGQPREPQVYTLPPSQEEMT | |
| sequence | KNQVSLTCLVKGFYPSDIAVEWESNGQPENNYKTTPP | |
| which is | VLDSDGSFFLYSRLTVDKSRWQEGNVFSCSVMHEAL | |
| underlined) | HNHYTQKSLSLSPGKIYIWAPLAGTCGVLLLSLVITLYC | |
| | KRGRKKLLYIFKQPFMRPVQTTQEEDGCSCRFPEEEE | |
| | GGCELRVKFSRSADAPAYKQGQNQLYNELNLGRREE | |
| | YDVLDKRRGRDPEMGGKPRRKNPQEGLYNELQKDK | |
| | MAEAYSEIGMKGERRRGKGHDGLYQGLSTATKDTYD | |
| | ALHMQALPPR | |
| CAR_EBV- | MEFGLSWLFLVAILKGVQCDIQMTQSPSSLSASVGDR | 144 |
| Gp350- | VTITCKASQSVGTNVAWFQQKPGKAPKALIYSASYRY | |
| 003_VL1- | SGVPSRFSGSGSGTDFTLTISSLQPEDFATYFCQQYN | |
| linker- | SYPSTFGGGTKLEIKSGGGGSGGGGSGGGGSQVQL | |
| VH1_V01 | VQSGAEVKKPGASVKVSCKASGYSFTDYNMNWVRQ | |
| | APGQGLEWIGVINPNFGTTSYNQKFKGRVTMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 145 |
| Gp350- | KVSCKASGYSFTDYNMNWVRQAPGQGLEWIGVINPN | |
| 003_VH1- | FGTTSYNQKFKGRVTMTVDTSTSTVYMELSSLRSEDT | |
| linker- | AVYYCARSGYYGSNRYWYFDVWGQGTTVTVSSSGG | |
| VL1_V01 | GGSGGGGSGGGGSDIQMTQSPSSLSASVGDRVTITC | |
| | KASQSVGTNVAWFQQKPGKAPKALIYSASYRYSGVP | |
| | SRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 146 |
| Gp350- | VTITCKASQSVGTNVAWFQQKPGKAPKALIYSASYRY | |
| 003_VL1- | SGVPSRFSGSGSGTDFTLTISSLQPEDFATYFCQQYN | |
| linker- | SYPSTFGGGTKLEIKSGGGGSGGGGSGGGGSQVQL | |
| VH2_V01 | VQSGAEVKKPGASVKVSCKASGYSFTDYNMNWVKQ | |
| | APGQGLEWIGVINPNFGTTSYNQKFKGRATMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 147 |
| Gp350- | KVSCKASGYSFTDYNMNWVKQAPGQGLEWIGVINPN | |
| 003_VH2- | FGTTSYNQKFKGRATMTVDTSTSTVYMELSSLRSEDT | |
| linker- | AVYYCARSGYYGSNRYWYFDVWGQGTTVTVSSSGG | |
| VL1_V01 | GGSGGGGSGGGGSDIQMTQSPSSLSASVGDRVTITC | |
| | KASQSVGTNVAWFQQKPGKAPKALIYSASYRYSGVP | |
| | SRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 148 |
| Gp350- | VTITCKASQSVGTNVAWFQQKPGKAPKALIYSASYRY | |
| 003_VL1- | SGVPSRFSGSGSGTDFTLTISSLQPEDFATYFCQQYN | |
| linker- | SYPSTFGGGTKLEIKSGGGGSGGGGSGGGGSQVQL | |
| VH3_V01 | VQSGAEVKKPGASVKVSCKASGYSFTDYNMNWVKQ | |
| | APGQGLEWIGVINPNFGTTSYNQKFKGRATLTVDQST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 149 |
| Gp350- | KVSCKASGYSFTDYNMNWVKQAPGQGLEWIGVINPN | |
| 003_VH3- | FGTTSYNQKFKGRATLTVDQSTSTVYMELSSLRSEDT | |
| linker- | AVYYCARSGYYGSNRYWYFDVWGQGTTVTVSSSGG | |
| VL1_V01 | GGSGGGGSGGGGSDIQMTQSPSSLSASVGDRVTITC | |
| | KASQSVGTNVAWFQQKPGKAPKALIYSASYRYSGVP | |
| | SRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 150 |
| Gp350- | VTITCKASQSVGTNVAWYQQKPGKAPKALIYSASYRY | |
| 003_VL2- | SGVPDRFSGSGSGTDFTLTISSLQPEDFATYFCQQYN | |
| linker- | SYPSTFGGGTKLEIKSGGGGSGGGGSGGGGSQVQL | |
| VH1_V01 | VQSGAEVKKPGASVKVSCKASGYSFTDYNMNWVRQ | |
| | APGQGLEWIGVINPNFGTTSYNQKFKGRVTMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 151 |
| Gp350- | KVSCKASGYSFTDYNMNWVRQAPGQGLEWIGVINPN | |
| 003_VH1- | FGTTSYNQKFKGRVTMTVDTSTSTVYMELSSLRSEDT | |
| linker- | AVYYCARSGYYGSNRYWYFDVWGQGTTVTVSSSGG | |
| VL2_V01 | GGSGGGGSGGGGSDIQMTQSPSSLSASVGDRVTITC | |
| | KASQSVGTNVAWYQQKPGKAPKALIYSASYRYSGVP | |
| | DRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 152 |
| Gp350- | VTITCKASQSVGTNVAWYQQKPGKAPKALIYSASYRY | |
| 003_VL2- | SGVPDRFSGSGSGTDFTLTISSLQPEDFATYFCQQYN | |
| linker- | SYPSTFGGGTKLEIKSGGGGSGGGGSGGGGSQVQL | |
| VH2_V01 | VQSGAEVKKPGASVKVSCKASGYSFTDYNMNWVKQ | |
| | APGQGLEWIGVINPNFGTTSYNQKFKGRATMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 153 |
| Gp350- | KVSCKASGYSFTDYNMNWVKQAPGQGLEWIGVINPN | |
| 003_VH2- | FGTTSYNQKFKGRATMTVDTSTSTVYMELSSLRSEDT | |
| linker- | AVYYCARSGYYGSNRYWYFDVWGQGTTVTVSSSGG | |
| VL2_V01 | GGSGGGGSGGGGSDIQMTQSPSSLSASVGDRVTITC | |
| | KASQSVGTNVAWYQQKPGKAPKALIYSASYRYSGVP | |
| | DRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 154 |
| Gp350- | VTITCKASQSVGTNVAWYQQKPGKAPKALIYSASYRY | |
| 003_VL2- | SGVPDRFSGSGSGTDFTLTISSLQPEDFATYFCQQYN | |
| linker- | SYPSTFGGGTKLEIKSGGGGSGGGGGGGGSQVQL | |
| VH3_V01 | VQSGAEVKKPGASVKVSCKASGYSFTDYNMNWVKQ | |
| | APGQGLEWIGVINPNFGTTSYNQKFKGRATLTVDQST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 155 |
| Gp350- | KVSCKASGYSFTDYNMNWVKQAPGQGLEWIGVINPN | |
| 003_VH3- | FGTTSYNQKFKGRATLTVDQSTSTVYMELSSLRSEDT | |
| linker- | AVYYCARSGYYGSNRYWYFDVWGQGTTVTVSSSGG | |
| VL2_V01 | GGSGGGGSGGGGSDIQMTQSPSSLSASVGDRVTITC | |
| | KASQSVGTNVAWYQQKPGKAPKALIYSASYRYSGVP | |
| | DRFSGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 156 |
| Gp350- | VTITCKASQSVGTNVAWYQQKPGKAPKALIYSASYRY | |
| 003_VL3- | SGVPDRFTGSGSGTDFTLTISSLQPEDFATYFCQQYN | |
| linker- | SYPSTFGGGTKLEIKSGGGGSGGGGSGGGGSQVQL | |
| VH1_V01 | VQSGAEVKKPGASVKVSCKASGYSFTDYNMNWVRQ | |
| | APGQGLEWIGVINPNFGTTSYNQKFKGRVTMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 157 |
| Gp350- | KVSCKASGYSFTDYNMNWVRQAPGQGLEWIGVINPN | |
| 003_VH1- | FGTTSYNQKFKGRVTMTVDTSTSTVYMELSSLRSEDT | |
| linker- | AVYYCARSGYYGSNRYWYFDVWGQGTTVTVSSSGG | |
| VL3_V01 | GGSGGGGSGGGGSDIQMTQSPSSLSASVGDRVTITC | |
| | KASQSVGTNVAWYQQKPGKAPKALIYSASYRYSGVP | |
| | DRFTGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 158 |
| Gp350-003- | VTITCKASQSVGTNVAWYQQKPGKAPKALIYSASYRY | |
| VL3-linker- | SGVPDRFTGSGSGTDFTLTISSLQPEDFATYFCQQYN | |
| VH2_V01 | SYPSTFGGGTKLEIKSGGGGSGGGGSGGGGSQVQL | |
| | VQSGAEVKKPGASVKVSCKASGYSFTDYNMNWVKQ | |
| | APGQGLEWIGVINPNFGTTSYNQKFKGRATMTVDTST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 159 |
| Gp350-003- | KVSCKASGYSFTDYNMNWVKQAPGQGLEWIGVINPN | |
| VH2-linker- | FGTTSYNQKFKGRATMTVDTSTSTVYMELSSLRSEDT | |
| VL3_V01 | AVYYCARSGYYGSNRYWYFDVWGQGTTVTVSSSGG | |
| | GGSGGGGSGGGGSDIQMTQSPSSLSASVGDRVTITC | |
| | KASQSVGTNVAWYQQKPGKAPKALIYSASYRYSGVP | |
| | DRFTGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| CAR_EBV- | MEFGLSWLFLVAILKGVQCDIQMTQSPSSLSASVGDR | 160 |
| Gp350-003- | VTITCKASQSVGTNVAWYQQKPGKAPKALIYSASYRY | |
| VL3-linker- | SGVPDRFTGSGSGTDFTLTISSLQPEDFATYFCQQYN | |
| VH3_V01 | SYPSTFGGGTKLEIKSGGGGSGGGGSGGGGSQVQL | |
| | VQSGAEVKKPGASVKVSCKASGYSFTDYNMNWVKQ | |
| | APGQGLEWIGVINPNFGTTSYNQKFKGRATLTVDQST | |
| | STVYMELSSLRSEDTAVYYCARSGYYGSNRYWYFDV | |
| | WGQGTTVTVSSGSTTTPAPRPPTPAPTIASQPLSLRP | |
| | EACRPAAGGAVHTRGLDFACDIYIWAPLAGTCGVLLL | |
| | SLVITLYCKRGRKKLLYIFKQPFMRPVQTTQEEDGCSC | |
| | RFPEEEEGGCELRVKFSRSADAPAYKQGQNQLYNEL | |
| | NLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| CAR_EBV- | MEFGLSWLFLVAILKGVQCQVQLVQSGAEVKKPGASV | 161 |
| Gp350-003- | KVSCKASGYSFTDYNMNWVKQAPGQGLEWIGVINPN | |
| VH3-linker- | FGTTSYNQKFKGRATLTVDQSTSTVYMELSSLRSEDT | |
| VL3_V01 | AVYYCARSGYYGSNRYWYFDVWGQGTTVTVSSSGG | |
| | GGSGGGGSGGGGSDIQMTQSPSSLSASVGDRVTITC | |
| | KASQSVGTNVAWYQQKPGKAPKALIYSASYRYSGVP | |
| | DRFTGSGSGTDFTLTISSLQPEDFATYFCQQYNSYPS | |
| | TFGGGTKLEIKGSTTTPAPRPPTPAPTIASQPLSLRPE | |
| | ACRPAAGGAVHTRGLDFACDIYIWAPLAGTCGVLLLSL | |
| | VITLYCKRGRKKLLYIFKQPFMRPVQTTQEEDGCSCRF | |
| | PEEEEGGCELRVKFSRSADAPAYKQGQNQLYNELNL | |
| | GRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |

TABLE 6
CAR nucleotide sequences.

| | | SEQ |
|---|---|---|
| Name | Nucleotide Sequences | ID NO: |
| CAR EBV- | ATGGAGTTCGGCCTGAGCTGGCTGTTTCTGGTGGC | 71 |
| Gp350- | CATCCTGAAGGGCGTGCAGTGCGACGTGGTCATGA | |
| 001_VL- | CCCAGACACCACTGAGCCTGCCCGTGAGCCTGGGC | |
| linker-VH_V01 | GATCAGGCCTCCATCTCTTGTCGGAGCTCCCAGTCT | |
| (CAR | CTGGCCCACAGCAACGGCAATACATACCTGCACTG | |
| comprising | GTATCTGCAGAAGCCAGGCCAGAGCCCCAAGCTGC | |
| EBV-Gp350- | TGATCTACAAGGTGTCCAACCGGTTCTCTGGAGTGC | |
| 001 in VL- | CAGACCGGTTCAACGGCAGCGGCTCCGGCACCGAT | |
| linker-VH | TTCACACTGAAGATCTCCAGGGTGGAGGCAGAGGA | |
| orientation, | CCTGGGCGTGTATTTTTGCTCTCAGAGCACCCACTT | |
| and V01 | CCCCTGGACATTTGGCGGCGGCACCAAGCTGGAGA | |
| sequence | TCAAGTCCGGCGGGGGGGGCTCCGGCGGCGGCGG | |
| which is | CTCTGGCGGCGGCGGCAGCCAGATCCAGCTGGTG | |
| underlined) | CAGTCCGGCCCTGATCTGAAGAAGACCGGCGAGAC | |
| | AGTGAAGATCAGCTGTAAGGCCTCCGGCTACACATT | |
| | CACCACATATGGCGTGTCTTGGGTGAAGCAGGCCC | |
| | CTGGCAAGGTGCTGAAGTGGATGGGCTGGATCAAC | |
| | ACCTACTCTGGCGTGCCAAGATATGCCGACGATTTC | |
| | AAGGGCCGCTTCGCCTTTAGCCTGGAGACCTCCGC | |
| | CTCTACAGCCTACCTGCAGATCAACAATCTGAAGAA | |
| | TGAGGACACCGCCACATATTTTTGCGCCCGGTTCTT | |
| | TTACGGCTATGACCACACCATGGATTACTGGGGCC | |
| | AGGGCACCTCCGTGACAGTGTCTAGCGGATCCACC | |
| | ACAACACCGGCGCCACGCCCACCAACACCAGCACC | |
| | TACCATCGCAAGCCAGCCACTGTCCCTGCGGCCCG | |
| | AGGCCTGTAGGCCAGCAGCAGGAGGAGCAGTGCA | |
| | CACCAGGGGCCTGGACTTTGCCTGCGACATCTACAT | |
| | CTGGGCACCACTGGCAGGAACATGTGGCGTGCTGC | |
| | TGCTGTCCCTGGTCATCACCCTGTATTGCAAGCGC | |
| | GGCCGGAAGAAGCTGCTGTACATCTTCAAGCAGCC | |
| | CTTCATGCGGCCCGTGCAGACAACCCAGGAGGAGG | |
| | ACGGCTGCAGCTGTAGGTTCCCTGAAGAGGAGGAG | |
| | GGAGGATGTGAGCTGAGGGTGAAGTTTTCCCGGTC | |
| | TGCCGATGCCCCAGCCTATAAGCAGGGCCAGAATC | |
| | AGCTGTACAACGAGCTGAATCTGGGCCGGAGAGAG | |
| | GAGTATGACGTGCTGGATAAGAGGAGGGGAAGAGA | |
| | TCCCGAGATGGGAGGCAAGCCCAGAAGAAAGAACC | |
| | CTCAGGAGGGCCTGTATAATGAGCTGCAGAAGGAC | |
| | AAGATGGCCGAGGCCTACAGCGAGATCGGCATGAA | |
| | GGGAGAGAGGCGCCGGGGCAAGGGACACGATGGC | |
| | CTGTACCAGGGCCTGTCAACAGCAACTAAAGATACT | |
| | TACGACGCACTGCACATGCAGGCTCTGCCACCCCG | |
| | A | |
| CAR_EBV- | ATGGAGTTCGGCCTGAGCTGGCTGTTTCTGGTGGC | 72 |
| Gp350- | CATCCTGAAGGGCGTGCAGTGCGACGTGGTCATGA | |
| 001_VL- | CCCAGACACCACTGAGCCTGCCCGTGAGCCTGGGC | |
| linker-VH_V02 | GATCAGGCCTCCATCTCTTGTCGGAGCTCCCAGTCT | |
| (CAR | CTGGCCCACAGCAACGGCAATACATACCTGCACTG | |
| comprising | GTATCTGCAGAAGCCAGGCCAGAGCCCCAAGCTGC | |
| EBV-Gp350- | TGATCTACAAGGTGTCCAACCGGTTCTCTGGAGTGC | |
| 001 in VL- | CAGACCGGTTCAACGGCAGCGGCTCCGGCACCGAT | |
| linker-VH | TTCACACTGAAGATCTCCAGGGTGGAGGCAGAGGA | |
| orientation, | CCTGGGCGTGTATTTTTGCTCTCAGAGCACCCACTT | |
| and V02 | CCCCTGGACATTTGGGGGGGGCACCAAGCTGGAGA | |
| sequence | TCAAGTCCGGCGGCGGCGGCTCCGGCGGCGGCGG | |
| which is | CTCTGGCGGGGGGGGCAGCCAGATCCAGCTGGTG | |
| underlined) | CAGTCCGGCCCTGATCTGAAGAAGACCGGCGAGAC | |
| | AGTGAAGATCAGCTGTAAGGCCTCCGGCTACACATT | |
| | CACCACATATGGCGTGTCTTGGGTGAAGCAGGCCC | |
| | CTGGCAAGGTGCTGAAGTGGATGGGCTGGATCAAC | |
| | ACCTACTCTGGCGTGCCAAGATATGCCGACGATTTC | |
| | AAGGGCCGCTTCGCCTTTAGCCTGGAGACCTCCGC | |
| | CTCTACAGCCTACCTGCAGATCAACAATCTGAAGAA | |
| | TGAGGACACCGCCACATATTTTTGCGCCCGGTTCTT | |
| | TTACGGCTATGACCACACCATGGATTACTGGGGCC | |
| | AGGGCACCTCCGTGACAGTGTCTAGCGGATCCGAG | |
| | AGCAAGTACGGCCCCCCCTGCCCCCCCTGCCCCAT | |
| | CTACATCTGGGCACCACTGGCAGGAACATGTGGCG | |
| | TGCTGCTGCTGTCCCTGGTCATCACCCTGTATTGCA | |
| | AGCGCGGCCGGAAGAAGCTGCTGTACATCTTCAAG | |
| | CAGCCCTTCATGCGGCCCGTGCAGACAACCCAGGA | |
| | GGAGGACGGCTGCAGCTGTAGGTTCCCTGAAGAGG | |
| | AGGAGGGAGGATGTGAGCTGAGGGTGAAGTTTTCC | |
| | CGGTCTGCCGATGCCCCAGCCTATAAGCAGGGCCA | |
| | GAATCAGCTGTACAACGAGCTGAATCTGGGCCGGA | |
| | GAGAGGAGTATGACGTGCTGGATAAGAGGAGGGGA | |
| | AGAGATCCCGAGATGGGAGGCAAGCCCAGAAGAAA | |
| | GAACCCTCAGGAGGGCCTGTATAATGAGCTGCAGA | |
| | AGGACAAGATGGCCGAGGCCTACAGCGAGATCGG | |
| | CATGAAGGGAGAGAGGCGCCGGGGCAAGGGACAC | |
| | GATGGCCTGTACCAGGGCCTGTCAACAGCAACTAA | |
| | AGATACTTACGACGCACTGCACATGCAGGCTCTGC | |
| | CACCCCGA | |
| CAR_EBV- | ATGGAGTTCGGCCTGAGCTGGCTGTTTCTGGTGGC | 73 |
| Gp350- | CATCCTGAAGGGCGTGCAGTGCGACGTGGTCATGA | |
| 001_VL- | CCCAGACACCACTGAGCCTGCCCGTGAGCCTGGGC | |
| linker-VH_V04 | GATCAGGCCTCCATCTCTTGTCGGAGCTCCCAGTCT | |
| (CAR | CTGGCCCACAGCAACGGCAATACATACCTGCACTG | |
| comprising | GTATCTGCAGAAGCCAGGCCAGAGCCCCAAGCTGC | |
| EBV-Gp350- | TGATCTACAAGGTGTCCAACCGGTTCTCTGGAGTGC | |
| 001 in VL- | CAGACCGGTTCAACGGCAGCGGCTCCGGCACCGAT | |
| linker-VH | TTCACACTGAAGATCTCCAGGGTGGAGGCAGAGGA | |
| orientation, | CCTGGGCGTGTATTTTTGCTCTCAGAGCACCCACTT | |
| and V04 | CCCCTGGACATTTGGGGGGGGCACCAAGCTGGAGA | |
| sequence | TCAAGTCCGGCGGCGGCGGCTCCGGCGGCGGCGG | |
| which is | CTCTGGCGGCGGCGGCAGCCAGATCCAGCTGGTG | |
| underlined) | CAGTCCGGCCCTGATCTGAAGAAGACCGGCGAGAC | |
| | AGTGAAGATCAGCTGTAAGGCCTCCGGCTACACATT | |
| | CACCACATATGGCGTGTCTTGGGTGAAGCAGGCCC | |
| | CTGGCAAGGTGCTGAAGTGGATGGGCTGGATCAAC | |
| | ACCTACTCTGGCGTGCCAAGATATGCCGACGATTTC | |
| | AAGGGCCGCTTCGCCTTTAGCCTGGAGACCTCCGC | |
| | CTCTACAGCCTACCTGCAGATCAACAATCTGAAGAA | |
| | TGAGGACACCGCCACATATTTTTGCGCCCGGTTCTT | |
| | TTACGGCTATGACCACACCATGGATTACTGGGGCC | |
| | AGGGCACCTCCGTGACAGTGTCTAGCGGATCCGAG | |
| | AGCAAGTACGGCCCCCCCTGCCCCCCCTGCCCCG | |
| | CCCCCCCCGTGGCCGGCCCCAGCGTGTTCCTGTTC | |
| | CCCCCCAAGCCCAAGGACACCCTGATGATCAGCCG | |
| | CACCCCCGAGGTGACCTGCGTGGTGGTGGACGTG | |
| | AGCCAGGAGGACCCCGAGGTGCAGTTCAACTGGTA | |
| | CGTGGACGGCGTGGAGGTGCACAACGCCAAGACC | |
| | AAGCCCCGCGAGGAGCAGTTCCAGAGCACCTACCG | |
| | CGTGGTGAGCGTGCTGACCGTGCTGCACCAGGACT | |
| | GGCTGAACGGCAAGGAGTACAAGTGCAAGGTGAGC | |
| | AACAAGGGCCTGCCCAGCAGCATCGAGAAGACCAT | |
| | CAGCAAGGCCAAGGGCCAGCCCCGCGAGCCCCAG | |
| | GTGTACACCCTGCCCCCCAGCCAGGAGGAGATGAC | |
| | CAAGAACCAGGTGAGCCTGACCTGCCTGGTGAAGG | |
| | GCTTCTACCCCAGCGACATCGCCGTGGAGTGGGAG | |
| | AGCAACGGCCAGCCCGAGAACAACTACAAGACCAC | |
| | CCCCCCCGTGCTGGACAGCGACGGCAGCTTCTTCC | |
| | TGTACAGCCGCCTGACCGTGGACAAGAGCCGCTGG | |
| | CAGGAGGGCAACGTGTTCAGCTGCAGCGTGATGCA | |
| | CGAGGCCCTGCACAACCACTACACCCAGAAGAGCC | |
| | TGAGCCTGAGCCCCGGCAAGATCTACATCTGGGCA | |
| | CCACTGGCAGGAACATGTGGCGTGCTGCTGCTGTC | |
| | CCTGGTCATCACCCTGTATTGCAAGCGCGGCCGGA | |
| | AGAAGCTGCTGTACATCTTCAAGCAGCCCTTCATGC | |
| | GGCCCGTGCAGACAACCCAGGAGGAGGACGGCTG | |
| | CAGCTGTAGGTTCCCTGAAGAGGAGGAGGGAGGAT | |
| | GTGAGCTGAGGGTGAAGTTTTCCCGGTCTGCCGAT | |
| | GCCCCAGCCTATAAGCAGGGCCAGAATCAGCTGTA | |
| | CAACGAGCTGAATCTGGGCCGGAGAGAGGAGTATG | |
| | ACGTGCTGGATAAGAGGAGGGGAAGAGATCCCGAG | |
| | ATGGGAGGCAAGCCCAGAAGAAAGAACCCTCAGGA | |
| | GGGCCTGTATAATGAGCTGCAGAAGGACAAGATGG | |
| | CCGAGGCCTACAGCGAGATCGGCATGAAGGGAGA | |
| | GAGGCGCCGGGGCAAGGGACACGATGGCCTGTAC | |
| | CAGGGCCTGTCAACAGCAACTAAAGATACTTACGAC | |
| | GCACTGCACATGCAGGCTCTGCCACCCCGA | |
| CAR_EBV- | ATGGAGTTCGGCCTGTCCTGGCTGTTTCTGGTGGC | 74 |
| Gp350- | CATCCTGAAGGGCGTGCAGTGCCAGATCCAGCTGG | |
| 001_VH- | TGCAGTCTGGCCCCGACCTGAAGAAGACCGGCGAG | |
| linker-VL_V01 | ACAGTGAAGATCAGCTGTAAGGCCTCCGGCTACAC | |
| (CAR | ATTCACCACATATGGCGTGAGCTGGGTGAAGCAGG | |
| comprising | CACCTGGCAAGGTGCTGAAGTGGATGGGCTGGATC | |
| EBV-Gp350- | AACACCTACTCTGGCGTGCCAAGATATGCCGACGA | |
| 001 in VH- | TTTCAAGGGCCGCTTCGCCTTTAGCCTGGAGACCT | |
| linker-VL | CCGCCTCTACAGCCTACCTGCAGATCAACAATCTGA | |
| orientation, | AGAATGAGGATACCGCCACATATTTTTGCGCCCGGT | |
| and V01 | TCTTTTACGGCTATGACCACACAATGGATTACTGGG | |
| sequence | GCCAGGGCACCTCCGTGACAGTGAGCTCCTCCGGC | |
| which is | GGCGGCGGCTCCGGCGGCGGCGGCTCTGGCGGC | |
| underlined) | GGCGGCAGCGACGTGGTCATGACCCAGACACCCCT | |
| | GTCTCTGCCTGTGAGCCTGGGCGATCAGGCCAGCA | |
| | TCTCCTGTAGGTCTAGCCAGTCCCTGGCCCACTCTA | |
| | ACGGCAATACCTACCTGCACTGGTATCTGCAGAAG | |
| | CCAGGCCAGAGCCCCAAGCTGCTGATCTACAAGGT | |
| | GAGCAACCGGTTCTCCGGAGTGCCAGACCGGTTCA | |
| | ACGGCTCTGGCAGCGGCACCGATTTCACACTGAAG | |
| | ATCTCCCGGGTGGAGGCAGAGGACCTGGGCGTGT | |
| | ATTTTTGCTCCCAGTCTACCCACTTCCCTTGGACATT | |
| | TGGCGGCGGCACCAAGCTGGAGATCAAGGGATCCA | |
| | CCACAACACCGGCGCCACGCCCACCAACACCAGCA | |
| | CCTACCATCGCAAGCCAGCCACTGTCCCTGCGGCC | |
| | CGAGGCCTGTAGGCCAGCAGCAGGAGGAGCAGTG | |
| | CACACCAGGGGCCTGGACTTTGCCTGCGACATCTA | |
| | CATCTGGGCACCACTGGCAGGAACATGTGGCGTGC | |
| | TGCTGCTGTCCCTGGTCATCACCCTGTATTGCAAGC | |
| | GCGGCCGGAAGAAGCTGCTGTACATCTTCAAGCAG | |
| | CCCTTCATGCGGCCCGTGCAGACAACCCAGGAGGA | |
| | GGACGGCTGCAGCTGTAGGTTCCCTGAAGAGGAGG | |
| | AGGGAGGATGTGAGCTGAGGGTGAAGTTTTCCCGG | |
| | TCTGCCGATGCCCCAGCCTATAAGCAGGGCCAGAA | |
| | TCAGCTGTACAACGAGCTGAATCTGGGCCGGAGAG | |
| | AGGAGTATGACGTGCTGGATAAGAGGAGGGGAAGA | |
| | GATCCCGAGATGGGAGGCAAGCCCAGAAGAAAGAA | |
| | CCCTCAGGAGGGCCTGTATAATGAGCTGCAGAAGG | |
| | ACAAGATGGCCGAGGCCTACAGCGAGATCGGCATG | |
| | AAGGGAGAGAGGCGCCGGGGCAAGGGACACGATG | |
| | GCCTGTACCAGGGCCTGTCAACAGCAACTAAAGAT | |
| | ACTTACGACGCACTGCACATGCAGGCTCTGCCACC | |
| | CCGA | |
| CAR_EBV- | ATGGAGTTCGGCCTGTCCTGGCTGTTTCTGGTGGC | 75 |
| Gp350- | CATCCTGAAGGGCGTGCAGTGCCAGATCCAGCTGG | |
| 001_VH- | TGCAGTCTGGCCCCGACCTGAAGAAGACCGGCGAG | |
| linker-VL_V02 | ACAGTGAAGATCAGCTGTAAGGCCTCCGGCTACAC | |
| (CAR | ATTCACCACATATGGCGTGAGCTGGGTGAAGCAGG | |
| comprising | CACCTGGCAAGGTGCTGAAGTGGATGGGCTGGATC | |
| EBV-Gp350- | AACACCTACTCTGGCGTGCCAAGATATGCCGACGA | |
| 001 in VH- | TTTCAAGGGCCGCTTCGCCTTTAGCCTGGAGACCT | |
| linker-VL | CCGCCTCTACAGCCTACCTGCAGATCAACAATCTGA | |
| orientation, | AGAATGAGGATACCGCCACATATTTTTGCGCCCGGT | |
| and V02 | TCTTTTACGGCTATGACCACACAATGGATTACTGGG | |
| sequence | GCCAGGGCACCTCCGTGACAGTGAGCTCCTCCGGC | |
| which is | GGCGGCGGCTCCGGCGGCGGCGGCTCTGGCGGC | |
| underlined) | GGCGGCAGCGACGTGGTCATGACCCAGACACCCCT | |
| | GTCTCTGCCTGTGAGCCTGGGCGATCAGGCCAGCA | |
| | TCTCCTGTAGGTCTAGCCAGTCCCTGGCCCACTCTA | |
| | ACGGCAATACCTACCTGCACTGGTATCTGCAGAAG | |
| | CCAGGCCAGAGCCCCAAGCTGCTGATCTACAAGGT | |
| | GAGCAACCGGTTCTCCGGAGTGCCAGACCGGTTCA | |
| | ACGGCTCTGGCAGCGGCACCGATTTCACACTGAAG | |
| | ATCTCCCGGGTGGAGGCAGAGGACCTGGGCGTGT | |
| | ATTTTTGCTCCCAGTCTACCCACTTCCCTTGGACATT | |
| | TGGCGGCGGCACCAAGCTGGAGATCAAGGGATCC | |
| | GAGAGCAAGTACGGCCCCCCCTGCCCCCCCTGCC | |
| | CCATCTACATCTGGGCACCACTGGCAGGAACATGT | |
| | GGCGTGCTGCTGCTGTCCCTGGTCATCACCCTGTA | |
| | TTGCAAGCGCGGCCGGAAGAAGCTGCTGTACATCT | |
| | TCAAGCAGCCCTTCATGCGGCCCGTGCAGACAACC | |
| | CAGGAGGAGGACGGCTGCAGCTGTAGGTTCCCTGA | |
| | AGAGGAGGAGGGAGGATGTGAGCTGAGGGTGAAG | |
| | TTTTCCCGGTCTGCCGATGCCCCAGCCTATAAGCA | |
| | GGGCCAGAATCAGCTGTACAACGAGCTGAATCTGG | |
| | GCCGGAGAGAGGAGTATGACGTGCTGGATAAGAGG | |
| | AGGGGAAGAGATCCCGAGATGGGAGGCAAGCCCA | |
| | GAAGAAAGAACCCTCAGGAGGGCCTGTATAATGAG | |
| | CTGCAGAAGGACAAGATGGCCGAGGCCTACAGCGA | |
| | GATCGGCATGAAGGGAGAGAGGCGCCGGGGCAAG | |
| | GGACACGATGGCCTGTACCAGGGCCTGTCAACAGC | |
| | AACTAAAGATACTTACGACGCACTGCACATGCAGGC | |
| | TCTGCCACCCCGA | |
| CAR_EBV- | ATGGAGTTCGGCCTGTCCTGGCTGTTTCTGGTGGC | |
| Gp350- | CATCCTGAAGGGCGTGCAGTGCCAGATCCAGCTGG | |
| 001_VH- | TGCAGTCTGGCCCCGACCTGAAGAAGACCGGCGAG | |
| linker-VL_V04 | ACAGTGAAGATCAGCTGTAAGGCCTCCGGCTACAC | |
| (CAR | ATTCACCACATATGGCGTGAGCTGGGTGAAGCAGG | |
| comprising | CACCTGGCAAGGTGCTGAAGTGGATGGGCTGGATC | |
| EBV-Gp350- | AACACCTACTCTGGCGTGCCAAGATATGCCGACGA | |
| 001 in VH- | TTTCAAGGGCCGCTTCGCCTTTAGCCTGGAGACCT | |
| linker-VL | CCGCCTCTACAGCCTACCTGCAGATCAACAATCTGA | |
| orientation, | AGAATGAGGATACCGCCACATATTTTTGCGCCCGGT | |
| and V04 | TCTTTTACGGCTATGACCACACAATGGATTACTGGG | |
| sequence | GCCAGGGCACCTCCGTGACAGTGAGCTCCTCCGGC | |
| which is | GGCGGCGGCTCCGGCGGCGGCGGCTCTGGCGGC | |
| underlined) | GGCGGCAGCGACGTGGTCATGACCCAGACACCCCT | |
| | GTCTCTGCCTGTGAGCCTGGGCGATCAGGCCAGCA | |
| | TCTCCTGTAGGTCTAGCCAGTCCCTGGCCCACTCTA | |
| | ACGGCAATACCTACCTGCACTGGTATCTGCAGAAG | |
| | CCAGGCCAGAGCCCCAAGCTGCTGATCTACAAGGT | |
| | GAGCAACCGGTTCTCCGGAGTGCCAGACCGGTTCA | |
| | ACGGCTCTGGCAGGGGCACCGATTTCACACTGAAG | |
| | ATCTCCCGGGTGGAGGCAGAGGACCTGGGCGTGT | |
| | ATTTTTGCTCCCAGTCTACCCACTTCCCTTGGACATT | |
| | TGGCGGGGGCACCAAGCTGGAGATCAAGGGATCC | |
| | GAGAGCAAGTACGGCCCCCCCTGCCCCCCCTGCC | |
| | CCGCCCCCCCCGTGGCCGGCCCCAGCGTGTTCCT | |
| | GTTCCCCCCCAAGCCCAAGGACACCCTGATGATCA | |
| | GCCGCACCCCCGAGGTGACCTGCGTGGTGGTGGA | |
| | CGTGAGCCAGGAGGACCCCGAGGTGCAGTTCAACT | |
| | GGTACGTGGACGGCGTGGAGGTGCACAACGCCAA | |
| | GACCAAGCCCCGCGAGGAGCAGTTCCAGAGCACCT | |
| | ACCGCGTGGTGAGCGTGCTGACCGTGCTGCACCAG | |
| | GACTGGCTGAACGGCAAGGAGTACAAGTGCAAGGT | |
| | GAGCAACAAGGGCCTGCCCAGCAGCATCGAGAAGA | |
| | CCATCAGCAAGGCCAAGGGCCAGCCCCGCGAGCC | |
| | CCAGGTGTACACCCTGCCCCCCAGCCAGGAGGAGA | |
| | TGACCAAGAACCAGGTGAGCCTGACCTGCCTGGTG | |
| | AAGGGCTTCTACCCCAGCGACATCGCCGTGGAGTG | |
| | GGAGAGCAACGGCCAGCCCGAGAACAACTACAAGA | |
| | CCACCCCCCCCGTGCTGGACAGCGACGGCAGCTTC | |
| | TTCCTGTACAGCCGCCTGACCGTGGACAAGAGCCG | |
| | CTGGCAGGAGGGCAACGTGTTCAGCTGCAGCGTGA | |
| | TGCACGAGGCCCTGCACAACCACTACACCCAGAAG | |
| | AGCCTGAGCCTGAGCCCCGGCAAGATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 77 |
| Gp350- | CATCCTGAAGGGCGTGCAGTGCGACGTGGTCATGA | |
| 002_VL- | CCCAGACACCCCTGTCTCTGCCTGTGAGCCTGGGC | |
| linker-VH_V01 | GATCAGGCCTCTATCAGCTGTAGCTCCTCTCAGTCC | |
| (CAR | | |
| comprising | CTGGTGCACTCTAACGGCAATACCTACCTGCACTG | |
| EBV-Gp350- | GTATCTGCAGCGGCCAGGCCAGTCTCCCAAGCTGC | |
| 002 in VL- | TGATCTACAAGGTGAGCAACCGGTTCTCCGGCGTG | |
| linker-VH | CCAGACAGATTTTCCGGCTCTGGCAGCGGCACCGA | |
| orientation, | TTTCACACTGAAGATCTCCAGGGTGGAGGCAGAGG | |
| and V01 | ACCTGGGCGTGTATTTCTGCTCCCAGTCTACCCACT | |
| sequence | TTCCCTGGACAATCGGCGGCGGCATCAAGCTGGAG | |
| which is | ATCAAGTCCGGCGGCGGCGGCTCCGGCGGCGGCG | |
| underlined) | GCTCTGGCGGCGGCGGCAGCCAGATCCAGCTGGT | |
| | GCAGAGCGGCCCTGAGCTGAAGAAGCCAGGCGAG | |
| | ACCGTGAAGATCTCTTGTAAGGCCAGCGGCTACAC | |
| | CTTCACAATCTATGGAATGTCCTGGGTGAAGCAGGC | |
| | ACCAGGCAAGGGACTGAGGTGGATGGGCTGGATCA | |
| | ATACATACTCCGGCGTGCCTAAGTATGCCGACGACT | |
| | TCAAGGGCCACTTCGCCTTTTCTCTGGAGACCAGC | |
| | GCCTCCACAGCCTACCTGAGGATCAACAATCTGAA | |
| | GGACGAGGATACCGCCACATATTTCTGCGCCCGCT | |
| | TTTACTATGGCTACGACCACGCCATGGATTATTGGG | |
| | GCCAGGGCACCTCCGTGATCGTGAGCTCCGGATCC | |
| | ACCACAACACCGGCGCCACGCCCACCAACACCAGC | |
| | ACCTACCATCGCAAGCCAGCCACTGTCCCTGCGGC | |
| | CCGAGGCCTGTAGGCCAGCAGCAGGAGGAGCAGT | |
| | GCACACCAGGGGCCTGGACTTTGCCTGCGACATCT | |
| | ACATCTGGGCACCACTGGCAGGAACATGTGGCGTG | |
| | CTGCTGCTGTCCCTGGTCATCACCCTGTATTGCAAG | |
| | CGCGGCCGGAAGAAGCTGCTGTACATCTTCAAGCA | |
| | GCCCTTCATGCGGCCCGTGCAGACAACCCAGGAGG | |
| | AGGACGGCTGCAGCTGTAGGTTCCCTGAAGAGGAG | |
| | GAGGGAGGATGTGAGCTGAGGGTGAAGTTTTCCCG | |
| | GTCTGCCGATGCCCCAGCCTATAAGCAGGGCCAGA | |
| | ATCAGCTGTACAACGAGCTGAATCTGGGCCGGAGA | |
| | GAGGAGTATGACGTGCTGGATAAGAGGAGGGGAAG | |
| | AGATCCCGAGATGGGAGGCAAGCCCAGAAGAAAGA | |
| | ACCCTCAGGAGGGCCTGTATAATGAGCTGCAGAAG | |
| | GACAAGATGGCCGAGGCCTACAGCGAGATCGGCAT | |
| | GAAGGGAGAGAGGCGCCGGGGCAAGGGACACGAT | |
| | GGCCTGTACCAGGGCCTGTCAACAGCAACTAAAGA | |
| | TACTTACGACGCACTGCACATGCAGGCTCTGCCAC | |
| | CCCGA | |
| CAR_EBV- | ATGGAGTTCGGCCTGAGCTGGCTGTTTCTGGTGGC | 78 |
| Gp350- | CATCCTGAAGGGCGTGCAGTGCGACGTGGTCATGA | |
| 002_VL- | CCCAGACACCCCTGTCTCTGCCTGTGAGCCTGGGC | |
| linker-VH_V02 | GATCAGGCCTCTATCAGCTGTAGCTCCTCTCAGTCC | |
| (CAR | CTGGTGCACTCTAACGGCAATACCTACCTGCACTG | |
| comprising | GTATCTGCAGCGGCCAGGCCAGTCTCCCAAGCTGC | |
| EBV-Gp350- | TGATCTACAAGGTGAGCAACCGGTTCTCCGGCGTG | |
| 002 in VL- | CCAGACAGATTTTCCGGCTCTGGCAGCGGCACCGA | |
| linker-VH | TTTCACACTGAAGATCTCCAGGGTGGAGGCAGAGG | |
| orientation, | ACCTGGGCGTGTATTTCTGCTCCCAGTCTACCCACT | |
| and V02 | TTCCCTGGACAATCGGCGGCGGCATCAAGCTGGAG | |
| sequence | ATCAAGTCCGGCGGGGGGGGCTCCGGCGGCGGCG | |
| which is | GCTCTGGGGGGGGCGGCAGCCAGATCCAGCTGGT | |
| underlined) | GCAGAGCGGCCCTGAGCTGAAGAAGCCAGGCGAG | |
| | ACCGTGAAGATCTCTTGTAAGGCCAGCGGCTACAC | |
| | CTTCACAATCTATGGAATGTCCTGGGTGAAGCAGGC | |
| | ACCAGGCAAGGGACTGAGGTGGATGGGCTGGATCA | |
| | ATACATACTCCGGCGTGCCTAAGTATGCCGACGACT | |
| | TCAAGGGCCACTTCGCCTTTTCTCTGGAGACCAGC | |
| | GCCTCCACAGCCTACCTGAGGATCAACAATCTGAA | |
| | GGACGAGGATACCGCCACATATTTCTGCGCCCGCT | |
| | TTTACTATGGCTACGACCACGCCATGGATTATTGGG | |
| | GCCAGGGCACCTCCGTGATCGTGAGCTCCGGATCC | |
| | GAGAGCAAGTACGGCCCCCCCTGCCCCCCCTGCC | |
| | CCATCTACATCTGGGCACCACTGGCAGGAACATGT | |
| | GGCGTGCTGCTGCTGTCCCTGGTCATCACCCTGTA | |
| | TTGCAAGCGCGGCCGGAAGAAGCTGCTGTACATCT | |
| | TCAAGCAGCCCTTCATGCGGCCCGTGCAGACAACC | |
| | CAGGAGGAGGACGGCTGCAGCTGTAGGTTCCCTGA | |
| | AGAGGAGGAGGGAGGATGTGAGCTGAGGGTGAAG | |
| | TTTTCCCGGTCTGCCGATGCCCCAGCCTATAAGCA | |
| | GGGCCAGAATCAGCTGTACAACGAGCTGAATCTGG | |
| | GCCGGAGAGAGGAGTATGACGTGCTGGATAAGAGG | |
| | AGGGGAAGAGATCCCGAGATGGGAGGCAAGCCCA | |
| | GAAGAAAGAACCCTCAGGAGGGCCTGTATAATGAG | |
| | CTGCAGAAGGACAAGATGGCCGAGGCCTACAGCGA | |
| | GATCGGCATGAAGGGAGAGAGGCGCCGGGGCAAG | |
| | GGACACGATGGCCTGTACCAGGGCCTGTCAACAGC | |
| | AACTAAAGATACTTACGACGCACTGCACATGCAGGC | |
| | TCTGCCACCCCGA | |
| CAR_EBV- | ATGGAGTTCGGCCTGAGCTGGCTGTTTCTGGTGGC | 79 |
| Gp350- | CATCCTGAAGGGCGTGCAGTGCGACGTGGTCATGA | |
| 002_VL- | CCCAGACACCCCTGTCTCTGCCTGTGAGCCTGGGC | |
| linker-VH_ V04 | GATCAGGCCTCTATCAGCTGTAGCTCCTCTCAGTCC | |
| (CAR | CTGGTGCACTCTAACGGCAATACCTACCTGCACTG | |
| comprising | GTATCTGCAGCGGCCAGGCCAGTCTCCCAAGCTGC | |
| EBV-Gp350- | TGATCTACAAGGTGAGCAACCGGTTCTCCGGCGTG | |
| 002 in VL- | CCAGACAGATTTTCCGGCTCTGGCAGCGGCACCGA | |
| linker-VH | TTTCACACTGAAGATCTCCAGGGTGGAGGCAGAGG | |
| orientation, | ACCTGGGCGTGTATTTCTGCTCCCAGTCTACCCACT | |
| and V04 | TTCCCTGGACAATCGGGGGGGGCATCAAGCTGGAG | |
| sequence | ATCAAGTCCGGCGGGGGGGGCTCCGGCGGCGGCG | |
| which is | GCTCTGGCGGCGGCGGCAGCCAGATCCAGCTGGT | |
| underlined) | GCAGAGCGGCCCTGAGCTGAAGAAGCCAGGCGAG | |
| | ACCGTGAAGATCTCTTGTAAGGCCAGCGGCTACAC | |
| | CTTCACAATCTATGGAATGTCCTGGGTGAAGCAGGC | |
| | ACCAGGCAAGGGACTGAGGTGGATGGGCTGGATCA | |
| | ATACATACTCCGGCGTGCCTAAGTATGCCGACGACT | |
| | TCAAGGGCCACTTCGCCTTTTCTCTGGAGACCAGC | |
| | GCCTCCACAGCCTACCTGAGGATCAACAATCTGAA | |
| | GGACGAGGATACCGCCACATATTTCTGCGCCCGCT | |
| | TTTACTATGGCTACGACCACGCCATGGATTATTGGG | |
| | GCCAGGGCACCTCCGTGATCGTGAGCTCCGGATCC | |
| | GAGAGCAAGTACGGCCCCCCCTGCCCCCCCTGCC | |
| | CCGCCCCCCCCGTGGCCGGCCCCAGCGTGTTCCT | |
| | GTTCCCCCCCAAGCCCAAGGACACCCTGATGATCA | |
| | GCCGCACCCCCGAGGTGACCTGCGTGGTGGTGGA | |
| | CGTGAGCCAGGAGGACCCCGAGGTGCAGTTCAACT | |
| | GGTACGTGGACGGCGTGGAGGTGCACAACGCCAA | |
| | GACCAAGCCCCGCGAGGAGCAGTTCCAGAGCACCT | |
| | ACCGCGTGGTGAGCGTGCTGACCGTGCTGCACCAG | |
| | GACTGGCTGAACGGCAAGGAGTACAAGTGCAAGGT | |
| | GAGCAACAAGGGCCTGCCCAGCAGCATCGAGAAGA | |
| | CCATCAGCAAGGCCAAGGGCCAGCCCCGCGAGCC | |
| | CCAGGTGTACACCCTGCCCCCCAGCCAGGAGGAGA | |
| | TGACCAAGAACCAGGTGAGCCTGACCTGCCTGGTG | |
| | AAGGGCTTCTACCCCAGCGACATCGCCGTGGAGTG | |
| | GGAGAGCAACGGCCAGCCCGAGAACAACTACAAGA | |
| | CCACCCCCCCCGTGCTGGACAGCGACGGCAGCTTC | |
| | TTCCTGTACAGCCGCCTGACCGTGGACAAGAGCCG | |
| | CTGGCAGGAGGGCAACGTGTTCAGCTGCAGCGTGA | |
| | TGCACGAGGCCCTGCACAACCACTACACCCAGAAG | |
| | AGCCTGAGCCTGAGCCCCGGCAAGATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 80 |
| Gp350- | CATCCTGAAGGGCGTGCAGTGCCAGATCCAGCTGG | |
| 002_VH- | TGCAGTCCGGCCCTGAGCTGAAGAAGCCAGGCGA | |
| linker-VL_V01 | GACCGTGAAGATCTCTTGTAAGGCCAGCGGCTACA | |
| (CAR | CCTTCACAATCTATGGAATGAGCTGGGTGAAGCAG | |
| comprising | GCACCAGGCAAGGGACTGAGGTGGATGGGCTGGA | |
| EBV-Gp350- | TCAACACATACTCCGGCGTGCCTAAGTATGCCGAC | |
| 002 in VH- | GACTTCAAGGGCCACTTCGCCTTTTCTCTGGAGACC | |
| linker-VL | TCTGCCAGCACAGCCTACCTGAGGATCAACAATCTG | |
| orientation, | AAGGACGAGGATACCGCCACATATTTCTGCGCCCG | |
| and V01 | CTTTTACTATGGCTACGACCACGCCATGGATTATTG | |
| sequence | GGGCCAGGGCACCTCCGTGATCGTGAGCTCCTCCG | |
| which is | GCGGCGGCGGCTCCGGCGGGGGGGGCTCTGGCG | |
| underlined) | GCGGCGGCAGCGACGTGGTCATGACCCAGACACC | |
| | ACTGTCCCTGCCCGTGAGCCTGGGCGATCAGGCCT | |
| | CCATCTCTTGTTCTAGCTCCCAGAGCCTGGTGCACT | |
| | CCAACGGCAATACCTACCTGCACTGGTATCTGCAG | |
| | CGGCCAGGCCAGTCTCCCAAGCTGCTGATCTACAA | |
| | GGTGTCTAATCGGTTCAGCGGAGTGCCAGACCGGT | |
| | TCAGCGGCTCCGGCTCTGGAACCGATTTCACACTG | |
| | AAGATCTCCAGGGTGGAGGCAGAGGACCTGGGCG | |
| | TGTATTTCTGCAGCCAGTCCACCCACTTTCCCTGGA | |
| | CAATCGGGGGGGGCATCAAGCTGGAGATCAAGGGA | |
| | TCCACCACAACACCGGCGCCACGCCCACCAACACC | |
| | AGCACCTACCATCGCAAGCCAGCCACTGTCCCTGC | |
| | GGCCCGAGGCCTGTAGGCCAGCAGCAGGAGGAGC | |
| | AGTGCACACCAGGGGCCTGGACTTTGCCTGCGACA | |
| | TCTACATCTGGGCACCACTGGCAGGAACATGTGGC | |
| | GTGCTGCTGCTGTCCCTGGTCATCACCCTGTATTGC | |
| | AAGCGCGGCCGGAAGAAGCTGCTGTACATCTTCAA | |
| | GCAGCCCTTCATGCGGCCCGTGCAGACAACCCAGG | |
| | AGGAGGACGGCTGCAGCTGTAGGTTCCCTGAAGAG | |
| | GAGGAGGGAGGATGTGAGCTGAGGGTGAAGTTTTC | |
| | CCGGTCTGCCGATGCCCCAGCCTATAAGCAGGGCC | |
| | AGAATCAGCTGTACAACGAGCTGAATCTGGGCCGG | |
| | AGAGAGGAGTATGACGTGCTGGATAAGAGGAGGGG | |
| | AAGAGATCCCGAGATGGGAGGCAAGCCCAGAAGAA | |
| | AGAACCCTCAGGAGGGCCTGTATAATGAGCTGCAG | |
| | AAGGACAAGATGGCCGAGGCCTACAGCGAGATCGG | |
| | CATGAAGGGAGAGAGGCGCCGGGGCAAGGGACAC | |
| | GATGGCCTGTACCAGGGCCTGTCAACAGCAACTAA | |
| | AGATACTTACGACGCACTGCACATGCAGGCTCTGC | |
| | CACCCCGA | |
| CAR_EBV- | ATGGAGTTCGGCCTGAGCTGGCTGTTTCTGGTGGC | 81 |
| Gp350- | CATCCTGAAGGGCGTGCAGTGCCAGATCCAGCTGG | |
| 002_VH- | TGCAGTCCGGCCCTGAGCTGAAGAAGCCAGGCGA | |
| linker-VL_V02 | GACCGTGAAGATCTCTTGTAAGGCCAGCGGCTACA | |
| (CAR | CCTTCACAATCTATGGAATGAGCTGGGTGAAGCAG | |
| comprising | GCACCAGGCAAGGGACTGAGGTGGATGGGCTGGA | |
| EBV-Gp350- | TCAACACATACTCCGGCGTGCCTAAGTATGCCGAC | |
| 002 in VH- | GACTTCAAGGGCCACTTCGCCTTTTCTCTGGAGACC | |
| linker-VL | TCTGCCAGCACAGCCTACCTGAGGATCAACAATCTG | |
| orientation, | AAGGACGAGGATACCGCCACATATTTCTGCGCCCG | |
| and V02 | CTTTTACTATGGCTACGACCACGCCATGGATTATTG | |
| sequence | GGGCCAGGGCACCTCCGTGATCGTGAGCTCCTCCG | |
| which is | GCGGCGGCGGCTCCGGCGGCGGCGGCTCTGGCG | |
| underlined) | GCGGCGGCAGCGACGTGGTCATGACCCAGACACC | |
| | ACTGTCCCTGCCCGTGAGCCTGGGCGATCAGGCCT | |
| | CCATCTCTTGTTCTAGCTCCCAGAGCCTGGTGCACT | |
| | CCAACGGCAATACCTACCTGCACTGGTATCTGCAG | |
| | CGGCCAGGCCAGTCTCCCAAGCTGCTGATCTACAA | |
| | GGTGTCTAATCGGTTCAGCGGAGTGCCAGACCGGT | |
| | TCAGCGGCTCCGGCTCTGGAACCGATTTCACACTG | |
| | AAGATCTCCAGGGTGGAGGCAGAGGACCTGGGCG | |
| | TGTATTTCTGCAGCCAGTCCACCCACTTTCCCTGGA | |
| | CAATCGGCGGCGGCATCAAGCTGGAGATCAAGGGA | |
| | TCCGAGAGCAAGTACGGCCCCCCCTGCCCCCCCTG | |
| | CCCCATCTACATCTGGGCACCACTGGCAGGAACAT | |
| | GTGGCGTGCTGCTGCTGTCCCTGGTCATCACCCTG | |
| | TATTGCAAGCGCGGCCGGAAGAAGCTGCTGTACAT | |
| | CTTCAAGCAGCCCTTCATGCGGCCCGTGCAGACAA | |
| | CCCAGGAGGAGGACGGCTGCAGCTGTAGGTTCCCT | |
| | GAAGAGGAGGAGGGAGGATGTGAGCTGAGGGTGA | |
| | AGTTTTCCCGGTCTGCCGATGCCCCAGCCTATAAG | |
| | CAGGGCCAGAATCAGCTGTACAACGAGCTGAATCT | |
| | GGGCCGGAGAGAGGAGTATGACGTGCTGGATAAGA | |
| | GGAGGGGAAGAGATCCCGAGATGGGAGGCAAGCC | |
| | CAGAAGAAAGAACCCTCAGGAGGGCCTGTATAATG | |
| | AGCTGCAGAAGGACAAGATGGCCGAGGCCTACAGC | |
| | GAGATCGGCATGAAGGGAGAGAGGCGCCGGGGCA | |
| | AGGGACACGATGGCCTGTACCAGGGCCTGTCAACA | |
| | GCAACTAAAGATACTTACGACGCACTGCACATGCAG | |
| | GCTCTGCCACCCCGA | |
| CAR_EBV- | ATGGAGTTCGGCCTGAGCTGGCTGTTTCTGGTGGC | 82 |
| Gp350- | CATCCTGAAGGGCGTGCAGTGCCAGATCCAGCTGG | |
| 002_VH- | TGCAGTCCGGCCCTGAGCTGAAGAAGCCAGGCGA | |
| linker-VL_V04 | GACCGTGAAGATCTCTTGTAAGGCCAGCGGCTACA | |
| (CAR | CCTTCACAATCTATGGAATGAGCTGGGTGAAGCAG | |
| comprising | GCACCAGGCAAGGGACTGAGGTGGATGGGCTGGA | |
| EBV-Gp350- | TCAACACATACTCCGGCGTGCCTAAGTATGCCGAC | |
| 002 in VH- | GACTTCAAGGGCCACTTCGCCTTTTCTCTGGAGACC | |
| linker-VL | TCTGCCAGCACAGCCTACCTGAGGATCAACAATCTG | |
| orientation, | AAGGACGAGGATACCGCCACATATTTCTGCGCCCG | |
| and V04 | CTTTTACTATGGCTACGACCACGCCATGGATTATTG | |
| sequence | GGGCCAGGGCACCTCCGTGATCGTGAGCTCCTCCG | |
| which is | GCGGCGGCGGCTCCGGCGGCGGCGGCTCTGGCG | |
| underlined) | GCGGCGGCAGCGACGTGGTCATGACCCAGACACC | |
| | ACTGTCCCTGCCCGTGAGCCTGGGCGATCAGGCCT | |
| | CCATCTCTTGTTCTAGCTCCCAGAGCCTGGTGCACT | |
| | CCAACGGCAATACCTACCTGCACTGGTATCTGCAG | |
| | CGGCCAGGCCAGTCTCCCAAGCTGCTGATCTACAA | |
| | GGTGTCTAATCGGTTCAGCGGAGTGCCAGACCGGT | |
| | TCAGCGGCTCCGGCTCTGGAACCGATTTCACACTG | |
| | AAGATCTCCAGGGTGGAGGCAGAGGACCTGGGCG | |
| | TGTATTTCTGCAGCCAGTCCACCCACTTTCCCTGGA | |
| | CAATCGGCGGCGGCATCAAGCTGGAGATCAAGGGA | |
| | TCCGAGAGCAAGTACGGCCCCCCCTGCCCCCCCTG | |
| | CCCCGCCCCCCCCGTGGCCGGCCCCAGCGTGTTC | |
| | CTGTTCCCCCCCAAGCCCAAGGACACCCTGATGAT | |
| | CAGCCGCACCCCCGAGGTGACCTGCGTGGTGGTG | |
| | GACGTGAGCCAGGAGGACCCCGAGGTGCAGTTCAA | |
| | CTGGTACGTGGACGGCGTGGAGGTGCACAACGCC | |
| | AAGACCAAGCCCCGCGAGGAGCAGTTCCAGAGCAC | |
| | CTACCGCGTGGTGAGCGTGCTGACCGTGCTGCACC | |
| | AGGACTGGCTGAACGGCAAGGAGTACAAGTGCAAG | |
| | GTGAGCAACAAGGGCCTGCCCAGCAGCATCGAGAA | |
| | GACCATCAGCAAGGCCAAGGGCCAGCCCCGCGAG | |
| | CCCCAGGTGTACACCCTGCCCCCCAGCCAGGAGGA | |
| | GATGACCAAGAACCAGGTGAGCCTGACCTGCCTGG | |
| | TGAAGGGCTTCTACCCCAGCGACATCGCCGTGGAG | |
| | TGGGAGAGCAACGGCCAGCCCGAGAACAACTACAA | |
| | GACCACCCCCCCCGTGCTGGACAGCGACGGCAGC | |
| | TTCTTCCTGTACAGCCGCCTGACCGTGGACAAGAG | |
| | CCGCTGGCAGGAGGGCAACGTGTTCAGCTGCAGC | |
| | GTGATGCACGAGGCCCTGCACAACCACTACACCCA | |
| | GAAGAGCCTGAGCCTGAGCCCCGGCAAGATCTACA | |
| | TCTGGGCACCACTGGCAGGAACATGTGGCGTGCTG | |
| | CTGCTGTCCCTGGTCATCACCCTGTATTGCAAGCGC | |
| | GGCCGGAAGAAGCTGCTGTACATCTTCAAGCAGCC | |
| | CTTCATGCGGCCCGTGCAGACAACCCAGGAGGAGG | |
| | ACGGCTGCAGCTGTAGGTTCCCTGAAGAGGAGGAG | |
| | GGAGGATGTGAGCTGAGGGTGAAGTTTTCCCGGTC | |
| | TGCCGATGCCCCAGCCTATAAGCAGGGCCAGAATC | |
| | AGCTGTACAACGAGCTGAATCTGGGCCGGAGAGAG | |
| | GAGTATGACGTGCTGGATAAGAGGAGGGGAAGAGA | |
| | TCCCGAGATGGGAGGCAAGCCCAGAAGAAAGAACC | |
| | CTCAGGAGGGCCTGTATAATGAGCTGCAGAAGGAC | |
| | AAGATGGCCGAGGCCTACAGCGAGATCGGCATGAA | |
| | GGGAGAGAGGCGCCGGGGCAAGGGACACGATGGC | |
| | CTGTACCAGGGCCTGTCAACAGCAACTAAAGATACT | |
| | TACGACGCACTGCACATGCAGGCTCTGCCACCCCG | |
| | A | |
| CAR_EBV- | ATGGAGTTCGGCCTGAGCTGGCTGTTTCTGGTGGC | 83 |
| Gp350- | CATCCTGAAGGGCGTGCAGTGCGACATCGTGATGA | |
| 003_VL- | CCCAGAGCCAGAAGTTCATGTCTACAAGCGTGGGC | |
| linker-VH_V01 | GATCGGGTGTCCGTGACCTGTAAGGCCTCCCAGTC | |
| (CAR | TGTGGGCACAAACGTGGCCTGGTATCAGCAGAAGC | |
| comprising | CCGGCCAGTCTCCTAAGGCCCTGATCTATAGCGCC | |
| EBV-Gp350- | TCCTACCGGTATTCCGGCGTGCCTGACAGATTCAC | |
| 003 in VL- | CGGCTCTGGCAGCGGCACAGACTTCACCCTGACAA | |
| linker-VH | TCTCTAACGTGCAGAGCGAGGACCTGGCCGAGTAC | |
| orientation, | TTCTGCCAGCAGTACAATTCCTATCCATCTACCTTTG | |
| and V01 | GCGGCGGCACAAAGCTGGAGATCAAGAGCGGAGG | |
| sequence | AGGAGGCTCCGGAGGAGGAGGCTCTGGCGGCGGC | |
| which is | GGCAGCGAGTTCCAGCTGCAGCAGAGCGGACCAG | |
| underlined) | ACCTGGTGAAGCCAGGAGCCTCCGTGAAGATCAGC | |
| | TGTAAGGCCAGCGGCTACTCCTTTACCGATTATAAC | |
| | ATGAATTGGGTGAAGCAGAGCAACGGCAAGAATCT | |
| | GGAGTGGATCGGCGTGATCAACCCCAATTTCGGCA | |
| | CCACATCCTACAATCAGAAGTTTAAGGGCAAGGCCA | |
| | CCCTGACAGTGGACCAGAGCTCCTCTACCGCCTAT | |
| | ATGCAGCTGAACTCTCTGACAAGCGAGGATTCCGC | |
| | CGTGTACTATTGCGCCAGGTCCGGCTACTATGGCT | |
| | CTAATCGCTACTGGTATTTTGACGTGTGGGGCACCG | |
| | GCACCACAGTGACAGTGAGCTCCGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 84 |
| Gp350- | CATCCTGAAGGGCGTGCAGTGCGACATCGTGATGA | |
| 003_VL- | CCCAGAGCCAGAAGTTCATGTCTACAAGCGTGGGC | |
| linker-VH_V02 | GATCGGGTGTCCGTGACCTGTAAGGCCTCCCAGTC | |
| (CAR | TGTGGGCACAAACGTGGCCTGGTATCAGCAGAAGC | |
| comprising | CCGGCCAGTCTCCTAAGGCCCTGATCTATAGCGCC | |
| EBV-Gp350- | TCCTACCGGTATTCCGGCGTGCCTGACAGATTCAC | |
| 003 in VL- | CGGCTCTGGCAGCGGCACAGACTTCACCCTGACAA | |
| linker-VH | TCTCTAACGTGCAGAGCGAGGACCTGGCCGAGTAC | |
| orientation, | TTCTGCCAGCAGTACAATTCCTATCCATCTACCTTTG | |
| and V02 | GCGGCGGCACAAAGCTGGAGATCAAGAGCGGAGG | |
| sequence | AGGAGGCTCCGGAGGAGGAGGCTCTGGGGGGGGC | |
| which is | GGCAGCGAGTTCCAGCTGCAGCAGAGCGGACCAG | |
| underlined) | ACCTGGTGAAGCCAGGAGCCTCCGTGAAGATCAGC | |
| | TGTAAGGCCAGCGGCTACTCCTTTACCGATTATAAC | |
| | ATGAATTGGGTGAAGCAGAGCAACGGCAAGAATCT | |
| | GGAGTGGATCGGCGTGATCAACCCCAATTTCGGCA | |
| | CCACATCCTACAATCAGAAGTTTAAGGGCAAGGCCA | |
| | CCCTGACAGTGGACCAGAGCTCCTCTACCGCCTAT | |
| | ATGCAGCTGAACTCTCTGACAAGCGAGGATTCCGC | |
| | CGTGTACTATTGCGCCAGGTCCGGCTACTATGGCT | |
| | CTAATCGCTACTGGTATTTTGACGTGTGGGGCACCG | |
| | GCACCACAGTGACAGTGAGCTCCGGATCCGAGAGC | |
| | AAGTACGGCCCCCCCTGCCCCCCCTGCCCCATCTA | |
| | CATCTGGGCACCACTGGCAGGAACATGTGGCGTGC | |
| | TGCTGCTGTCCCTGGTCATCACCCTGTATTGCAAGC | |
| | GCGGCCGGAAGAAGCTGCTGTACATCTTCAAGCAG | |
| | CCCTTCATGCGGCCCGTGCAGACAACCCAGGAGGA | |
| | GGACGGCTGCAGCTGTAGGTTCCCTGAAGAGGAGG | |
| | AGGGAGGATGTGAGCTGAGGGTGAAGTTTTCCCGG | |
| | TCTGCCGATGCCCCAGCCTATAAGCAGGGCCAGAA | |
| | TCAGCTGTACAACGAGCTGAATCTGGGCCGGAGAG | |
| | AGGAGTATGACGTGCTGGATAAGAGGAGGGGAAGA | |
| | GATCCCGAGATGGGAGGCAAGCCCAGAAGAAAGAA | |
| | CCCTCAGGAGGGCCTGTATAATGAGCTGCAGAAGG | |
| | ACAAGATGGCCGAGGCCTACAGCGAGATCGGCATG | |
| | AAGGGAGAGAGGCGCCGGGGCAAGGGACACGATG | |
| | GCCTGTACCAGGGCCTGTCAACAGCAACTAAAGAT | |
| | ACTTACGACGCACTGCACATGCAGGCTCTGCCACC | |
| | CCGA | |
| CAR_EBV- | ATGGAGTTCGGCCTGAGCTGGCTGTTTCTGGTGGC | 85 |
| Gp350- | CATCCTGAAGGGCGTGCAGTGCGACATCGTGATGA | |
| 003_VL- | CCCAGAGCCAGAAGTTCATGTCTACAAGCGTGGGC | |
| linker-VH_ V04 | GATCGGGTGTCCGTGACCTGTAAGGCCTCCCAGTC | |
| (CAR | TGTGGGCACAAACGTGGCCTGGTATCAGCAGAAGC | |
| comprising | CCGGCCAGTCTCCTAAGGCCCTGATCTATAGCGCC | |
| EBV-Gp350- | TCCTACCGGTATTCCGGCGTGCCTGACAGATTCAC | |
| 003 in VL- | CGGCTCTGGCAGCGGCACAGACTTCACCCTGACAA | |
| linker-VH | TCTCTAACGTGCAGAGCGAGGACCTGGCCGAGTAC | |
| orientation, | TTCTGCCAGCAGTACAATTCCTATCCATCTACCTTTG | |
| and V04 | GCGGCGGCACAAAGCTGGAGATCAAGAGCGGAGG | |
| sequence | AGGAGGCTCCGGAGGAGGAGGCTCTGGCGGCGGC | |
| which is | GGCAGCGAGTTCCAGCTGCAGCAGAGCGGACCAG | |
| underlined) | ACCTGGTGAAGCCAGGAGCCTCCGTGAAGATCAGC | |
| | TGTAAGGCCAGCGGCTACTCCTTTACCGATTATAAC | |
| | ATGAATTGGGTGAAGCAGAGCAACGGCAAGAATCT | |
| | GGAGTGGATCGGCGTGATCAACCCCAATTTCGGCA | |
| | CCACATCCTACAATCAGAAGTTTAAGGGCAAGGCCA | |
| | CCCTGACAGTGGACCAGAGCTCCTCTACCGCCTAT | |
| | ATGCAGCTGAACTCTCTGACAAGCGAGGATTCCGC | |
| | CGTGTACTATTGCGCCAGGTCCGGCTACTATGGCT | |
| | CTAATCGCTACTGGTATTTTGACGTGTGGGGCACCG | |
| | GCACCACAGTGACAGTGAGCTCCGGATCCGAGAGC | |
| | AAGTACGGCCCCCCCTGCCCCCCCTGCCCCGCCC | |
| | CCCCCGTGGCCGGCCCCAGCGTGTTCCTGTTCCCC | |
| | CCCAAGCCCAAGGACACCCTGATGATCAGCCGCAC | |
| | CCCCGAGGTGACCTGCGTGGTGGTGGACGTGAGC | |
| | CAGGAGGACCCCGAGGTGCAGTTCAACTGGTACGT | |
| | GGACGGCGTGGAGGTGCACAACGCCAAGACCAAG | |
| | CCCCGCGAGGAGCAGTTCCAGAGCACCTACCGCGT | |
| | GGTGAGCGTGCTGACCGTGCTGCACCAGGACTGG | |
| | CTGAACGGCAAGGAGTACAAGTGCAAGGTGAGCAA | |
| | CAAGGGCCTGCCCAGCAGCATCGAGAAGACCATCA | |
| | GCAAGGCCAAGGGCCAGCCCCGCGAGCCCCAGGT | |
| | GTACACCCTGCCCCCCAGCCAGGAGGAGATGACCA | |
| | AGAACCAGGTGAGCCTGACCTGCCTGGTGAAGGGC | |
| | TTCTACCCCAGCGACATCGCCGTGGAGTGGGAGAG | |
| | CAACGGCCAGCCCGAGAACAACTACAAGACCACCC | |
| | CCCCCGTGCTGGACAGCGACGGCAGCTTCTTCCTG | |
| | TACAGCCGCCTGACCGTGGACAAGAGCCGCTGGCA | |
| | GGAGGGCAACGTGTTCAGCTGCAGCGTGATGCACG | |
| | AGGCCCTGCACAACCACTACACCCAGAAGAGCCTG | |
| | AGCCTGAGCCCCGGCAAGATCTACATCTGGGCACC | |
| | ACTGGCAGGAACATGTGGCGTGCTGCTGCTGTCCC | |
| | TGGTCATCACCCTGTATTGCAAGCGCGGCCGGAAG | |
| | AAGCTGCTGTACATCTTCAAGCAGCCCTTCATGCGG | |
| | CCCGTGCAGACAACCCAGGAGGAGGACGGCTGCA | |
| | GCTGTAGGTTCCCTGAAGAGGAGGAGGGAGGATGT | |
| | GAGCTGAGGGTGAAGTTTTCCCGGTCTGCCGATGC | |
| | CCCAGCCTATAAGCAGGGCCAGAATCAGCTGTACA | |
| | ACGAGCTGAATCTGGGCCGGAGAGAGGAGTATGAC | |
| | GTGCTGGATAAGAGGAGGGGAAGAGATCCCGAGAT | |
| | GGGAGGCAAGCCCAGAAGAAAGAACCCTCAGGAG | |
| | GGCCTGTATAATGAGCTGCAGAAGGACAAGATGGC | |
| | CGAGGCCTACAGCGAGATCGGCATGAAGGGAGAG | |
| | AGGCGCCGGGGCAAGGGACACGATGGCCTGTACC | |
| | AGGGCCTGTCAACAGCAACTAAAGATACTTACGACG | |
| | CACTGCACATGCAGGCTCTGCCACCCCGA | |
| CAR_EBV- | ATGGAGTTCGGCCTGAGCTGGCTGTTTCTGGTGGC | 86 |
| Gp350- | CATCCTGAAGGGCGTGCAGTGCGAGTTCCAGCTGC | |
| 003_VH- | AGCAGAGCGGACCAGACCTGGTGAAGCCAGGAGC | |
| linker-VL_V01 | CAGCGTGAAGATCAGCTGTAAGGCCTCTGGCTACA | |
| (CAR | GCTTTACCGATTATAACATGAATTGGGTGAAGCAGA | |
| comprising | GCAACGGCAAGAATCTGGAGTGGATCGGCGTGATC | |
| EBV-Gp350- | AACCCCAATTTCGGCACCACATCCTACAACCAGAAG | |
| 003 in VH- | TTTAAGGGCAAGGCCACCCTGACAGTGGACCAGAG | |
| linker-VL | CTCCTCTACCGCCTATATGCAGCTGAACTCCCTGAC | |
| orientation, | ATCTGAGGATAGCGCCGTGTACTATTGCGCCAGGT | |
| and V01 | CCGGCTACTATGGCTCTAATCGCTACTGGTATTTCG | |
| sequence | ACGTGTGGGGCACCGGCACCACAGTGACAGTGAG | |
| which is | CTCCTCTGGAGGAGGAGGCAGCGGCGGAGGAGGC | |
| underlined) | TCCGGGGGGGGCGGCTCTGACATCGTGATGACCCA | |
| | GAGCCAGAAGTTTATGAGCACATCCGTGGGCGATC | |
| | GGGTGTCCGTGACCTGCAAGGCCTCTCAGAGCGTG | |
| | GGCACAAATGTGGCCTGGTATCAGCAGAAGCCCGG | |
| | CCAGTCTCCTAAGGCCCTGATCTATTCCGCCTCTTA | |
| | CCGGTATTCCGGCGTGCCTGACAGATTCACCGGCA | |
| | GCGGCTCCGGCACAGACTTCACCCTGACAATCTCT | |
| | AACGTGCAGAGCGAGGATCTGGCCGAGTACTTCTG | |
| | TCAGCAGTACAATTCCTATCCATCTACCTTTGGCGG | |
| | CGGCACAAAGCTGGAGATCAAGGGATCCACCACAA | |
| | CACCGGCGCCACGCCCACCAACACCAGCACCTACC | |
| | ATCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 87 |
| Gp350- | CATCCTGAAGGGCGTGCAGTGCGAGTTCCAGCTGC | |
| 003_VH- | AGCAGAGCGGACCAGACCTGGTGAAGCCAGGAGC | |
| linker-VL_V02 | CAGCGTGAAGATCAGCTGTAAGGCCTCTGGCTACA | |
| (CAR | GCTTTACCGATTATAACATGAATTGGGTGAAGCAGA | |
| comprising | GCAACGGCAAGAATCTGGAGTGGATCGGCGTGATC | |
| EBV-Gp350- | AACCCCAATTTCGGCACCACATCCTACAACCAGAAG | |
| 003 in VH- | TTTAAGGGCAAGGCCACCCTGACAGTGGACCAGAG | |
| linker-VL | CTCCTCTACCGCCTATATGCAGCTGAACTCCCTGAC | |
| orientation, | ATCTGAGGATAGCGCCGTGTACTATTGCGCCAGGT | |
| and V02 | CCGGCTACTATGGCTCTAATCGCTACTGGTATTTCG | |
| sequence | ACGTGTGGGGCACCGGCACCACAGTGACAGTGAG | |
| which is | CTCCTCTGGAGGAGGAGGCAGCGGCGGAGGAGGC | |
| underlined) | TCCGGCGGCGGCGGCTCTGACATCGTGATGACCCA | |
| | GAGCCAGAAGTTTATGAGCACATCCGTGGGCGATC | |
| | GGGTGTCCGTGACCTGCAAGGCCTCTCAGAGCGTG | |
| | GGCACAAATGTGGCCTGGTATCAGCAGAAGCCCGG | |
| | CCAGTCTCCTAAGGCCCTGATCTATTCCGCCTCTTA | |
| | CCGGTATTCCGGCGTGCCTGACAGATTCACCGGCA | |
| | GCGGCTCCGGCACAGACTTCACCCTGACAATCTCT | |
| | AACGTGCAGAGCGAGGATCTGGCCGAGTACTTCTG | |
| | TCAGCAGTACAATTCCTATCCATCTACCTTTGGCGG | |
| | CGGCACAAAGCTGGAGATCAAGGGATCCGAGAGCA | |
| | AGTACGGCCCCCCCTGCCCCCCCTGCCCCATCTAC | |
| | ATCTGGGCACCACTGGCAGGAACATGTGGCGTGCT | |
| | GCTGCTGTCCCTGGTCATCACCCTGTATTGCAAGC | |
| | GCGGCCGGAAGAAGCTGCTGTACATCTTCAAGCAG | |
| | CCCTTCATGCGGCCCGTGCAGACAACCCAGGAGGA | |
| | GGACGGCTGCAGCTGTAGGTTCCCTGAAGAGGAGG | |
| | AGGGAGGATGTGAGCTGAGGGTGAAGTTTTCCCGG | |
| | TCTGCCGATGCCCCAGCCTATAAGCAGGGCCAGAA | |
| | TCAGCTGTACAACGAGCTGAATCTGGGCCGGAGAG | |
| | AGGAGTATGACGTGCTGGATAAGAGGAGGGGAAGA | |
| | GATCCCGAGATGGGAGGCAAGCCCAGAAGAAAGAA | |
| | CCCTCAGGAGGGCCTGTATAATGAGCTGCAGAAGG | |
| | ACAAGATGGCCGAGGCCTACAGCGAGATCGGCATG | |
| | AAGGGAGAGAGGCGCCGGGGCAAGGGACACGATG | |
| | GCCTGTACCAGGGCCTGTCAACAGCAACTAAAGAT | |
| | ACTTACGACGCACTGCACATGCAGGCTCTGCCACC | |
| | CCGA | |
| CAR EBV- | ATGGAGTTCGGCCTGAGCTGGCTGTTTCTGGTGGC | 88 |
| Gp350- | CATCCTGAAGGGCGTGCAGTGCGAGTTCCAGCTGC | |
| 003_VH- | AGCAGAGCGGACCAGACCTGGTGAAGCCAGGAGC | |
| linker-VL_V04 | CAGCGTGAAGATCAGCTGTAAGGCCTCTGGCTACA | |
| (CAR | GCTTTACCGATTATAACATGAATTGGGTGAAGCAGA | |
| comprising | GCAACGGCAAGAATCTGGAGTGGATCGGCGTGATC | |
| EBV-Gp350- | AACCCCAATTTCGGCACCACATCCTACAACCAGAAG | |
| 003 in VH- | TTTAAGGGCAAGGCCACCCTGACAGTGGACCAGAG | |
| linker-VL | CTCCTCTACCGCCTATATGCAGCTGAACTCCCTGAC | |
| orientation, | ATCTGAGGATAGCGCCGTGTACTATTGCGCCAGGT | |
| and V04 | CCGGCTACTATGGCTCTAATCGCTACTGGTATTTCG | |
| sequence | ACGTGTGGGGCACCGGCACCACAGTGACAGTGAG | |
| which is | CTCCTCTGGAGGAGGAGGCAGGGGCGGAGGAGGC | |
| underlined) | TCCGGCGGGGGGGGCTCTGACATCGTGATGACCCA | |
| | GAGCCAGAAGTTTATGAGCACATCCGTGGGCGATC | |
| | GGGTGTCCGTGACCTGCAAGGCCTCTCAGAGCGTG | |
| | GGCACAAATGTGGCCTGGTATCAGCAGAAGCCCGG | |
| | CCAGTCTCCTAAGGCCCTGATCTATTCCGCCTOTTA | |
| | CCGGTATTCCGGCGTGCCTGACAGATTCACCGGCA | |
| | GCGGCTCCGGCACAGACTTCACCCTGACAATCTCT | |
| | AACGTGCAGAGCGAGGATCTGGCCGAGTACTTCTG | |
| | TCAGCAGTACAATTCCTATCCATCTACCTTTGGCGG | |
| | CGGCACAAAGCTGGAGATCAAGGGATCCGAGAGCA | |
| | AGTACGGCCCCCCCTGCCCCCCCTGCCCCGCCCC | |
| | CCCCGTGGCCGGCCCCAGCGTGTTCCTGTTCCCCC | |
| | CCAAGCCCAAGGACACCCTGATGATCAGCCGCACC | |
| | CCCGAGGTGACCTGCGTGGTGGTGGACGTGAGCC | |
| | AGGAGGACCCCGAGGTGCAGTTCAACTGGTACGTG | |
| | GACGGCGTGGAGGTGCACAACGCCAAGACCAAGC | |
| | CCCGCGAGGAGCAGTTCCAGAGCACCTACCGCGTG | |
| | GTGAGCGTGCTGACCGTGCTGCACCAGGACTGGCT | |
| | GAACGGCAAGGAGTACAAGTGCAAGGTGAGCAACA | |
| | AGGGCCTGCCCAGCAGCATCGAGAAGACCATCAGC | |
| | AAGGCCAAGGGCCAGCCCCGCGAGCCCCAGGTGT | |
| | ACACCCTGCCCCCCAGCCAGGAGGAGATGACCAAG | |
| | AACCAGGTGAGCCTGACCTGCCTGGTGAAGGGCTT | |
| | CTACCCCAGCGACATCGCCGTGGAGTGGGAGAGCA | |
| | ACGGCCAGCCCGAGAACAACTACAAGACCACCCCC | |
| | CCCGTGCTGGACAGCGACGGCAGCTTCTTCCTGTA | |
| | CAGCCGCCTGACCGTGGACAAGAGCCGCTGGCAG | |
| | GAGGGCAACGTGTTCAGCTGCAGCGTGATGCACGA | |
| | GGCCCTGCACAACCACTACACCCAGAAGAGCCTGA | |
| | GCCTGAGCCCCGGCAAGATCTACATCTGGGCACCA | |
| | CTGGCAGGAACATGTGGCGTGCTGCTGCTGTCCCT | |
| | GGTCATCACCCTGTATTGCAAGCGCGGCCGGAAGA | |
| | AGCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGTCCTGGCTGTTTCTGGTGGC | 89 |
| Gp350- | CATCCTGAAGGGCGTGCAGTGCCAGGCAGTGGTGA | |
| 004_VL- | CACAGGAGAGCGCCCTGACCACATCCCCTGGAGAG | |
| linker-VH_V01 | ACCGTGACACTGACCTGTAGGAGCTCCACCGGCGC | |
| (CAR | CGTGACCACAAGCAACTACGCCAATTGGGTGCAGG | |
| comprising | AGAAGCCAGACCACCTGTTCACAGGCCTGATCGGA | |
| EBV-Gp350- | GGCACCAACAATAGGGCACCAGGCGTGCCTGCCAG | |
| 004 in VL- | ATTTTCTGGCAGCCTGATCGGCGACAAGGCCGCCC | |
| linker-VH | TGACAATCACCGGAGCACAGACCGAGGATGAGGCC | |
| orientation, | ATCTACTTCTGCGTGCTGTGGTATTCTAACCTGTGG | |
| and V01 | GTGTTTGGCGGCGGCACAAAGCTGACCGTGCTGTC | |
| sequence | TGGAGGAGGAGGCAGCGGCGGAGGAGGCTCCGGC | |
| which is | GGCGGCGGCTCTGAGGTGCAGCTGCAGCAGAGCG | |
| underlined) | GACCAGTGCTGGAGAAGCCAGGAGCCTCCGTGAAG | |
| | ATGTCTTGTAAGGCCAGCGGCTATACATTCACCGAT | |
| | TACTATATGAACTGGGTGAAGCAGTCCCACGGCAA | |
| | GTCTCTGGAGTGGATCGGCGTGATCAACCCCTACA | |
| | ATGGCGGCACCTCCCACAATGAGAAGCTGAAGGGC | |
| | AAGGCCACACTGACCGTGGACAAGTCTAGCTCCAC | |
| | AGCCTATATGGAGCTGAACTCCCTGACCTCTGAGG | |
| | ATAGCGCCGTGTACTATTGCGCCAGAGGCAATTACA | |
| | GCGCCTGGTTTGCCTATTGGGGCCAGGGCACACTG | |
| | GTGACCGTGTCCGCCGGATCCACCACAACACCGGC | |
| | GCCACGCCCACCAACACCAGCACCTACCATCGCAA | |
| | GCCAGCCACTGTCCCTGCGGCCCGAGGCCTGTAG | |
| | GCCAGCAGCAGGAGGAGCAGTGCACACCAGGGGC | |
| | CTGGACTTTGCCTGCGACATCTACATCTGGGCACCA | |
| | CTGGCAGGAACATGTGGCGTGCTGCTGCTGTCCCT | |
| | GGTCATCACCCTGTATTGCAAGCGCGGCCGGAAGA | |
| | AGCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGTCCTGGCTGTTTCTGGTGGC | 90 |
| Gp350- | CATCCTGAAGGGCGTGCAGTGCCAGGCAGTGGTGA | |
| 004_VL- | CACAGGAGAGCGCCCTGACCACATCCCCTGGAGAG | |
| linker-VH_V02 | ACCGTGACACTGACCTGTAGGAGCTCCACCGGCGC | |
| (CAR | CGTGACCACAAGCAACTACGCCAATTGGGTGCAGG | |
| comprising | AGAAGCCAGACCACCTGTTCACAGGCCTGATCGGA | |
| EBV-Gp350- | GGCACCAACAATAGGGCACCAGGCGTGCCTGCCAG | |
| 004 in VL- | ATTTTCTGGCAGCCTGATCGGCGACAAGGCCGCCC | |
| linker-VH | TGACAATCACCGGAGCACAGACCGAGGATGAGGCC | |
| orientation, | ATCTACTTCTGCGTGCTGTGGTATTCTAACCTGTGG | |
| and V02 | GTGTTTGGCGGCGGCACAAAGCTGACCGTGCTGTC | |
| sequence | TGGAGGAGGAGGCAGCGGCGGAGGAGGCTCCGGC | |
| which is | GGCGGCGGCTCTGAGGTGCAGCTGCAGCAGAGCG | |
| underlined) | GACCAGTGCTGGAGAAGCCAGGAGCCTCCGTGAAG | |
| | ATGTCTTGTAAGGCCAGCGGCTATACATTCACCGAT | |
| | TACTATATGAACTGGGTGAAGCAGTCCCACGGCAA | |
| | GTCTCTGGAGTGGATCGGCGTGATCAACCCCTACA | |
| | ATGGCGGCACCTCCCACAATGAGAAGCTGAAGGGC | |
| | AAGGCCACACTGACCGTGGACAAGTCTAGCTCCAC | |
| | AGCCTATATGGAGCTGAACTCCCTGACCTCTGAGG | |
| | ATAGCGCCGTGTACTATTGCGCCAGAGGCAATTACA | |
| | GCGCCTGGTTTGCCTATTGGGGCCAGGGCACACTG | |
| | GTGACCGTGTCCGCCGGATCCGAGAGCAAGTACGG | |
| | CCCCCCCTGCCCCCCCTGCCCCATCTACATCTGGG | |
| | CACCACTGGCAGGAACATGTGGCGTGCTGCTGCTG | |
| | TCCCTGGTCATCACCCTGTATTGCAAGCGCGGCCG | |
| | GAAGAAGCTGCTGTACATCTTCAAGCAGCCCTTCAT | |
| | GCGGCCCGTGCAGACAACCCAGGAGGAGGACGGC | |
| | TGCAGCTGTAGGTTCCCTGAAGAGGAGGAGGGAGG | |
| | ATGTGAGCTGAGGGTGAAGTTTTCCCGGTCTGCCG | |
| | ATGCCCCAGCCTATAAGCAGGGCCAGAATCAGCTG | |
| | TACAACGAGCTGAATCTGGGCCGGAGAGAGGAGTA | |
| | TGACGTGCTGGATAAGAGGAGGGGAAGAGATCCCG | |
| | AGATGGGAGGCAAGCCCAGAAGAAAGAACCCTCAG | |
| | GAGGGCCTGTATAATGAGCTGCAGAAGGACAAGAT | |
| | GGCCGAGGCCTACAGCGAGATCGGCATGAAGGGA | |
| | GAGAGGCGCCGGGGCAAGGGACACGATGGCCTGT | |
| | ACCAGGGCCTGTCAACAGCAACTAAAGATACTTACG | |
| | ACGCACTGCACATGCAGGCTCTGCCACCCCGA | |
| CAR_EBV- | ATGGAGTTCGGCCTGTCCTGGCTGTTTCTGGTGGC | 91 |
| Gp350- | CATCCTGAAGGGCGTGCAGTGCCAGGCAGTGGTGA | |
| 004_VL- | CACAGGAGAGCGCCCTGACCACATCCCCTGGAGAG | |
| linker-VH_V04 | ACCGTGACACTGACCTGTAGGAGCTCCACCGGCGC | |
| (CAR | CGTGACCACAAGCAACTACGCCAATTGGGTGCAGG | |
| comprising | AGAAGCCAGACCACCTGTTCACAGGCCTGATCGGA | |
| EBV-Gp350- | GGCACCAACAATAGGGCACCAGGCGTGCCTGCCAG | |
| 004 in VL- | ATTTTCTGGCAGCCTGATCGGCGACAAGGCCGCCC | |
| linker-VH | TGACAATCACCGGAGCACAGACCGAGGATGAGGCC | |
| orientation, | ATCTACTTCTGCGTGCTGTGGTATTCTAACCTGTGG | |
| and V04 | GTGTTTGGCGGCGGCACAAAGCTGACCGTGCTGTC | |
| sequence | TGGAGGAGGAGGCAGCGGCGGAGGAGGCTCCGGC | |
| which is | GGCGGCGGCTCTGAGGTGCAGCTGCAGCAGAGCG | |
| underlined) | GACCAGTGCTGGAGAAGCCAGGAGCCTCCGTGAAG | |
| | ATGTCTTGTAAGGCCAGCGGCTATACATTCACCGAT | |
| | TACTATATGAACTGGGTGAAGCAGTCCCACGGCAA | |
| | GTCTCTGGAGTGGATCGGCGTGATCAACCCCTACA | |
| | ATGGCGGCACCTCCCACAATGAGAAGCTGAAGGGC | |
| | AAGGCCACACTGACCGTGGACAAGTCTAGCTCCAC | |
| | AGCCTATATGGAGCTGAACTCCCTGACCTCTGAGG | |
| | ATAGCGCCGTGTACTATTGCGCCAGAGGCAATTACA | |
| | GCGCCTGGTTTGCCTATTGGGGCCAGGGCACACTG | |
| | GTGACCGTGTCCGCCGGATCCGAGAGCAAGTACGG | |
| | CCCCCCCTGCCCCCCCTGCCCCGCCCCCCCCGTG | |
| | GCCGGCCCCAGCGTGTTCCTGTTCCCCCCCAAGCC | |
| | CAAGGACACCCTGATGATCAGCCGCACCCCCGAGG | |
| | TGACCTGCGTGGTGGTGGACGTGAGCCAGGAGGA | |
| | CCCCGAGGTGCAGTTCAACTGGTACGTGGACGGCG | |
| | TGGAGGTGCACAACGCCAAGACCAAGCCCCGCGA | |
| | GGAGCAGTTCCAGAGCACCTACCGCGTGGTGAGCG | |
| | TGCTGACCGTGCTGCACCAGGACTGGCTGAACGGC | |
| | AAGGAGTACAAGTGCAAGGTGAGCAACAAGGGCCT | |
| | GCCCAGCAGCATCGAGAAGACCATCAGCAAGGCCA | |
| | AGGGCCAGCCCCGCGAGCCCCAGGTGTACACCCT | |
| | GCCCCCCAGCCAGGAGGAGATGACCAAGAACCAG | |
| | GTGAGCCTGACCTGCCTGGTGAAGGGCTTCTACCC | |
| | CAGCGACATCGCCGTGGAGTGGGAGAGCAACGGC | |
| | CAGCCCGAGAACAACTACAAGACCACCCCCCCCGT | |
| | GCTGGACAGCGACGGCAGCTTCTTCCTGTACAGCC | |
| | GCCTGACCGTGGACAAGAGCCGCTGGCAGGAGGG | |
| | CAACGTGTTCAGCTGCAGCGTGATGCACGAGGCCC | |
| | TGCACAACCACTACACCCAGAAGAGCCTGAGCCTG | |
| | AGCCCCGGCAAGATCTACATCTGGGCACCACTGGC | |
| | AGGAACATGTGGCGTGCTGCTGCTGTCCCTGGTCA | |
| | TCACCCTGTATTGCAAGCGCGGCCGGAAGAAGCTG | |
| | CTGTACATCTTCAAGCAGCCCTTCATGCGGCCCGT | |
| | GCAGACAACCCAGGAGGAGGACGGCTGCAGCTGT | |
| | AGGTTCCCTGAAGAGGAGGAGGGAGGATGTGAGCT | |
| | GAGGGTGAAGTTTTCCCGGTCTGCCGATGCCCCAG | |
| | CCTATAAGCAGGGCCAGAATCAGCTGTACAACGAG | |
| | CTGAATCTGGGCCGGAGAGAGGAGTATGACGTGCT | |
| | GGATAAGAGGAGGGGAAGAGATCCCGAGATGGGA | |
| | GGCAAGCCCAGAAGAAAGAACCCTCAGGAGGGCCT | |
| | GTATAATGAGCTGCAGAAGGACAAGATGGCCGAGG | |
| | CCTACAGCGAGATCGGCATGAAGGGAGAGAGGCG | |
| | CCGGGGCAAGGGACACGATGGCCTGTACCAGGGC | |
| | CTGTCAACAGCAACTAAAGATACTTACGACGCACTG | |
| | CACATGCAGGCTCTGCCACCCCGA | |
| CAR_EBV- | ATGGAGTTCGGCCTGTCCTGGCTGTTTCTGGTGGC | 92 |
| Gp350- | CATCCTGAAGGGCGTGCAGTGCGAGGTGCAGCTGC | |
| 004_VH- | AGCAGTCTGGACCAGTGCTGGAGAAGCCAGGAGCC | |
| linker-VL_V01 | AGCGTGAAGATGTCCTGTAAGGCCTCTGGCTACAC | |
| (CAR | CTTCACAGACTACTATATGAACTGGGTGAAGCAGAG | |
| comprising | CCACGGCAAGTCCCTGGAGTGGATCGGCGTGATCA | |
| EBV-Gp350- | ACCCCTACAATGGCGGCACCAGCCACAATGAGAAG | |
| 004 in VH- | CTGAAGGGCAAGGCCACCCTGACAGTGGACAAGAG | |
| linker-VL | CTCCTCTACAGCCTATATGGAGCTGAACAGCCTGAC | |
| orientation, | CTCCGAGGATTCTGCCGTGTACTATTGCGCCAGGG | |
| and V01 | GCAATTACTCCGCCTGGTTTGCCTATTGGGGCCAG | |
| sequence | GGCACACTGGTGACCGTGTCTGCCAGCGGCGGCG | |
| which is | GCGGCTCTGGAGGAGGAGGCAGCGGCGGAGGAG | |
| underlined) | GCTCCCAGGCCGTGGTGACACAGGAGTCTGCCCTG | |
| | ACCACAAGCCCTGGCGAGACCGTGACACTGACCTG | |
| | TCGGAGCTCCACCGGAGCAGTGACCACAAGCAACT | |
| | ATGCCAATTGGGTGCAGGAGAAGCCAGATCACCTG | |
| | TTCACAGGCCTGATCGGAGGCACCAACAATAGGGC | |
| | ACCAGGCGTGCCTGCAAGATTTTCCGGCTCTCTGAT | |
| | CGGCGACAAGGCCGCCCTGACAATCACCGGAGCA | |
| | CAGACCGAGGATGAGGCCATCTACTTCTGCGTGCT | |
| | GTGGTATTCCAACCTGTGGGTGTTTGGCGGCGGCA | |
| | CAAAGCTGACCGTGCTGGGATCCACCACAACACCG | |
| | GCGCCACGCCCACCAACACCAGCACCTACCATCGC | |
| | AAGCCAGCCACTGTCCCTGCGGCCCGAGGCCTGTA | |
| | GGCCAGCAGCAGGAGGAGCAGTGCACACCAGGGG | |
| | CCTGGACTTTGCCTGCGACATCTACATCTGGGCACC | |
| | ACTGGCAGGAACATGTGGCGTGCTGCTGCTGTCCC | |
| | TGGTCATCACCCTGTATTGCAAGCGCGGCCGGAAG | |
| | AAGCTGCTGTACATCTTCAAGCAGCCCTTCATGCGG | |
| | CCCGTGCAGACAACCCAGGAGGAGGACGGCTGCA | |
| | GCTGTAGGTTCCCTGAAGAGGAGGAGGGAGGATGT | |
| | GAGCTGAGGGTGAAGTTTTCCCGGTCTGCCGATGC | |
| | CCCAGCCTATAAGCAGGGCCAGAATCAGCTGTACA | |
| | ACGAGCTGAATCTGGGCCGGAGAGAGGAGTATGAC | |
| | GTGCTGGATAAGAGGAGGGGAAGAGATCCCGAGAT | |
| | GGGAGGCAAGCCCAGAAGAAAGAACCCTCAGGAG | |
| | GGCCTGTATAATGAGCTGCAGAAGGACAAGATGGC | |
| | CGAGGCCTACAGCGAGATCGGCATGAAGGGAGAG | |
| | AGGCGCCGGGGCAAGGGACACGATGGCCTGTACC | |
| | AGGGCCTGTCAACAGCAACTAAAGATACTTACGACG | |
| | CACTGCACATGCAGGCTCTGCCACCCCGA | |
| CAR_EBV- | ATGGAGTTCGGCCTGTCCTGGCTGTTTCTGGTGGC | 93 |
| Gp350- | CATCCTGAAGGGCGTGCAGTGCGAGGTGCAGCTGC | |
| 004_VH- | AGCAGTCTGGACCAGTGCTGGAGAAGCCAGGAGCC | |
| linker-VL_V02 | AGCGTGAAGATGTCCTGTAAGGCCTCTGGCTACAC | |
| (CAR | CTTCACAGACTACTATATGAACTGGGTGAAGCAGAG | |
| comprising | CCACGGCAAGTCCCTGGAGTGGATCGGCGTGATCA | |
| EBV-Gp350- | ACCCCTACAATGGCGGCACCAGCCACAATGAGAAG | |
| 004 in VH- | CTGAAGGGCAAGGCCACCCTGACAGTGGACAAGAG | |
| linker-VL | CTCCTCTACAGCCTATATGGAGCTGAACAGCCTGAC | |
| orientation, | CTCCGAGGATTCTGCCGTGTACTATTGCGCCAGGG | |
| and V02 | GCAATTACTCCGCCTGGTTTGCCTATTGGGGCCAG | |
| sequence | GGCACACTGGTGACCGTGTCTGCCAGCGGCGGCG | |
| which is | GCGGCTCTGGAGGAGGAGGCAGCGGCGGAGGAG | |
| underlined) | GCTCCCAGGCCGTGGTGACACAGGAGTCTGCCCTG | |
| | ACCACAAGCCCTGGCGAGACCGTGACACTGACCTG | |
| | TCGGAGCTCCACCGGAGCAGTGACCACAAGCAACT | |
| | ATGCCAATTGGGTGCAGGAGAAGCCAGATCACCTG | |
| | TTCACAGGCCTGATCGGAGGCACCAACAATAGGGC | |
| | ACCAGGCGTGCCTGCAAGATTTTCCGGCTCTCTGAT | |
| | CGGCGACAAGGCCGCCCTGACAATCACCGGAGCA | |
| | CAGACCGAGGATGAGGCCATCTACTTCTGCGTGCT | |
| | GTGGTATTCCAACCTGTGGGTGTTTGGCGGCGGCA | |
| | CAAAGCTGACCGTGCTGGGATCCGAGAGCAAGTAC | |
| | GGCCCCCCCTGCCCCCCCTGCCCCATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGTCCTGGCTGTTTCTGGTGGC | 94 |
| Gp350- | CATCCTGAAGGGCGTGCAGTGCGAGGTGCAGCTGC | |
| 004_VH- | AGCAGTCTGGACCAGTGCTGGAGAAGCCAGGAGCC | |
| linker-VL_V04 | AGCGTGAAGATGTCCTGTAAGGCCTCTGGCTACAC | |
| (CAR | CTTCACAGACTACTATATGAACTGGGTGAAGCAGAG | |
| comprising | CCACGGCAAGTCCCTGGAGTGGATCGGCGTGATCA | |
| EBV-Gp350- | ACCCCTACAATGGCGGCACCAGCCACAATGAGAAG | |
| 004 in VH- | CTGAAGGGCAAGGCCACCCTGACAGTGGACAAGAG | |
| linker-VL | CTCCTCTACAGCCTATATGGAGCTGAACAGCCTGAC | |
| orientation, | CTCCGAGGATTCTGCCGTGTACTATTGCGCCAGGG | |
| and V04 | GCAATTACTCCGCCTGGTTTGCCTATTGGGGCCAG | |
| sequence | GGCACACTGGTGACCGTGTCTGCCAGCGGCGGCG | |
| which is | GCGGCTCTGGAGGAGGAGGCAGCGGCGGAGGAG | |
| underlined) | GCTCCCAGGCCGTGGTGACACAGGAGTCTGCCCTG | |
| | ACCACAAGCCCTGGCGAGACCGTGACACTGACCTG | |
| | TCGGAGCTCCACCGGAGCAGTGACCACAAGCAACT | |
| | ATGCCAATTGGGTGCAGGAGAAGCCAGATCACCTG | |
| | TTCACAGGCCTGATCGGAGGCACCAACAATAGGGC | |
| | ACCAGGCGTGCCTGCAAGATTTTCCGGCTCTCTGAT | |
| | CGGCGACAAGGCCGCCCTGACAATCACCGGAGCA | |
| | CAGACCGAGGATGAGGCCATCTACTTCTGCGTGCT | |
| | GTGGTATTCCAACCTGTGGGTGTTTGGCGGCGGCA | |
| | CAAAGCTGACCGTGCTGGGATCCGAGAGCAAGTAC | |
| | GGCCCCCCCTGCCCCCCCTGCCCCGCCCCCCCCG | |
| | TGGCCGGCCCCAGCGTGTTCCTGTTCCCCCCCAAG | |
| | CCCAAGGACACCCTGATGATCAGCCGCACCCCCGA | |
| | GGTGACCTGCGTGGTGGTGGACGTGAGCCAGGAG | |
| | GACCCCGAGGTGCAGTTCAACTGGTACGTGGACGG | |
| | CGTGGAGGTGCACAACGCCAAGACCAAGCCCCGC | |
| | GAGGAGCAGTTCCAGAGCACCTACCGCGTGGTGAG | |
| | CGTGCTGACCGTGCTGCACCAGGACTGGCTGAACG | |
| | GCAAGGAGTACAAGTGCAAGGTGAGCAACAAGGGC | |
| | CTGCCCAGCAGCATCGAGAAGACCATCAGCAAGGC | |
| | CAAGGGCCAGCCCCGCGAGCCCCAGGTGTACACC | |
| | CTGCCCCCCAGCCAGGAGGAGATGACCAAGAACCA | |
| | GGTGAGCCTGACCTGCCTGGTGAAGGGCTTCTACC | |
| | CCAGCGACATCGCCGTGGAGTGGGAGAGCAACGG | |
| | CCAGCCCGAGAACAACTACAAGACCACCCCCCCCG | |
| | TGCTGGACAGCGACGGCAGCTTCTTCCTGTACAGC | |
| | CGCCTGACCGTGGACAAGAGCCGCTGGCAGGAGG | |
| | GCAACGTGTTCAGCTGCAGCGTGATGCACGAGGCC | |
| | CTGCACAACCACTACACCCAGAAGAGCCTGAGCCT | |
| | GAGCCCCGGCAAGATCTACATCTGGGCACCACTGG | |
| | CAGGAACATGTGGCGTGCTGCTGCTGTCCCTGGTC | |
| | ATCACCCTGTATTGCAAGCGCGGCCGGAAGAAGCT | |
| | GCTGTACATCTTCAAGCAGCCCTTCATGCGGCCCG | |
| | TGCAGACAACCCAGGAGGAGGACGGCTGCAGCTGT | |
| | AGGTTCCCTGAAGAGGAGGAGGGAGGATGTGAGCT | |
| | GAGGGTGAAGTTTTCCCGGTCTGCCGATGCCCCAG | |
| | CCTATAAGCAGGGCCAGAATCAGCTGTACAACGAG | |
| | CTGAATCTGGGCCGGAGAGAGGAGTATGACGTGCT | |
| | GGATAAGAGGAGGGGAAGAGATCCCGAGATGGGA | |
| | GGCAAGCCCAGAAGAAAGAACCCTCAGGAGGGCCT | |
| | GTATAATGAGCTGCAGAAGGACAAGATGGCCGAGG | |
| | CCTACAGCGAGATCGGCATGAAGGGAGAGAGGCG | |
| | CCGGGGCAAGGGACACGATGGCCTGTACCAGGGC | |
| | CTGTCAACAGCAACTAAAGATACTTACGACGCACTG | |
| | CACATGCAGGCTCTGCCACCCCGA | |
| CAR_EBV- | ATGGAGTTCGGCCTGTCTTGGCTGTTTCTGGTGGC | 95 |
| Gp350- | CATCCTGAAGGGCGTGCAGTGCCAGATCGTGCTGA | |
| 005_VL- | CACAGAGCCCTGCAATCATGAGCGCCTCCCTGGGA | |
| linker-VH_V01 | GAGAGGGTGACCATGACATGTACCGCCAGCTCCTC | |
| (CAR | TGTGAGCTCCTCTTACCTGCACTGGTATCAGCAGAA | |
| comprising | GCCTGGCAGCTCCCCAAAGCTGTGGATCTACTCTG | |
| EBV-Gp350- | CCAGCAACCTGGCCTCCGGCGTGCCCCCTAGGTTC | |
| 005 in VL- | TCCGGCTCTGGCAGCGGCACATCCTACTCTCTGAC | |
| linker-VH | CATCTCTAGCATGGAGGCCGAGGACGCCGCCACCT | |
| orientation, | ACTATTGCCACCAGTATCACCGCTCCCCACCCTGGA | |
| and V01 | CATTTGGCGGAGGCACCAAGCTGGAGATCAAGAGC | |
| sequence | GGAGGAGGAGGCTCTGGAGGAGGAGGCAGCGGAG | |
| which is | GAGGAGGCTCCCAGGTGCACCTGCAGCAGAGCGG | |
| underlined) | ACCAGAGCTGGTGAAGCCTGGAGCCTCCGTGAAGA | |
| | TCAGCTGTAAGGCCAGCGGCTACAACTTCAATGATT | |
| | ACTACATCAACTGGGTGAAGCAGAGGCCAGGACAG | |
| | GGCCTGGAGTGGATCGGATGGATCTTCCCAGGACG | |
| | GGGCAGAAGCAATTACTCCGAGAAGTTTAAGGGCA | |
| | AGGCCACACTGACCGTGGACAAGTCCTCTAGCACA | |
| | GCCTATGTGCTGCTGTCCTCTCTGACCTCTGAGGAT | |
| | AGCGCCGTGTACTTCTGCGCCAGGGGCACCGCACA | |
| | GGCAGGATTTCCATATTGGGGACAGGGCACACTGG | |
| | TGACCGTGAGCGCCGGATCCACCACAACACCGGCG | |
| | CCACGCCCACCAACACCAGCACCTACCATCGCAAG | |
| | CCAGCCACTGTCCCTGCGGCCCGAGGCCTGTAGG | |
| | CCAGCAGCAGGAGGAGCAGTGCACACCAGGGGCC | |
| | TGGACTTTGCCTGCGACATCTACATCTGGGCACCAC | |
| | TGGCAGGAACATGTGGCGTGCTGCTGCTGTCCCTG | |
| | GTCATCACCCTGTATTGCAAGCGCGGCCGGAAGAA | |
| | GCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGTCTTGGCTGTTTCTGGTGGC | 96 |
| Gp350- | CATCCTGAAGGGCGTGCAGTGCCAGATCGTGCTGA | |
| 005_VL- | CACAGAGCCCTGCAATCATGAGCGCCTCCCTGGGA | |
| linker-VH_V02 | GAGAGGGTGACCATGACATGTACCGCCAGCTCCTC | |
| (CAR | TGTGAGCTCCTCTTACCTGCACTGGTATCAGCAGAA | |
| comprising | GCCTGGCAGCTCCCCAAAGCTGTGGATCTACTCTG | |
| EBV-Gp350- | CCAGCAACCTGGCCTCCGGCGTGCCCCCTAGGTTC | |
| 005 in VL- | TCCGGCTCTGGCAGCGGCACATCCTACTCTCTGAC | |
| linker-VH | CATCTCTAGCATGGAGGCCGAGGACGCCGCCACCT | |
| orientation, | ACTATTGCCACCAGTATCACCGCTCCCCACCCTGGA | |
| and V02 | CATTTGGCGGAGGCACCAAGCTGGAGATCAAGAGC | |
| sequence | GGAGGAGGAGGCTCTGGAGGAGGAGGCAGCGGAG | |
| which is | GAGGAGGCTCCCAGGTGCACCTGCAGCAGAGCGG | |
| underlined) | ACCAGAGCTGGTGAAGCCTGGAGCCTCCGTGAAGA | |
| | TCAGCTGTAAGGCCAGCGGCTACAACTTCAATGATT | |
| | ACTACATCAACTGGGTGAAGCAGAGGCCAGGACAG | |
| | GGCCTGGAGTGGATCGGATGGATCTTCCCAGGACG | |
| | GGGCAGAAGCAATTACTCCGAGAAGTTTAAGGGCA | |
| | AGGCCACACTGACCGTGGACAAGTCCTCTAGCACA | |
| | GCCTATGTGCTGCTGTCCTCTCTGACCTCTGAGGAT | |
| | AGCGCCGTGTACTTCTGCGCCAGGGGCACCGCACA | |
| | GGCAGGATTTCCATATTGGGGACAGGGCACACTGG | |
| | TGACCGTGAGCGCCGGATCCGAGAGCAAGTACGG | |
| | CCCCCCCTGCCCCCCCTGCCCCATCTACATCTGGG | |
| | CACCACTGGCAGGAACATGTGGCGTGCTGCTGCTG | |
| | TCCCTGGTCATCACCCTGTATTGCAAGCGCGGCCG | |
| | GAAGAAGCTGCTGTACATCTTCAAGCAGCCCTTCAT | |
| | GCGGCCCGTGCAGACAACCCAGGAGGAGGACGGC | |
| | TGCAGCTGTAGGTTCCCTGAAGAGGAGGAGGGAGG | |
| | ATGTGAGCTGAGGGTGAAGTTTTCCCGGTCTGCCG | |
| | ATGCCCCAGCCTATAAGCAGGGCCAGAATCAGCTG | |
| | TACAACGAGCTGAATCTGGGCCGGAGAGAGGAGTA | |
| | TGACGTGCTGGATAAGAGGAGGGGAAGAGATCCCG | |
| | AGATGGGAGGCAAGCCCAGAAGAAAGAACCCTCAG | |
| | GAGGGCCTGTATAATGAGCTGCAGAAGGACAAGAT | |
| | GGCCGAGGCCTACAGCGAGATCGGCATGAAGGGA | |
| | GAGAGGCGCCGGGGCAAGGGACACGATGGCCTGT | |
| | ACCAGGGCCTGTCAACAGCAACTAAAGATACTTACG | |
| | ACGCACTGCACATGCAGGCTCTGCCACCCCGA | |
| CAR_EBV- | ATGGAGTTCGGCCTGTCTTGGCTGTTTCTGGTGGC | 97 |
| Gp350- | CATCCTGAAGGGCGTGCAGTGCCAGATCGTGCTGA | |
| 005_VL- | CACAGAGCCCTGCAATCATGAGCGCCTCCCTGGGA | |
| linker-VH_V04 | GAGAGGGTGACCATGACATGTACCGCCAGCTCCTC | |
| (CAR | TGTGAGCTCCTCTTACCTGCACTGGTATCAGCAGAA | |
| comprising | GCCTGGCAGCTCCCCAAAGCTGTGGATCTACTCTG | |
| EBV-Gp350- | CCAGCAACCTGGCCTCCGGCGTGCCCCCTAGGTTC | |
| 005 in VL- | TCCGGCTCTGGCAGCGGCACATCCTACTCTCTGAC | |
| linker-VH | CATCTCTAGCATGGAGGCCGAGGACGCCGCCACCT | |
| orientation, | ACTATTGCCACCAGTATCACCGCTCCCCACCCTGGA | |
| and V04 | CATTTGGCGGAGGCACCAAGCTGGAGATCAAGAGC | |
| sequence | GGAGGAGGAGGCTCTGGAGGAGGAGGCAGCGGAG | |
| which is | GAGGAGGCTCCCAGGTGCACCTGCAGCAGAGCGG | |
| underlined) | ACCAGAGCTGGTGAAGCCTGGAGCCTCCGTGAAGA | |
| | TCAGCTGTAAGGCCAGCGGCTACAACTTCAATGATT | |
| | ACTACATCAACTGGGTGAAGCAGAGGCCAGGACAG | |
| | GGCCTGGAGTGGATCGGATGGATCTTCCCAGGACG | |
| | GGGCAGAAGCAATTACTCCGAGAAGTTTAAGGGCA | |
| | AGGCCACACTGACCGTGGACAAGTCCTCTAGCACA | |
| | GCCTATGTGCTGCTGTCCTCTCTGACCTCTGAGGAT | |
| | AGCGCCGTGTACTTCTGCGCCAGGGGCACCGCACA | |
| | GGCAGGATTTCCATATTGGGGACAGGGCACACTGG | |
| | TGACCGTGAGCGCCGGATCCGAGAGCAAGTACGG | |
| | CCCCCCCTGCCCCCCCTGCCCCGCCCCCCCCGTG | |
| | GCCGGCCCCAGCGTGTTCCTGTTCCCCCCCAAGCC | |
| | CAAGGACACCCTGATGATCAGCCGCACCCCCGAGG | |
| | TGACCTGCGTGGTGGTGGACGTGAGCCAGGAGGA | |
| | CCCCGAGGTGCAGTTCAACTGGTACGTGGACGGCG | |
| | TGGAGGTGCACAACGCCAAGACCAAGCCCCGCGA | |
| | GGAGCAGTTCCAGAGCACCTACCGCGTGGTGAGCG | |
| | TGCTGACCGTGCTGCACCAGGACTGGCTGAACGGC | |
| | AAGGAGTACAAGTGCAAGGTGAGCAACAAGGGCCT | |
| | GCCCAGCAGCATCGAGAAGACCATCAGCAAGGCCA | |
| | AGGGCCAGCCCCGCGAGCCCCAGGTGTACACCCT | |
| | GCCCCCCAGCCAGGAGGAGATGACCAAGAACCAG | |
| | GTGAGCCTGACCTGCCTGGTGAAGGGCTTCTACCC | |
| | CAGCGACATCGCCGTGGAGTGGGAGAGCAACGGC | |
| | CAGCCCGAGAACAACTACAAGACCACCCCCCCCGT | |
| | GCTGGACAGCGACGGCAGCTTCTTCCTGTACAGCC | |
| | GCCTGACCGTGGACAAGAGCCGCTGGCAGGAGGG | |
| | CAACGTGTTCAGCTGCAGCGTGATGCACGAGGCCC | |
| | TGCACAACCACTACACCCAGAAGAGCCTGAGCCTG | |
| | AGCCCCGGCAAGATCTACATCTGGGCACCACTGGC | |
| | AGGAACATGTGGCGTGCTGCTGCTGTCCCTGGTCA | |
| | TCACCCTGTATTGCAAGCGCGGCCGGAAGAAGCTG | |
| | CTGTACATCTTCAAGCAGCCCTTCATGCGGCCCGT | |
| | GCAGACAACCCAGGAGGAGGACGGCTGCAGCTGT | |
| | AGGTTCCCTGAAGAGGAGGAGGGAGGATGTGAGCT | |
| | GAGGGTGAAGTTTTCCCGGTCTGCCGATGCCCCAG | |
| | CCTATAAGCAGGGCCAGAATCAGCTGTACAACGAG | |
| | CTGAATCTGGGCCGGAGAGAGGAGTATGACGTGCT | |
| | GGATAAGAGGAGGGGAAGAGATCCCGAGATGGGA | |
| | GGCAAGCCCAGAAGAAAGAACCCTCAGGAGGGCCT | |
| | GTATAATGAGCTGCAGAAGGACAAGATGGCCGAGG | |
| | CCTACAGCGAGATCGGCATGAAGGGAGAGAGGCG | |
| | CCGGGGCAAGGGACACGATGGCCTGTACCAGGGC | |
| | CTGTCAACAGCAACTAAAGATACTTACGACGCACTG | |
| | CACATGCAGGCTCTGCCACCCCGA | |
| CAR_EBV- | ATGGAGTTCGGCCTGAGCTGGCTGTTTCTGGTGGC | 98 |
| Gp350- | CATCCTGAAGGGCGTGCAGTGCCAGGTGCACCTGC | |
| 005_VH- | AGCAGTCCGGACCAGAGCTGGTGAAGCCTGGAGC | |
| linker-VL_V01 | CAGCGTGAAGATCAGCTGTAAGGCCTCTGGCTACA | |
| (CAR | ACTTCAATGACTACTATATCAACTGGGTGAAGCAGA | |
| comprising | GGCCAGGACAGGGCCTGGAGTGGATCGGATGGAT | |
| EBV-Gp350- | CTTCCCAGGACGGGGCCGGAGCAATTACAGCGAGA | |
| 005 in VH- | AGTTTAAGGGCAAGGCCACCCTGACAGTGGACAAG | |
| linker-VL | AGCTCCTCTACAGCCTATGTGCTGCTGAGCTCCCTG | |
| orientation, | ACCTCCGAGGATTCTGCCGTGTACTTCTGCGCCCG | |
| and V01 | GGGCACAGCACAGGCAGGCTTTCCTTATTGGGGAC | |
| sequence | AGGGCACACTGGTGACCGTGAGCGCCTCCGGAGG | |
| which is | AGGAGGCAGCGGAGGAGGCGGCTCCGGCGGCGG | |
| underlined) | CGGCTCTCAGATCGTGCTGACCCAGAGCCCAGCAA | |
| | TCATGTCTGCCAGCCTGGGAGAGAGGGTGACCATG | |
| | ACATGTACCGCCTCTAGCTCCGTGTCTAGCTCCTAC | |
| | CTGCACTGGTATCAGCAGAAGCCTGGCTCTAGCCC | |
| | AAAGCTGTGGATCTATTCCGCCTCTAACCTGGCCAG | |
| | CGGCGTGCCCCCTAGGTTCAGCGGCTCCGGCTCTG | |
| | GCACAAGCTACTCCCTGACCATCTCCTCTATGGAGG | |
| | CCGAGGATGCCGCCACCTACTATTGCCACCAGTAT | |
| | CACCGCTCCCCACCCTGGACATTTGGCGGAGGCAC | |
| | CAAGCTGGAGATCAAGGGATCCACCACAACACCGG | |
| | CGCCACGCCCACCAACACCAGCACCTACCATCGCA | |
| | AGCCAGCCACTGTCCCTGCGGCCCGAGGCCTGTAG | |
| | GCCAGCAGCAGGAGGAGCAGTGCACACCAGGGGC | |
| | CTGGACTTTGCCTGCGACATCTACATCTGGGCACCA | |
| | CTGGCAGGAACATGTGGCGTGCTGCTGCTGTCCCT | |
| | GGTCATCACCCTGTATTGCAAGCGCGGCCGGAAGA | |
| | AGCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGAGCTGGCTGTTTCTGGTGGC | 99 |
| Gp350- | CATCCTGAAGGGCGTGCAGTGCCAGGTGCACCTGC | |
| 005_VH- | AGCAGTCCGGACCAGAGCTGGTGAAGCCTGGAGC | |
| linker-VL_V02 | CAGCGTGAAGATCAGCTGTAAGGCCTCTGGCTACA | |
| (CAR | ACTTCAATGACTACTATATCAACTGGGTGAAGCAGA | |
| comprising | GGCCAGGACAGGGCCTGGAGTGGATCGGATGGAT | |
| EBV-Gp350- | CTTCCCAGGACGGGGCCGGAGCAATTACAGCGAGA | |
| 005 in VH- | AGTTTAAGGGCAAGGCCACCCTGACAGTGGACAAG | |
| linker-VL | AGCTCCTCTACAGCCTATGTGCTGCTGAGCTCCCTG | |
| orientation, | ACCTCCGAGGATTCTGCCGTGTACTTCTGCGCCCG | |
| and V02 | GGGCACAGCACAGGCAGGCTTTCCTTATTGGGGAC | |
| sequence | AGGGCACACTGGTGACCGTGAGCGCCTCCGGAGG | |
| which is | AGGAGGCAGCGGAGGAGGCGGCTCCGGCGGCGG | |
| underlined) | CGGCTCTCAGATCGTGCTGACCCAGAGCCCAGCAA | |
| | TCATGTCTGCCAGCCTGGGAGAGAGGGTGACCATG | |
| | ACATGTACCGCCTCTAGCTCCGTGTCTAGCTCCTAC | |
| | CTGCACTGGTATCAGCAGAAGCCTGGCTCTAGCCC | |
| | AAAGCTGTGGATCTATTCCGCCTCTAACCTGGCCAG | |
| | CGGCGTGCCCCCTAGGTTCAGCGGCTCCGGCTCTG | |
| | GCACAAGCTACTCCCTGACCATCTCCTCTATGGAGG | |
| | CCGAGGATGCCGCCACCTACTATTGCCACCAGTAT | |
| | CACCGCTCCCCACCCTGGACATTTGGCGGAGGCAC | |
| | CAAGCTGGAGATCAAGGGATCCGAGAGCAAGTACG | |
| | GCCCCCCCTGCCCCCCCTGCCCCATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 100 |
| Gp350- | CATCCTGAAGGGCGTGCAGTGCCAGGTGCACCTGC | |
| 005_VH- | AGCAGTCCGGACCAGAGCTGGTGAAGCCTGGAGC | |
| linker-VL_V04 | CAGCGTGAAGATCAGCTGTAAGGCCTCTGGCTACA | |
| (CAR | ACTTCAATGACTACTATATCAACTGGGTGAAGCAGA | |
| comprising | GGCCAGGACAGGGCCTGGAGTGGATCGGATGGAT | |
| EBV-Gp350- | CTTCCCAGGACGGGGCCGGAGCAATTACAGCGAGA | |
| 005 in VH- | AGTTTAAGGGCAAGGCCACCCTGACAGTGGACAAG | |
| linker-VL | AGCTCCTCTACAGCCTATGTGCTGCTGAGCTCCCTG | |
| orientation, | ACCTCCGAGGATTCTGCCGTGTACTTCTGCGCCCG | |
| and V04 | GGGCACAGCACAGGCAGGCTTTCCTTATTGGGGAC | |
| sequence | AGGGCACACTGGTGACCGTGAGCGCCTCCGGAGG | |
| which is | AGGAGGCAGCGGAGGAGGCGGCTCCGGCGGCGG | |
| underlined) | CGGCTCTCAGATCGTGCTGACCCAGAGCCCAGCAA | |
| | TCATGTCTGCCAGCCTGGGAGAGAGGGTGACCATG | |
| | ACATGTACCGCCTCTAGCTCCGTGTCTAGCTCCTAC | |
| | CTGCACTGGTATCAGCAGAAGCCTGGCTCTAGCCC | |
| | AAAGCTGTGGATCTATTCCGCCTCTAACCTGGCCAG | |
| | CGGCGTGCCCCCTAGGTTCAGCGGCTCCGGCTCTG | |
| | GCACAAGCTACTCCCTGACCATCTCCTCTATGGAGG | |
| | CCGAGGATGCCGCCACCTACTATTGCCACCAGTAT | |
| | CACCGCTCCCCACCCTGGACATTTGGCGGAGGCAC | |
| | CAAGCTGGAGATCAAGGGATCCGAGAGCAAGTACG | |
| | GCCCCCCCTGCCCCCCCTGCCCCGCCCCCCCCGT | |
| | GGCCGGCCCCAGCGTGTTCCTGTTCCCCCCCAAGC | |
| | CCAAGGACACCCTGATGATCAGCCGCACCCCCGAG | |
| | GTGACCTGCGTGGTGGTGGACGTGAGCCAGGAGG | |
| | ACCCCGAGGTGCAGTTCAACTGGTACGTGGACGGC | |
| | GTGGAGGTGCACAACGCCAAGACCAAGCCCCGCG | |
| | AGGAGCAGTTCCAGAGCACCTACCGCGTGGTGAGC | |
| | GTGCTGACCGTGCTGCACCAGGACTGGCTGAACGG | |
| | CAAGGAGTACAAGTGCAAGGTGAGCAACAAGGGCC | |
| | TGCCCAGCAGCATCGAGAAGACCATCAGCAAGGCC | |
| | AAGGGCCAGCCCCGCGAGCCCCAGGTGTACACCC | |
| | TGCCCCCCAGCCAGGAGGAGATGACCAAGAACCAG | |
| | GTGAGCCTGACCTGCCTGGTGAAGGGCTTCTACCC | |
| | CAGCGACATCGCCGTGGAGTGGGAGAGCAACGGC | |
| | CAGCCCGAGAACAACTACAAGACCACCCCCCCCGT | |
| | GCTGGACAGCGACGGCAGCTTCTTCCTGTACAGCC | |
| | GCCTGACCGTGGACAAGAGCCGCTGGCAGGAGGG | |
| | CAACGTGTTCAGCTGCAGCGTGATGCACGAGGCCC | |
| | TGCACAACCACTACACCCAGAAGAGCCTGAGCCTG | |
| | AGCCCCGGCAAGATCTACATCTGGGCACCACTGGC | |
| | AGGAACATGTGGCGTGCTGCTGCTGTCCCTGGTCA | |
| | TCACCCTGTATTGCAAGCGCGGCCGGAAGAAGCTG | |
| | CTGTACATCTTCAAGCAGCCCTTCATGCGGCCCGT | |
| | GCAGACAACCCAGGAGGAGGACGGCTGCAGCTGT | |
| | AGGTTCCCTGAAGAGGAGGAGGGAGGATGTGAGCT | |
| | GAGGGTGAAGTTTTCCCGGTCTGCCGATGCCCCAG | |
| | CCTATAAGCAGGGCCAGAATCAGCTGTACAACGAG | |
| | CTGAATCTGGGCCGGAGAGAGGAGTATGACGTGCT | |
| | GGATAAGAGGAGGGGAAGAGATCCCGAGATGGGA | |
| | GGCAAGCCCAGAAGAAAGAACCCTCAGGAGGGCCT | |
| | GTATAATGAGCTGCAGAAGGACAAGATGGCCGAGG | |
| | CCTACAGCGAGATCGGCATGAAGGGAGAGAGGCG | |
| | CCGGGGCAAGGGACACGATGGCCTGTACCAGGGC | |
| | CTGTCAACAGCAACTAAAGATACTTACGACGCACTG | |
| | CACATGCAGGCTCTGCCACCCCGA | |
| CAR_EBV- | ATGGAGTTCGGCCTGAGCTGGCTGTTTCTGGTGGC | 162 |
| Gp350- | CATCCTGAAGGGCGTGCAGTGCGACATCCAGATGA | |
| 003_VL1- | CCCAGAGCCCTAGCTCGCTGTCCGCCTCCGTAGGT | |
| linker- | GATCGGGTCACCATCACCTGTAAGGCGTCACAGAG | |
| VH1_V01 | TGTGGGCACCAACGTGGCGTGGTTCCAGCAGAAGC | |
| | CGGGCAAGGCACCCAAGGCCCTCATTTACTCCGCC | |
| | TCCTACAGGTACTCCGGGGTGCCATCCCGCTTCAG | |
| | CGGCTCCGGTTCTGGCACCGACTTTACTCTGACCAT | |
| | CTCTTCTCTGCAGCCGGAGGACTTCGCCACCTACTT | |
| | TTGCCAACAGTACAACAGCTACCCCTCTACCTTCGG | |
| | CGGAGGCACCAAGCTGGAGATCAAGTCCGGCGGA | |
| | GGTGGCTCTGGAGGCGGTGGATCTGGTGGTGGCG | |
| | GATCACAGGTCCAGCTAGTGCAGAGCGGCGCGGA | |
| | GGTGAAGAAGCCCGGGGCTTCGGTGAAAGTTTCAT | |
| | GCAAAGCTTCCGGCTATTCCTTCACTGATTACAACA | |
| | TGAATTGGGTCCGCCAGGCCCCTGGACAGGGCTTG | |
| | GAATGGATCGGTGTCATTAATCCCAACTTCGGAACC | |
| | ACAAGCTACAACCAGAAATTTAAGGGCCGAGTGACA | |
| | ATGACGGTAGACACTTCGACCTCCACCGTGTACATG | |
| | GAGCTGAGTTCTCTTCGCTCGGAGGACACCGCCGT | |
| | GTATTACTGTGCTCGCTCCGGCTACTACGGCAGCA | |
| | ACCGCTATTGGTACTTCGACGTGTGGGGCCAGGGA | |
| | ACGACCGTCACCGTGTCCTCTGGATCCACCACAAC | |
| | ACCGGCGCCACGCCCACCAACACCAGCACCTACCA | |
| | TCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 163 |
| Gp350- | CATCCTGAAGGGCGTGCAGTGCCAGGTGCAGCTGG | |
| 003_VH1- | TGCAGAGCGGCGCCGAGGTGAAGAAGCCGGGAGC | |
| linker- | TTCCGTTAAGGTGTCGTGCAAGGCCTCTGGCTACTC | |
| VL1_V01 | CTTCACCGACTACAACATGAACTGGGTCCGCCAGG | |
| | CCCCTGGACAGGGATTGGAGTGGATTGGTGTGATT | |
| | AATCCAAATTTCGGTACCACAAGCTACAACCAGAAA | |
| | TTTAAAGGTCGAGTGACTATGACAGTCGATACTTCG | |
| | ACCTCTACCGTGTACATGGAGCTGTCCTCCCTACGT | |
| | AGCGAAGACACCGCGGTTTACTACTGTGCCCGGTC | |
| | GGGCTATTACGGCTCCAACCGCTATTGGTACTTTGA | |
| | CGTGTGGGGCCAGGGCACGACCGTCACCGTATCGT | |
| | CCTCTGGCGGAGGTGGCTCTGGAGGCGGTGGATC | |
| | TGGTGGTGGCGGATCAGACATCCAGATGACCCAGA | |
| | GCCCTTCTAGTCTTTCCGCTTCCGTGGGAGACAGG | |
| | GTCACTATCACCTGCAAAGCGTCACAGAGCGTGGG | |
| | CACCAACGTGGCTTGGTTCCAGCAGAAGCCCGGCA | |
| | AGGCACCCAAGGCGCTCATCTACTCGGCTAGCTAC | |
| | CGCTACAGCGGCGTCCCCTCCCGCTTCTCTGGCTC | |
| | GGGGAGTGGGACGGACTTCACCCTGACCATCTCTT | |
| | CTCTGCAGCCGGAGGACTTTGCCACCTACTTCTGTC | |
| | AACAGTACAACTCCTATCCCTCCACCTTCGGCGGC | |
| | GGCACCAAGCTGGAGATCAAGGGATCCACCACAAC | |
| | ACCGGCGCCACGCCCACCAACACCAGCACCTACCA | |
| | TCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 164 |
| Gp350- | CATCCTGAAGGGCGTGCAGTGCGACATCCAGATGA | |
| 003_VL1- | CCCAGAGCCCTTCTTCTCTTTCCGCCTCCGTGGGG | |
| linker- | GACAGGGTCACCATCACCTGCAAGGCTTCTCAGTC | |
| VH2_V01 | AGTGGGCACCAACGTGGCGTGGTTCCAGCAGAAGC | |
| | CTGGCAAGGCCCCCAAGGCGCTGATTTACTCTGCA | |
| | AGCTACCGTTACAGTGGCGTCCCGTCCCGCTTTTCA | |
| | GGCTCCGGCTCGGGCACCGACTTTACCCTGACCAT | |
| | CTCTTCCCTACAGCCAGAGGATTTCGCCACATATTT | |
| | CTGTCAACAGTACAACTCCTACCCCTCTACCTTCGG | |
| | CGGTGGGACCAAGCTGGAGATCAAGAGCGGCGGA | |
| | GGTGGCTCTGGAGGCGGTGGATCTGGTGGTGGCG | |
| | GATCACAGGTGCAGTTGGTGCAGAGCGGCGCCGA | |
| | GGTGAAGAAGCCCGGGGCTTCCGTTAAGGTGTCCT | |
| | GTAAAGCATCTGGCTACTCCTTCACAGATTACAATA | |
| | TGAACTGGGTCAAGCAGGCCCCGGGACAGGGCCT | |
| | CGAATGGATCGGTGTGATTAATCCAAACTTCGGCAC | |
| | GACCAGCTACAACCAGAAATTTAAAGGTCGGGCTAC | |
| | TATGACTGTTGACACTTCCACTTCGACCGTCTACAT | |
| | GGAGCTGAGTAGCCTGCGCTCGGAGGACACCGCG | |
| | GTGTACTACTGCGCCCGCAGTGGCTATTACGGCAG | |
| | CAACCGCTATTGGTACTTCGACGTGTGGGGCCAGG | |
| | GAACTACGGTAACCGTGTCGTCCGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 165 |
| Gp350- | CATCCTGAAGGGCGTGCAGTGCCAGGTCCAGCTGG | |
| 003_VH2- | TGCAGAGTGGCGCCGAGGTGAAGAAGCCCGGCGC | |
| linker- | TTCCGTAAAGGTGTCATGCAAGGCTTCTGGCTATTC | |
| VL1_V01 | TTTCACCGACTACAATATGAACTGGGTCAAGCAGGC | |
| | CCCGGGACAGGGTTTGGAGTGGATCGGTGTGATTA | |
| | ATCCCAACTTCGGAACTACCAGCTACAACCAGAAAT | |
| | TTAAAGGCCGGGCGACTATGACAGTCGACACCTCG | |
| | ACCTCCACAGTGTACATGGAGCTGAGCTCGCTGCG | |
| | CTCCGAGGACACGGCCGTGTATTACTGTGCTCGCT | |
| | CGGGCTACTACGGCAGCAACCGCTATTGGTACTTC | |
| | GACGTGTGGGGCCAGGGCACGACCGTCACCGTGT | |
| | CGAGCAGTGGCGGAGGTGGCTCTGGAGGCGGTGG | |
| | ATCTGGTGGTGGCGGATCAGACATCCAGATGACCC | |
| | AGAGCCCTTCCAGTCTCAGCGCATCCGTGGGTGAT | |
| | CGTGTTACCATCACCTGCAAAGCCTCCCAGAGCGT | |
| | GGGCACCAACGTGGCTTGGTTCCAGCAGAAGCCTG | |
| | GCAAGGCCCCCAAGGCGCTAATCTACTCGGCGAGC | |
| | TACCGCTACTCCGGGGTCCCATCCAGGTTTTCAGG | |
| | ATCTGGTTCTGGCACTGACTTTACTCTTACTATTTCT | |
| | TCTCTGCAGCCGGAAGATTTCGCCACCTACTTCTGT | |
| | CAACAGTACAACTCCTACCCCTCTACCTTCGGCGGC | |
| | GGCACCAAGCTGGAGATCAAGGGATCCACCACAAC | |
| | ACCGGCGCCACGCCCACCAACACCAGCACCTACCA | |
| | TCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 166 |
| Gp350- | CATCCTGAAGGGCGTGCAGTGCGACATCCAGATGA | |
| 003_VL1- | CCCAGAGCCCTTCCTCTCTATCCGCCTCCGTAGGT | |
| linker- | GATAGGGTTACAATCACCTGCAAAGCATCCCAGTCC | |
| VH3_V01 | GTGGGCACGAACGTGGCTTGGTTCCAGCAGAAGCC | |
| | CGGCAAAGCTCCCAAGGCCCTTATCTACAGTGCGA | |
| | GCTACCGCTACAGTGGGGTGCCTTCCCGCTTTTCA | |
| | GGGTCTGGCTCTGGTACTGACTTCACCCTGACCAT | |
| | CTCTAGCCTGCAGCCGGAAGATTTCGCCACCTACTT | |
| | TTGTCAACAGTACAACTCCTATCCCTCTACCTTCGG | |
| | CGGCGGCACTAAGCTGGAGATCAAGTCTGGCGGAG | |
| | GTGGCTCTGGAGGCGGTGGATCTGGTGGTGGCGG | |
| | ATCACAGGTGCAGCTGGTGCAGAGCGGCGCGGAG | |
| | GTGAAGAAGCCGGGTGCTTCCGTGAAGGTGTCGTG | |
| | TAAGGCGTCCGGATACTCCTTCACCGACTACAACAT | |
| | GAACTGGGTCAAGCAGGCCCCCGGACAGGGCCTC | |
| | GAGTGGATTGGCGTCATTAATCCAAATTTCGGCACG | |
| | ACCAGCTACAACCAGAAATTTAAGGGCCGCGCCAC | |
| | TCTGACTGTCGACCAGAGCACCTCGACCGTGTACA | |
| | TGGAGCTGTCCTCATTGCGGTCGGAGGACACCGCC | |
| | GTGTATTACTGCGCCCGTAGTGGCTACTACGGCAG | |
| | CAACCGCTATTGGTACTTCGACGTGTGGGGCCAGG | |
| | GCACCACAGTCACCGTGTCCTCCGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 167 |
| Gp350- | CATCCTGAAGGGCGTGCAGTGCCAGGTGCAGCTGG | |
| 003_VH3- | TGCAGAGCGGCGCCGAGGTGAAGAAGCCTGGCGC | |
| linker- | TTCTGTAAAGGTGTCATGCAAGGCTTCCGGCTACTC | |
| VL1_V01 | CTTCACCGACTACAACATGAACTGGGTCAAGCAGG | |
| | CCCCCGGACAGGGACTCGAGTGGATCGGTGTGATT | |
| | AATCCAAATTTCGGGACCACAAGCTACAACCAGAAA | |
| | TTTAAAGGCCGCGCCACTCTTACAGTTGACCAGTCG | |
| | ACCTCCACCGTGTACATGGAGCTGAGCTCCCTGCG | |
| | CTCGGAGGACACCGCCGTGTATTACTGTGCTCGTT | |
| | CTGGGTACTACGGCAGCAACCGCTATTGGTACTTC | |
| | GACGTGTGGGGCCAGGGCACGACGGTCACTGTCT | |
| | CGTCCAGTGGCGGAGGTGGCTCTGGAGGCGGTGG | |
| | ATCTGGTGGTGGCGGATCAGACATCCAGATGACTC | |
| | AGAGCCCTTCCAGTTTGTCGGCGTCCGTGGGTGAT | |
| | AGGGTCACCATCACCTGCAAAGCATCCCAGAGCGT | |
| | GGGCACCAACGTGGCGTGGTTCCAGCAGAAGCCC | |
| | GGCAAGGCCCCGAAGGCGCTCATCTACTCCGCCTC | |
| | CTACCGGTACTCTGGTGTCCCCTCTCGCTTCTCAGG | |
| | GTCGGGCTCCGGTACTGATTTCACCCTGACCATTTC | |
| | TTCTCTGCAGCCGGAAGACTTTGCCACCTATTTTTG | |
| | TCAACAGTACAACTCCTACCCCTCTACCTTCGGCGG | |
| | CGGCACCAAGCTGGAGATCAAGGGATCCACCACAA | |
| | CACCGGCGCCACGCCCACCAACACCAGCACCTACC | |
| | ATCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 168 |
| Gp350- | CATCCTGAAGGGCGTGCAGTGCGACATCCAGATGA | |
| 003_VL2- | CCCAGAGCCCTTCCTCTCTGAGCGCATCCGTAGGT | |
| linker- | GATCGAGTTACCATCACCTGCAAGGCCTCTCAGTCT | |
| VH1_V01 | GTGGGGACCAACGTCGCCTGGTACCAGCAGAAGCC | |
| | TGGCAAGGCGCCCAAGGCGCTTATCTACAGTGCTT | |
| | CTTACCGCTACAGCGGCGTCCCCGACCGCTTTTCG | |
| | GGGTCCGGCTCGGGCACTGATTTCACCCTGACGAT | |
| | TTCCTCCTTGCAGCCGGAGGACTTCGCCACTTATTT | |
| | CTGTCAACAGTACAACAGCTACCCCTCTACCTTCGG | |
| | CGGAGGCACCAAGCTGGAGATCAAGAGCGGCGGA | |
| | GGTGGCTCTGGAGGCGGTGGATCTGGTGGTGGCG | |
| | GATCACAGGTGCAGCTGGTGCAGTCCGGGGCTGA | |
| | GGTGAAGAAGCCCGGGGCGTCCGTGAAAGTTTCAT | |
| | GCAAAGCCTCGGGCTACTCCTTCACCGACTACAAC | |
| | ATGAACTGGGTGCGCCAGGCCCCGGGACAGGGGC | |
| | TAGAATGGATCGGTGTGATTAATCCAAATTTTGGTA | |
| | CTACAAGCTACAACCAGAAATTTAAGGGCCGGGTCA | |
| | CTATGACTGTCGACACCAGCACCTCCACCGTGTACA | |
| | TGGAGCTGTCGAGTCTCAGGTCCGAGGACACAGCC | |
| | GTGTACTACTGTGCTCGTTCTGGTTATTACGGCAGC | |
| | AACCGCTATTGGTACTTCGACGTGTGGGGCCAGGG | |
| | CACGACCGTCACCGTGTCGTCTGGATCCACCACAA | |
| | CACCGGCGCCACGCCCACCAACACCAGCACCTACC | |
| | ATCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 169 |
| Gp350- | CATCCTGAAGGGCGTGCAGTGCCAGGTGCAGCTAG | |
| 003_VH1- | TGCAGAGTGGCGCCGAGGTGAAGAAGCCCGGGGC | |
| linker- | ATCGGTTAAGGTGTCATGCAAAGCGAGCGGATACT | |
| VL2_V01 | CCTTCACCGACTACAACATGAATTGGGTCCGACAG | |
| | GCCCCTGGACAGGGACTTGAGTGGATCGGTGTCAT | |
| | TAATCCAAACTTTGGCACCACATCATACAACCAGAA | |
| | ATTTAAGGGCCGTGTTACGATGACCGTCGACACGT | |
| | CGACCTCCACCGTGTACATGGAGCTGAGCTCTCTG | |
| | CGCTCGGAAGACACCGCCGTGTATTACTGTGCTCG | |
| | CAGCGGCTACTACGGCAGCAACCGCTATTGGTACT | |
| | TCGACGTGTGGGGCCAGGGCACTACAGTCACCGTG | |
| | TCCTCTTCTGGCGGAGGTGGCTCTGGAGGCGGTGG | |
| | ATCTGGTGGTGGOGGATCAGACATCCAGATGACCC | |
| | AGAGCCCTTCCAGCTTGTCCGCCTCCGTAGGTGAT | |
| | CGGGTGACCATCACCTGCAAAGCCTCCCAGTCCGT | |
| | GGGCACCAACGTGGCGTGGTACCAGCAGAAGCCC | |
| | GGCAAGGCCCCGAAGGCGCTCATTTACTCTGCTAG | |
| | CTACAGGTACAGTGGTGTCCCCGACCGCTTCTCTG | |
| | GCTCCGGCTCCGGGACTGATTTCACCCTGACTATCT | |
| | CTTCCCTGCAGCCGGAGGACTTTGCTACTTATTTCT | |
| | GTCAACAGTACAACTCCTACCCCTCTACCTTCGGCG | |
| | GCGGGACCAAGCTGGAGATCAAGGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGTCCTGGCTGTTTCTGGTGGC | 170 |
| Gp350- | CATCCTGAAGGGCGTGCAGTGCGATATACAAATGA | |
| 003_VL2- | CTCAATCCCCCAGTTCCCTTAGCGCCAGCGTGGGA | |
| linker- | GATCGGGTGACTATTACGTGCAAAGCTTCACAATCT | |
| VH2_V01 | GTAGGCACTAACGTCGCCTGGTATCAACAGAAGCC | |
| | AGGAAAGGCGCCCAAGGCACTGATTTACTCCGCGT | |
| | CCTACAGGTATTCCGGAGTCCCAGACCGGTTCAGT | |
| | GGCAGTGGCTCCGGAACCGACTTTACTCTGACGAT | |
| | CAGCAGCCTTCAACCAGAGGATTTTGCAACCTACTT | |
| | TTGCCAACAGTACAATAGTTATCCATCTACGTTCGG | |
| | TGGCGGCACGAAACTCGAAATTAAATCCGGTGGTG | |
| | GCGGCAGCGGTGGGGGGGGGTCCGGCGGTGGTG | |
| | GATCACAAGTGCAACTTGTACAAAGCGGAGCTGAA | |
| | GTTAAGAAGCCCGGGGCCAGTGTCAAGGTCTCATG | |
| | TAAGGCCAGCGGCTATTCTTTTACTGACTATAATAT | |
| | GAACTGGGTGAAACAGGCGCCTGGCCAAGGCCTC | |
| | GAGTGGATTGGAGTAATAAATCCGAATTTTGGTACT | |
| | ACAAGTTATAACCAGAAGTTTAAGGGCCGCGCCACT | |
| | ATGACTGTTGATACTTCCACGAGTACGGTGTACATG | |
| | GAGCTTAGTTCACTTAGGTCCGAGGATACGGCCGT | |
| | CTATTATTGTGCCAGATCAGGATACTATGGATCTAA | |
| | CCGGTACTGGTATTTTGACGTATGGGGCCAAGGAA | |
| | CTACCGTTACCGTGTCATCAGGATCCACCACAACAC | |
| | CGGCGCCACGCCCACCAACACCAGCACCTACCATC | |
| | GCAAGCCAGCCACTGTCCCTGCGGCCCGAGGCCT | |
| | GTAGGCCAGCAGCAGGAGGAGCAGTGCACACCAG | |
| | GGGCCTGGACTTTGCCTGCGACATCTACATCTGGG | |
| | CACCACTGGCAGGAACATGTGGCGTGCTGCTGCTG | |
| | TCCCTGGTCATCACCCTGTATTGCAAGCGCGGCCG | |
| | GAAGAAGCTGCTGTACATCTTCAAGCAGCCCTTCAT | |
| | GCGGCCCGTGCAGACAACCCAGGAGGAGGACGGC | |
| | TGCAGCTGTAGGTTCCCTGAAGAGGAGGAGGGAGG | |
| | ATGTGAGCTGAGGGTGAAGTTTTCCCGGTCTGCCG | |
| | ATGCCCCAGCCTATAAGCAGGGCCAGAATCAGCTG | |
| | TACAACGAGCTGAATCTGGGCCGGAGAGAGGAGTA | |
| | TGACGTGCTGGATAAGAGGAGGGGAAGAGATCCCG | |
| | AGATGGGAGGCAAGCCCAGAAGAAAGAACCCTCAG | |
| | GAGGGCCTGTATAATGAGCTGCAGAAGGACAAGAT | |
| | GGCCGAGGCCTACAGCGAGATCGGCATGAAGGGA | |
| | GAGAGGCGCCGGGGCAAGGGACACGATGGCCTGT | |
| | ACCAGGGCCTGTCAACAGCAACTAAAGATACTTACG | |
| | ACGCACTGCACATGCAGGCTCTGCCACCCCGA | |
| CAR_EBV- | ATGGAGTTCGGCCTGAGCTGGCTGTTTCTGGTGGC | 171 |
| Gp350- | CATCCTGAAGGGCGTGCAGTGCCAGGTCCAGCTGG | |
| 003_VH2- | TGCAGAGCGGTGCCGAGGTGAAGAAGCCTGGAGC | |
| linker- | GTCGGTAAAGGTGTCATGCAAAGCGTCCGGCTACT | |
| VL2_V01 | CCTTCACCGACTACAACATGAACTGGGTCAAGCAG | |
| | GCCCCGGGACAGGGGCTAGAGTGGATTGGCGTGA | |
| | TTAATCCAAATTTCGGTACCACATCTTACAACCAGAA | |
| | ATTTAAAGGCCGTGCAACTATGACTGTTGATACCTC | |
| | CACGAGCACTGTCTACATGGAGCTGTCTTCTTTGCG | |
| | CTCGGAGGACACCGCCGTGTACTACTGTGCCCGCA | |
| | GCGGCTATTACGGCAGCAACCGCTATTGGTACTTC | |
| | GACGTGTGGGGCCAGGGCACGACTGTCACCGTGT | |
| | CGTCCTCTGGCGGAGGTGGCTCTGGAGGCGGTGG | |
| | ATCTGGTGGTGGCGGATCAGACATCCAGATGACCC | |
| | AGAGCCCTAGCAGCCTTTCCGCTTCTGTGGGGGAT | |
| | CGGGTGACTATCACCTGCAAGGCTTCCCAGAGTGT | |
| | GGGCACCAACGTGGCTTGGTACCAGCAGAAGCCCG | |
| | GCAAGGCTCCCAAGGCGCTCATCTATAGTGCCAGC | |
| | TACCGCTACTCCGGCGTTCCCGACAGGTTTTCAGG | |
| | GTCGGGCTCCGGCACAGACTTCACCCTGACCATCT | |
| | CGTCCCTGCAGCCGGAAGATTTCGCCACCTACTTTT | |
| | GTCAACAGTACAACTCCTATCCCTCTACCTTCGGCG | |
| | GCGGGACCAAGCTGGAGATCAAGGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGTCCTGGCTGTTTCTGGTGGC | 172 |
| Gp350- | CATCCTGAAGGGCGTGCAGTGCGATATCCAGATGA | |
| 003_VL2- | CCCAATCACCTAGCAGCTTGTCTGCTTCAGTAGGCG | |
| linker- | ATCGGGTGACCATTACGTGTAAAGCCTCACAAAGTG | |
| VH3_V01 | TAGGGACAAACGTTGCCTGGTATCAACAAAAGCCC | |
| | GGCAAAGCACCTAAGGCACTCATCTATAGTGCTTCT | |
| | TATCGATATTCCGGAGTGCCTGATAGGTTTTCCGGG | |
| | AGCGGCTCCGGAACAGATTTCACACTCACCATTTCC | |
| | TCTCTGCAACCTGAGGACTTTGCTACATATTTCTGC | |
| | CAACAGTATAATTCATATCCATCAACATTCGGGGGG | |
| | GGCACGAAGCTGGAAATTAAATCAGGGGGGGGCG | |
| | GTAGTGGTGGAGGAGGCAGTGGTGGTGGCGGGAG | |
| | TCAAGTACAACTCGTGCAAAGCGGCGCTGAGGTCA | |
| | AGAAACCCGGAGCTTCTGTAAAAGTATCATGCAAAG | |
| | CTTCCGGCTATTCCTTTACCGATTATAATATGAACTG | |
| | GGTTAAACAAGCTCCTGGCCAAGGCCTTGAGTGGA | |
| | TCGGTGTCATAAATCCTAATTTTGGCACAACGAGTT | |
| | ATAACCAAAAGTTCAAAGGAAGAGCGACTTTGACGG | |
| | TCGATCAATCCACAAGCACAGTATATATGGAGTTGA | |
| | GCAGCCTTCGGAGCGAGGACACTGCAGTCTATTAT | |
| | TGTGCGCGGAGCGGGTATTATGGGTCTAACCGTTA | |
| | CTGGTACTTTGACGTGTGGGGTCAAGGAACAACTG | |
| | TCACTGTATCATCAGGATCCACCACAACACCGGCG | |
| | CCACGCCCACCAACACCAGCACCTACCATCGCAAG | |
| | CCAGCCACTGTCCCTGCGGCCCGAGGCCTGTAGG | |
| | CCAGCAGCAGGAGGAGCAGTGCACACCAGGGGCC | |
| | TGGACTTTGCCTGCGACATCTACATCTGGGCACCAC | |
| | TGGCAGGAACATGTGGCGTGCTGCTGCTGTCCCTG | |
| | GTCATCACCCTGTATTGCAAGCGCGGCCGGAAGAA | |
| | GCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGAGCTGGCTGTTTCTGGTGGC | 173 |
| Gp350- | CATCCTGAAGGGCGTGCAGTGCCAGGTCCAGCTGG | |
| 003_VH3- | TGCAGAGCGGCGCCGAGGTGAAGAAGCCCGGCGC | |
| linker- | TTCCGTGAAGGTGTCATGCAAGGCTTCCGGCTATTC | |
| VL2_V01 | CTTCACAGATTATAACATGAATTGGGTTAAGCAGGC | |
| | CCCGGGACAGGGCCTCGAGTGGATTGGGGTGATTA | |
| | ATCCAAACTTCGGTACAACTAGCTACAACCAGAAAT | |
| | TTAAGGGCCGGGCCACTCTGACTGTGGACCAGAGC | |
| | ACCTCCACGGTGTACATGGAGCTGAGCAGTCTACG | |
| | TAGCGAGGACACCGCAGTCTATTACTGTGCCCGCT | |
| | CGGGCTACTACGGCAGCAACCGCTATTGGTACTTC | |
| | GACGTGTGGGGCCAGGGAACGACCGTCACCGTATC | |
| | TAGTTCCGGCGGAGGTGGCTCTGGAGGCGGTGGA | |
| | TCTGGTGGTGGCGGATCAGACATCCAGATGACCCA | |
| | GTCCCCCTCTTCTCTTTCCGCGTCTGTTGGGGACAG | |
| | GGTGACTATCACCTGCAAAGCTAGCCAGAGCGTGG | |
| | GCACCAACGTGGCGTGGTACCAGCAGAAGCCTGG | |
| | GAAGGCGCCTAAAGCCCTGATCTACTCCGCTTCCTA | |
| | CCGCTACTCTGGCGTCCCCGACCGCTTTTCAGGTT | |
| | CTGGCTCCGGCACCGACTTCACCCTGACCATCTCG | |
| | TCGTTGCAGCCGGAAGATTTCGCCACCTACTTTTGT | |
| | CAACAGTACAACTCGTACCCCTCCACCTTCGGCGG | |
| | AGGCACCAAGCTGGAGATCAAGGGATCCACCACAA | |
| | CACCGGCGCCACGCCCACCAACACCAGCACCTACC | |
| | ATCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 174 |
| Gp350- | CATCCTGAAGGGCGTGCAGTGCGACATCCAGATGA | |
| 003_VL3- | CCCAGAGCCCAAGCTCTCTTTCGGCCTCCGTGGGG | |
| linker- | GATAGGGTTACAATCACCTGCAAGGCCTCACAGAG | |
| VH1_V01 | CGTCGGCACCAACGTGGCGTGGTACCAGCAGAAGC | |
| | CGGGAAAAGCTCCTAAGGCGCTCATTTACTCCGCTA | |
| | GCTACCGTTACTCTGGCGTCCCCGACCGCTTTACC | |
| | GGCTCTGGGTCTGGTACTGATTTCACCCTGACCATC | |
| | AGTTCTCTGCAGCCGGAAGACTTTGCCACCTATTTC | |
| | TGTCAACAGTACAACAGCTACCCTTCCACCTTCGGC | |
| | GGCGGTACTAAGCTGGAGATCAAATCCGGCGGAGG | |
| | TGGCTCTGGAGGCGGTGGATCTGGTGGTGGCGGA | |
| | TCACAGGTCCAGCTGGTGCAGAGTGGCGCGGAGG | |
| | TGAAGAAGCCCGGCGCTTCTGTGAAGGTGTCATGC | |
| | AAGGCATCCGGCTACTCCTTCACCGACTACAACATG | |
| | AATTGGGTCCGCCAGGCCCCCGGACAGGGATTGGA | |
| | GTGGATCGGTGTGATTAATCCCAACTTCGGCACGA | |
| | CCAGCTACAACCAGAAATTTAAGGGCCGAGTGACG | |
| | ATGACTGTTGATACCTCCACTTCGACCGTGTACATG | |
| | GAGCTGAGCTCCCTACGGTCGGAGGACACCGCCGT | |
| | GTACTACTGTGCTCGCTCGGGCTATTACGGCAGCA | |
| | ACCGCTATTGGTACTTCGACGTGTGGGGCCAGGGG | |
| | ACCACAGTCACTGTATCTTCGGGATCCACCACAACA | |
| | CCGGCGCCACGCCCACCAACACCAGCACCTACCAT | |
| | CGCAAGCCAGCCACTGTCCCTGCGGCCCGAGGCC | |
| | TGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 175 |
| Gp350- | CATCCTGAAGGGCGTGCAGTGCCAGGTCCAGCTGG | |
| 003_VH1- | TGCAGAGGGGGGCCGAGGTGAAGAAGCCCGGCGC | |
| linker- | GTCCGTAAAGGTGTCATGCAAAGCTTCTGGCTACTC | |
| VL3_V01 | CTTCACCGACTACAACATGAACTGGGTGCGCCAGG | |
| | CCCCTGGACAGGGCCTAGAGTGGATCGGTGTGATT | |
| | AATCCAAATTTCGGCACGACCAGCTACAACCAGAAA | |
| | TTTAAGGGCCGGGTCACTATGACTGTTGACACCTCC | |
| | ACATCGACCGTGTACATGGAGCTGTCCTCTCTTCGC | |
| | TCCGAGGACACGGCCGTGTATTACTGTGCTCGCTC | |
| | GGGGTACTACGGCAGCAACCGCTATTGGTACTTCG | |
| | ACGTGTGGGGCCAGGGAACTACAGTCACTGTTAGC | |
| | TCGTCTGGCGGAGGTGGCTCTGGAGGCGGTGGAT | |
| | CTGGTGGTGGCGGATCAGACATCCAGATGACCCAG | |
| | AGCCCCTCTTCTTTGTCCGCCTCCGTGGGTGATCG | |
| | AGTGACCATCACCTGCAAAGCATCCCAGTCAGTGG | |
| | GCACCAACGTGGCGTGGTACCAGCAGAAGCCTGGC | |
| | AAGGCGCCCAAGGCCCTCATCTACTCCGCTAGCTA | |
| | CAGGTACAGTGGTGTCCCCGACCGCTTCACCGGCT | |
| | CGGGCTCCGGTACTGACTTCACCCTGACCATTTCTT | |
| | CTCTGCAGCCGGAAGATTTCGCCACCTACTTTTGTC | |
| | AACAGTACAACAGCTATCCGTCCACCTTCGGCGGA | |
| | GGGACCAAGCTGGAGATCAAGGGATCCACCACAAC | |
| | ACCGGCGCCACGCCCACCAACACCAGCACCTACCA | |
| | TCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGTCCTGGCTGTTTCTGGTGGC | 176 |
| Gp350-003- | CATCCTGAAGGGCGTGCAGTGCGATATTCAAATGA | |
| VL3-linker- | CACAAAGTCCTTCCAGTTTGTCCGCATCTGTCGGTG | |
| VH2_V01 | ACAGAGTGACAATTACTTGCAAAGCGTCTCAATCTG | |
| | TTGGGACAAACGTCGCATGGTATCAACAAAAGCCA | |
| | GGTAAGGCACCCAAAGCACTGATATATTCCGCAAG | |
| | CTACCGTTATTCCGGGGTGCCAGACCGGTTTACCG | |
| | GCTCTGGTAGTGGAACCGACTTCACCCTTACTATCT | |
| | CTTCTCTGCAACCCGAGGATTTCGCCACCTACTTTT | |
| | GCCAACAATATAATTCATACCCAAGCACTTTCGGCG | |
| | GTGGGACGAAATTAGAGATAAAGTCTGGGGGGGGC | |
| | GGGTCTGGCGGAGGAGGTTCAGGGGGGGGTGGAA | |
| | GTCAAGTGCAATTGGTGCAGAGCGGTGCAGAAGTA | |
| | AAGAAACCTGGTGCTTCAGTCAAGGTCAGCTGTAAA | |
| | GCCTCAGGATACTCTTTTACGGACTATAATATGAATT | |
| | GGGTAAAACAAGCTCCAGGTCAGGGGCTCGAATGG | |
| | ATTGGTGTGATAAATCCCAACTTTGGGACTACGAGT | |
| | TACAACCAGAAGTTCAAGGGAAGGGCCACGATGAC | |
| | TGTGGATACGTCTACAAGTACTGTATACATGGAGCT | |
| | GAGCTCACTCCGCAGCGAAGATACGGCCGTGTATT | |
| | ACTGTGCCAGAAGCGGCTACTATGGATCTAACCGAT | |
| | ACTGGTATTTCGATGTCTGGGGTCAGGGTACTACTG | |
| | TTACCGTCAGCTCAGGATCCACCACAACACCGGCG | |
| | CCACGCCCACCAACACCAGCACCTACCATCGCAAG | |
| | CCAGCCACTGTCCCTGCGGCCCGAGGCCTGTAGG | |
| | CCAGCAGCAGGAGGAGCAGTGCACACCAGGGGCC | |
| | TGGACTTTGCCTGCGACATCTACATCTGGGCACCAC | |
| | TGGCAGGAACATGTGGCGTGCTGCTGCTGTCCCTG | |
| | GTCATCACCCTGTATTGCAAGCGCGGCCGGAAGAA | |
| | GCTGCTGTACATCTTCAAGCAGCCCTTCATGCGGC | |
| | CCGTGCAGACAACCCAGGAGGAGGACGGCTGCAG | |
| | CTGTAGGTTCCCTGAAGAGGAGGAGGGAGGATGTG | |
| | AGCTGAGGGTGAAGTTTTCCCGGTCTGCCGATGCC | |
| | CCAGCCTATAAGCAGGGCCAGAATCAGCTGTACAA | |
| | CGAGCTGAATCTGGGCCGGAGAGAGGAGTATGACG | |
| | TGCTGGATAAGAGGAGGGGAAGAGATCCCGAGATG | |
| | GGAGGCAAGCCCAGAAGAAAGAACCCTCAGGAGG | |
| | GCCTGTATAATGAGCTGCAGAAGGACAAGATGGCC | |
| | GAGGCCTACAGCGAGATCGGCATGAAGGGAGAGA | |
| | GGCGCCGGGGCAAGGGACACGATGGCCTGTACCA | |
| | GGGCCTGTCAACAGCAACTAAAGATACTTACGACG | |
| | CACTGCACATGCAGGCTCTGCCACCCCGA | |
| CAR_EBV- | ATGGAGTTCGGCCTGAGCTGGCTGTTTCTGGTGGC | 177 |
| Gp350-003- | CATCCTGAAGGGCGTGCAGTGCCAGGTGCAGCTGG | |
| VH2-linker- | TGCAGAGCGGCGCCGAGGTGAAGAAGCCTGGGGC | |
| VL3_V01 | TTCCGTAAAGGTGTCATGCAAAGCTAGCGGCTACTC | |
| | CTTCACTGACTACAACATGAACTGGGTCAAGCAGGC | |
| | CCCGGGACAGGGTTTGGAGTGGATTGGGGTGATTA | |
| | ATCCAAATTTTGGTACGACCAGCTACAACCAGAAAT | |
| | TTAAGGGCCGTGCCACTATGACTGTCGATACATCGA | |
| | CCTCCACCGTCTACATGGAGCTGTCCTCTCTGCGCT | |
| | CGGAAGACACCGCCGTGTACTACTGTGCTCGCTCA | |
| | GGCTATTACGGCAGCAACCGCTATTGGTACTTCGAC | |
| | GTGTGGGGCCAGGGCACCACAGTCACCGTGTCGA | |
| | GCTCCGGCGGAGGTGGCTCTGGAGGCGGTGGATC | |
| | TGGTGGTGGCGGATCAGACATCCAGATGACCCAGT | |
| | CCCCTTCTTCTCTTTCCGCCTCCGTGGGTGATCGG | |
| | GTGACCATCACCTGCAAGGCCAGTCAGTCTGTGGG | |
| | CACCAACGTGGCGTGGTACCAGCAGAAGCCCGGCA | |
| | AAGCACCCAAGGCGCTAATCTACAGTGCCAGCTAC | |
| | AGGTACTCGGGCGTTCCCGACCGCTTCACCGGCTC | |
| | CGGATCTGGTACGGACTTCACTCTGACCATCTCGTC | |
| | CCTCCAGCCGGAGGACTTTGCGACTTATTTCTGTCA | |
| | ACAGTACAACAGCTATCCCTCTACCTTCGGCGGGG | |
| | GGACCAAGCTGGAGATCAAGGGATCCACCACAACA | |
| | CCGGCGCCACGCCCACCAACACCAGCACCTACCAT | |
| | CGCAAGCCAGCCACTGTCCCTGCGGCCCGAGGCC | |
| | TGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |
| CAR_EBV- | ATGGAGTTCGGCCTGAGCTGGCTGTTTCTGGTGGC | 178 |
| Gp350-003- | CATCCTGAAGGGCGTGCAGTGCGACATCCAGATGA | |
| VL3-linker- | CCCAGAGCCCGAGCTCCCTTTCGGCGTCCGTGGGC | |
| VH3_V01 | GATCGAGTGACCATCACCTGCAAAGCCAGCCAGAG | |
| | TGTCGGCACCAACGTGGCTTGGTACCAGCAGAAGC | |
| | CCGGCAAGGCTCCCAAGGCGCTCATCTATAGTGCG | |
| | AGTTACCGCTACTCTGGGGTGCCCGACCGCTTCAC | |
| | CGGGTCCGGCTCCGGCACCGACTTCACATTGACTA | |
| | TTTCTTCTCTGCAGCCGGAAGATTTCGCCACCTACT | |
| | TTTGTCAACAGTACAACAGCTATCCCTCTACCTTCG | |
| | GCGGCGGGACCAAGCTGGAGATCAAGTCCGGCGG | |
| | AGGTGGCTCTGGAGGCGGTGGATCTGGTGGTGGC | |
| | GGATCACAGGTCCAGCTGGTGCAGTCCGGGGCAG | |
| | AGGTGAAGAAGCCTGGGGCTTCCGTTAAGGTGTCA | |
| | TGCAAGGCCTCGGGCTACTCCTTCACCGATTACAAC | |
| | ATGAACTGGGTCAAGCAGGCCCCTGGACAGGGTCT | |
| | CGAGTGGATCGGTGTGATTAATCCAAATTTCGGTAC | |
| | TACTAGCTACAACCAGAAATTTAAAGGCCGTGCCAC | |
| | TCTGACCGTTGACCAGAGCACCTCCACCGTCTACAT | |
| | GGAGCTGTCATCGCTGCGCTCGGAGGACACCGCC | |
| | GTGTACTACTGTGCTCGGTCTGGCTATTACGGCAG | |
| | CAACCGCTATTGGTACTTCGACGTGTGGGGCCAGG | |
| | GCACGACGGTAACAGTGTCCAGCGGATCCACCACA | |
| | ACACCGGCGCCACGCCCACCAACACCAGCACCTAC | |
| | CATCGCAAGCCAGCCACTGTCCCTGCGGCCCGAGG | |
| | CCTGTAGGCCAGCAGCAGGAGGAGCAGTGCACAC | |
| | CAGGGGCCTGGACTTTGCCTGCGACATCTACATCTG | |
| | GGCACCACTGGCAGGAACATGTGGCGTGCTGCTGC | |
| | TGTCCCTGGTCATCACCCTGTATTGCAAGCGCGGC | |
| | CGGAAGAAGCTGCTGTACATCTTCAAGCAGCCCTTC | |
| | ATGCGGCCCGTGCAGACAACCCAGGAGGAGGACG | |
| | GCTGCAGCTGTAGGTTCCCTGAAGAGGAGGAGGGA | |
| | GGATGTGAGCTGAGGGTGAAGTTTTCCCGGTCTGC | |
| | CGATGCCCCAGCCTATAAGCAGGGCCAGAATCAGC | |
| | TGTACAACGAGCTGAATCTGGGCCGGAGAGAGGAG | |
| | TATGACGTGCTGGATAAGAGGAGGGGAAGAGATCC | |
| | CGAGATGGGAGGCAAGCCCAGAAGAAAGAACCCTC | |
| | AGGAGGGCCTGTATAATGAGCTGCAGAAGGACAAG | |
| | ATGGCCGAGGCCTACAGCGAGATCGGCATGAAGG | |
| | GAGAGAGGCGCCGGGGCAAGGGACACGATGGCCT | |
| | GTACCAGGGCCTGTCAACAGCAACTAAAGATACTTA | |
| | CGACGCACTGCACATGCAGGCTCTGCCACCCCGA | |
| CAR_EBV- | ATGGAGTTCGGCCTGAGCTGGCTGTTTCTGGTGGC | 179 |
| Gp350-003- | CATCCTGAAGGGCGTGCAGTGCCAGGTGCAGCTGG | |
| VH3-linker- | TGCAGAGCGGGGCCGAGGTGAAGAAGCCTGGCGC | |
| VL3_V01 | GTCCGTTAAGGTGTCATGCAAAGCATCTGGCTACTC | |
| | CTTCACCGACTACAATATGAACTGGGTCAAGCAGGC | |
| | CCCGGGTCAGGGTTTGGAGTGGATTGGTGTGATTA | |
| | ATCCCAACTTCGGAACTACCAGCTACAACCAGAAAT | |
| | TTAAAGGAAGGGCTACCCTGACCGTGGACCAGTCG | |
| | ACCTCGACCGTCTACATGGAACTGTCTAGCCTGCG | |
| | CTCGGAGGATACCGCCGTGTACTACTGTGCCCGTT | |
| | CCGGCTATTACGGCAGCAACCGCTATTGGTACTTC | |
| | GACGTGTGGGGCCAGGGCACGACTGTCACCGTGT | |
| | CCTCGTCCGGGGGAGGTGGCTCTGGAGGCGGTGG | |
| | ATCTGGTGGTGGOGGATCAGACATCCAGATGACCC | |
| | AGAGCCCTTCCTCTCTTTCCGCTTCCGTGGGTGATC | |
| | GCGTAACTATCACCTGCAAGGCTTCCCAGAGCGTT | |
| | GGGACCAACGTGGCGTGGTACCAGCAGAAGCCCG | |
| | GCAAGGCCCCCAAGGCGCTCATCTACTCGGCTAGC | |
| | TACCGGTACAGTGGCGTCCCCGACCGCTTTACCGG | |
| | CTCCGGCTCCGGCACAGATTTCACACTAACTATCTC | |
| | TTCTCTGCAGCCGGAGGACTTTGCCACGTATTTCTG | |
| | TCAACAGTACAACTCCTATCCAAGTACCTTCGGCGG | |
| | CGGTACCAAGCTGGAGATCAAGGGATCCACCACAA | |
| | CACCGGCGCCACGCCCACCAACACCAGCACCTACC | |
| | ATCGCAAGCCAGCCACTGTCCCTGCGGCCCGAGGC | |
| | CTGTAGGCCAGCAGCAGGAGGAGCAGTGCACACCA | |
| | GGGGCCTGGACTTTGCCTGCGACATCTACATCTGG | |
| | GCACCACTGGCAGGAACATGTGGCGTGCTGCTGCT | |
| | GTCCCTGGTCATCACCCTGTATTGCAAGCGCGGCC | |
| | GGAAGAAGCTGCTGTACATCTTCAAGCAGCCCTTCA | |
| | TGCGGCCCGTGCAGACAACCCAGGAGGAGGACGG | |
| | CTGCAGCTGTAGGTTCCCTGAAGAGGAGGAGGGAG | |
| | GATGTGAGCTGAGGGTGAAGTTTTCCCGGTCTGCC | |
| | GATGCCCCAGCCTATAAGCAGGGCCAGAATCAGCT | |
| | GTACAACGAGCTGAATCTGGGCCGGAGAGAGGAGT | |
| | ATGACGTGCTGGATAAGAGGAGGGGAAGAGATCCC | |
| | GAGATGGGAGGCAAGCCCAGAAGAAAGAACCCTCA | |
| | GGAGGGCCTGTATAATGAGCTGCAGAAGGACAAGA | |
| | TGGCCGAGGCCTACAGCGAGATCGGCATGAAGGG | |
| | AGAGAGGCGCCGGGGCAAGGGACACGATGGCCTG | |
| | TACCAGGGCCTGTCAACAGCAACTAAAGATACTTAC | |
| | GACGCACTGCACATGCAGGCTCTGCCACCCCGA | |

### Example 4: Generation of CAR T-Cells Comprising Anti-EBV Gp350 Scfv

To generate anti-EBV Gp350 CAR-T cells, PBMCs were first purified from huffy coat samples using Ficoll gradient density medium. T cells were purified from PBMCs using a commercially available T cell isolation kit. In short, 3^{rd} generation of recombinant lentivirus packaging system was employed for CAR encoding lentivirus vector preparation. 72 hours after transfer plasmid and 3 helper plasmids co-transfected into HEK293T cells, the virus particles in the culture supernatants were concentrated with Takara Lenti-X™ Concentrator following product manual. Then the lentivirus was applied to infect enriched T cells for CART cells generation, which had been preactivated with TransACT (Miltenyi Biotec) for 2 days in TexMACS media supplemented with IL-7 and IL-15, 10 ng/ml or 5 ng/ml respectively. The CART cell immunophenotypic characterization during process were analyzed with antibodies from BioLengend and Jackson Immunoresearch Laboratories, to study effector/memory and exhaustion status (CD3, CD4, CD8, CD45RA, CD62L, CCR7, Lags, Tim3, PD-1), using LIVE/DEAD™ Violet Viability kit for living cells grouping (Chimeric Antigen Receptor T Cells, Development and Production, Springer (2020)).

FACS studies were performed to confirm that T cells were successfully transduced with anti-EBV Gp350 CARs which are expressed on the surface of primary T-cells and can recognize recombinant EBV Gp350 (data not shown).

Cytotoxic activity of an exemplary anti-EBV Gp350 CAR-T cells (CAR_EBV-Gp350-003) were evaluated. Specifically, anti-EBV Gp350 CAR-T cell were incubated with PCI-gp350 cell line expressing gp350 (or with PCI, cell line without gp350 expression) at effector-to-target (E:T) ratio ranging from 5:1 to 10: in T cell expansion media. Cell viability was measured and cytotoxic activity were calculated in terms of % cytotoxicity.

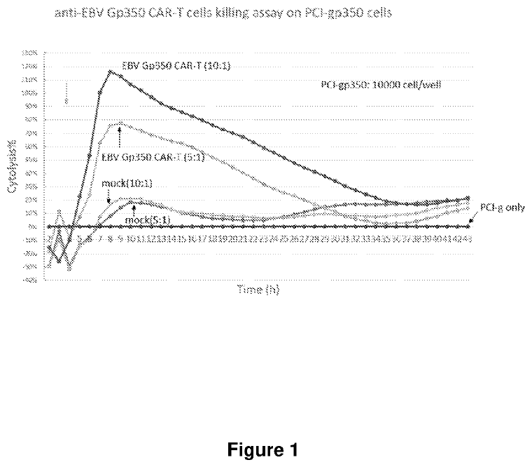

As shown in FIG. 1, the exemplary anti-EBV Gp350 CAR-T cells (CAR_EBV-Gp350-003) demonstrated potent killing on PCI-gp350 cells but did not show detectable activity in PCI-g cells without gp350 expression.

### Example 5: ZNP Composition (and ZNP-EBV-VLP Composition) Promotes T Cell Proliferation, Cytokine Release and/or Cytotoxicity In Vitro

Purified T cells from human peripheral blood mononuclear cells (PBMC) (or CAR-T cells) were pre-stained with CellTrace CFSE (CarboxyFluoroscein Succinimidyl Ester) staining solution following the manufacturer's instruction, for in vitro labelling of T cells to trace T cell proliferation (multiple cell generations) using dye dilution by flow cytometry. The T cells were placed in coincubation for 4 days with PBMCs and ZNP composition described in Example 1 at defined working concentrations (50, 100 or 200 μg/ml), or together with ZNP-EBV-VLP. T cell proliferation was analysed using a flow cytometer with 488 nm excitation and emission filters appropriate for fluorescein.

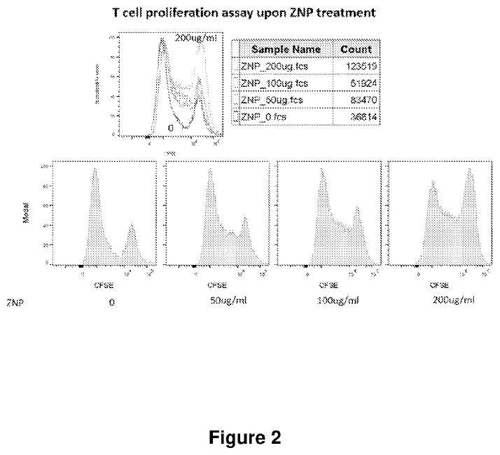

As shown in FIG. 2, co-incubation of ZNP composition prepared in Example 1 with T cells and PBMCs promotes activation and proliferation of T cells in a ZNP concentration dependent manner, suggesting the ZNP composition (or ZNP-EBV-VLP) can be useful in enhancing efficacy of T cell therapy including CAR-T cells.

The promotion of cytokine release and in vitro cytotoxicity from activated T cells (or CAR-T cells) by co-treatment with the ZNP composition (or ZNP-EBV-VLP) will be performed. Activated T cells (or CAR-T cells) and target cancer cells will be co-cultured for 24 hours before supernatant analysis with Enzyme-Linked Immunosorbent Assay (ELISA) to quantify cytokines released with the ELISA MAX™ Standard Set. The xCELLigence system will be utilized for assessment of T cell-mediated cytotoxicity at different effector-to-target (E:T) ratio. Cell-mediated killing will be quantified over the next 48 h reading electrical impedance every 30 min. Percent-specific lysis values will be calculated using GraphPad Prism Software v6 for each replicate at each time point.

### Example 6: Cytokine-Release Assay and Cell Killing Assay to Assess the Activity of Anti-EBV Gp350 CAR T-Cells Alone, and in Combination with ZNP Composition or ZNP-EBV-VLP Composition

xCELLigence Real-Time Cell Analysis for Cytotoxicity Assay: The xCELLigence system is being utilized for assessment of T cell-mediated cytotoxicity. 1×10⁴ target expressing cells will be plated in each well of an E-Plate and grown overnight, quantifying electrical impedance using the RTCA SP Analyzer system. Approximately 24 h later, 50 μL of anti-EBV Gp350_CAR-T cells will be added at different effector-to-target (E:T) ratio or 50 μL of media or 10% Triton-X 100 will be added as negative and positive controls, respectively. Cell-mediated killing will be quantified over the next 48 h reading electrical impedance every 30 min. Percent-specific lysis values will be calculated using GraphPad Prism Software v6 for each replicate at each time point.

Cytokine-Release Assay: CAR-T cells and target cancer cells will be co-cultured for 24 hours in 96 well plate at defined E:T ratio. Then the supernatants will be analyzed with Enzyme-Linked Immunosorbent Assay (ELISA) to quantify cytokines released following the ELISA MAX™ Standard Set manufacturer's instructions. When necessary, supernatants will be centrifuged to remove debris prior to analysis.

### Example 7: VLP of Epstein-Barr Virus (EBV) Enhances Cytotoxicity of Anti-EBV Gp350 CAR-T Cells

Cytotoxic activity of an exemplary anti-EBV Gp350 CAR-T cells (CAR_EBV-Gp350-003) with or without combination with VLP of EBV prepared in Example 2 (EBV-VLP particles comprising gp350, BKRF4, BVRF1, BDLF3, BZLF2, BXLF2, BNRF1, BALF4 and BZLF1) was evaluated. The xCELLigence system is being utilized for assessment of T cell-mediated cytotoxicity.

Specifically, target cells (PCI-gp350 cell line expressing gp350, or negative control PCI cells) were first seeded in xCELLigence E-plate 96 one day before, at 1×10⁶ cells/well in 130 μl DMEM medium+10% FBS. Separately, 1×10⁴ of Raji cells (human B lymphoblastoid cell line as VLP targeting cell) were incubated with 1.25×10⁶; 1.25×10⁷, or 1.25×10⁸ of EBV VLPs respectively, in 50 μl RPMI medium+10% FBS at 37° C. for 1 hour before loading to the xCELLigence E-plate. After incubation, the anti-EBV Gp350 CAR-T cells and the Raji cells (1×10⁴ cells) were then seeded to the xCELLigence E-plate for co-culturing with target cells at effector-to-target (E:T) ratio of 5:1. Cell-mediated killing were quantified over the next 68 hours reading electrical impedance every 30 min. Specific cytolysis (%) were calculated using GraphPad Prism Software v6 for each replicate at each time point.

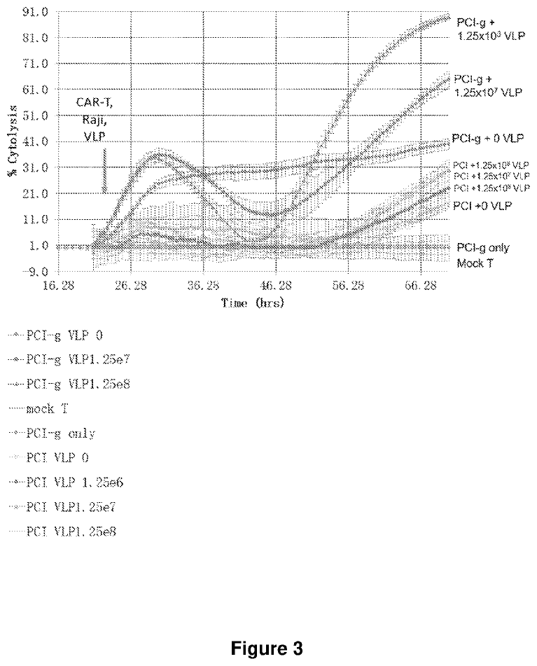

As shown in FIG. 3, co-incubating of EBV VLPs with Raji cells significantly enhances the cytotoxicity (% cytolysis) of anti-EBV Gp350 CAR-T cells to target cells PCI-gp350 cell line in the VLP quantity correlated manner (PCI-g+1.25×10⁷ VLP, PCI-g+1.25×10⁸ VLP compared to PCI-g+0 VLP). In contrast, EBV VLPs do not increase cytotoxicity (% cytolysis) of anti-EBV Gp350 CAR-T cells to control PCI cells.

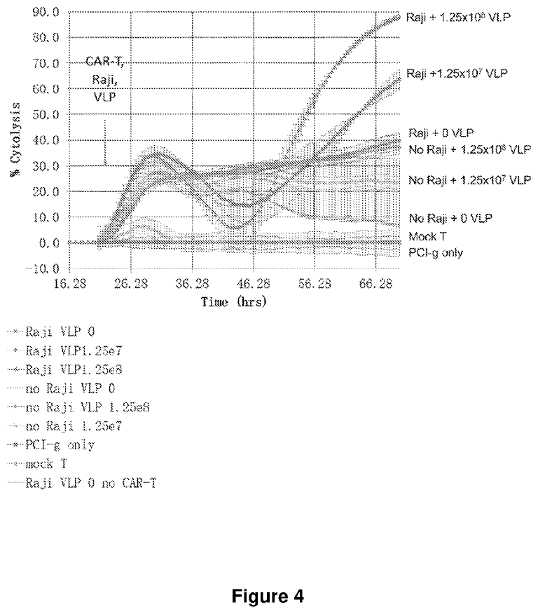

FIG. 4 further shows that the enhanced cytotoxicity of anti-EBV Gp350 CAR-T cells to target PCI-gp350 cells in the presence of EBV VLPs is dependent on the co-incubation with VLP-targeting Raji cells. Sample of 1.25×10⁸ VLP co-incubated with Raji cells (Raji+1.25×10⁸ VLP) shows much higher cytotoxicity than the sample of 1.25×10⁸ VLP without co-incubated Raji cells (No Raji+1.25×10⁸ VLP).

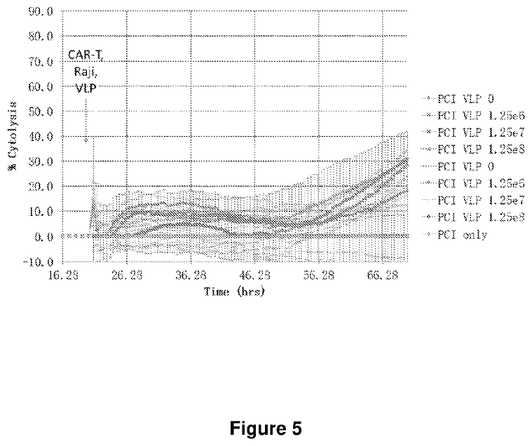

FIG. 5 shows that anti-EBV Gp350 CAR-T cells show little or low cytotoxicity to PCI cell line without gp350 antigen, in the absence or in the presence of EBV VLPs.

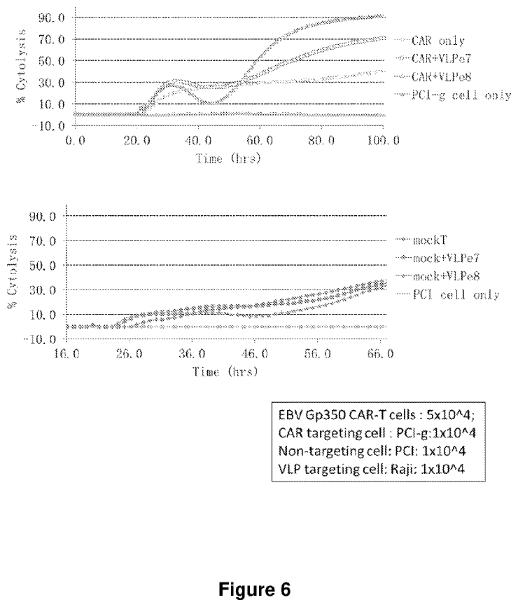

FIG. 6 shows results of another set of experiments of co-incubating VLP of EBV with Raji cells which significantly enhances the cytotoxicity (% cytolysis) of anti-EBV Gp350 CAR-T cells to target cells PCI-gp350 cell line (PCI-g) in the VLP-concentration manner (CAR+VLPe7 or CAR+VLPe8, compared to CAR only). In contrast, VLP of EBV does not increase cytotoxicity (% cytolysis) of anti-EBV Gp350 CAR-T cells to control PCI cells. VLP of EBV alone does not increase cytotoxicity (% cytolysis) of mock T cells or PCI cell without gp350 expression.

### Example 8: In Vivo Assessment of CAR T-Cells Comprising Anti-EBV Gp350 Scfv in Tumor Mouse Models

Xenograft models of nasopharyngeal carcinoma were established by subcutaneous (s.c.) injection of 4×10⁶/100 μl nasopharyngeal carcinoma cells (C666-1, engineered to express gp350 protein and a luciferase tracker) into 6-week-old NOD-scid IL2Rγnull mice (NOG) mice (Charles River). On the next day, mice were examined by IVIS imaging before being separated into groups for intravenous administration of T cells.

To conduct imaging check, mice were anesthetized with inhaled isoflurane and were maintained with 1.5-2% isofluorane during imaging procedures. Luciferase-based bioluminescence imaging was performed with an IVIS Lumina Series III imaging system equipped with a camera box and warming stage. After intraperitoneal injection of 150 mg/kg D-luciferin dissolved in phosphate buffered saline (PBS) for 15 minutes, mice images were captured and bioluminescence intensity was quantitated and analyzed. Identical regions of interest (ROI) over each mouse were selected for total flux values determination, presented in photons (p)/second (sec).

Gp350-targeting CAR-T cells were prepared following in-house protocol with PBMC from healthy donors. Upon T cell administration, 4×10⁶/100 μl or 2×10⁶/100 μl of CAR-T cells, Mock T cells or PBS were IV injected into the C666-1 mouse model. Body weight and tumor growth were monitored 2 to 3 times per week. On Day 7, 14, 18, or 21, bioluminescence intensity level was examined to monitor changes in tumor cells. An autopsy was performed on 18 or 24 days, blood was collected for FACS testing; tumors and major organs were collected and weighed.

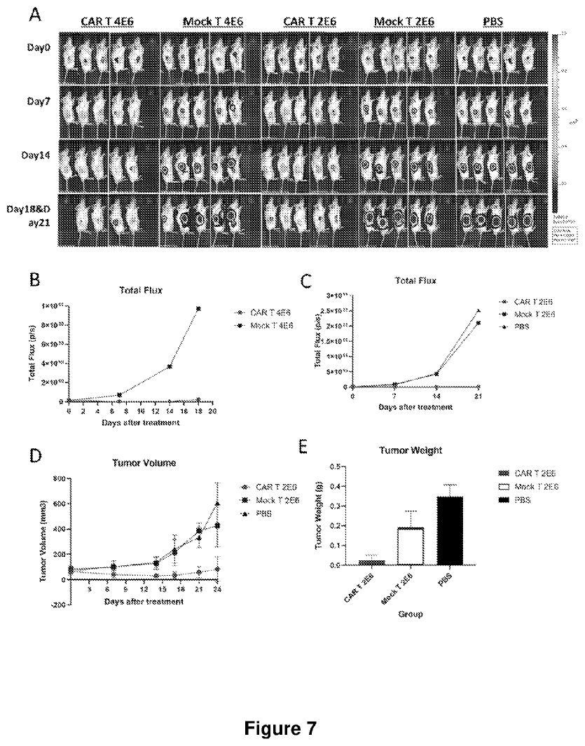

FIG. 7 shows that Gp350 CAR-T cells inhibited nasopharyngeal carcinoma cells C666-1 growth and suppressed tumor formation in vivo. C666-1 (engineered with gp350 and luciferase expression) cells were subcutaneously injected into mice followed by 4×10⁶ (4E6) or 2×10⁶ (4E6) gp350 CAR-T or Mock T cells administration. FIG. 7A shows results of tumor cells growth luminescence imaging at Day 0, 7, 14, 18 (4×10⁶ groups) or 21 (2×10⁶ and PBS groups) time points were recorded and compared, showing the specific tumor formation suppression by gp350 CAR-T cells. FIG. 7B shows average tumor cell luminescence intensity change at Day 0, 7, 14, 18 or 21 post CAR-T injection for 4×10⁶ (4E6) CAR-T cell or Mock T cells treated groups. FIG. 7C shows average tumor cell luminescence intensity change at Day 0, 7, 14, 18 or 21 post CAR-T injection for 2×10⁶ (2E6) of CAR-T cell, Mock T cells, or PBS treated groups. FIG. 7D shows the average tumor volume change pattern post CAR-T injection on Day 0, 7, 14, 18 or 21. FIG. 7E shows dissection and evaluation of tumor tissue weight in mice across different experimental groups, showing reduction of tumor growth in CAR-T treat group.

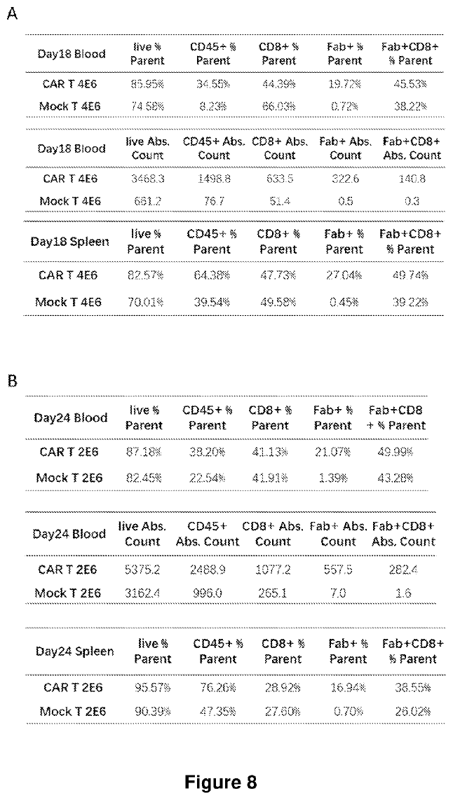

FIG. 8 further shows cell percentage and counts comparison of human CD45+, CD8+, CAR+, and CAR+/CD8+ cells in blood or spleen samples from of 4×10⁶ (4E6) CAR T and Mock T treatment groups at Day 18 (FIG. 8A), and 2×10⁶ (2E6) CAR T cells and Mock T cells treatment groups at Day 24 (FIG. 8B). The results shows that CAR-T cells was found existence in blood and spleen while suppressing tumor formation.

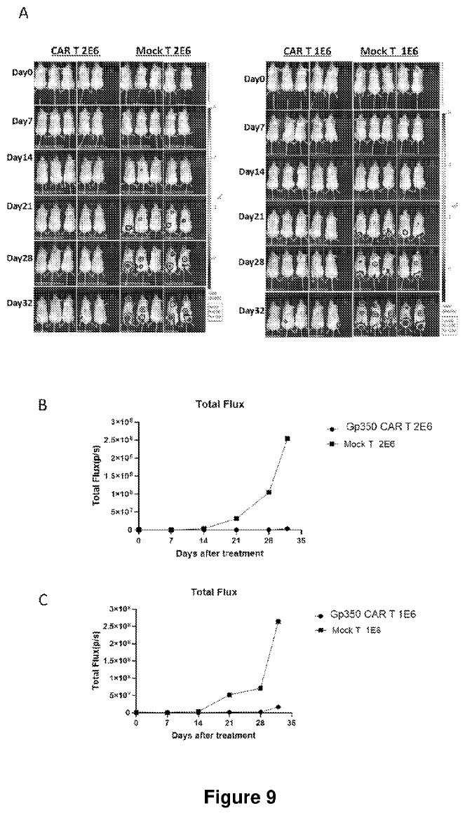

For in vivo studies in T-cell lymphoma models, 2×10⁶/100 μl Jurkat-gp350-luc cells were injected 5 or 7 days for tumor formation in mice before gp350-targeting CAR-T cells treatment. In the former group, mice were examined by IVIS imaging and being administrated with 1×10⁶ (1E6)/100 μl CAR-T cell or Mock T cell; in the latter group, mice were examined by IVIS imaging and being administrated with 2×10⁶ (2E6)/100 μl CAR-T or Mock T respectively. The mice were weighed and observed 2 to 3 times a week. On days 7, 14, and 21, bioluminescence intensity level was examined to monitor changes in tumor cells. The result shows that Gp350 CAR-T cells inhibit T-cell lymphoma cell growth and suppressed tumor formation in vivo (FIG. 9). FIG. 9A shows tumor profile of T-cell lymphoma mice model which were injected with Jurkat-gp350-luc cells 5 or 7 days for tumor formation. The mice were examined by IVIS imaging and being administrated with 2×10⁶ (2E6) or 1×10⁶ (1E6) of CAR-T cells or Mock T cells respectively. Tumor cells growth luminescence imaging at Day 0, 7, 14, 21, 28, 32 time points were recorded and compared, showing the specific tumor formation suppression by gp350 CAR-T cells. FIGS. 9B and 9C respectively shows the average tumor cell luminescence intensity change along the time course in 2×10⁶ (2E6) or 1×10⁶ (1E6) of CAR-T, or Mock T groups post CAR-T injection.

### Example 9: Tumor Shrinkage Induced by CAR T-Cells Comprising Anti-EBV Gp350 in Patient Diagnosed with B-Cell Acute Lymphoblastic Leukemia with Central Nervous System Involvement

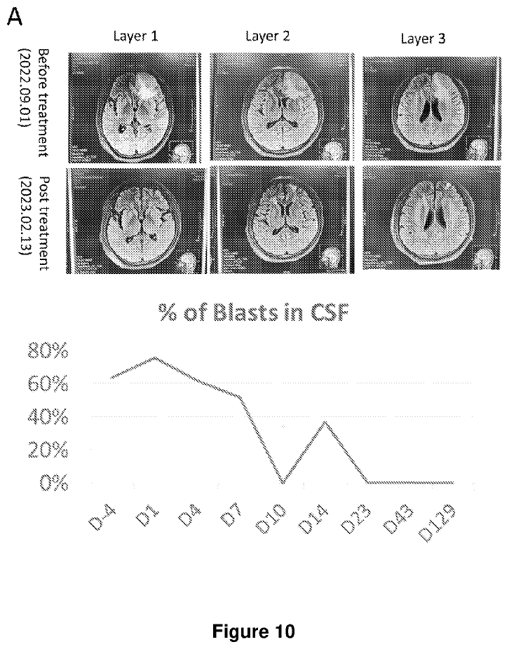

A trial study was conducted to test the safety and efficacy of EBV Gp350-specific CAR-T cells for treating human subjects. ZYH, a male aged 36 who was diagnosed with B-cell acute lymphoblastic leukemia with central nervous system involvement was administered with about 2.87×10⁶ of EBV Gp350-specific CAR-T cells. FIG. 10 (A) shows MRI imaging result which showed the lesion in the brain before treatment (top panel) and after treatment (bottom panel), which almost disappeared after treatment; FIG. 10 (B) shows that the percentages of aberrant blasts in CSF dropped from day 1 to day 129 after treatment. After the treatment, the subject was discharged from hospital without CRS, ICANS and significant abnormality of heart, liver and kidney function tests.

1-20. (canceled)

21. A method of increasing anti-cancer response of an immune cell therapy in an individual, the method comprising administering to the individual an effective amount of an immunogenic composition comprising a VLP of Epstein Barr virus (EBV).

22. A method of treating an individual having cancer, the method comprising:
(a) administering to the individual an effective amount of the immunogenic composition comprising a VLP of Epstein Barr virus (EBV); and
(b) administering to the individual an immune cell therapy.

23-36. (canceled)

37. The method of claim 21, wherein the VLP comprises glycoprotein 350/220 (gp350) protein or fragment thereof, wherein optionally the VLP further comprises one, two, three or more polypeptide sequences that is/are at least 80%, at least 90%, at least 95%, at least 96%, at least 97%, at least 98%, at least 99% identical or 100% identical to polypeptide sequences selected from the group consisting of SEQ ID NOs: 102 to 117; wherein more preferably said EBV-VLP comprises the EBV proteins of gp350, BKRF4, BVRF1, BDLF3, BZLF2, BXLF2, BNRF1, BALF4 and BZLF1, or their functional variants thereof that is at least 80%, at least 90%, at least 95%, at least 96%, at least 97%, at least 98%, at least 99% identical or 100% identical to the polypeptide sequences described herein.

38. The method of claim 21, wherein the immune cell therapy is a CAR-T cell therapy, wherein optionally said composition increases anti-cancer effect of the cell therapy.

39. The method of claim 38, wherein the antigen that is recognized by the CAR is an Epstein-Barr virus antigen (EBV antigen), wherein preferably said EBV antigen is EBV glycoprotein 350/220 (gp350/220).

40. The method of claim 21, wherein the immune cell comprises CAR that comprises an antigen-binding domain comprising a heavy chain variable region (VH) and/or light chain variable region (VL) as provided in Table 1 and Table 2, or a specific VH and VL combination as provided in Table 1 and Table 2; wherein preferably said antigen-binding domain comprises:
(1) a heavy chain variable region (VH) having the polypeptide sequence of SEQ ID NO: 1 or a variant in which 1, 2 or 3 amino acids are substituted related to the sequence, and a light chain variable region (VL) having the polypeptide sequence of SEQ ID NO: 6 or a variant in which 1, 2 or 3 amino acids are substituted related to the sequence;
(2) a heavy chain variable region (VH) having the polypeptide sequence of SEQ ID NO: 2 or a variant in which 1, 2 or 3 amino acids are substituted related to the sequence, and a light chain variable region (VL) having the polypeptide sequence of SEQ ID NO: 7 or a variant in which 1, 2 or 3 amino acids are substituted related to the sequence;
(3) a heavy chain variable region (VH) having the polypeptide sequence of SEQ ID NO: 3 or a variant in which 1, 2 or 3 amino acids are substituted related to the sequence, and a light chain variable region (VL) having the polypeptide sequence of SEQ ID NO: 8 or a variant in which 1, 2 or 3 amino acids are substituted related to the sequence;
(4) a heavy chain variable region (VH) having the polypeptide sequence of SEQ ID NO:4 or a variant in which 1, 2 or 3 amino acids are substituted related to the sequence, and a light chain variable region (VL) having the polypeptide sequence of SEQ ID NO: 9 or a variant in which 1, 2 or 3 amino acids are substituted related to the sequence;
(5) a heavy chain variable region (VH) having the polypeptide sequence of SEQ ID NO:5 or a variant in which 1, 2 or 3 amino acids are substituted related to the sequence, and a light chain variable region (VL) having the polypeptide sequence of SEQ ID NO: 10 or a variant in which 1, 2 or 3 amino acids are substituted related to the sequence;
(6) a heavy chain variable region (VH) having the polypeptide sequence of SEQ ID NO:138 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(7) a heavy chain variable region (VH) having the polypeptide sequence of SEQ ID NO:138 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(8) a heavy chain variable region (VH) having the polypeptide sequence of SEQ ID NO:138 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(9) a heavy chain variable region (VH) having the polypeptide sequence of SEQ ID NO:139 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(10) a heavy chain variable region (VH) having the polypeptide sequence of SEQ ID NO:139 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence;
(12) a heavy chain variable region (VH) having the polypeptide sequence of SEQ ID NO:139 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence;
(13) a heavy chain variable region (VH) having the polypeptide sequence of SEQ ID NO:140 or a variant in which 1, 2 or 3 amino acids are substituted related to the sequence, and a light chain variable region (VL) having the polypeptide sequence of SEQ ID NO: 141 or a variant in which 1, 2 or 3 amino acids are substituted related to the sequence;
(14) a heavy chain variable region (VH) having the polypeptide sequence of SEQ ID NO:140 or a variant in which 1, 2 or 3 amino acids are substituted related to the sequence, and a light chain variable region (VL) having the polypeptide sequence of SEQ ID NO: 142 or a variant in which 1, 2 or 3 amino acids are substituted related to the sequence; or
(15) a heavy chain variable region (VH) having the polypeptide sequence of SEQ ID NO:140 or a variant in which 1, 2 or 3 amino acids are substituted related to the sequence, and a light chain variable region (VL) having the polypeptide sequence of SEQ ID NO: 143 or a variant in which 1, 2 or 3 amino acids are substituted related to the sequence.

41. The method according to claim 21, wherein the immune cell comprises a CAR that comprises an antigen-binding domain comprising one, two, three or more HCDRs and/or one, two, three or more LCDR as provided in Table 3 and Table 4, or a specific HCDR1-3 and LCDR1-3 combination as provided in Table 3 and Table 4; wherein preferably said antigen binding domain that binds EBV gp350/220 comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 11, 12, 13, 26, 27 and 28, respectively;
(2) SEQ ID NOs: 14, 15, 16, 29, 30 and 31, respectively;
(3) SEQ ID NOs: 17, 18, 19, 32, 33 and 34, respectively;
(4) SEQ ID NOs: 20, 21, 22, 35, 36 and 37, respectively; or
(5) SEQ ID NOs: 23, 24, 25, 38, 39 and 40, respectively; or
an EBV glycoprotein 350/220 antigen-binding domain which competes therewith or binds to the same epitope as that in the EBV glycoprotein 350/220 to which any one of the antigen-binding domain (1) to (5) of the CAR as described herein bind.

42. The method according to claim 21, wherein the immune cell comprises a CAR comprising any one of the CAR amino acid sequences as provided in Table 5.

43. The method of claim 21, wherein the immunogenic composition is administered with an engineered immune cell (preferably CAR-T cell or engineered TCR-T cell), wherein optionally said immunogenic composition is to be administered to the subject before, after or simultaneously with the immune cell.

44. The method of 21, wherein the immunogenic composition comprises a tumor-associated antigen or an antigen associated with a virus, wherein optionally said immunogen is a VLP of Epstein Barr virus (EBV) that comprises glycoprotein 350/220 (gp350) protein or fragment thereof.

45. The method of claim 21, wherein the immunogenic composition comprises an aminoglycoside antibiotic stabilizer selected from kanamycin, Streptomycin, Dihydrostreptomycin, Mannoside Streptomycin, Amikacin, Amikacin, Dibekacin, Vietomycin, Gentamycin, and any combination thereof; and preferably said stabilizer is kanamycin.

46. The method according to claim 21, wherein the immunogenic composition comprises a non-aminoglycoside amine stabilizer selected from the group consisting of a polyethylene glycol monomethyl ether, polyethylene glycol, polyethyleneimine, folic acid, galactose, polylysine, protamine, shell oligosaccharide, chitosan, spermine, glucosamine, and any combination thereof; preferably said stabilizer is a polyethylene glycol monomethyl ether, polyethylene glycol, polyethyleneimine, folic acid, galactose, polylysine, chitin, chitosan or glucosamine; more preferably said stabilizer is a polyethylene glycol monomethyl ether, polyethylene glycol, polyethyleneimine, ε-polylysine, hexylglucosamine or acetylglucosamine.

47. The method according to claim 21, wherein the immunogenic composition comprises a cation selected from the group consisting of calcium, cadmium, lithium, magnesium, cerium, cesium, chromium, cobalt, deuterium, gallium, iodine, iron, zinc, and any combination thereof; and preferably said cation is calcium.

48. The method according to claim 21, wherein said immune cell is a T cell that expresses a CAR comprising an extracellular domain, a transmembrane domain, and an intracellular domain, wherein the extracellular domain comprises an antigen-binding domain that specifically binds an antigen expressed on the surface of the cancer, wherein preferably said antigen-binding domain is a scFv domain.

49. The method according to claim 48, wherein the intracellular domain comprises at least one costimulatory domain, optionally wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptors, ICAM-1, B7-H3, CDS, ICAM-1, GITR, BAFFR, HVEM (LIGHTR), KIRDS2, SLAMF7, NKp80 (KLRF1), NKp44, NKp30, NKp46, CD19, CD4, CD8alpha., CD8beta, IL-2R beta, IL-2R gamma, IL-7R alpha, ITGA4, VLA1, CD49a, ITGA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof; optionally wherein a) the costimulatory domain comprises a signaling region of 4-1BB/CD137 or b) wherein the costimulatory domain comprises a signaling region of CD28.

50. The method according to claim 48, wherein a) the CAR comprises two or more costimulatory domains, or b) wherein the CAR comprises two costimulatory domains, wherein one of the two costimulatory domains is CD28 and the other costimulatory domain is selected from 4-1BB/CD137 and OX40.

51. The method according to claim 48, wherein the intracellular domain of the CAR comprises at least one activating domain, optionally wherein the activating domain comprises CD3, preferably said CD3 is CD3 zeta.

52. The method according to claim 21, wherein the immune cell is a T cell, Natural Killer (NK) cell, TCR-expressing cell, dendritic cell, gamma delta T cell, or NK-T cell, preferable the immune cell is a CAR-T cell.

53. The method according to claim 21, wherein the cancer is an EBV-associated cancer, selected from a lymphoproliferative disorder (LPD), such as B-cell lymphoma, including Burkitt lymphoma (BL), Hodgkin lymphoma (HL), a diffuse large B cell lymphoma (DLBCL), T-cell lymphoma, NK/T-cell lymphoma, or a post-transplant lymphoproliferative disorder (PTLD), or an epithelial carcinoma (nasopharyngeal, lung, breast), a lymphoepithelioma, a carcinoma with lymphoid stroma (GCLS, e.g. gastric carcinoma) or a glioma.

54. The method according to claim 21, wherein the immunogenic composition comprises a polyinosinic-polycytidylic acid (PIC).


SEQUENCE LISTING (179 sequences)

SEQ ID NO: 1 - protein, 120 aa, synthetic construct
QIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSS

SEQ ID NO: 2 - protein, 120 aa, synthetic construct
QIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSS

SEQ ID NO: 3 - protein, 123 aa, synthetic construct
EFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSS

SEQ ID NO: 4 - protein, 118 aa, synthetic construct
EVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSA

SEQ ID NO: 5 - protein, 118 aa, synthetic construct
QVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSA

SEQ ID NO: 6 - protein, 112 aa, synthetic construct
DVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIK

SEQ ID NO: 7 - protein, 112 aa, synthetic construct
DVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIK

SEQ ID NO: 8 - protein, 107 aa, synthetic construct
DIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIK

SEQ ID NO: 9 - protein, 109 aa, synthetic construct
QAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVL

SEQ ID NO: 10 - protein, 109 aa, synthetic construct
QIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIK

SEQ ID NO: 11 - protein, 5 aa, synthetic construct
TYGVS

SEQ ID NO: 12 - protein, 17 aa, synthetic construct
WINTYSGVPRYADDFKG

SEQ ID NO: 13 - protein, 11 aa, synthetic construct
FFYGYDHTMDY

SEQ ID NO: 14 - protein, 5 aa, synthetic construct
IYGMS

SEQ ID NO: 15 - protein, 17 aa, synthetic construct
WINTYSGVPKYADDFKG

SEQ ID NO: 16 - protein, 11 aa, synthetic construct
FYYGYDHAMDY

SEQ ID NO: 17 - protein, 5 aa, synthetic construct
DYNMN

SEQ ID NO: 18 - protein, 17 aa, synthetic construct
VINPNFGTTSYNQKFKG

SEQ ID NO: 19 - protein, 14 aa, synthetic construct
SGYYGSNRYWYFDV

SEQ ID NO: 20 - protein, 5 aa, synthetic construct
DYYMN

SEQ ID NO: 21 - protein, 17 aa, synthetic construct
VINPYNGGTSHNEKLKG

SEQ ID NO: 22 - protein, 9 aa, synthetic construct
GNYSAWFAY

SEQ ID NO: 23 - protein, 5 aa, synthetic construct
DYYIN

SEQ ID NO: 24 - protein, 17 aa, synthetic construct
WIFPGRGRSNYSEKFKG

SEQ ID NO: 25 - protein, 9 aa, synthetic construct
GTAQAGFPY

SEQ ID NO: 26 - protein, 16 aa, synthetic construct
RSSQSLAHSNGNTYLH

SEQ ID NO: 27 - protein, 7 aa, synthetic construct
KVSNRFS

SEQ ID NO: 28 - protein, 9 aa, synthetic construct
SQSTHFPWT

SEQ ID NO: 29 - protein, 16 aa, synthetic construct
SSSQSLVHSNGNTYLH

SEQ ID NO: 30 - protein, 7 aa, synthetic construct
KVSNRFS

SEQ ID NO: 31 - protein, 9 aa, synthetic construct
SQSTHFPWT

SEQ ID NO: 32 - protein, 11 aa, synthetic construct
KASQSVGTNVA

SEQ ID NO: 33 - protein, 7 aa, synthetic construct
SASYRYS

SEQ ID NO: 34 - protein, 9 aa, synthetic construct
QQYNSYPST

SEQ ID NO: 35 - protein, 14 aa, synthetic construct
RSSTGAVTTSNYAN

SEQ ID NO: 36 - protein, 7 aa, synthetic construct
GTNNRAP

SEQ ID NO: 37 - protein, 9 aa, synthetic construct
VLWYSNLWV

SEQ ID NO: 38 - protein, 12 aa, synthetic construct
TASSSVSSSYLH

SEQ ID NO: 39 - protein, 7 aa, synthetic construct
SASNLAS

SEQ ID NO: 40 - protein, 10 aa, synthetic construct
HQYHRSPPWT

SEQ ID NO: 41 - protein, 492 aa, synthetic construct
MEFGLSWLFLVAILKGVQCDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKSGGGGSGGGGSGGGGSQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 459 aa, synthetic construct
MEFGLSWLFLVAILKGVQCDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKSGGGGSGGGGSGGGGSQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 675 aa, synthetic construct
MEFGLSWLFLVAILKGVQCDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKSGGGGSGGGGSGGGGSQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 492 aa, synthetic construct
MEFGLSWLFLVAILKGVQCQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSSGGGGSGGGGSGGGGSDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 459 aa, synthetic construct
MEFGLSWLFLVAILKGVQCQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSSGGGGSGGGGSGGGGSDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 675 aa, synthetic construct
MEFGLSWLFLVAILKGVQCQIQLVQSGPDLKKTGETVKISCKASGYTFTTYGVSWVKQAPGKVLKWMGWINTYSGVPRYADDFKGRFAFSLETSASTAYLQINNLKNEDTATYFCARFFYGYDHTMDYWGQGTSVTVSSSGGGGSGGGGSGGGGSDVVMTQTPLSLPVSLGDQASISCRSSQSLAHSNGNTYLHWYLQKPGQSPKLLIYKVSNRFSGVPDRFNGSGSGTDFTLKISRVEAEDLGVYFCSQSTHFPWTFGGGTKLEIKGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 492 aa, synthetic construct
MEFGLSWLFLVAILKGVQCDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKSGGGGSGGGGSGGGGSQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 459 aa, synthetic construct
MEFGLSWLFLVAILKGVQCDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKSGGGGSGGGGSGGGGSQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 675 aa, synthetic construct
MEFGLSWLFLVAILKGVQCDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKSGGGGSGGGGSGGGGSQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 492 aa, synthetic construct
MEFGLSWLFLVAILKGVQCQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSSGGGGSGGGGSGGGGSDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 459 aa, synthetic construct
MEFGLSWLFLVAILKGVQCQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSSGGGGSGGGGSGGGGSDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 675 aa, synthetic construct
MEFGLSWLFLVAILKGVQCQIQLVQSGPELKKPGETVKISCKASGYTFTIYGMSWVKQAPGKGLRWMGWINTYSGVPKYADDFKGHFAFSLETSASTAYLRINNLKDEDTATYFCARFYYGYDHAMDYWGQGTSVIVSSSGGGGSGGGGSGGGGSDVVMTQTPLSLPVSLGDQASISCSSSQSLVHSNGNTYLHWYLQRPGQSPKLLIYKVSNRFSGVPDRFSGSGSGTDFTLKISRVEAEDLGVYFCSQSTHFPWTIGGGIKLEIKGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 490 aa, synthetic construct
MEFGLSWLFLVAILKGVQCDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKSGGGGSGGGGSGGGGSEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 457 aa, synthetic construct
MEFGLSWLFLVAILKGVQCDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKSGGGGSGGGGSGGGGSEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 673 aa, synthetic construct
MEFGLSWLFLVAILKGVQCDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKSGGGGSGGGGSGGGGSEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 490 aa, synthetic construct
MEFGLSWLFLVAILKGVQCEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSSGGGGSGGGGSGGGGSDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 457 aa, synthetic construct
MEFGLSWLFLVAILKGVQCEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSSGGGGSGGGGSGGGGSDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 673 aa, synthetic construct
MEFGLSWLFLVAILKGVQCEFQLQQSGPDLVKPGASVKISCKASGYSFTDYNMNWVKQSNGKNLEWIGVINPNFGTTSYNQKFKGKATLTVDQSSSTAYMQLNSLTSEDSAVYYCARSGYYGSNRYWYFDVWGTGTTVTVSSSGGGGSGGGGSGGGGSDIVMTQSQKFMSTSVGDRVSVTCKASQSVGTNVAWYQQKPGQSPKALIYSASYRYSGVPDRFTGSGSGTDFTLTISNVQSEDLAEYFCQQYNSYPSTFGGGTKLEIKGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 487 aa, synthetic construct
MEFGLSWLFLVAILKGVQCQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLSGGGGSGGGGSGGGGSEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSAGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 454 aa, synthetic construct
MEFGLSWLFLVAILKGVQCQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLSGGGGSGGGGSGGGGSEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSAGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 670 aa, synthetic construct
MEFGLSWLFLVAILKGVQCQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLSGGGGSGGGGSGGGGSEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSAGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 487 aa, synthetic construct
MEFGLSWLFLVAILKGVQCEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSASGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 454 aa, synthetic construct
MEFGLSWLFLVAILKGVQCEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSASGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 670 aa, synthetic construct
MEFGLSWLFLVAILKGVQCEVQLQQSGPVLEKPGASVKMSCKASGYTFTDYYMNWVKQSHGKSLEWIGVINPYNGGTSHNEKLKGKATLTVDKSSSTAYMELNSLTSEDSAVYYCARGNYSAWFAYWGQGTLVTVSASGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCVLWYSNLWVFGGGTKLTVLGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 487 aa, synthetic construct
MEFGLSWLFLVAILKGVQCQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKSGGGGSGGGGSGGGGSQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSAGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 454 aa, synthetic construct
MEFGLSWLFLVAILKGVQCQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKSGGGGSGGGGSGGGGSQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSAGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 670 aa, synthetic construct
MEFGLSWLFLVAILKGVQCQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKSGGGGSGGGGSGGGGSQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSAGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 487 aa, synthetic construct
MEFGLSWLFLVAILKGVQCQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSASGGGGSGGGGSGGGGSQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 454 aa, synthetic construct
MEFGLSWLFLVAILKGVQCQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSASGGGGSGGGGSGGGGSQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKGSESKYGPPCPPCPIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 670 aa, synthetic construct
MEFGLSWLFLVAILKGVQCQVHLQQSGPELVKPGASVKISCKASGYNFNDYYINWVKQRPGQGLEWIGWIFPGRGRSNYSEKFKGKATLTVDKSSSTAYVLLSSLTSEDSAVYFCARGTAQAGFPYWGQGTLVTVSASGGGGSGGGGSGGGGSQIVLTQSPAIMSASLGERVTMTCTASSSVSSSYLHWYQQKPGSSPKLWIYSASNLASGVPPRFSGSGSGTSYSLTISSMEAEDAATYYCHQYHRSPPWTFGGGTKLEIKGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 1476 bp, synthetic construct
  source 1..1476: mol_type = other DNA
atggagttcggcctgagctggctgtttctggtggccatcctgaagggcgtgcagtgcgacgtggtcatgacccagacaccactgagcctgcccgtgagcctgggcgatcaggcctccatctcttgtcggagctcccagtctctggcccacagcaacggcaatacatacctgcactggtatctgcagaagccaggccagagccccaagctgctgatctacaaggtgtccaaccggttctctggagtgccagaccggttcaacggcagcggctccggcaccgatttcacactgaagatctccagggtggaggcagaggacctgggcgtgtatttttgctctcagagcacccacttcccctggacatttggcggcggcaccaagctggagatcaagtccggcggcggcggctccggcggcggcggctctggcggcggcggcagccagatccagctggtgcagtccggccctgatctgaagaagaccggcgagacagtgaagatcagctgtaaggcctccggctacacattcaccacatatggcgtgtcttgggtgaagcaggcccctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggacaccgccacatatttttgcgcccggttcttttacggctatgaccacaccatggattactggggccagggcacctccgtgacagtgtctagcggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 72 - DNA, 1377 bp, synthetic construct
  source 1..1377: mol_type = other DNA
atggagttcggcctgagctggctgtttctggtggccatcctgaagggcgtgcagtgcgacgtggtcatgacccagacaccactgagcctgcccgtgagcctgggcgatcaggcctccatctcttgtcggagctcccagtctctggcccacagcaacggcaatacatacctgcactggtatctgcagaagccaggccagagccccaagctgctgatctacaaggtgtccaaccggttctctggagtgccagaccggttcaacggcagcggctccggcaccgatttcacactgaagatctccagggtggaggcagaggacctgggcgtgtatttttgctctcagagcacccacttcccctggacatttggcggcggcaccaagctggagatcaagtccggcggcggcggctccggcggcggcggctctggcggcggcggcagccagatccagctggtgcagtccggccctgatctgaagaagaccggcgagacagtgaagatcagctgtaaggcctccggctacacattcaccacatatggcgtgtcttgggtgaagcaggcccctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggacaccgccacatatttttgcgcccggttcttttacggctatgaccacaccatggattactggggccagggcacctccgtgacagtgtctagcggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 73 - DNA, 2025 bp, synthetic construct
  source 1..2025: mol_type = other DNA
atggagttcggcctgagctggctgtttctggtggccatcctgaagggcgtgcagtgcgacgtggtcatgacccagacaccactgagcctgcccgtgagcctgggcgatcaggcctccatctcttgtcggagctcccagtctctggcccacagcaacggcaatacatacctgcactggtatctgcagaagccaggccagagccccaagctgctgatctacaaggtgtccaaccggttctctggagtgccagaccggttcaacggcagcggctccggcaccgatttcacactgaagatctccagggtggaggcagaggacctgggcgtgtatttttgctctcagagcacccacttcccctggacatttggcggcggcaccaagctggagatcaagtccggcggcggcggctccggcggcggcggctctggcggcggcggcagccagatccagctggtgcagtccggccctgatctgaagaagaccggcgagacagtgaagatcagctgtaaggcctccggctacacattcaccacatatggcgtgtcttgggtgaagcaggcccctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggacaccgccacatatttttgcgcccggttcttttacggctatgaccacaccatggattactggggccagggcacctccgtgacagtgtctagcggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 74 - DNA, 1476 bp, synthetic construct
  source 1..1476: mol_type = other DNA
atggagttcggcctgtcctggctgtttctggtggccatcctgaagggcgtgcagtgccagatccagctggtgcagtctggccccgacctgaagaagaccggcgagacagtgaagatcagctgtaaggcctccggctacacattcaccacatatggcgtgagctgggtgaagcaggcacctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggataccgccacatatttttgcgcccggttcttttacggctatgaccacacaatggattactggggccagggcacctccgtgacagtgagctcctccggcggcggcggctccggcggcggcggctctggcggcggcggcagcgacgtggtcatgacccagacacccctgtctctgcctgtgagcctgggcgatcaggccagcatctcctgtaggtctagccagtccctggcccactctaacggcaatacctacctgcactggtatctgcagaagccaggccagagccccaagctgctgatctacaaggtgagcaaccggttctccggagtgccagaccggttcaacggctctggcagcggcaccgatttcacactgaagatctcccgggtggaggcagaggacctgggcgtgtatttttgctcccagtctacccacttcccttggacatttggcggcggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 75 - DNA, 1377 bp, synthetic construct
  source 1..1377: mol_type = other DNA
atggagttcggcctgtcctggctgtttctggtggccatcctgaagggcgtgcagtgccagatccagctggtgcagtctggccccgacctgaagaagaccggcgagacagtgaagatcagctgtaaggcctccggctacacattcaccacatatggcgtgagctgggtgaagcaggcacctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggataccgccacatatttttgcgcccggttcttttacggctatgaccacacaatggattactggggccagggcacctccgtgacagtgagctcctccggcggcggcggctccggcggcggcggctctggcggcggcggcagcgacgtggtcatgacccagacacccctgtctctgcctgtgagcctgggcgatcaggccagcatctcctgtaggtctagccagtccctggcccactctaacggcaatacctacctgcactggtatctgcagaagccaggccagagccccaagctgctgatctacaaggtgagcaaccggttctccggagtgccagaccggttcaacggctctggcagcggcaccgatttcacactgaagatctcccgggtggaggcagaggacctgggcgtgtatttttgctcccagtctacccacttcccttggacatttggcggcggcaccaagctggagatcaagggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 76 - DNA, 2025 bp, synthetic construct
  source 1..2025: mol_type = other DNA
atggagttcggcctgtcctggctgtttctggtggccatcctgaagggcgtgcagtgccagatccagctggtgcagtctggccccgacctgaagaagaccggcgagacagtgaagatcagctgtaaggcctccggctacacattcaccacatatggcgtgagctgggtgaagcaggcacctggcaaggtgctgaagtggatgggctggatcaacacctactctggcgtgccaagatatgccgacgatttcaagggccgcttcgcctttagcctggagacctccgcctctacagcctacctgcagatcaacaatctgaagaatgaggataccgccacatatttttgcgcccggttcttttacggctatgaccacacaatggattactggggccagggcacctccgtgacagtgagctcctccggcggcggcggctccggcggcggcggctctggcggcggcggcagcgacgtggtcatgacccagacacccctgtctctgcctgtgagcctgggcgatcaggccagcatctcctgtaggtctagccagtccctggcccactctaacggcaatacctacctgcactggtatctgcagaagccaggccagagccccaagctgctgatctacaaggtgagcaaccggttctccggagtgccagaccggttcaacggctctggcagcggcaccgatttcacactgaagatctcccgggtggaggcagaggacctgggcgtgtatttttgctcccagtctacccacttcccttggacatttggcggcggcaccaagctggagatcaagggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 77 - DNA, 1476 bp, synthetic construct
  source 1..1476: mol_type = other DNA
atggagttcggcctgagctggctgtttctggtggccatcctgaagggcgtgcagtgcgacgtggtcatgacccagacacccctgtctctgcctgtgagcctgggcgatcaggcctctatcagctgtagctcctctcagtccctggtgcactctaacggcaatacctacctgcactggtatctgcagcggccaggccagtctcccaagctgctgatctacaaggtgagcaaccggttctccggcgtgccagacagattttccggctctggcagcggcaccgatttcacactgaagatctccagggtggaggcagaggacctgggcgtgtatttctgctcccagtctacccactttccctggacaatcggcggcggcatcaagctggagatcaagtccggcggcggcggctccggcggcggcggctctggcggcggcggcagccagatccagctggtgcagagcggccctgagctgaagaagccaggcgagaccgtgaagatctcttgtaaggccagcggctacaccttcacaatctatggaatgtcctgggtgaagcaggcaccaggcaagggactgaggtggatgggctggatcaatacatactccggcgtgcctaagtatgccgacgacttcaagggccacttcgccttttctctggagaccagcgcctccacagcctacctgaggatcaacaatctgaaggacgaggataccgccacatatttctgcgcccgcttttactatggctacgaccacgccatggattattggggccagggcacctccgtgatcgtgagctccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 78 - DNA, 1377 bp, synthetic construct
  source 1..1377: mol_type = other DNA
atggagttcggcctgagctggctgtttctggtggccatcctgaagggcgtgcagtgcgacgtggtcatgacccagacacccctgtctctgcctgtgagcctgggcgatcaggcctctatcagctgtagctcctctcagtccctggtgcactctaacggcaatacctacctgcactggtatctgcagcggccaggccagtctcccaagctgctgatctacaaggtgagcaaccggttctccggcgtgccagacagattttccggctctggcagcggcaccgatttcacactgaagatctccagggtggaggcagaggacctgggcgtgtatttctgctcccagtctacccactttccctggacaatcggcggcggcatcaagctggagatcaagtccggcggcggcggctccggcggcggcggctctggcggcggcggcagccagatccagctggtgcagagcggccctgagctgaagaagccaggcgagaccgtgaagatctcttgtaaggccagcggctacaccttcacaatctatggaatgtcctgggtgaagcaggcaccaggcaagggactgaggtggatgggctggatcaatacatactccggcgtgcctaagtatgccgacgacttcaagggccacttcgccttttctctggagaccagcgcctccacagcctacctgaggatcaacaatctgaaggacgaggataccgccacatatttctgcgcccgcttttactatggctacgaccacgccatggattattggggccagggcacctccgtgatcgtgagctccggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 79 - DNA, 2025 bp, synthetic construct
  source 1..2025: mol_type = other DNA
atggagttcggcctgagctggctgtttctggtggccatcctgaagggcgtgcagtgcgacgtggtcatgacccagacacccctgtctctgcctgtgagcctgggcgatcaggcctctatcagctgtagctcctctcagtccctggtgcactctaacggcaatacctacctgcactggtatctgcagcggccaggccagtctcccaagctgctgatctacaaggtgagcaaccggttctccggcgtgccagacagattttccggctctggcagcggcaccgatttcacactgaagatctccagggtggaggcagaggacctgggcgtgtatttctgctcccagtctacccactttccctggacaatcggcggcggcatcaagctggagatcaagtccggcggcggcggctccggcggcggcggctctggcggcggcggcagccagatccagctggtgcagagcggccctgagctgaagaagccaggcgagaccgtgaagatctcttgtaaggccagcggctacaccttcacaatctatggaatgtcctgggtgaagcaggcaccaggcaagggactgaggtggatgggctggatcaatacatactccggcgtgcctaagtatgccgacgacttcaagggccacttcgccttttctctggagaccagcgcctccacagcctacctgaggatcaacaatctgaaggacgaggataccgccacatatttctgcgcccgcttttactatggctacgaccacgccatggattattggggccagggcacctccgtgatcgtgagctccggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 80 - DNA, 1476 bp, synthetic construct
  source 1..1476: mol_type = other DNA
atggagttcggcctgagctggctgtttctggtggccatcctgaagggcgtgcagtgccagatccagctggtgcagtccggccctgagctgaagaagccaggcgagaccgtgaagatctcttgtaaggccagcggctacaccttcacaatctatggaatgagctgggtgaagcaggcaccaggcaagggactgaggtggatgggctggatcaacacatactccggcgtgcctaagtatgccgacgacttcaagggccacttcgccttttctctggagacctctgccagcacagcctacctgaggatcaacaatctgaaggacgaggataccgccacatatttctgcgcccgcttttactatggctacgaccacgccatggattattggggccagggcacctccgtgatcgtgagctcctccggcggcggcggctccggcggcggcggctctggcggcggcggcagcgacgtggtcatgacccagacaccactgtccctgcccgtgagcctgggcgatcaggcctccatctcttgttctagctcccagagcctggtgcactccaacggcaatacctacctgcactggtatctgcagcggccaggccagtctcccaagctgctgatctacaaggtgtctaatcggttcagcggagtgccagaccggttcagcggctccggctctggaaccgatttcacactgaagatctccagggtggaggcagaggacctgggcgtgtatttctgcagccagtccacccactttccctggacaatcggcggcggcatcaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 81 - DNA, 1377 bp, synthetic construct
  source 1..1377: mol_type = other DNA
atggagttcggcctgagctggctgtttctggtggccatcctgaagggcgtgcagtgccagatccagctggtgcagtccggccctgagctgaagaagccaggcgagaccgtgaagatctcttgtaaggccagcggctacaccttcacaatctatggaatgagctgggtgaagcaggcaccaggcaagggactgaggtggatgggctggatcaacacatactccggcgtgcctaagtatgccgacgacttcaagggccacttcgccttttctctggagacctctgccagcacagcctacctgaggatcaacaatctgaaggacgaggataccgccacatatttctgcgcccgcttttactatggctacgaccacgccatggattattggggccagggcacctccgtgatcgtgagctcctccggcggcggcggctccggcggcggcggctctggcggcggcggcagcgacgtggtcatgacccagacaccactgtccctgcccgtgagcctgggcgatcaggcctccatctcttgttctagctcccagagcctggtgcactccaacggcaatacctacctgcactggtatctgcagcggccaggccagtctcccaagctgctgatctacaaggtgtctaatcggttcagcggagtgccagaccggttcagcggctccggctctggaaccgatttcacactgaagatctccagggtggaggcagaggacctgggcgtgtatttctgcagccagtccacccactttccctggacaatcggcggcggcatcaagctggagatcaagggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 82 - DNA, 2025 bp, synthetic construct
  source 1..2025: mol_type = other DNA
atggagttcggcctgagctggctgtttctggtggccatcctgaagggcgtgcagtgccagatccagctggtgcagtccggccctgagctgaagaagccaggcgagaccgtgaagatctcttgtaaggccagcggctacaccttcacaatctatggaatgagctgggtgaagcaggcaccaggcaagggactgaggtggatgggctggatcaacacatactccggcgtgcctaagtatgccgacgacttcaagggccacttcgccttttctctggagacctctgccagcacagcctacctgaggatcaacaatctgaaggacgaggataccgccacatatttctgcgcccgcttttactatggctacgaccacgccatggattattggggccagggcacctccgtgatcgtgagctcctccggcggcggcggctccggcggcggcggctctggcggcggcggcagcgacgtggtcatgacccagacaccactgtccctgcccgtgagcctgggcgatcaggcctccatctcttgttctagctcccagagcctggtgcactccaacggcaatacctacctgcactggtatctgcagcggccaggccagtctcccaagctgctgatctacaaggtgtctaatcggttcagcggagtgccagaccggttcagcggctccggctctggaaccgatttcacactgaagatctccagggtggaggcagaggacctgggcgtgtatttctgcagccagtccacccactttccctggacaatcggcggcggcatcaagctggagatcaagggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 83 - DNA, 1470 bp, synthetic construct
  source 1..1470: mol_type = other DNA
atggagttcggcctgagctggctgtttctggtggccatcctgaagggcgtgcagtgcgacatcgtgatgacccagagccagaagttcatgtctacaagcgtgggcgatcgggtgtccgtgacctgtaaggcctcccagtctgtgggcacaaacgtggcctggtatcagcagaagcccggccagtctcctaaggccctgatctatagcgcctcctaccggtattccggcgtgcctgacagattcaccggctctggcagcggcacagacttcaccctgacaatctctaacgtgcagagcgaggacctggccgagtacttctgccagcagtacaattcctatccatctacctttggcggcggcacaaagctggagatcaagagcggaggaggaggctccggaggaggaggctctggcggcggcggcagcgagttccagctgcagcagagcggaccagacctggtgaagccaggagcctccgtgaagatcagctgtaaggccagcggctactcctttaccgattataacatgaattgggtgaagcagagcaacggcaagaatctggagtggatcggcgtgatcaaccccaatttcggcaccacatcctacaatcagaagtttaagggcaaggccaccctgacagtggaccagagctcctctaccgcctatatgcagctgaactctctgacaagcgaggattccgccgtgtactattgcgccaggtccggctactatggctctaatcgctactggtattttgacgtgtggggcaccggcaccacagtgacagtgagctccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 84 - DNA, 1371 bp, synthetic construct
  source 1..1371: mol_type = other DNA
atggagttcggcctgagctggctgtttctggtggccatcctgaagggcgtgcagtgcgacatcgtgatgacccagagccagaagttcatgtctacaagcgtgggcgatcgggtgtccgtgacctgtaaggcctcccagtctgtgggcacaaacgtggcctggtatcagcagaagcccggccagtctcctaaggccctgatctatagcgcctcctaccggtattccggcgtgcctgacagattcaccggctctggcagcggcacagacttcaccctgacaatctctaacgtgcagagcgaggacctggccgagtacttctgccagcagtacaattcctatccatctacctttggcggcggcacaaagctggagatcaagagcggaggaggaggctccggaggaggaggctctggcggcggcggcagcgagttccagctgcagcagagcggaccagacctggtgaagccaggagcctccgtgaagatcagctgtaaggccagcggctactcctttaccgattataacatgaattgggtgaagcagagcaacggcaagaatctggagtggatcggcgtgatcaaccccaatttcggcaccacatcctacaatcagaagtttaagggcaaggccaccctgacagtggaccagagctcctctaccgcctatatgcagctgaactctctgacaagcgaggattccgccgtgtactattgcgccaggtccggctactatggctctaatcgctactggtattttgacgtgtggggcaccggcaccacagtgacagtgagctccggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 85 - DNA, 2019 bp, synthetic construct
  source 1..2019: mol_type = other DNA
atggagttcggcctgagctggctgtttctggtggccatcctgaagggcgtgcagtgcgacatcgtgatgacccagagccagaagttcatgtctacaagcgtgggcgatcgggtgtccgtgacctgtaaggcctcccagtctgtgggcacaaacgtggcctggtatcagcagaagcccggccagtctcctaaggccctgatctatagcgcctcctaccggtattccggcgtgcctgacagattcaccggctctggcagcggcacagacttcaccctgacaatctctaacgtgcagagcgaggacctggccgagtacttctgccagcagtacaattcctatccatctacctttggcggcggcacaaagctggagatcaagagcggaggaggaggctccggaggaggaggctctggcggcggcggcagcgagttccagctgcagcagagcggaccagacctggtgaagccaggagcctccgtgaagatcagctgtaaggccagcggctactcctttaccgattataacatgaattgggtgaagcagagcaacggcaagaatctggagtggatcggcgtgatcaaccccaatttcggcaccacatcctacaatcagaagtttaagggcaaggccaccctgacagtggaccagagctcctctaccgcctatatgcagctgaactctctgacaagcgaggattccgccgtgtactattgcgccaggtccggctactatggctctaatcgctactggtattttgacgtgtggggcaccggcaccacagtgacagtgagctccggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 86 - DNA, 1470 bp, synthetic construct
  source 1..1470: mol_type = other DNA
atggagttcggcctgagctggctgtttctggtggccatcctgaagggcgtgcagtgcgagttccagctgcagcagagcggaccagacctggtgaagccaggagccagcgtgaagatcagctgtaaggcctctggctacagctttaccgattataacatgaattgggtgaagcagagcaacggcaagaatctggagtggatcggcgtgatcaaccccaatttcggcaccacatcctacaaccagaagtttaagggcaaggccaccctgacagtggaccagagctcctctaccgcctatatgcagctgaactccctgacatctgaggatagcgccgtgtactattgcgccaggtccggctactatggctctaatcgctactggtatttcgacgtgtggggcaccggcaccacagtgacagtgagctcctctggaggaggaggcagcggcggaggaggctccggcggcggcggctctgacatcgtgatgacccagagccagaagtttatgagcacatccgtgggcgatcgggtgtccgtgacctgcaaggcctctcagagcgtgggcacaaatgtggcctggtatcagcagaagcccggccagtctcctaaggccctgatctattccgcctcttaccggtattccggcgtgcctgacagattcaccggcagcggctccggcacagacttcaccctgacaatctctaacgtgcagagcgaggatctggccgagtacttctgtcagcagtacaattcctatccatctacctttggcggcggcacaaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 87 - DNA, 1371 bp, synthetic construct
  source 1..1371: mol_type = other DNA
atggagttcggcctgagctggctgtttctggtggccatcctgaagggcgtgcagtgcgagttccagctgcagcagagcggaccagacctggtgaagccaggagccagcgtgaagatcagctgtaaggcctctggctacagctttaccgattataacatgaattgggtgaagcagagcaacggcaagaatctggagtggatcggcgtgatcaaccccaatttcggcaccacatcctacaaccagaagtttaagggcaaggccaccctgacagtggaccagagctcctctaccgcctatatgcagctgaactccctgacatctgaggatagcgccgtgtactattgcgccaggtccggctactatggctctaatcgctactggtatttcgacgtgtggggcaccggcaccacagtgacagtgagctcctctggaggaggaggcagcggcggaggaggctccggcggcggcggctctgacatcgtgatgacccagagccagaagtttatgagcacatccgtgggcgatcgggtgtccgtgacctgcaaggcctctcagagcgtgggcacaaatgtggcctggtatcagcagaagcccggccagtctcctaaggccctgatctattccgcctcttaccggtattccggcgtgcctgacagattcaccggcagcggctccggcacagacttcaccctgacaatctctaacgtgcagagcgaggatctggccgagtacttctgtcagcagtacaattcctatccatctacctttggcggcggcacaaagctggagatcaagggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 88 - DNA, 2019 bp, synthetic construct
  source 1..2019: mol_type = other DNA
atggagttcggcctgagctggctgtttctggtggccatcctgaagggcgtgcagtgcgagttccagctgcagcagagcggaccagacctggtgaagccaggagccagcgtgaagatcagctgtaaggcctctggctacagctttaccgattataacatgaattgggtgaagcagagcaacggcaagaatctggagtggatcggcgtgatcaaccccaatttcggcaccacatcctacaaccagaagtttaagggcaaggccaccctgacagtggaccagagctcctctaccgcctatatgcagctgaactccctgacatctgaggatagcgccgtgtactattgcgccaggtccggctactatggctctaatcgctactggtatttcgacgtgtggggcaccggcaccacagtgacagtgagctcctctggaggaggaggcagcggcggaggaggctccggcggcggcggctctgacatcgtgatgacccagagccagaagtttatgagcacatccgtgggcgatcgggtgtccgtgacctgcaaggcctctcagagcgtgggcacaaatgtggcctggtatcagcagaagcccggccagtctcctaaggccctgatctattccgcctcttaccggtattccggcgtgcctgacagattcaccggcagcggctccggcacagacttcaccctgacaatctctaacgtgcagagcgaggatctggccgagtacttctgtcagcagtacaattcctatccatctacctttggcggcggcacaaagctggagatcaagggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 89 - DNA, 1461 bp, synthetic construct
  source 1..1461: mol_type = other DNA
atggagttcggcctgtcctggctgtttctggtggccatcctgaagggcgtgcagtgccaggcagtggtgacacaggagagcgccctgaccacatcccctggagagaccgtgacactgacctgtaggagctccaccggcgccgtgaccacaagcaactacgccaattgggtgcaggagaagccagaccacctgttcacaggcctgatcggaggcaccaacaatagggcaccaggcgtgcctgccagattttctggcagcctgatcggcgacaaggccgccctgacaatcaccggagcacagaccgaggatgaggccatctacttctgcgtgctgtggtattctaacctgtgggtgtttggcggcggcacaaagctgaccgtgctgtctggaggaggaggcagcggcggaggaggctccggcggcggcggctctgaggtgcagctgcagcagagcggaccagtgctggagaagccaggagcctccgtgaagatgtcttgtaaggccagcggctatacattcaccgattactatatgaactgggtgaagcagtcccacggcaagtctctggagtggatcggcgtgatcaacccctacaatggcggcacctcccacaatgagaagctgaagggcaaggccacactgaccgtggacaagtctagctccacagcctatatggagctgaactccctgacctctgaggatagcgccgtgtactattgcgccagaggcaattacagcgcctggtttgcctattggggccagggcacactggtgaccgtgtccgccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 90 - DNA, 1362 bp, synthetic construct
  source 1..1362: mol_type = other DNA
atggagttcggcctgtcctggctgtttctggtggccatcctgaagggcgtgcagtgccaggcagtggtgacacaggagagcgccctgaccacatcccctggagagaccgtgacactgacctgtaggagctccaccggcgccgtgaccacaagcaactacgccaattgggtgcaggagaagccagaccacctgttcacaggcctgatcggaggcaccaacaatagggcaccaggcgtgcctgccagattttctggcagcctgatcggcgacaaggccgccctgacaatcaccggagcacagaccgaggatgaggccatctacttctgcgtgctgtggtattctaacctgtgggtgtttggcggcggcacaaagctgaccgtgctgtctggaggaggaggcagcggcggaggaggctccggcggcggcggctctgaggtgcagctgcagcagagcggaccagtgctggagaagccaggagcctccgtgaagatgtcttgtaaggccagcggctatacattcaccgattactatatgaactgggtgaagcagtcccacggcaagtctctggagtggatcggcgtgatcaacccctacaatggcggcacctcccacaatgagaagctgaagggcaaggccacactgaccgtggacaagtctagctccacagcctatatggagctgaactccctgacctctgaggatagcgccgtgtactattgcgccagaggcaattacagcgcctggtttgcctattggggccagggcacactggtgaccgtgtccgccggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 91 - DNA, 2010 bp, synthetic construct
  source 1..2010: mol_type = other DNA
atggagttcggcctgtcctggctgtttctggtggccatcctgaagggcgtgcagtgccaggcagtggtgacacaggagagcgccctgaccacatcccctggagagaccgtgacactgacctgtaggagctccaccggcgccgtgaccacaagcaactacgccaattgggtgcaggagaagccagaccacctgttcacaggcctgatcggaggcaccaacaatagggcaccaggcgtgcctgccagattttctggcagcctgatcggcgacaaggccgccctgacaatcaccggagcacagaccgaggatgaggccatctacttctgcgtgctgtggtattctaacctgtgggtgtttggcggcggcacaaagctgaccgtgctgtctggaggaggaggcagcggcggaggaggctccggcggcggcggctctgaggtgcagctgcagcagagcggaccagtgctggagaagccaggagcctccgtgaagatgtcttgtaaggccagcggctatacattcaccgattactatatgaactgggtgaagcagtcccacggcaagtctctggagtggatcggcgtgatcaacccctacaatggcggcacctcccacaatgagaagctgaagggcaaggccacactgaccgtggacaagtctagctccacagcctatatggagctgaactccctgacctctgaggatagcgccgtgtactattgcgccagaggcaattacagcgcctggtttgcctattggggccagggcacactggtgaccgtgtccgccggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 92 - DNA, 1461 bp, synthetic construct
  source 1..1461: mol_type = other DNA
atggagttcggcctgtcctggctgtttctggtggccatcctgaagggcgtgcagtgcgaggtgcagctgcagcagtctggaccagtgctggagaagccaggagccagcgtgaagatgtcctgtaaggcctctggctacaccttcacagactactatatgaactgggtgaagcagagccacggcaagtccctggagtggatcggcgtgatcaacccctacaatggcggcaccagccacaatgagaagctgaagggcaaggccaccctgacagtggacaagagctcctctacagcctatatggagctgaacagcctgacctccgaggattctgccgtgtactattgcgccaggggcaattactccgcctggtttgcctattggggccagggcacactggtgaccgtgtctgccagcggcggcggcggctctggaggaggaggcagcggcggaggaggctcccaggccgtggtgacacaggagtctgccctgaccacaagccctggcgagaccgtgacactgacctgtcggagctccaccggagcagtgaccacaagcaactatgccaattgggtgcaggagaagccagatcacctgttcacaggcctgatcggaggcaccaacaatagggcaccaggcgtgcctgcaagattttccggctctctgatcggcgacaaggccgccctgacaatcaccggagcacagaccgaggatgaggccatctacttctgcgtgctgtggtattccaacctgtgggtgtttggcggcggcacaaagctgaccgtgctgggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 93 - DNA, 1362 bp, synthetic construct
  source 1..1362: mol_type = other DNA
atggagttcggcctgtcctggctgtttctggtggccatcctgaagggcgtgcagtgcgaggtgcagctgcagcagtctggaccagtgctggagaagccaggagccagcgtgaagatgtcctgtaaggcctctggctacaccttcacagactactatatgaactgggtgaagcagagccacggcaagtccctggagtggatcggcgtgatcaacccctacaatggcggcaccagccacaatgagaagctgaagggcaaggccaccctgacagtggacaagagctcctctacagcctatatggagctgaacagcctgacctccgaggattctgccgtgtactattgcgccaggggcaattactccgcctggtttgcctattggggccagggcacactggtgaccgtgtctgccagcggcggcggcggctctggaggaggaggcagcggcggaggaggctcccaggccgtggtgacacaggagtctgccctgaccacaagccctggcgagaccgtgacactgacctgtcggagctccaccggagcagtgaccacaagcaactatgccaattgggtgcaggagaagccagatcacctgttcacaggcctgatcggaggcaccaacaatagggcaccaggcgtgcctgcaagattttccggctctctgatcggcgacaaggccgccctgacaatcaccggagcacagaccgaggatgaggccatctacttctgcgtgctgtggtattccaacctgtgggtgtttggcggcggcacaaagctgaccgtgctgggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 94 - DNA, 2010 bp, synthetic construct
  source 1..2010: mol_type = other DNA
atggagttcggcctgtcctggctgtttctggtggccatcctgaagggcgtgcagtgcgaggtgcagctgcagcagtctggaccagtgctggagaagccaggagccagcgtgaagatgtcctgtaaggcctctggctacaccttcacagactactatatgaactgggtgaagcagagccacggcaagtccctggagtggatcggcgtgatcaacccctacaatggcggcaccagccacaatgagaagctgaagggcaaggccaccctgacagtggacaagagctcctctacagcctatatggagctgaacagcctgacctccgaggattctgccgtgtactattgcgccaggggcaattactccgcctggtttgcctattggggccagggcacactggtgaccgtgtctgccagcggcggcggcggctctggaggaggaggcagcggcggaggaggctcccaggccgtggtgacacaggagtctgccctgaccacaagccctggcgagaccgtgacactgacctgtcggagctccaccggagcagtgaccacaagcaactatgccaattgggtgcaggagaagccagatcacctgttcacaggcctgatcggaggcaccaacaatagggcaccaggcgtgcctgcaagattttccggctctctgatcggcgacaaggccgccctgacaatcaccggagcacagaccgaggatgaggccatctacttctgcgtgctgtggtattccaacctgtgggtgtttggcggcggcacaaagctgaccgtgctgggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 95 - DNA, 1461 bp, synthetic construct
  source 1..1461: mol_type = other DNA
atggagttcggcctgtcttggctgtttctggtggccatcctgaagggcgtgcagtgccagatcgtgctgacacagagccctgcaatcatgagcgcctccctgggagagagggtgaccatgacatgtaccgccagctcctctgtgagctcctcttacctgcactggtatcagcagaagcctggcagctccccaaagctgtggatctactctgccagcaacctggcctccggcgtgccccctaggttctccggctctggcagcggcacatcctactctctgaccatctctagcatggaggccgaggacgccgccacctactattgccaccagtatcaccgctccccaccctggacatttggcggaggcaccaagctggagatcaagagcggaggaggaggctctggaggaggaggcagcggaggaggaggctcccaggtgcacctgcagcagagcggaccagagctggtgaagcctggagcctccgtgaagatcagctgtaaggccagcggctacaacttcaatgattactacatcaactgggtgaagcagaggccaggacagggcctggagtggatcggatggatcttcccaggacggggcagaagcaattactccgagaagtttaagggcaaggccacactgaccgtggacaagtcctctagcacagcctatgtgctgctgtcctctctgacctctgaggatagcgccgtgtacttctgcgccaggggcaccgcacaggcaggatttccatattggggacagggcacactggtgaccgtgagcgccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 96 - DNA, 1362 bp, synthetic construct
  source 1..1362: mol_type = other DNA
atggagttcggcctgtcttggctgtttctggtggccatcctgaagggcgtgcagtgccagatcgtgctgacacagagccctgcaatcatgagcgcctccctgggagagagggtgaccatgacatgtaccgccagctcctctgtgagctcctcttacctgcactggtatcagcagaagcctggcagctccccaaagctgtggatctactctgccagcaacctggcctccggcgtgccccctaggttctccggctctggcagcggcacatcctactctctgaccatctctagcatggaggccgaggacgccgccacctactattgccaccagtatcaccgctccccaccctggacatttggcggaggcaccaagctggagatcaagagcggaggaggaggctctggaggaggaggcagcggaggaggaggctcccaggtgcacctgcagcagagcggaccagagctggtgaagcctggagcctccgtgaagatcagctgtaaggccagcggctacaacttcaatgattactacatcaactgggtgaagcagaggccaggacagggcctggagtggatcggatggatcttcccaggacggggcagaagcaattactccgagaagtttaagggcaaggccacactgaccgtggacaagtcctctagcacagcctatgtgctgctgtcctctctgacctctgaggatagcgccgtgtacttctgcgccaggggcaccgcacaggcaggatttccatattggggacagggcacactggtgaccgtgagcgccggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 97 - DNA, 2010 bp, synthetic construct
  source 1..2010: mol_type = other DNA
atggagttcggcctgtcttggctgtttctggtggccatcctgaagggcgtgcagtgccagatcgtgctgacacagagccctgcaatcatgagcgcctccctgggagagagggtgaccatgacatgtaccgccagctcctctgtgagctcctcttacctgcactggtatcagcagaagcctggcagctccccaaagctgtggatctactctgccagcaacctggcctccggcgtgccccctaggttctccggctctggcagcggcacatcctactctctgaccatctctagcatggaggccgaggacgccgccacctactattgccaccagtatcaccgctccccaccctggacatttggcggaggcaccaagctggagatcaagagcggaggaggaggctctggaggaggaggcagcggaggaggaggctcccaggtgcacctgcagcagagcggaccagagctggtgaagcctggagcctccgtgaagatcagctgtaaggccagcggctacaacttcaatgattactacatcaactgggtgaagcagaggccaggacagggcctggagtggatcggatggatcttcccaggacggggcagaagcaattactccgagaagtttaagggcaaggccacactgaccgtggacaagtcctctagcacagcctatgtgctgctgtcctctctgacctctgaggatagcgccgtgtacttctgcgccaggggcaccgcacaggcaggatttccatattggggacagggcacactggtgaccgtgagcgccggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 98 - DNA, 1461 bp, synthetic construct
  source 1..1461: mol_type = other DNA
atggagttcggcctgagctggctgtttctggtggccatcctgaagggcgtgcagtgccaggtgcacctgcagcagtccggaccagagctggtgaagcctggagccagcgtgaagatcagctgtaaggcctctggctacaacttcaatgactactatatcaactgggtgaagcagaggccaggacagggcctggagtggatcggatggatcttcccaggacggggccggagcaattacagcgagaagtttaagggcaaggccaccctgacagtggacaagagctcctctacagcctatgtgctgctgagctccctgacctccgaggattctgccgtgtacttctgcgcccggggcacagcacaggcaggctttccttattggggacagggcacactggtgaccgtgagcgcctccggaggaggaggcagcggaggaggcggctccggcggcggcggctctcagatcgtgctgacccagagcccagcaatcatgtctgccagcctgggagagagggtgaccatgacatgtaccgcctctagctccgtgtctagctcctacctgcactggtatcagcagaagcctggctctagcccaaagctgtggatctattccgcctctaacctggccagcggcgtgccccctaggttcagcggctccggctctggcacaagctactccctgaccatctcctctatggaggccgaggatgccgccacctactattgccaccagtatcaccgctccccaccctggacatttggcggaggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 99 - DNA, 1362 bp, synthetic construct
  source 1..1362: mol_type = other DNA
atggagttcggcctgagctggctgtttctggtggccatcctgaagggcgtgcagtgccaggtgcacctgcagcagtccggaccagagctggtgaagcctggagccagcgtgaagatcagctgtaaggcctctggctacaacttcaatgactactatatcaactgggtgaagcagaggccaggacagggcctggagtggatcggatggatcttcccaggacggggccggagcaattacagcgagaagtttaagggcaaggccaccctgacagtggacaagagctcctctacagcctatgtgctgctgagctccctgacctccgaggattctgccgtgtacttctgcgcccggggcacagcacaggcaggctttccttattggggacagggcacactggtgaccgtgagcgcctccggaggaggaggcagcggaggaggcggctccggcggcggcggctctcagatcgtgctgacccagagcccagcaatcatgtctgccagcctgggagagagggtgaccatgacatgtaccgcctctagctccgtgtctagctcctacctgcactggtatcagcagaagcctggctctagcccaaagctgtggatctattccgcctctaacctggccagcggcgtgccccctaggttcagcggctccggctctggcacaagctactccctgaccatctcctctatggaggccgaggatgccgccacctactattgccaccagtatcaccgctccccaccctggacatttggcggaggcaccaagctggagatcaagggatccgagagcaagtacggccccccctgccccccctgccccatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 100 - DNA, 2010 bp, synthetic construct
  source 1..2010: mol_type = other DNA
atggagttcggcctgagctggctgtttctggtggccatcctgaagggcgtgcagtgccaggtgcacctgcagcagtccggaccagagctggtgaagcctggagccagcgtgaagatcagctgtaaggcctctggctacaacttcaatgactactatatcaactgggtgaagcagaggccaggacagggcctggagtggatcggatggatcttcccaggacggggccggagcaattacagcgagaagtttaagggcaaggccaccctgacagtggacaagagctcctctacagcctatgtgctgctgagctccctgacctccgaggattctgccgtgtacttctgcgcccggggcacagcacaggcaggctttccttattggggacagggcacactggtgaccgtgagcgcctccggaggaggaggcagcggaggaggcggctccggcggcggcggctctcagatcgtgctgacccagagcccagcaatcatgtctgccagcctgggagagagggtgaccatgacatgtaccgcctctagctccgtgtctagctcctacctgcactggtatcagcagaagcctggctctagcccaaagctgtggatctattccgcctctaacctggccagcggcgtgccccctaggttcagcggctccggctctggcacaagctactccctgaccatctcctctatggaggccgaggatgccgccacctactattgccaccagtatcaccgctccccaccctggacatttggcggaggcaccaagctggagatcaagggatccgagagcaagtacggccccccctgccccccctgccccgccccccccgtggccggccccagcgtgttcctgttcccccccaagcccaaggacaccctgatgatcagccgcacccccgaggtgacctgcgtggtggtggacgtgagccaggaggaccccgaggtgcagttcaactggtacgtggacggcgtggaggtgcacaacgccaagaccaagccccgcgaggagcagttccagagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgcacaaccactacacccagaagagcctgagcctgagccccggcaagatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 101 - protein, 16 aa, synthetic construct
SGGGGSGGGGSGGGGS

SEQ ID NO: 102 - protein, 907 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 103 - protein, 501 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTES

SEQ ID NO: 104 - protein, 386 aa, synthetic construct
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 105 - protein, 497 aa, synthetic construct
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 106 - protein, 641 aa, synthetic construct
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 107 - protein, 316 aa, synthetic construct
GGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 108 - protein, 813 aa, synthetic construct
GGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQEMGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 109 - protein, 706 aa, synthetic construct
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAHVVLAIILYFIAFALGIFLVHKIVMFFL

SEQ ID NO: 110 - protein, 679 aa, synthetic construct
MQLLCVFCLVLLWEVGAASLSEVKLHLDIEGHASHYTIPWTELMAKVPGLSPEALWREANVTEDLASMLNRYKLIYKTSGTLGIALAEPVDIPAVSEGSMQVDASKVHPGVISGLNSPACMLSAPLEKQLFYYIGTMLPNTRPHSYVFYQLRCHLSYVALSINGDKFQYTGAMTSKFLMGTYKRVTEKGDEHVLSLVFGKTKDLPDLRGPFSYPSLTSAQSGDYSLVIVTTFVHYANFHNYFVPNLKDMFSRAVTMTAASYARYVLQKLVLLEMKGGCREPELDTETLTTMFEVSVAFFKVGHAVGETGNGCVDLRWLAKSFFELTVLKDIIGICYGATVKGMQSYGLERLAAMLMATVKMEELGHLTTEKQEYALRLATVGYPKAGVYSGLIGGATSVLLSAYNRHPLFQPLHTVMRETLFIGSHVVLRELRLNVTTQGPNLALYQLLSTALCSALEIGEVLRGLALGTESGLFSPCYLSLRFDLTRDKLLSMAPQEATLDQAAVSNAVDGFLGRLSLEREDRDAWHLPAYKCVDRLDKVLMIIPLINVTFIISSDREVRGSALYEASTTYLSSSLFLSPVIMNKCSQGAVAGEPRQIPKIQNFTRTQKSCIFCGFALLSYDEKEGLETTTYITSQEVQNSILSSNYFDFDNLHVHYLLLTTNGTVMEIAGLYEERAH

SEQ ID NO: 111 - protein, 137 aa, synthetic construct
MRAVGVFLAICLVTIFVLPTWGNWAYPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 112 - protein, 112 aa, synthetic construct
YPCCHVTQLRAQHLLALENISDIYLVSNQTCDGFSLASLNSPKNGSNQLVISRCANGLNVVSFFISILKRSSSALTGHLRELLTTLETLYGSFSVEDLFGANLNRYAWHRGG

SEQ ID NO: 113 - protein, 857 aa, synthetic construct
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 114 - protein, 1318 aa, synthetic construct
MEERGRETQMPVARYGGPFIMVRLFGQDGEANIQEERLYELLSDPRSALGLDPGPLIAENLLLVALRGTNNDPRPQRQERARELALVGILLGNGEQGEHLGTESALEASGNNYVYAYGPDWMARPSTWSAEIQQFLRLLGATYVLRVEMGRQFGFEVHRSRPSFRQFQAINHLVLFDNALRKYDSGQVAAGFQRALLVAGPETADTRPDLRKLNEWVFGGRAAGGRQLADELKIVSALRDTYSGHLVLQPTETLDTWKVLSRDTRTAHSLEHGFIHAAGTIQANCPQLFMRRQHPGLFPFVNAIASSLGWYYQTATGPGADARAAARRQQAFQTRAAAECHAKSGVPVVAGFYRTINATLKGGEGLQPTMFNGELGAIKHQALDTVRYDYGHYLIMLGPFQPWSGLTAPPCPYAESSWAQAAVQTALELFSALYPAPCISGYARPPGPSAVIEHLGSLVPKGGLLLFLSHLPDDVKDGLGEMGPARATGPGMQQFVSSYFLNPACSNVFITVRQRGEKINGRTVLQALGRACDMAGCQHYVLGSTVPLGGLNFVNDLASPVSTAEMMDDFSPFFTVEFPPIQEEGASSPVPLDVDESMDISPSYELPWLSLESCLTSILSHPTVGSKEHLVRHTDRVSGGRVAQQPGVGPLDLPLADYAFVAHSQVWTRPGGAPPLPYRTWDRMTEKLLVSAKPGGENVKVSGTVITLGEQGYKVSLDLREGTRLAMAEALLNAACAPILDPEDVLLTLHLHLDPRRADNSAVMEAMTAASDYARGLGVKLTFGSASCPETGSSASNFMTVVASVSAPGEFSGPLITPVLQKTGSLLIAVRCGDGKIQGGSLFEQLFSDVATTPRAPEALSLKNLFRAVQQLVKSGIVLSGHDISDGGLVTCLVEMALAGQRGVTITMPVASDYLPEMFAEHPGLVFEVEERSVGEVLQTLRSMNMYPAVLGRVGEQGPDQMFEVQHGPETVLRQSLRLLLGTWSSFASEQYECLRPDRINRSMHVSDYGYNEALAVSPLTGKNLSPRRLVTEPDPRCQVAVLCAPGTRGHESLLAAFTNAGCLCRRVFFREVRDNTFLDKYVGLAIGGVHGARDSALAGRATVALINRFPALRDAILKFLNRPDTFSVALGELGVQVLAGLGAVGSTDNPPAPGVEVNVQRSPLILAPNASGMFESRWLNISIPATTSSVMLRGLRGCVLPCWVQGSCLGLQFTNLGMPYVLQNAHQIACHFHSNGTDAWRFAMNYPRNPTEQGNIAGLCSRDGRHLALLCDPSLCTDFWQWEHIPPAFGHPTGCSPWTLMFQAAHLWSLRHGRPSE

SEQ ID NO: 115 - protein, 1448 aa, synthetic construct
MEERGRETQMPVARYGGPFIMVRLFGQDGEANIQEERLYELLSDPRSALGLDPGPLIAENLLLVALRGTNNDPRPQRQERARELALVGILLGNGEQGEHLGTESALEASGQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSASGNNYVYAYGPDWMARPSTWSAEIQQFLRLLGATYVLRVEMGRQFGFEVHRSRPSFRQFQAINHLVLFDNALRKYDSGQVAAGFQRALLVAGPETADTRPDLRKLNEWVFGGRAAGGRQLADELKIVSALRDTYSGHLVLQPTETLDTWKVLSRDTRTAHSLEHGFIHAAGTIQANCPQLFMRRQHPGLFPFVNAIASSLGWYYQTATGPGADARAAARRQQAFQTRAAAECHAKSGVPVVAGFYRTINATLKGGEGLQPTMFNGELGAIKHQALDTVRYDYGHYLIMLGPFQPWSGLTAPPCPYAESSWAQAAVQTALELFSALYPAPCISGYARPPGPSAVIEHLGSLVPKGGLLLFLSHLPDDVKDGLGEMGPARATGPGMQQFVSSYFLNPACSNVFITVRQRGEKINGRTVLQALGRACDMAGCQHYVLGSTVPLGGLNFVNDLASPVSTAEMMDDFSPFFTVEFPPIQEEGASSPVPLDVDESMDISPSYELPWLSLESCLTSILSHPTVGSKEHLVRHTDRVSGGRVAQQPGVGPLDLPLADYAFVAHSQVWTRPGGAPPLPYRTWDRMTEKLLVSAKPGGENVKVSGTVITLGEQGYKVSLDLREGTRLAMAEALLNAACAPILDPEDVLLTLHLHLDPRRADNSAVMEAMTAASDYARGLGVKLTFGSASCPETGSSASNFMTVVASVSAPGEFSGPLITPVLQKTGSLLIAVRCGDGKIQGGSLFEQLFSDVATTPRAPEALSLKNLFRAVQQLVKSGIVLSGHDISDGGLVTCLVEMALAGQRGVTITMPVASDYLPEMFAEHPGLVFEVEERSVGEVLQTLRSMNMYPAVLGRVGEQGPDQMFEVQHGPETVLRQSLRLLLGTWSSFASEQYECLRPDRINRSMHVSDYGYNEALAVSPLTGKNLSPRRLVTEPDPRCQVAVLCAPGTRGHESLLAAFTNAGCLCRRVFFREVRDNTFLDKYVGLAIGGVHGARDSALAGRATVALINRFPALRDAILKFLNRPDTFSVALGELGVQVLAGLGAVGSTDNPPAPGVEVNVQRSPLILAPNASGMFESRWLNISIPATTSSVMLRGLRGCVLPCWVQGSCLGLQFTNLGMPYVLQNAHQIACHFHSNGTDAWRFAMNYPRNPTEQGNIAGLCSRDGRHLALLCDPSLCTDFWQWEHIPPAFGHPTGCSPWTLMFQAAHLWSLRHGRPSE

SEQ ID NO: 116 - protein, 1444 aa, synthetic construct
MEERGRETQMPVARYGGPFIMVRLFGQDGEANIQEERLYELLSDPRSALGLDPGPLIAENLLLVALRGTNNDPRPQRQERARELALVGILLGNGEQGEHLGTESALEASGTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAASGNNYVYAYGPDWMARPSTWSAEIQQFLRLLGATYVLRVEMGRQFGFEVHRSRPSFRQFQAINHLVLFDNALRKYDSGQVAAGFQRALLVAGPETADTRPDLRKLNEWVFGGRAAGGRQLADELKIVSALRDTYSGHLVLQPTETLDTWKVLSRDTRTAHSLEHGFIHAAGTIQANCPQLFMRRQHPGLFPFVNAIASSLGWYYQTATGPGADARAAARRQQAFQTRAAAECHAKSGVPVVAGFYRTINATLKGGEGLQPTMFNGELGAIKHQALDTVRYDYGHYLIMLGPFQPWSGLTAPPCPYAESSWAQAAVQTALELFSALYPAPCISGYARPPGPSAVIEHLGSLVPKGGLLLFLSHLPDDVKDGLGEMGPARATGPGMQQFVSSYFLNPACSNVFITVRQRGEKINGRTVLQALGRACDMAGCQHYVLGSTVPLGGLNFVNDLASPVSTAEMMDDFSPFFTVEFPPIQEEGASSPVPLDVDESMDISPSYELPWLSLESCLTSILSHPTVGSKEHLVRHTDRVSGGRVAQQPGVGPLDLPLADYAFVAHSQVWTRPGGAPPLPYRTWDRMTEKLLVSAKPGGENVKVSGTVITLGEQGYKVSLDLREGTRLAMAEALLNAACAPILDPEDVLLTLHLHLDPRRADNSAVMEAMTAASDYARGLGVKLTFGSASCPETGSSASNFMTVVASVSAPGEFSGPLITPVLQKTGSLLIAVRCGDGKIQGGSLFEQLFSDVATTPRAPEALSLKNLFRAVQQLVKSGIVLSGHDISDGGLVTCLVEMALAGQRGVTITMPVASDYLPEMFAEHPGLVFEVEERSVGEVLQTLRSMNMYPAVLGRVGEQGPDQMFEVQHGPETVLRQSLRLLLGTWSSFASEQYECLRPDRINRSMHVSDYGYNEALAVSPLTGKNLSPRRLVTEPDPRCQVAVLCAPGTRGHESLLAAFTNAGCLCRRVFFREVRDNTFLDKYVGLAIGGVHGARDSALAGRATVALINRFPALRDAILKFLNRPDTFSVALGELGVQVLAGLGAVGSTDNPPAPGVEVNVQRSPLILAPNASGMFESRWLNISIPATTSSVMLRGLRGCVLPCWVQGSCLGLQFTNLGMPYVLQNAHQIACHFHSNGTDAWRFAMNYPRNPTEQGNIAGLCSRDGRHLALLCDPSLCTDFWQWEHIPPAFGHPTGCSPWTLMFQAAHLWSLRHGRPSE

SEQ ID NO: 117 - protein, 1428 aa, synthetic construct
MEERGRETQMPVARYGGPFIMVRLFGQDGEANIQEERLYELLSDPRSALGLDPGPLIAENLLLVALRGTNNDPRPQRQERARELALVGILLGNGEQGEHLGTESALEASGNNAPPNENPYHARRGIKEHVIQNAFRASGIMAPPVVRMFMRERQLPQSTGRKPASGPKFENIAEGLRALLARSHVERTTDEGTWVARGTALAIPQCRLTPLSRLPFGMAPGNYVYAYGPDWMARPSTWSAEIQQFLRLLGATYVLRVEMGRQFGFEVHRSRPSFRQFQAINHLVLFDNALRKYDSGQVAAGFQRALLVAGPETADTRPDLRKLNEWVFGGRAAGGRQLADELKIVSALRDTYSGHLVLQPTETLDTWKVLSRDTRTAHSLEHGFIHAAGTIQANCPQLFMRRQHPGLFPFVNAIASSLGWYYQTATGPGADARAAARRQQAFQTRAAAECHAKSGVPVVAGFYRTINATLKGGEGLQPTMFNGELGAIKHQALDTVRYDYGHYLIMLGPFQPWSGLTAPPCPYAESSWAQAAVQTALELFSALYPAPCISGYARPPGPSAVIEHLGSLVPKGGLLLFLSHLPDDVKDGLGEMGPARATGPGMQQFVSSYFLNPACSNVFITVRQRGEKINGRTVLQALGRACDMAGCQHYVLGSTVPLGGLNFVNDLASPVSTAEMMDDFSPFFTVEFPPIQEEGASSPVPLDVDESMDISPSYELPWLSLESCLTSILSHPTVGSKEHLVRHTDRVSGGRVAQQPGVGPLDLPLADYAFVAHSQVWTRPGGAPPLPYRTWDRMTEKLLVSAKPGGENVKVSGTVITLGEQGYKVSLDLREGTRLAMAEALLNAACAPILDPEDVLLTLHLHLDPRRADNSAVMEAMTAASDYARGLGVKLTFGSASCPETGSSASNFMTVVASVSAPGEFSGPLITPVLQKTGSLLIAVRCGDGKIQGGSLFEQLFSDVATTPRAPEALSLKNLFRAVQQLVKSGIVLSGHDISDGGLVTCLVEMALAGQRGVTITMPVASDYLPEMFAEHPGLVFEVEERSVGEVLQTLRSMNMYPAVLGRVGEQGPDQMFEVQHGPETVLRQSLRLLLGTWSSFASEQYECLRPDRINRSMHVSDYGYNEALAVSPLTGKNLSPRRLVTEPDPRCQVAVLCAPGTRGHESLLAAFTNAGCLCRRVFFREVRDNTFLDKYVGLAIGGVHGARDSALAGRATVALINRFPALRDAILKFLNRPDTFSVALGELGVQVLAGLGAVGSTDNPPAPGVEVNVQRSPLILAPNASGMFESRWLNISIPATTSSVMLRGLRGCVLPCWVQGSCLGLQFTNLGMPYVLQNAHQIACHFHSNGTDAWRFAMNYPRNPTEQGNIAGLCSRDGRHLALLCDPSLCTDFWQWEHIPPAFGHPTGCSPWTLMFQAAHLWSLRHGRPSE

SEQ ID NO: 118 - protein, 7 aa, synthetic construct
GLSLTSN

SEQ ID NO: 119 - protein, 5 aa, synthetic construct
WSNGG

SEQ ID NO: 120 - protein, 11 aa, synthetic construct
PRYNSGYFFDY

SEQ ID NO: 121 - protein, 11 aa, synthetic construct
KASESVSTRMH

SEQ ID NO: 122 - protein, 7 aa, synthetic construct
KTSNLAS

SEQ ID NO: 123 - protein, 9 aa, synthetic construct
QQSWNGPLT

SEQ ID NO: 124 - protein, 7 aa, synthetic construct
GFSLTSY

SEQ ID NO: 125 - protein, 5 aa, synthetic construct
WSDGD

SEQ ID NO: 126 - protein, 29 aa, synthetic construct
LQSEDTATYYCARLQVFGYPGIRDYVMDA

SEQ ID NO: 127 - protein, 17 aa, synthetic construct
KSSQSLLSSRHQKNFLA

SEQ ID NO: 128 - protein, 7 aa, synthetic construct
HASTRQS

SEQ ID NO: 129 - protein, 9 aa, synthetic construct
LQHYTSPYT

SEQ ID NO: 130 - protein, 119 aa, synthetic construct
QVQLKESGPGLVQPSQTLSLTCTVSGLSLTSNGVSWIRQPPGKGLEWLGVIWSNGGTDYNSAIKSRLSFSRDTSKSQVFLKMNSLQTEDTAMYFCARPRYNSGYFFDYWGQGVMVIVSS

SEQ ID NO: 131 - protein, 107 aa, synthetic construct
DTVLTQSPALAVSPGERVTISCKASESVSTRMHWYRQKPGQQPKLLIYKTSNLASGVPARFSGSGSGTDFTLTIDPVEADDTATYFCQQSWNGPLTFGSGTKLEIKR

SEQ ID NO: 132 - protein, 124 aa, synthetic construct
QVQLKESGPGLVQPSQTLSLTCTVSGFSLTSYHVHWVRQPPGKGLEWMGVMWSDGDTLYNSALKSRLSISRDTSKSQVLLQMDSLQSEDTATYYCARLQVFGYPGIRDYVMDAWGQGASVTVSS

SEQ ID NO: 133 - protein, 114 aa, synthetic construct
DLVMTQSPFSLAVSEGEMVTIKCKSSQSLLSSRHQKNFLAWYRQKPGQSPKLLIYHASTRQSGVPDRFIGSGSGTDFTLTISDVQAEDLADYYCLQHYTSPYTFGAGTKLELKR

SEQ ID NO: 134 - protein, 567 aa, synthetic construct
MEFGLSWLFLVAILKGVQCQVQLKESGPGLVQPSQTLSLTCTVSGLSLTSNGVSWIRQPPGKGLEWLGVIWSNGGTDYNSAIKSRLSFSRDTSKSQVFLKMNSLQTEDTAMYFCARPRYNSGYFFDYWGQGVMVTVSSSGGGGSGGGGSGGGGSDTVLTQSPALAVSPGERVTISCKASESVSTRMHWYRQKPGQQPKLLIYKTSNLASGVPARFSGSGSGTDFTLTIDPVEADDTATYFCQQSWNGPLTFGSGTKLEIKGDPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 578 aa, synthetic construct
MEFGLSWLFLVAILKGVQCQVQLKESGPGLVQPSQTLSLTCTVSGFSLTSYHVHWVRQPPGKGLEWMGVMWSDGDTLYNSALKSRLSISRDTSKSQVLLQMDSLQSEDTATYYCARLQVFGYPGIRDYVMDAWGQGASVTVSSSGGGGSGGGGSGGGGSDLVMTQSPFSLAVSEGEMVTIKCKSSQSLLSSRHQKNFLAWYRQKPGQSPKLLIYHASTRQSGVPDRFIGSGSGTDFTLTISDVQAEDLADYYCLQHYTSPYTFGAGTKLELKGDPAESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDPTTPFTCRPCPL

SEQ ID NO: 136 - protein, 685 aa, synthetic construct
MEFGLSWLFLVAILKGVQCQVQLKESGPGLVQPSQTLSLTCTVSGLSLTSNGVSWIRQPPGKGLEWLGVIWSNGGTDYNSAIKSRLSFSRDTSKSQVFLKMNSLQTEDTAMYFCARPRYNSGYFFDYWGQGVMVTVSSSGGGGSGGGGSGGGGSDTVLTQSPALAVSPGERVTISCKASESVSTRMHWYRQKPGQQPKLLIYKTSNLASGVPARFSGSGSGTDFTLTIDPVEADDTATYFCQQSWNGPLTFGSGTKLEIKGDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVISTSGRPWPGLVGSFSCHWLSPFTATTGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 674 aa, synthetic construct
MEFGLSWLFLVAILKGVQCQVQLKESGPGLVQPSQTLSLTCTVSGFSLTSYHVHWVRQPPGKGLEWMGVMWSDGDTLYNSALKSRLSISRDTSKSQVLLQMDSLQSEDTATYYCARLQVFGYPGIRDYVMDAWGQGASVTVSSSGGGGSGGGGSGGGGSDLVMTQSPFSLAVSEGEMVTIKCKSSQSLLSSRHQKNFLAWYRQKPGQSPKLLIYHASTRQSGVPDRFIGSGSGTDFTLTISDVQAEDLADYYCLQHYTSPYTFGAGTKLELKGDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVISTSGRPWPGLVGSFSCHWLSPFTATTGKRGRKKLLYIFKQPFMRPVQTTQEEDGCSCRFPEEEEGGCERVKFSRSADAPAYQQGQNQLYNELNLGRREEYDVLDKRRGRDPEMGGKPRRKNPQEGLYNELQKDKMAEAYSEIGMKGERRRGKGHDGLY

SEQ ID NO: 138 - protein, 123 aa, synthetic construct
QVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSS

SEQ ID NO: 139 - protein, 123 aa, synthetic construct
QVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSS

SEQ ID NO: 140 - protein, 123 aa, synthetic construct
QVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSS

SEQ ID NO: 141 - protein, 107 aa, synthetic construct
DIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIK

SEQ ID NO: 142 - protein, 107 aa, synthetic construct
DIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIK

SEQ ID NO: 143 - protein, 107 aa, synthetic construct
DIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIK

SEQ ID NO: 144 - protein, 490 aa, synthetic construct
MEFGLSWLFLVAILKGVQCDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 490 aa, synthetic construct
MEFGLSWLFLVAILKGVQCQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 490 aa, synthetic construct
MEFGLSWLFLVAILKGVQCDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 490 aa, synthetic construct
MEFGLSWLFLVAILKGVQCDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWFQQKPGKAPKALIYSASYRYSGVPSRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 490 aa, synthetic construct
MEFGLSWLFLVAILKGVQCQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFSGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 490 aa, synthetic construct
MEFGLSWLFLVAILKGVQCQVQLVQSGAEVKKPGASVKVSCKASGYSFTDYNMNWVRQAPGQGLEWIGVINPNFGTTSYNQKFKGRVTMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATMTVDTSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 490 aa, synthetic construct
MEFGLSWLFLVAILKGVQCDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKSGGGGSGGGGSGGGGSQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 490 aa, synthetic construct
MEFGLSWLFLVAILKGVQCQVQLVQSGAEVKKPGASVKVSCKASGYSFTDYNMNWVKQAPGQGLEWIGVINPNFGTTSYNQKFKGRATLTVDQSTSTVYMELSSLRSEDTAVYYCARSGYYGSNRYWYFDVWGQGTTVTVSSSGGGGSGGGGSGGGGSDIQMTQSPSSLSASVGDRVTITCKASQSVGTNVAWYQQKPGKAPKALIYSASYRYSGVPDRFTGSGSGTDFTLTISSLQPEDFATYFCQQYNSYPSTFGGGTKLEIKGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - DNA, 1470 bp, synthetic construct
  source 1..1470: mol_type = other DNA
atggagttcggcctgagctggctgtttctggtggccatcctgaagggcgtgcagtgcgacatccagatgacccagagccctagctcgctgtccgcctccgtaggtgatcgggtcaccatcacctgtaaggcgtcacagagtgtgggcaccaacgtggcgtggttccagcagaagccgggcaaggcacccaaggccctcatttactccgcctcctacaggtactccggggtgccatcccgcttcagcggctccggttctggcaccgactttactctgaccatctcttctctgcagccggaggacttcgccacctacttttgccaacagtacaacagctacccctctaccttcggcggaggcaccaagctggagatcaagtccggcggaggtggctctggaggcggtggatctggtggtggcggatcacaggtccagctagtgcagagcggcgcggaggtgaagaagcccggggcttcggtgaaagtttcatgcaaagcttccggctattccttcactgattacaacatgaattgggtccgccaggcccctggacagggcttggaatggatcggtgtcattaatcccaacttcggaaccacaagctacaaccagaaatttaagggccgagtgacaatgacggtagacacttcgacctccaccgtgtacatggagctgagttctcttcgctcggaggacaccgccgtgtattactgtgctcgctccggctactacggcagcaaccgctattggtacttcgacgtgtggggccagggaacgaccgtcaccgtgtcctctggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 163 - DNA, 1470 bp, synthetic construct
  source 1..1470: mol_type = other DNA
atggagttcggcctgagctggctgtttctggtggccatcctgaagggcgtgcagtgccaggtgcagctggtgcagagcggcgccgaggtgaagaagccgggagcttccgttaaggtgtcgtgcaaggcctctggctactccttcaccgactacaacatgaactgggtccgccaggcccctggacagggattggagtggattggtgtgattaatccaaatttcggtaccacaagctacaaccagaaatttaaaggtcgagtgactatgacagtcgatacttcgacctctaccgtgtacatggagctgtcctccctacgtagcgaagacaccgcggtttactactgtgcccggtcgggctattacggctccaaccgctattggtactttgacgtgtggggccagggcacgaccgtcaccgtatcgtcctctggcggaggtggctctggaggcggtggatctggtggtggcggatcagacatccagatgacccagagcccttctagtctttccgcttccgtgggagacagggtcactatcacctgcaaagcgtcacagagcgtgggcaccaacgtggcttggttccagcagaagcccggcaaggcacccaaggcgctcatctactcggctagctaccgctacagcggcgtcccctcccgcttctctggctcggggagtgggacggacttcaccctgaccatctcttctctgcagccggaggactttgccacctacttctgtcaacagtacaactcctatccctccaccttcggcggcggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 164 - DNA, 1470 bp, synthetic construct
  source 1..1470: mol_type = other DNA
atggagttcggcctgagctggctgtttctggtggccatcctgaagggcgtgcagtgcgacatccagatgacccagagcccttcttctctttccgcctccgtgggggacagggtcaccatcacctgcaaggcttctcagtcagtgggcaccaacgtggcgtggttccagcagaagcctggcaaggcccccaaggcgctgatttactctgcaagctaccgttacagtggcgtcccgtcccgcttttcaggctccggctcgggcaccgactttaccctgaccatctcttccctacagccagaggatttcgccacatatttctgtcaacagtacaactcctacccctctaccttcggcggtgggaccaagctggagatcaagagcggcggaggtggctctggaggcggtggatctggtggtggcggatcacaggtgcagttggtgcagagcggcgccgaggtgaagaagcccggggcttccgttaaggtgtcctgtaaagcatctggctactccttcacagattacaatatgaactgggtcaagcaggccccgggacagggcctcgaatggatcggtgtgattaatccaaacttcggcacgaccagctacaaccagaaatttaaaggtcgggctactatgactgttgacacttccacttcgaccgtctacatggagctgagtagcctgcgctcggaggacaccgcggtgtactactgcgcccgcagtggctattacggcagcaaccgctattggtacttcgacgtgtggggccagggaactacggtaaccgtgtcgtccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 165 - DNA, 1470 bp, synthetic construct
  source 1..1470: mol_type = other DNA
atggagttcggcctgagctggctgtttctggtggccatcctgaagggcgtgcagtgccaggtccagctggtgcagagtggcgccgaggtgaagaagcccggcgcttccgtaaaggtgtcatgcaaggcttctggctattctttcaccgactacaatatgaactgggtcaagcaggccccgggacagggtttggagtggatcggtgtgattaatcccaacttcggaactaccagctacaaccagaaatttaaaggccgggcgactatgacagtcgacacctcgacctccacagtgtacatggagctgagctcgctgcgctccgaggacacggccgtgtattactgtgctcgctcgggctactacggcagcaaccgctattggtacttcgacgtgtggggccagggcacgaccgtcaccgtgtcgagcagtggcggaggtggctctggaggcggtggatctggtggtggcggatcagacatccagatgacccagagcccttccagtctcagcgcatccgtgggtgatcgtgttaccatcacctgcaaagcctcccagagcgtgggcaccaacgtggcttggttccagcagaagcctggcaaggcccccaaggcgctaatctactcggcgagctaccgctactccggggtcccatccaggttttcaggatctggttctggcactgactttactcttactatttcttctctgcagccggaagatttcgccacctacttctgtcaacagtacaactcctacccctctaccttcggcggcggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 166 - DNA, 1470 bp, synthetic construct
  source 1..1470: mol_type = other DNA
atggagttcggcctgagctggctgtttctggtggccatcctgaagggcgtgcagtgcgacatccagatgacccagagcccttcctctctatccgcctccgtaggtgatagggttacaatcacctgcaaagcatcccagtccgtgggcacgaacgtggcttggttccagcagaagcccggcaaagctcccaaggcccttatctacagtgcgagctaccgctacagtggggtgccttcccgcttttcagggtctggctctggtactgacttcaccctgaccatctctagcctgcagccggaagatttcgccacctacttttgtcaacagtacaactcctatccctctaccttcggcggcggcactaagctggagatcaagtctggcggaggtggctctggaggcggtggatctggtggtggcggatcacaggtgcagctggtgcagagcggcgcggaggtgaagaagccgggtgcttccgtgaaggtgtcgtgtaaggcgtccggatactccttcaccgactacaacatgaactgggtcaagcaggcccccggacagggcctcgagtggattggcgtcattaatccaaatttcggcacgaccagctacaaccagaaatttaagggccgcgccactctgactgtcgaccagagcacctcgaccgtgtacatggagctgtcctcattgcggtcggaggacaccgccgtgtattactgcgcccgtagtggctactacggcagcaaccgctattggtacttcgacgtgtggggccagggcaccacagtcaccgtgtcctccggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 167 - DNA, 1470 bp, synthetic construct
  source 1..1470: mol_type = other DNA
atggagttcggcctgagctggctgtttctggtggccatcctgaagggcgtgcagtgccaggtgcagctggtgcagagcggcgccgaggtgaagaagcctggcgcttctgtaaaggtgtcatgcaaggcttccggctactccttcaccgactacaacatgaactgggtcaagcaggcccccggacagggactcgagtggatcggtgtgattaatccaaatttcgggaccacaagctacaaccagaaatttaaaggccgcgccactcttacagttgaccagtcgacctccaccgtgtacatggagctgagctccctgcgctcggaggacaccgccgtgtattactgtgctcgttctgggtactacggcagcaaccgctattggtacttcgacgtgtggggccagggcacgacggtcactgtctcgtccagtggcggaggtggctctggaggcggtggatctggtggtggcggatcagacatccagatgactcagagcccttccagtttgtcggcgtccgtgggtgatagggtcaccatcacctgcaaagcatcccagagcgtgggcaccaacgtggcgtggttccagcagaagcccggcaaggccccgaaggcgctcatctactccgcctcctaccggtactctggtgtcccctctcgcttctcagggtcgggctccggtactgatttcaccctgaccatttcttctctgcagccggaagactttgccacctatttttgtcaacagtacaactcctacccctctaccttcggcggcggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 168 - DNA, 1470 bp, synthetic construct
  source 1..1470: mol_type = other DNA
atggagttcggcctgagctggctgtttctggtggccatcctgaagggcgtgcagtgcgacatccagatgacccagagcccttcctctctgagcgcatccgtaggtgatcgagttaccatcacctgcaaggcctctcagtctgtggggaccaacgtcgcctggtaccagcagaagcctggcaaggcgcccaaggcgcttatctacagtgcttcttaccgctacagcggcgtccccgaccgcttttcggggtccggctcgggcactgatttcaccctgacgatttcctccttgcagccggaggacttcgccacttatttctgtcaacagtacaacagctacccctctaccttcggcggaggcaccaagctggagatcaagagcggcggaggtggctctggaggcggtggatctggtggtggcggatcacaggtgcagctggtgcagtccggggctgaggtgaagaagcccggggcgtccgtgaaagtttcatgcaaagcctcgggctactccttcaccgactacaacatgaactgggtgcgccaggccccgggacaggggctagaatggatcggtgtgattaatccaaattttggtactacaagctacaaccagaaatttaagggccgggtcactatgactgtcgacaccagcacctccaccgtgtacatggagctgtcgagtctcaggtccgaggacacagccgtgtactactgtgctcgttctggttattacggcagcaaccgctattggtacttcgacgtgtggggccagggcacgaccgtcaccgtgtcgtctggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 169 - DNA, 1470 bp, synthetic construct
  source 1..1470: mol_type = other DNA
atggagttcggcctgagctggctgtttctggtggccatcctgaagggcgtgcagtgccaggtgcagctagtgcagagtggcgccgaggtgaagaagcccggggcatcggttaaggtgtcatgcaaagcgagcggatactccttcaccgactacaacatgaattgggtccgacaggcccctggacagggacttgagtggatcggtgtcattaatccaaactttggcaccacatcatacaaccagaaatttaagggccgtgttacgatgaccgtcgacacgtcgacctccaccgtgtacatggagctgagctctctgcgctcggaagacaccgccgtgtattactgtgctcgcagcggctactacggcagcaaccgctattggtacttcgacgtgtggggccagggcactacagtcaccgtgtcctcttctggcggaggtggctctggaggcggtggatctggtggtggcggatcagacatccagatgacccagagcccttccagcttgtccgcctccgtaggtgatcgggtgaccatcacctgcaaagcctcccagtccgtgggcaccaacgtggcgtggtaccagcagaagcccggcaaggccccgaaggcgctcatttactctgctagctacaggtacagtggtgtccccgaccgcttctctggctccggctccgggactgatttcaccctgactatctcttccctgcagccggaggactttgctacttatttctgtcaacagtacaactcctacccctctaccttcggcggcgggaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 170 - DNA, 1470 bp, synthetic construct
  source 1..1470: mol_type = other DNA
atggagttcggcctgtcctggctgtttctggtggccatcctgaagggcgtgcagtgcgatatacaaatgactcaatcccccagttcccttagcgccagcgtgggagatcgggtgactattacgtgcaaagcttcacaatctgtaggcactaacgtcgcctggtatcaacagaagccaggaaaggcgcccaaggcactgatttactccgcgtcctacaggtattccggagtcccagaccggttcagtggcagtggctccggaaccgactttactctgacgatcagcagccttcaaccagaggattttgcaacctacttttgccaacagtacaatagttatccatctacgttcggtggcggcacgaaactcgaaattaaatccggtggtggcggcagcggtgggggggggtccggcggtggtggatcacaagtgcaacttgtacaaagcggagctgaagttaagaagcccggggccagtgtcaaggtctcatgtaaggccagcggctattcttttactgactataatatgaactgggtgaaacaggcgcctggccaaggcctcgagtggattggagtaataaatccgaattttggtactacaagttataaccagaagtttaagggccgcgccactatgactgttgatacttccacgagtacggtgtacatggagcttagttcacttaggtccgaggatacggccgtctattattgtgccagatcaggatactatggatctaaccggtactggtattttgacgtatggggccaaggaactaccgttaccgtgtcatcaggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 171 - DNA, 1470 bp, synthetic construct
  source 1..1470: mol_type = other DNA
atggagttcggcctgagctggctgtttctggtggccatcctgaagggcgtgcagtgccaggtccagctggtgcagagcggtgccgaggtgaagaagcctggagcgtcggtaaaggtgtcatgcaaagcgtccggctactccttcaccgactacaacatgaactgggtcaagcaggccccgggacaggggctagagtggattggcgtgattaatccaaatttcggtaccacatcttacaaccagaaatttaaaggccgtgcaactatgactgttgatacctccacgagcactgtctacatggagctgtcttctttgcgctcggaggacaccgccgtgtactactgtgcccgcagcggctattacggcagcaaccgctattggtacttcgacgtgtggggccagggcacgactgtcaccgtgtcgtcctctggcggaggtggctctggaggcggtggatctggtggtggcggatcagacatccagatgacccagagccctagcagcctttccgcttctgtgggggatcgggtgactatcacctgcaaggcttcccagagtgtgggcaccaacgtggcttggtaccagcagaagcccggcaaggctcccaaggcgctcatctatagtgccagctaccgctactccggcgttcccgacaggttttcagggtcgggctccggcacagacttcaccctgaccatctcgtccctgcagccggaagatttcgccacctacttttgtcaacagtacaactcctatccctctaccttcggcggcgggaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 172 - DNA, 1470 bp, synthetic construct
  source 1..1470: mol_type = other DNA
atggagttcggcctgtcctggctgtttctggtggccatcctgaagggcgtgcagtgcgatatccagatgacccaatcacctagcagcttgtctgcttcagtaggcgatcgggtgaccattacgtgtaaagcctcacaaagtgtagggacaaacgttgcctggtatcaacaaaagcccggcaaagcacctaaggcactcatctatagtgcttcttatcgatattccggagtgcctgataggttttccgggagcggctccggaacagatttcacactcaccatttcctctctgcaacctgaggactttgctacatatttctgccaacagtataattcatatccatcaacattcggggggggcacgaagctggaaattaaatcaggggggggcggtagtggtggaggaggcagtggtggtggcgggagtcaagtacaactcgtgcaaagcggcgctgaggtcaagaaacccggagcttctgtaaaagtatcatgcaaagcttccggctattcctttaccgattataatatgaactgggttaaacaagctcctggccaaggccttgagtggatcggtgtcataaatcctaattttggcacaacgagttataaccaaaagttcaaaggaagagcgactttgacggtcgatcaatccacaagcacagtatatatggagttgagcagccttcggagcgaggacactgcagtctattattgtgcgcggagcgggtattatgggtctaaccgttactggtactttgacgtgtggggtcaaggaacaactgtcactgtatcatcaggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 173 - DNA, 1470 bp, synthetic construct
  source 1..1470: mol_type = other DNA
atggagttcggcctgagctggctgtttctggtggccatcctgaagggcgtgcagtgccaggtccagctggtgcagagcggcgccgaggtgaagaagcccggcgcttccgtgaaggtgtcatgcaaggcttccggctattccttcacagattataacatgaattgggttaagcaggccccgggacagggcctcgagtggattggggtgattaatccaaacttcggtacaactagctacaaccagaaatttaagggccgggccactctgactgtggaccagagcacctccacggtgtacatggagctgagcagtctacgtagcgaggacaccgcagtctattactgtgcccgctcgggctactacggcagcaaccgctattggtacttcgacgtgtggggccagggaacgaccgtcaccgtatctagttccggcggaggtggctctggaggcggtggatctggtggtggcggatcagacatccagatgacccagtccccctcttctctttccgcgtctgttggggacagggtgactatcacctgcaaagctagccagagcgtgggcaccaacgtggcgtggtaccagcagaagcctgggaaggcgcctaaagccctgatctactccgcttcctaccgctactctggcgtccccgaccgcttttcaggttctggctccggcaccgacttcaccctgaccatctcgtcgttgcagccggaagatttcgccacctacttttgtcaacagtacaactcgtacccctccaccttcggcggaggcaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 174 - DNA, 1470 bp, synthetic construct
  source 1..1470: mol_type = other DNA
atggagttcggcctgagctggctgtttctggtggccatcctgaagggcgtgcagtgcgacatccagatgacccagagcccaagctctctttcggcctccgtgggggatagggttacaatcacctgcaaggcctcacagagcgtcggcaccaacgtggcgtggtaccagcagaagccgggaaaagctcctaaggcgctcatttactccgctagctaccgttactctggcgtccccgaccgctttaccggctctgggtctggtactgatttcaccctgaccatcagttctctgcagccggaagactttgccacctatttctgtcaacagtacaacagctacccttccaccttcggcggcggtactaagctggagatcaaatccggcggaggtggctctggaggcggtggatctggtggtggcggatcacaggtccagctggtgcagagtggcgcggaggtgaagaagcccggcgcttctgtgaaggtgtcatgcaaggcatccggctactccttcaccgactacaacatgaattgggtccgccaggcccccggacagggattggagtggatcggtgtgattaatcccaacttcggcacgaccagctacaaccagaaatttaagggccgagtgacgatgactgttgatacctccacttcgaccgtgtacatggagctgagctccctacggtcggaggacaccgccgtgtactactgtgctcgctcgggctattacggcagcaaccgctattggtacttcgacgtgtggggccaggggaccacagtcactgtatcttcgggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 175 - DNA, 1470 bp, synthetic construct
  source 1..1470: mol_type = other DNA
atggagttcggcctgagctggctgtttctggtggccatcctgaagggcgtgcagtgccaggtccagctggtgcagagcggggccgaggtgaagaagcccggcgcgtccgtaaaggtgtcatgcaaagcttctggctactccttcaccgactacaacatgaactgggtgcgccaggcccctggacagggcctagagtggatcggtgtgattaatccaaatttcggcacgaccagctacaaccagaaatttaagggccgggtcactatgactgttgacacctccacatcgaccgtgtacatggagctgtcctctcttcgctccgaggacacggccgtgtattactgtgctcgctcggggtactacggcagcaaccgctattggtacttcgacgtgtggggccagggaactacagtcactgttagctcgtctggcggaggtggctctggaggcggtggatctggtggtggcggatcagacatccagatgacccagagcccctcttctttgtccgcctccgtgggtgatcgagtgaccatcacctgcaaagcatcccagtcagtgggcaccaacgtggcgtggtaccagcagaagcctggcaaggcgcccaaggccctcatctactccgctagctacaggtacagtggtgtccccgaccgcttcaccggctcgggctccggtactgacttcaccctgaccatttcttctctgcagccggaagatttcgccacctacttttgtcaacagtacaacagctatccgtccaccttcggcggagggaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 176 - DNA, 1470 bp, synthetic construct
  source 1..1470: mol_type = other DNA
atggagttcggcctgtcctggctgtttctggtggccatcctgaagggcgtgcagtgcgatattcaaatgacacaaagtccttccagtttgtccgcatctgtcggtgacagagtgacaattacttgcaaagcgtctcaatctgttgggacaaacgtcgcatggtatcaacaaaagccaggtaaggcacccaaagcactgatatattccgcaagctaccgttattccggggtgccagaccggtttaccggctctggtagtggaaccgacttcacccttactatctcttctctgcaacccgaggatttcgccacctacttttgccaacaatataattcatacccaagcactttcggcggtgggacgaaattagagataaagtctggcggcggcgggtctggcggaggaggttcaggggggggtggaagtcaagtgcaattggtgcagagcggtgcagaagtaaagaaacctggtgcttcagtcaaggtcagctgtaaagcctcaggatactcttttacggactataatatgaattgggtaaaacaagctccaggtcaggggctcgaatggattggtgtgataaatcccaactttgggactacgagttacaaccagaagttcaagggaagggccacgatgactgtggatacgtctacaagtactgtatacatggagctgagctcactccgcagcgaagatacggccgtgtattactgtgccagaagcggctactatggatctaaccgatactggtatttcgatgtctggggtcagggtactactgttaccgtcagctcaggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 177 - DNA, 1470 bp, synthetic construct
  source 1..1470: mol_type = other DNA
atggagttcggcctgagctggctgtttctggtggccatcctgaagggcgtgcagtgccaggtgcagctggtgcagagcggcgccgaggtgaagaagcctggggcttccgtaaaggtgtcatgcaaagctagcggctactccttcactgactacaacatgaactgggtcaagcaggccccgggacagggtttggagtggattggggtgattaatccaaattttggtacgaccagctacaaccagaaatttaagggccgtgccactatgactgtcgatacatcgacctccaccgtctacatggagctgtcctctctgcgctcggaagacaccgccgtgtactactgtgctcgctcaggctattacggcagcaaccgctattggtacttcgacgtgtggggccagggcaccacagtcaccgtgtcgagctccggcggaggtggctctggaggcggtggatctggtggtggcggatcagacatccagatgacccagtccccttcttctctttccgcctccgtgggtgatcgggtgaccatcacctgcaaggccagtcagtctgtgggcaccaacgtggcgtggtaccagcagaagcccggcaaagcacccaaggcgctaatctacagtgccagctacaggtactcgggcgttcccgaccgcttcaccggctccggatctggtacggacttcactctgaccatctcgtccctccagccggaggactttgcgacttatttctgtcaacagtacaacagctatccctctaccttcggcggggggaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 178 - DNA, 1470 bp, synthetic construct
  source 1..1470: mol_type = other DNA
atggagttcggcctgagctggctgtttctggtggccatcctgaagggcgtgcagtgcgacatccagatgacccagagcccgagctccctttcggcgtccgtgggcgatcgagtgaccatcacctgcaaagccagccagagtgtcggcaccaacgtggcttggtaccagcagaagcccggcaaggctcccaaggcgctcatctatagtgcgagttaccgctactctggggtgcccgaccgcttcaccgggtccggctccggcaccgacttcacattgactatttcttctctgcagccggaagatttcgccacctacttttgtcaacagtacaacagctatccctctaccttcggcggcgggaccaagctggagatcaagtccggcggaggtggctctggaggcggtggatctggtggtggcggatcacaggtccagctggtgcagtccggggcagaggtgaagaagcctggggcttccgttaaggtgtcatgcaaggcctcgggctactccttcaccgattacaacatgaactgggtcaagcaggcccctggacagggtctcgagtggatcggtgtgattaatccaaatttcggtactactagctacaaccagaaatttaaaggccgtgccactctgaccgttgaccagagcacctccaccgtctacatggagctgtcatcgctgcgctcggaggacaccgccgtgtactactgtgctcggtctggctattacggcagcaaccgctattggtacttcgacgtgtggggccagggcacgacggtaacagtgtccagcggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga

SEQ ID NO: 179 - DNA, 1470 bp, synthetic construct
  source 1..1470: mol_type = other DNA
atggagttcggcctgagctggctgtttctggtggccatcctgaagggcgtgcagtgccaggtgcagctggtgcagagcggggccgaggtgaagaagcctggcgcgtccgttaaggtgtcatgcaaagcatctggctactccttcaccgactacaatatgaactgggtcaagcaggccccgggtcagggtttggagtggattggtgtgattaatcccaacttcggaactaccagctacaaccagaaatttaaaggaagggctaccctgaccgtggaccagtcgacctcgaccgtctacatggaactgtctagcctgcgctcggaggataccgccgtgtactactgtgcccgttccggctattacggcagcaaccgctattggtacttcgacgtgtggggccagggcacgactgtcaccgtgtcctcgtccggcggaggtggctctggaggcggtggatctggtggtggcggatcagacatccagatgacccagagcccttcctctctttccgcttccgtgggtgatcgcgtaactatcacctgcaaggcttcccagagcgttgggaccaacgtggcgtggtaccagcagaagcccggcaaggcccccaaggcgctcatctactcggctagctaccggtacagtggcgtccccgaccgctttaccggctccggctccggcacagatttcacactaactatctcttctctgcagccggaggactttgccacgtatttctgtcaacagtacaactcctatccaagtaccttcggcggcggtaccaagctggagatcaagggatccaccacaacaccggcgccacgcccaccaacaccagcacctaccatcgcaagccagccactgtccctgcggcccgaggcctgtaggccagcagcaggaggagcagtgcacaccaggggcctggactttgcctgcgacatctacatctgggcaccactggcaggaacatgtggcgtgctgctgctgtccctggtcatcaccctgtattgcaagcgcggccggaagaagctgctgtacatcttcaagcagcccttcatgcggcccgtgcagacaacccaggaggaggacggctgcagctgtaggttccctgaagaggaggagggaggatgtgagctgagggtgaagttttcccggtctgccgatgccccagcctataagcagggccagaatcagctgtacaacgagctgaatctgggccggagagaggagtatgacgtgctggataagaggaggggaagagatcccgagatgggaggcaagcccagaagaaagaaccctcaggagggcctgtataatgagctgcagaaggacaagatggccgaggcctacagcgagatcggcatgaagggagagaggcgccggggcaagggacacgatggcctgtaccagggcctgtcaacagcaactaaagatacttacgacgcactgcacatgcaggctctgccaccccga